(19) United States
(12) Patent Application Publication
(10) Pub. No.: US 2014/0088298 A9
(43) Pub. Date: Mar. 27, 2014

(54) CONJUGATES COMPRISING HYDROXYALKYL STARCH AND A CYTOTOXIC AGENT AND PROCESS FOR THEIR PREPARATION

(71) Applicants: Helmut Knoller, Friedberg (DE); Dominik Heckmann, Friedberg (DE); Frank Hacket, Altenstadt (DE); Norbert Zander, Bad Nauheim (DE); Frank Nocken, Frankfurt am Main (DE); Saswata Lahiri, Gurgaon (IN); Nitin Gupta, Gurgaon (IN); Sunil Sanghani, Gurgaon (IN); Azim Abul, Gurgaon (IN); Hemant Kumar Singh, Gurgaon (IN); Sandeep Grewal, Gurgaon (IN); Sandeep Kaur, Gurgaon (IN)

(72) Inventors: Helmut Knoller, Friedberg (DE); Dominik Heckmann, Friedberg (DE); Frank Hacket, Altenstadt (DE); Norbert Zander, Bad Nauheim (DE); Frank Nocken, Frankfurt am Main (DE); Saswata Lahiri, Gurgaon (IN); Nitin Gupta, Gurgaon (IN); Sunil Sanghani, Gurgaon (IN); Azim Abul, Gurgaon (IN); Hemant Kumar Singh, Gurgaon (IN); Sandeep Grewal, Gurgaon (IN); Sandeep Kaur, Gurgaon (IN)

(73) Assignee: Fresenius Kabi Deutschland GmbH, Bad Homburg (DE)

(21) Appl. No.: 13/809,194

(22) PCT Filed: Jul. 11, 2011
(86) PCT No.: PCT/EP2011/003459

§ 371 Date: Mar. 28, 2013

Related U.S. Application Data
(60) Provisional application No. 61/363,112, filed on Jul. 9, 2010.

(30) Foreign Application Priority Data
Jul. 9, 2010 (EP) 10007108.3

(51) Int. Cl. A61K 47/36

(52) U.S. Cl. CPC A61K 47/36
USPC 536/18.1

(57) ABSTRACT

The present invention relates to a hydroxyalkyl starch conjugate and a method for preparing the same, said hydroxy-yalkyl starch conjugate comprising a hydroxyalkyl starch derivative and a cytotoxic agent, the cytotoxic agent comprising at least one secondary hydroxyl group, wherein the hydroxyalkyl starch is linked via said secondary hydroxyl group to the cytotoxic agent. The conjugate according to the present invention has a structure according to the following formula HAS′(-L-M)ₙ wherein M is a residue of the cytotoxic agent, L is a linking moiety, HAS′ is the residue of the hydroxyalkyl starch derivative, and n is greater than or equal to 1, and wherein the hydroxyalkyl starch derivative has a mean molecular weight (MW) above the renal threshold.

The present invention relates to hydroxyalkyl starch conjugates comprising a hydroxyalkyl starch derivative and a cytotoxic agent, the cytotoxic agent comprising at least one secondary hydroxyl group, wherein the hydroxyalkyl starch is linked via said secondary hydroxyl group to the cytotoxic agent. The conjugates according to the present invention have a structure according to the following formula

HAS′(-L-M)ₙ

wherein M is a residue of the cytotoxic agent, L is a linking moiety, HAS′ is the residue of the hydroxyalkyl starch derivative, and n is greater than or equal to 1, and wherein the hydroxyalkyl starch derivative has a mean molecular weight (MW) above the renal threshold, preferably in the range of from 60 to 800 kDa, more preferably of from 80 to 800 kDa, and a molar substitution (MS) in the range of from 0.6 to 1.5. Moreover, besides the conjugate, the invention relates to the method for preparing said conjugate and conjugates obtained or obtainable by said method. Further, the invention relates to hydroxyalkyl starch derivatives for the preparation of the hydroxyalkyl starch conjugates and a method for the preparation of these derivatives. Further, the invention relates to the HAS cytotoxic agent conjugates for the treatment of cancer as well as to pharmaceutical compositions comprising these conjugates for the treatment of cancer.

Hydroxyalkyl starch (HAS), in particular hydroxyethyl starch (HES), is a substituted derivative of the naturally occurring carbohydrate polymer amylopectin, which is present in corn starch at a concentration of up to 95% by weight, and is degraded by other amylases in the body. HES in particular exhibits advantageous biological properties and is used as a blood volume replacement agent and in hemodilution therapy in clinics (Sommermeyer et al., 1987, Krankenhauspharmazie, 8(8): 271-278; Weidler et al., 1991, Arzneimittelforschung/Drug Research, 41: 494-498).

Cytotoxic agents are natural or synthetic substances which decrease the cell growth. A major drawback of many cytotoxic agents is their extreme low water solubility which renders the in vivo administration of the agent extremely complicated. Thus, this poor water solubility usually has to be overcome by complex formulation techniques including various excipients, wherein these excipients usually also show toxic side effects. As an example, the emulsifier Cremophor EL and ethanol, which are used to formulate taxol-based agents in order to deliver the required dosis of these taxol-based agents in vivo, shows toxic effects such as vasodilation, dispnea, and hypotension. In particular, Cremophor EL has also been shown to cause severe anaphylactoid hypersensitivity reactions, hyperlipidaemia, abnormal lipoprotein patterns, aggregation of erythrocytes and peripheral neuropathy (“Cremophor EL: the drawbacks and advantages of vehicle selection for drug formulation”, European Journal of Cancer”, Volume 31, Issue 13, Pages 1590-1598). In fact, the maximum dose of, for example paclitaxel, a taxol-based cytotoxic agent that can be administered to mice by injection, is dictated by the acute lethal toxicity of said Cremophor EL vehicle.

This is one reason why the potential use of soluble prodrugs, in particular macromolecular prodrugs, as a means of administering biologically effective cytotoxic agents to mammals has been proposed. Such prodrugs include chemical derivatives of the cytotoxic agents which, upon administration, will eventually liberate the active parent compound in vivo. The use of such prodrugs allows the artisan to modify the onset and/or duration of action in vivo. In addition, the use of prodrugs was proposed to enhance the water solubility of the drug, to provide an advantageous targeting and/or an enhancement of the stability of the therapeutic agent. Further, such prodrugs were suggested to prolong the circulation lifetime, to provide an extended duration of activity, or to achieve a reduction of side effects and drug toxicity. A typical example in the preparation of prodrugs involves the conversion of alcohols or thioalcohols to either organic phosphates or esters (Remington's Pharmaceutical Science, 16^{th} ed., A. Ozols (ed.), 1980). Numerous reviews have described the potential application of macromolecules as high molecular weight carriers for cytotoxic agents yielding in polymeric prodrugs of said agents. It was proposed that by coupling the cytotoxic agents to polymers, it is possible to increase the molecular weight and size of the prodrugs so that the weight and size of the prodrugs are too high to be quickly removed by glomerular filtration in the kidney and that, as consequence, the plasma residence time can be drastically increased.

Most modifications to date have been carried out with polyethylene glycol or similar polymers with polyethylene glycol (PEG) being generally preferred as polymer because of its easy availability and the possibility to give defined products upon reaction of limited available functional groups for coupling to a cytotoxic agent being present in PEG.

For example, WO 93/24476 discloses conjugates between taxane-based drugs, such as paclitaxel, to polyethylene glycol as macromolecule. In these conjugates, paclitaxel is linked to the polyethylene glycol using an ester linkage.

Similarly, U.S. Pat. No. 5,977,163 describes the conjugation of taxane-based drugs, such as paclitaxel or docetaxel, to similar water soluble polymers such as polyglutamic acid or polyaspartic acid.

Likewise, polyethylene glycol conjugates with cytotoxic agents, such as camptothecins, are disclosed in WO 98/07713. According to WO 98/07713, the polymer is linked via a linker to a hydroxyl function of the cytotoxic agent providing an ester linkage which allows for a rapid hydrolysis of the polymer drug linkage in vivo to generate the parent drug. This is achieved by using a linker comprising an electron-withdrawing group in close proximity to the ester bond. No polysaccharide-based conjugates were disclosed in WO 98/07713.

U.S. Pat. No. 6,395,266 B1 discloses branched PEG polymers linked to various cytotoxic agents. The branched polymers are considered to be advantageous compared to linear PEG conjugates since a higher loading of parent drug per unit of polymer can be achieved. The actual activity of these conjugates in vivo for the treatment of cancer was, however, not shown.

Similar to U.S. Pat. No. 6,395,266 B1, EP 1 496 076 A1 discloses Y-shaped branched hydrophilic polymer derivatives conjugated to cytotoxic agents such as camptothecin. Again, the actual activity of these conjugates in vivo was not shown.

In a similar way, the following patent and non-patent literature discloses PEG conjugates: Greenwald et al., J. Med. Chem., 1996, 39: 424-431 and U.S. Pat. No. 5,840,900.

PEG, however, is known to have unpleasant or hazardous side effects such as induction of antibodies against PEG (N. J. Ganson, S. J. Kelly et al., Arthritis Research & Therapie 2006, 8:R12) and nephrotoxicity (G. A. Laine, S. M. Hamid Hossain et al., The Annals of Pharmacotherapy, 1995 November, Volume 29) on use of such PEG or PEG-related conjugates. In addition, the biological activity of the active ingredients is most often greatly reduced in some cases after the PEG coupling. Moreover, the metabolism of the degradation products of PEG conjugates is still substantially unknown and possibly represents a health risk. Further, the functional groups available for coupling to cytotoxic agents are limited, so a high loading of the polymer with the respective drug is not possible.

Thus there is still a need for physiologically well tolerated alternatives to such PEG conjugates with which the solubility of poorly soluble low molecular weight substances can be improved and/or the residence time of low molecular weight substances in the plasma can be increased and/or with which an optimized drug loading can be achieved. Further there is the need for macromolecular prodrugs which provide an advantageous targeting of the tumor and/or which, upon administration, will eventually liberate the active parent compound in vivo with improved pharmacodynamic properties.

It would be particularly desirable to provide prodrugs which take advantage of the so-called Enhanced Permeability and Retention (EPR) effect. This EPR effect describes the property by which certain sizes of molecules, such as macromolecules or liposomes, tend to accumulate in tumor tissue much more than they do in normal tissue (reference is made to respective passages of U.S. Pat. No. 6,624,142 B2; or to Vasey P. A., Kaye S. B., Morrison R, et al. (January 1999) “Phase I clinical and pharmacokinetic study of PK1 [N-(2-hydroxypropyl)methacrylamide copolymer doxorubicin]: first member of a new class of chemotherapeutic agents-drug-polymer conjugates. Cancer Research Campaign Phase I/II Committee”. Clinical Cancer Research 5 (1): 83-94). The general explanation for that effect is that tumor vessels are usually abnormal in form and architecture. This is due to the fact that, in order for tumor cells to grow quickly, they must stimulate the production of blood vessels.

Without wanting to be bound to any hypothesis, it is believed that the EPR effect allows for an enhanced or even substantially selective delivery of macromolecules to the tumor cells and as consequence, enrichment of the macromolecules in the tumor cells, when compared to the delivery of these molecules to normal tissue.

WO 03/074088 describes hydroxyalkyl starch conjugates with, for example, cytotoxic agents such as daunorubicin, wherein the cytotoxic agent is usually directly coupled via an amino group to the hydroxyalkyl starch yielding in 1:1 conjugates. The hydroxyalkyl starch is described as having a substitution range preferably in the range of from 0.2 to 0.8. No use of these conjugates in vivo was shown. Further, in WO 03/074088 no cleavable linkage between the cytotoxic agent and hydroxyalkyl starch was described, which, upon administration, would be suitable to readily liberate the active drug in vivo.

Thus, there is still the need to provide new prodrugs of cytotoxic agents being bound to advantageous polymers for the treatment of cancer in vivo.

Thus, it is an object of the present invention to provide novel conjugates comprising a polymer linked to a cytotoxic agent. Further, it is an object of the present invention to provide a method for preparing such conjugates. It is yet another object of the present invention to provide polymer derivatives suitable for being coupled to cytotoxic agents and a method for preparing the same. Additionally, it is an object of the present invention to provide pharmaceutical compositions comprising these novel conjugates as well as the use of the conjugates and the pharmaceutical composition, respectively, in the treatment of cancer.

Surprisingly, it was found that linking of a cytotoxic agent via a secondary hydroxyl group to a hydroxyalkyl starch derivative having a specific molecular weight MW as well as a specific molar substitution MS may lead to a conjugate showing at least one of the desired beneficial properties, such as improved drug solubility, and/or optimized drug residence time in vivo, and/or reduced toxicity, and/or high efficiency, and/or effective targeting of tumor tissue in vivo. Without wanting to be bound to any theory, it is believed that the specific biodegradable hydroxyalkyl starch polymers of the invention may exhibit an optimized size, characterized by specific values of MW, which is large enough to prevent the elimination of the intact conjugate—comprised of the polymer and the cytotoxic agent—through the kidney prior to any release of the cytotoxic agent. Thus, elimination of the conjugate in the kidney by filtration through pores may be avoided. Further, the specific biodegradable hydroxyalkyl starch polymers of the invention comprised in the conjugate may exhibit an optimized molar substitution MS, and/or the conjugate as such may exhibit a preferred overall chemical constitution, so as to allow for a degradability of the hydroxyalkyl starch polymer comprised in the conjugate and release of the cytotoxic agent in a favorable time range. Further, it is believed that in contrast to most of the polymers described in the prior art, such as polyethylene glycol and derivatives thereof, the polymer fragments obtained from degradation of the conjugate of the present invention can be removed from the bloodstream by the kidneys or degraded via the lysosomal pathway without leaving any unknown degradation products of the polymer in the body.

Without wanting to be bound to any theory as to how the conjugates of the invention might operate, it is further contemplated that at least some of the conjugates of the invention might be able to deliver the respective cytotoxic agent into extracellular tissue space, such as into tissue exhibiting an EPR effect. However, it has to be understood that it is not intended to limit the scope of the invention only to such conjugates which take advantage of the EPR effect; also conjugates which show, possibly additionally, different advantageous characteristics, such as advantageous activity and/or low toxicity in vivo due to alternative mechanisms, are encompassed by the present invention.

Thus, the present invention relates to a hydroxyalkyl starch (HAS) conjugate comprising a hydroxyalkyl starch derivative and a cytotoxic agent, said conjugate having a structure according to the formula

HAS′(-L-M)ₙ

wherein M is a residue of a cytotoxic agent, wherein the cytotoxic agent comprises a secondary hydroxyl group, L is a linking moiety (linking the residue of the HAS derivative and M), HAS′ is the residue of the hydroxyalkyl starch derivative, and n is greater than or equal to 1, preferably in the range of from 3 to 200 and wherein the hydroxyalkyl starch derivative has a mean molecular weight MW above the renal threshold, preferably in the range of from 60 to 800 kDa, more preferably of from 80 to 800 kDa, and a molar substitution MS in the range of from 0.6 to 1.5, and wherein the linking moiety L is linked to the secondary hydroxyl group of the cytotoxic agent.

Further, the present invention relates to a method for preparing a hydroxyalkyl starch (HAS) conjugate comprising a hydroxyalkyl starch derivative and a cytotoxic agent, said conjugate having a structure according the following formula

HAS′(-L-M)ₙ

wherein
M is a residue of a cytotoxic agent, said cytotoxic agent comprising a secondary hydroxyl group, L is a linking moiety, HAS′ is a residue of the hydroxyalkyl starch derivative, and n is greater than or equal to 1, preferably wherein n is in the range of from 3 to 200, said method comprising
(a) providing a hydroxyalkyl starch derivative having a mean molecular weight MW above the renal threshold, preferably in the range of from 60 to 800 kDa, more preferably of from 80 to 800 kDa, and a molar substitution MS in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent.

The term “linked to the secondary hydroxyl group of the cytotoxic agent” as used in the context of the present invention is denoted to mean that the cytotoxic agent is reacted via its secondary hydroxyl group. The resulting conjugated residue of the cytotoxic agent M is thus linked via an —O— group to linking moiety L wherein the oxygen of this —O— group corresponds to the oxygen of the reacted secondary hydroxyl group of the cytotoxic agent.

Moreover, the present invention relates to a hydroxyalkyl starch conjugate obtainable or obtained by the above-mentioned method.

Further, the present invention relates to a method for preparing a hydroxyalkyl starch derivative, preferably having a mean molecular weight MW above the renal threshold, preferably in the range of from 60 to 800 kDa, more preferably of from 80 to 800 kDa, and preferably having a molar substitution MS in the range of from 0.6 to 1.5, the hydroxyalkyl starch derivative comprising at least one structural unit, preferably 3 to 200 structural units, according to the following formula (I)

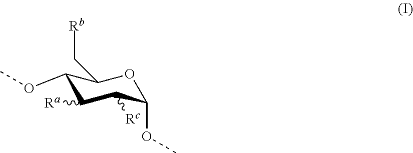

wherein R^{a}, R^{b} and R^{c} are, independently of each other, selected from the group consisting of —O—HAS″, —[O(CR^{w}R^{x})—(CR^{y}R^{z})]ₓ—OH, —[O—(CR^{w}R^{x})—(CR^{y}R^{z})]_{y}—Z¹, —[O—(CR^{w}R^{x})—(CR^{y}R^{z})]_{y}—[F¹]ₚ-L¹-Z¹, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety, HAS″ is the remainder of HAS and wherein Z¹ is a functional group capable of being reacted with a functional group of a further compound and wherein at least one of R^{a}, R^{b} and R^{c} comprises the functional group Z¹, and wherein Z¹ is preferably —SH, said method comprising
(a1) providing a hydroxyalkyl starch, preferably having a mean molecular weight MW above the renal threshold, preferably in the range of from 60 to 800 kDa, more preferably of from 80 to 800 kDa, and preferably having a molar substitution MS in the range of from 0.6 to 1.5, comprising the structural unit according to the following formula (II)

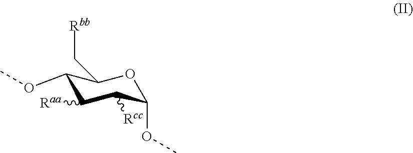

wherein R^{aa}, R^{bb} and R^{cc} are independently of each other selected from the group consisting of —[O—(CR^{w}R^{x})—(CR^{y}R^{z})]ₓ—OH and —O—HAS″,
(a2) introducing at least one functional group Z¹ into the hydroxyalkyl starch by
   (i) coupling the hydroxyalkyl starch via at least one hydroxyl group to at least one suitable linker comprising the functional group Z¹ or a precursor of the functional group Z¹, or
   (ii) displacing a hydroxyl group present in the hydroxyalkyl starch in a substitution reaction with a precursor of the functional group Z¹ or with a bifunctional linker comprising the functional group Z¹ or a precursor thereof.

Further, the present invention also relates to a hydroxyalkyl starch derivative obtainable or obtained by said method.

The term “at least one suitable linker comprising a precursor of the functional group Z¹” as used in the context of the present invention is denoted to mean a linker comprising a functional group which is capable of being transformed in at least one further step to give the functional group Z¹. The term “precursor” used in the context of “displacing the hydroxyl group of hydroxyalkyl starch with a precursor”, is denoted to mean a reagent which is capable of displacing the hydroxyl group, thereby forming a functional group Z¹ or a group, which can be modified in at least one further step to give the functional group Z¹.

Further, the present invention also relates to a hydroxyalkyl starch derivative, preferably having a mean molecular weight MW above the renal threshold, preferably in the range of from 60 to 800 kDa, more preferably of from 80 to 800 kDa, and preferably having a molar substitution in the range of from 0.6 to 1.5, said hydroxyalkyl starch derivative comprising at least one structural unit, preferably 3 to 200 structural units, according to the following formula (I)

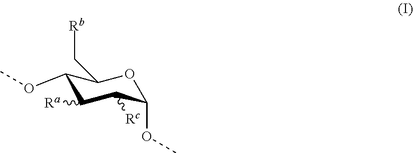

wherein R^{a}, R^{b} and R^{c} are independently of each other selected from the group consisting of —O—HAS″, —[O—CH₂—CH₂]_{S}—OH, —[O—CH₂—CH₂]ₜ—Z¹ and —[O—CH₂—CH₂]ₜ—[F¹]ₚ-L¹-Z¹, wherein at least one R^{a}, R^{b} and R^{c} is —[O—CH₂—CH₂]ₜ—Z¹ or —[O—CH₂—CH₂]ₜ—[F¹]ₚ-L¹-Z¹, wherein s is in the range of from 0 to 4, wherein t is in the range of from 0 to 4, p is 0 or 1, and wherein Z¹ is —SH.

According to yet another embodiment of the present invention, the present invention relates to a pharmaceutical compound or composition comprising the hydroxyalkyl starch conjugate or the hydroxyalkyl starch conjugate obtainable or obtained by the above-mentioned method. Further, the present invention relates to the hydroxyalkyl starch conjugate as described above, or the pharmaceutical composition as described above, for the use as a medicament, in particular for the treatment of cancer. Further, the present invention relates to the use of the hydroxyalkyl starch conjugate as described above, or the pharmaceutical composition as described above for the manufacture of a medicament for the treatment of cancer. Moreover, the present invention relates to a method of treating a patient suffering from cancer comprising administering a therapeutically effective amount of the hydroxyalkyl starch conjugate as described above, or the pharmaceutical composition as described above.

### The Hydroxyalkyl Starch

In the context of the present invention, the term “hydroxyalkyl starch” (HAS) refers to a starch derivative having a constitution according to the following formula (III)

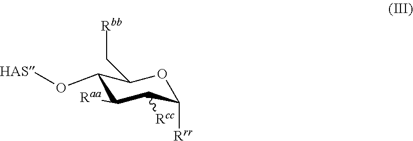

wherein the explicitly shown ring structure is either a terminal or a non-terminal saccharide unit of the HAS molecule and wherein HAS″ is a remainder, i.e. a residual portion of the hydroxyalkyl starch molecule, said residual portion forming, together with the explicitly shown ring structure containing the residues R^{aa}, R^{bb} and R^{cc} and R^{rr} the overall HAS molecule. In formula (III), R^{aa}, R^{bb} and R^{cc} are independently of each other hydroxyl, a linear or branched hydroxyalkyl group, or —O—HAS″, in particular R^{aa}, R^{bb} and R^{cc} are independently of each other —O—HAS″ or —[O—(CR^{w}R^{x})—(CR^{y}R^{z})]ₓ—OH, wherein R^{w}, R^{x}, R^{y} and R^{z} are independently of each other selected from the group consisting of hydrogen and alkyl, x is an integer in the range of from 0 to 20, preferably in the range of from 0 to 4. Preferably, R^{aa}, R^{bb} and R^{cc} are independently of each other —O—HAS″ or —[O—CH₂—CH₂]ₛ—OH with s being in the range of from 0 to 4. In particular, R^{aa}, R^{bb} and R^{cc} are independently of each other —OH, —O—CH₂—CH₂—OH (2-hydroxyethyl), or —O—HAS″. Residue R^{rr} is —O—HAS″ in case the explicitly shown ring structure is a non-terminal saccharide unit of the HAS molecule. In case the explicitly shown ring structure is a terminal saccharide unit of the HAS molecule, R^{rr} is —OH, and formula (III) shows this terminal saccharide unit in its hemiacetal form. This hemiacetal form, depending on e.g. the solvent, may be in equilibrium with the free aldehyde form as shown in the scheme below:

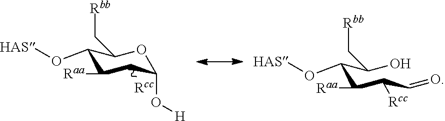

The term —O—HAS″ as used in the context of the residue R^{rr} as described above is, in addition to the remainder HAS″ shown at the left hand side of formula (III), a further remainder of the HAS molecule which is linked as residue R^{rr} to the explicitly shown ring structure of formula (III)

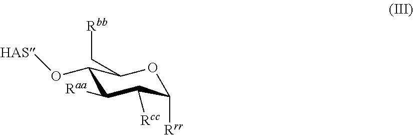

and forms, together with the residue HAS″ shown at the left hand side of formula (III) and the explicitly shown ring structure the overall HAS molecule.

Each remainder HAS″ discussed above comprises, preferably essentially consists of—apart from terminal saccharide units—one or more repeating units according to formula (IIIa)

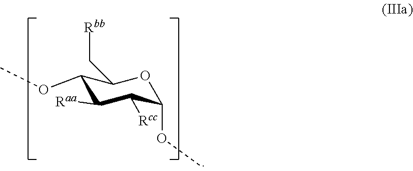

According to the present invention, the HAS molecule shown in formula (III) is either linear or comprises at least one branching point, depending on whether at least one of the residues R^{aa}, R^{bb} and R^{cc} of a given saccharide unit comprises yet a further remainder —O—HAS″. If none of the residues R^{aa}, R^{bb} and R^{cc} of a given saccharide unit comprises yet a further remainder —O—HAS″, apart from the HAS″ shown at the left hand side of formula (III), and optionally apart from HAS″ contained in R^{rr}, the HAS molecule is linear.

Hydroxyalkyl starch comprising two or more different hydroxyalkyl groups is also conceivable. The at least one hydroxyalkyl group comprised in the hydroxyalkyl starch may contain one or more, in particular two or more, hydroxyl groups. According to a preferred embodiment, the at least one hydroxyalkyl group contains only one hydroxyl group.

The term “hydroxyalkyl starch” as used in the present invention also includes starch derivatives wherein the alkyl group is suitably mono- or polysubstituted. Such suitable substituents are preferably halogen, especially fluorine, and/or an aryl group. Yet further, instead of alkyl groups, HAS may comprise also linear or branched substituted or unsubstituted alkenyl groups.

Hydroxyalkyl starch may be an ether derivative of starch, as described above. However, besides of said ether derivatives, also other starch derivatives are comprised by the present invention, for example derivatives which comprise esterified hydroxyl groups. These derivatives may be, for example, derivatives of unsubstituted mono- or dicarboxylic acids with preferably 2 to 12 carbon atoms or of substituted derivatives thereof. Especially useful are derivatives of unsubstituted monocarboxylic acids with 2 to 6 carbon atoms, especially derivatives of acetic acid. In this context, acetyl starch, butyryl starch and propynyl starch are preferred.

Furthermore, derivatives of unsubstituted dicarboxylic acids with 2 to 6 carbon atoms are preferred. In the case of derivatives of dicarboxylic acid, it is useful that the second carboxy group of the dicarboxylic acid is also esterified. Furthermore, derivatives of monoalkyl esters of dicarboxylic acids are also suitable in the context of the present invention. For the substituted mono- or dicarboxylic acids, the substitute group may be preferably the same as mentioned above for substituted alkyl residues. Techniques for the esterification of starch are known in the art (cf. for example Klemm, D. et al., Comprehensive Cellulose Chemistry, vol. 2, 1998, Wiley VCH, Weinheim, New York, especially Chapter 4.4, Esterification of Cellulose (ISBN 3-527-29489-9)).

According to a preferred embodiment of the present invention, a hydroxyalkyl starch (HAS) according to the above-mentioned formula (III)

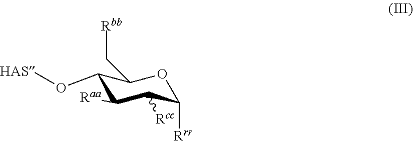

is employed. The saccharide units comprised in HAS″, apart from terminal saccharide units, may be the same or different, and preferably have the structure according to the formula (IIIa)

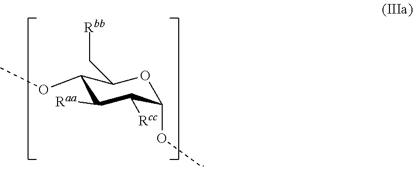

as shown above.

According to the invention, the term “hydroxyalkyl starch” is preferably a hydroxyethyl starch, hydroxypropyl starch or hydroxybutyl starch, wherein hydroxyethyl starch is particularly preferred.

Thus, according to the present invention, the hydroxyalkyl starch (HAS) is preferably a hydroxyethyl starch (HES), the hydroxyethyl starch preferably having a structure according to the following formula (III)

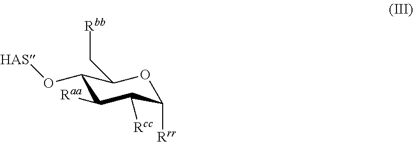

wherein R^{aa}, R^{bb} and R^{cc} are independently of each other selected from the group consisting of —O—HES″, and —[O—CH₂—CH₂]ₛ—OH, wherein s is in the range of from 0 to 4 and wherein in case the hydroxyalkyl starch is hydroxyethyl starch, HAS″ is the remainder of the hydroxyethyl starch and could be abbreviated with HES″. Residue R^{rr} is either —O—HAS″ (which in case the hydroxyalkyl starch is hydroxyethyl starch could be abbreviated with —O—HES″) or, in case the formula (III) shows the terminal saccharide unit of HES, R^{rr} is —OH. For the sake of consistency, the abbreviation “HAS” is used throughout all formulas in the context of the present invention, and if HAS is concretized as HES, it is explicitly mentioned in the corresponding portion of the text.

### The Term “Hydroxyalkyl Starch Derivative”

In the context of the present invention, the term “hydroxyalkyl starch derivative” refers to a derivative of starch being functionalized with at least one functional group Z¹, said group being a functional group capable of being linked to a further compound, in particular to the linking moiety L comprised in the structural unit -L-M which in turn is comprised in the above-defined conjugate having a structure according to the following formula

HAS′(-L-M)ₙ.

In accordance with the above-mentioned definition of HAS, the hydroxyalkyl starch derivative preferably comprises at least one structural unit according to the following formula (I)

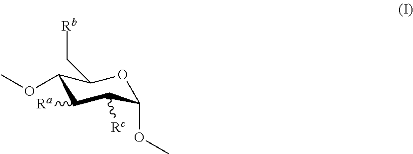

wherein at least one of R^{a}, R^{b} or R^{c} comprises the functional group Z¹ and wherein R^{a}, R^{b} and are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—Z¹, —[O—(CR^{w}R^{x})—(CR^{y}R^{z})]_{y}—[F¹]ₚ-L¹-Z¹, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety and Z¹ is a functional group which is capable of being linked to a further compound, in particular to the linking moiety L comprised in the structural unit -L-M.

In particular, a hydroxyalkyl starch derivative which comprises at least one structural unit according to the following formula (I)

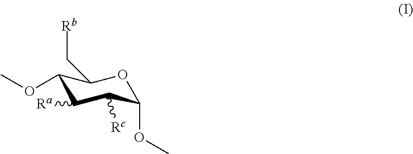

has preferably a structure according to the following formula (IV)

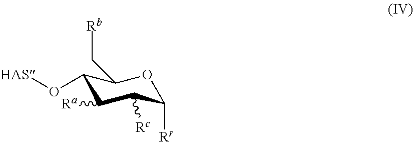

wherein R^{r} is —O—HAS″ or, in case the ring structure of formula (IV) shows the terminal saccharide unit of HAS, R^{r} is —OH, and wherein HAS″ is a remainder of the hydroxyalkyl starch derivative.

Analogously to the above-discussed definition of the term HAS″ in the context of the hydroxyalkyl starch as such, the term “remainder of the hydroxyalkyl starch derivative” is denoted to mean a linear or branched chain of the hydroxyalkyl starch derivative, being linked to the oxygen groups as shown in formula (IV) or being comprised in the residues R^{a}, R^{b} or R^{c} of formula (I), wherein said linear or branched chains comprise at least one structural unit according to formula (I)

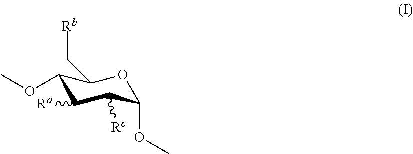

wherein at least one of R^{a}, R^{b} or R^{c} comprises the functional group Z¹ and/or one or more structural units of the formula (Ib)

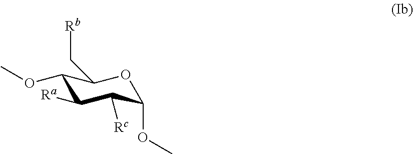

wherein R^{a}, R^{b} and R^{c} are, independently of each other, selected from the group consisting of —O—HAS″ and —[O—(CR^{w}R^{x})—(CR^{y}R^{z})]ₓ—OH, wherein R^{w}, R^{x}, R^{y} and R^{z} are as described above.

In case the hydroxyalkyl starch derivative has a linear starch backbone, none of R^{a}, R^{b} or R^{c} comprises a further group —O—HAS″. In case at least one of R^{a}, R^{b} or R^{c} is —O—HAS″, the hydroxyalkyl starch derivative comprises at least one branching point.

In particular, in case, the structural unit is the reducing sugar moiety of the hydroxyalkyl starch derivative, the terminal structural unit has a structure according to the following formula (Ia)

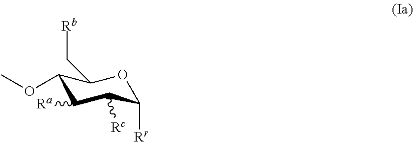

wherein R^{r} is —OH or a group comprising the functional group Z¹. R^{r} is preferably selected from the group consisting of —OH, —Z¹ and —[F¹]ₚ-L¹-Z¹, most preferably R^{r} is —OH, the reducing end of the hydroxyalkyl starch thus being present in unmodified form.

In the above-mentioned formula (Ia), the bond “ [symbol] ” represents a bond with non-defined stereochemistry, i.e. this term represents a bond encompassing both possible stereochemistries. Preferably, the stereochemistry in most building blocks, preferably in all building blocks of the HAS derivative is defined according to the formulas (Ib) and (IVa)

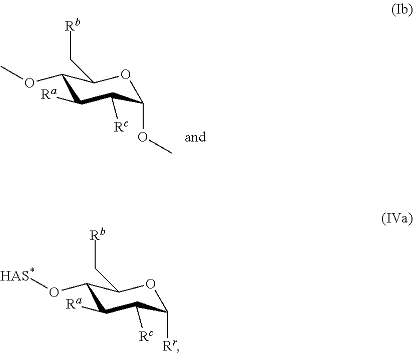

respectively.

According to a preferred embodiment of the present invention, the hydroxyalkyl starch (HAS) derivative is a hydroxyethyl starch (HES) derivative.

Therefore, the present invention also describes a hydroxyalkyl starch derivative as described above, and a method for preparing said hydroxyalkyl starch derivative, and a conjugate comprising said hydroxyalkyl starch derivative and a cytotoxic agent, and a conjugate obtained or obtainable by the above-mentioned method wherein the conjugate comprises said hydroxyalkyl starch derivative and a cytotoxic agent, wherein the hydroxyalkyl starch derivative is a hydroxyethyl starch derivative.

Accordingly, in case the hydroxyalkyl starch (HAS) is hydroxyethyl starch (HES), the HAS derivative preferably comprises at least one structural unit, preferably 3 to 200 structural units, according to the following formula (I)

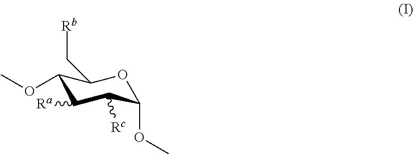

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ—[F¹]ₚ-L¹-Z¹, wherein at least one R^{a}, R^{b} and R^{c} is —[O—CH₂—CH₂]ₜ—Z¹ or —[O—CH₂—CH₂]ₜ—[F¹]ₚ-L¹-Z¹, wherein s is in the range of from 0 to 4, wherein t is in the range of from 0 to 4, and wherein p is 0 or 1.

### The Amount of Functional Groups Z¹ Present in the Hydroxyalkyl Starch Derivative

As regards the amount of functional groups Z¹ present in a given hydroxyalkyl starch derivative, preferably 0.15% to 2% of all residues R^{a}, R^{b} and R^{c} present in the hydroxyalkyl starch derivative contain the functional group Z¹.

More preferably, 0.15% to 2% of all residues R^{a}, R^{b} and R^{c} present in the hydroxyalkyl starch derivative have the structure —[O—(CR^{w}R^{x})—(CR^{y}R^{z})]_{y}—Z¹ or —[O—(CR^{w}R^{x})—(CR^{y}R^{x})]_{y}—[F¹]ₚ-L¹-Z¹.

According to a particularly preferred embodiment, R^{a}, R^{b} and R^{c} are selected from the group consisting of —O—HAS″, —[O—(CR^{w}R^{x})—(CR^{y}R^{z})]ₓ—OH and —[O—(CR^{w}R^{x})—(CR^{y}R^{z})]_{y}—Z¹, wherein 0.15% to 2% of all residues R^{a}, R^{b} and R^{c} present in the hydroxyalkyl starch derivative have the structure —[O—(CR^{w}R^{x})—(CR^{y}R^{z})]_{y}—Z¹.

According to an alternative preferred embodiment, R^{a}, R^{b} and R^{c} are selected from the group consisting of —O—HAS″, —[O—(CR^{w}R^{x})—(CR^{y}R^{z})]ₓ—OH and —[O—(CR^{w}R^{x})—(CR^{y}R^{z})]_{y}—[F¹]ₚ-L¹-Z¹, wherein 0.15% to 2% of all residues R^{a}, R^{b} and R^{c} present in the hydroxyalkyl starch derivative have the structure —[O—(CR^{w}R^{x})—(CR^{y}R^{z})]_{y}—[F¹]ₚ-L¹-Z¹.

The term “Residue of the Hydroxyalkyl Starch Derivative”

The term “residue of the hydroxyalkyl starch derivative” (HAS′) refers to a hydroxyalkyl starch derivative being incorporated into a hydroxyalkyl starch conjugate. Within the meaning of the present invention the term “a conjugate comprising a hydroxyalkyl starch derivative” thus refers to a conjugate comprising a residue of a hydroxyalkyl starch derivative being incorporated into the conjugate and thus being linked to the linking moiety L comprised in the conjugate having a structure according to the following formula

HAS′(-L-M)ₙ.

Upon incorporation into the conjugate, the hydroxyalkyl starch derivative is coupled via at least one of its functional groups Z¹ to the crosslinking compound L (which is further reacted with M) or to the derivative of the cytotoxic agent having the structure -L-M, as described hereinabove and hereinunder, thereby forming a covalent linkage between the residue of the hydroxyalkyl starch derivative and L or -L-M, wherein the functional group X is formed upon reaction of Z¹ with L or -L-M, respectively.

Analogously to the above-discussed definition of the term “hydroxyalkyl starch derivative”, the term “residue of a hydroxyalkyl starch derivative” refers to a derivative of starch being linked via at least one functional group X via a linking moiety to a further compound, in particular via the at least one linking moiety L comprised in the structural unit -L-M which in turn is comprised in above-defined conjugate having a structure according to the following formula

HAS′(-L-M)ₙ.

In accordance with the above-mentioned definition of the hydroxyalkyl starch derivative, the residue of the hydroxyalkyl starch derivative preferably comprises at least one structural unit according to the following formula (I)

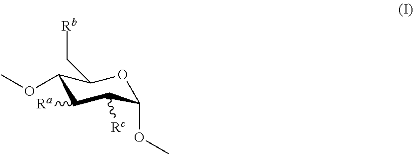

wherein R^{a}, R^{b} and R^{c} are, independently of each other, selected from the group consisting of —O—HAS″, —[O(CR^{w}R^{x})—(CR^{y}R^{z})]ₓ—OH, —[O—(CR^{w}R^{x})—(CR^{y}R^{z})]_{y}—X—, and —[O—(CR^{w}R^{x})—(CR^{y}R^{z})]_{y}—[F¹]ₚ-L¹-X—, and wherein at least one of R^{a}, R^{b} or R^{c} comprises the functional group —[O—(CR^{w}R^{x})—(CR^{y}R^{z})]_{y}—X— or —[O—(CR^{w}R^{x})—(CR^{y}R^{z})]_{y}—[F¹]ₚ-L¹-X—, and wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety and X is a functional group which is linked to a further compound, in particular to the linking moiety L comprised in the structural unit -L-M.

Besides the at least one structural unit according to formula (I),

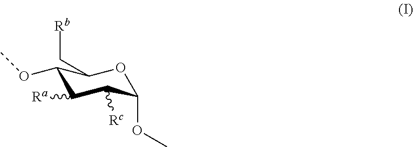

wherein at least one of R^{a}, R^{b} or R^{c} comprises the functional group —[O—(CR^{w}R^{x})—(CR^{y}R^{z})]_{y}—X— or —[O—(CR^{w}R^{x})—(CR^{y}R^{z})]_{y}—[F¹]ₚ-L¹-X—, the residue of the hydroxyalkyl starch preferably comprises one or more structural units of the formula (Ib)

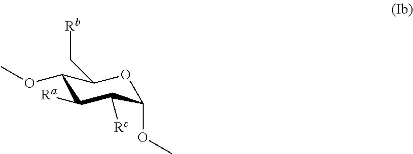

wherein R^{a}, R^{b} and R^{c} are, independently of each other, selected from the group consisting of —O—HAS″ and —[O—(CR^{w}R^{x})—(CR^{y}R^{z})]ₓ—OH.

As disclosed above, preferably 0.15% to 2% of all residues R^{a}, R^{b} and R^{c} present in the hydroxyalkyl starch derivative contain the functional group Z¹. Further, preferably all functional groups Z¹ being present in a given hydroxyalkyl starch derivative are coupled according to the coupling reaction of step (b) as defined hereinabove, thereby forming the covalent linkage via functional group X. Consequently, preferably 0.15% to 2% of all residues R^{a}, R^{b} and R^{c} present in the residue of the hydroxyalkyl starch derivative contain the functional group X. Thus, preferably 0.15% to 2% of all residues R^{a}, R^{b} and R^{c} present in the residue of the conjugate of the present invention contain the functional group X.

However, in case the hydroxyalkyl starch derivative comprises at least two functional groups Z¹, it may be possible that in step (b) not all of these functional groups Z¹ reacted with the crosslinking compound L, which in turn is reacted (either prior to or after the reaction with the HAS derivative) with the cytotoxic agent, giving a conjugate in which the HAS derivative is linked via the linking moiety L to the residue of the cytotoxic agent M. Thus, embodiments are encompassed in which not all functional groups are coupled to the crosslinking compound L or to the derivative of the cytotoxic agent -L-M. The residue of the hydroxyalkyl starch derivative present in the conjugate of the invention may thus comprise at least one unreacted functional group Z¹. Further, in case the hydroxyalkyl starch derivative is reacted with the crosslinking compound L which comprises the functional groups K¹ and K² as described above, prior to the coupling reaction to the cytotoxic agent, the residue of the hydroxyalkyl starch derivative present in the conjugate of the present invention may comprise at least one unreacted functional group K². All conjugates mentioned hereinunder and above, may comprise such unreacted groups.

To avoid possible side effects due to the presence of such unreacted functional groups Z¹ and/or unreacted functional groups K², the hydroxyalkyl starch conjugate may be further reacted with a suitable compound allowing for capping Z¹ and/or K² with a capping reagent D* in a preferably subsequent step (c) as described hereinunder in detail.

Thus, a hydroxyalkyl starch derivative comprised in a conjugate according to the invention mentioned hereinunder or above may comprise at least one structural unit according to formula (I)

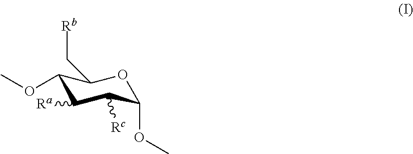

wherein one or more of R^{a}, R^{b} or R^{c} is —[O—(CR^{w}R^{x})—(CR^{y}R^{z})]_{y}—X-(L)_{beta}-D or —[O—(CR^{w}R^{x})—(CR^{y}R^{z})]_{y}—[F¹]-L¹-X-(L)_{beta}-D, wherein D is a capping group, L is the linking moiety comprised in the conjugate, beta is 0 or 1, preferably 0, and X is the functional group being formed upon reaction of at least one functional group Z¹ with a capping reagent D* thereby forming the structural unit —X-D (in this case beta is 0) or X is the functional group which is formed upon reaction of Z¹ with the crosslinking compound L, as described above, which in turn may be reacted via its functional group K² with a capping reagent D*, as described above, thereby forming the structural unit -L-D.

As regards the amount of functional groups X being linked to the functional moiety -L-M present in a given hydroxyalkyl starch conjugate, preferably at least 50%, more preferably at least 75%, more preferably at least 90%, more preferably at least 95%, most preferably at least 99%, of all functional groups X present in the conjugate of the present invention are linked to the functional moiety -L-M.

Alternatively, the conjugates of the present invention may also be described by the formula

[D-(L)_{beta}-]_{gamma}HAS*(-L-M)ₙ

wherein beta is 0 or 1, preferably 0, and wherein generally 0≦gamma<n, preferably wherein 0≦gamma<<<n, especially preferably wherein gamma is 0, wherein the residue of the hydroxyalkyl starch derivative HAS* comprises at least one structural unit according to formula (I),

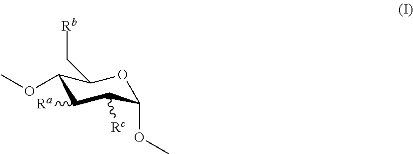

wherein at least one of R^{a}, R^{b} or R^{c} comprises the functional group X, and wherein the residue of the hydroxyalkyl starch HAS* preferably comprises one or more structural units of the formula (Ib)

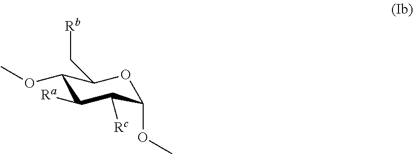

wherein R^{a}, R^{b} and R^{c} are, independently of each other, selected from the group consisting of —O—HAS″ and —[O—(CR^{w}R^{x})—(CR^{y}R^{z})]ₓ—OH, and wherein HAS* comprises no structural units —[O—(CR^{w}R^{x})—(CR^{y}R^{z})]_{y}—X-(L)_{beta}-D or —[O—(CR^{w}R^{x})—(CR^{y}R^{z})]_{y}—[F¹]ₚ-L¹-X-(L)_{bet}-D.

### Substitution Pattern Molar Substitution (MS) and Degree of Substitution (DS)

HAS, in particular HES, is mainly characterized by the molecular weight distribution, the degree of substitution and the ratio of C₂:C₆ substitution. There are two possibilities of describing the substitution degree.

The degree of substitution (DS) of HAS is described relatively to the portion of substituted glucose monomers with respect to all glucose moieties.

The substitution pattern of HAS can also be described as the molar substitution (MS), wherein the number of hydroxyethyl groups per glucose moiety is counted.

In the context of the present invention, the substitution pattern of the hydroxyalkyl starch (HAS), preferably HES, is referred to as MS, as described above, wherein the number of hydroxyalkyl groups present per sugar moiety is counted (see also Sommermeyer et al., 1987, Krankenhauspharmazie, 8(8): 271-278, in particular page 273). The MS is determined by gaschromatography after total hydrolysis of the hydroxyalkyl starch molecule.

The MS values of the respective hydroxyalkyl starch, in particular hydroxyethyl starch starting materials, are given since it is assumed that the MS value is not affected during the derivatization procedures as well as during the coupling step of the present invention.

The MS value corresponds to the degradability of the hydroxyalkyl starch via alpha-amylase. The higher the MS value, the lower the degradability of the hydroxyalkyl starch. It was surprisingly found that the MS of the hydroxyalkyl starch derivative present in the conjugates according to the invention should preferably be in the range of from 0.6 to 1.5 to provide conjugates with advantageous properties. Without wanting to be bound to any theory, it is believed that a MS in the above mentioned range combined with the specific molecular weight range of the conjugates results in conjugates with an optimized enrichment of the cytotoxic agent in the tumor and/or residence time in the plasma allowing for a controlled release of the cytotoxic agent prior to the degradation of the polymer and the subsequent removal of polymer fragments through the kidney.

According to a preferred embodiment of the present invention, the molar substitution MS is in the range of from 0.70 to 1.45, more preferably in the range of from 0.80 to 1.40, more preferably in the range of from 0.85 to 1.35, such as 0.85, 0.90, 0.95, 1.0, 1.05, 1.1, 1.15, 1.2, 1.25, 1.3 or 1.35. According to an even more preferred embodiment, the MS is in the range of from 0.90 to 1.10, most preferably in the range of from 0.95 to 1.05.

Thus, the present invention also relates to a method for preparing a conjugate comprising a hydroxyalkyl starch derivative and a cytotoxic agent, as described above, and a conjugate obtained or obtainable by said method, wherein the hydroxyalkyl starch derivative has a molar substitution MS in the range of from 0.60 to 1.50, preferably in the range of from 0.70 to 1.45, more preferably in the range of from 0.80 to 1.40, more preferably in the range of from 0.85 to 1.35, more preferably in the range of from 0.90 to 1.10 and most preferably in the range of from 0.95 to 1.05.

Likewise, the present invention also relates to a hydroxyalkyl starch (HAS) conjugate comprising a hydroxyalkyl starch derivative and a cytotoxic agent, wherein the hydroxyalkyl starch derivative has a molar substitution MS in the range of from 0.60 to 1.50, preferably in the range of from 0.70 to 1.45, more preferably in the range of from 0.80 to 1.40, more preferably in the range of from 0.85 to 1.35, more preferably in the range of from 0.90 to 1.10 and most preferably in the range of from 0.95 to 1.05. Likewise, the present invention relates to a pharmaceutical composition comprising the hydroxyalkyl starch conjugate, as described above, or the hydroxyalkyl starch conjugate obtained or obtainable by the above described method.

Further, the present invention also describes a method for preparing a hydroxyalkyl starch derivative, as described above, as well as a hydroxyalkyl starch derivative as such, or a hydroxyalkyl starch derivative obtained or obtainable by said method, wherein the hydroxyalkyl starch derivative has a molar substitution MS in the range of from 0.60 to 1.50, preferably in the range of from 0.70 to 1.45, more preferably in the range of from 0.80 to 1.40, more preferably in the range of from 0.85 to 1.35, more preferably in the range of from 0.90 to 1.10 and most preferably in the range of from 0.95 to 1.05.

As far as the ratio of C₂:C₆ substitution is concerned, i.e. the degree of substitution (DS) of HAS, said substitution is preferably in the range of from 2 to 20, more preferably in the range of from 2 to 15 and even more preferably in the range of from 3 to 12, with respect to the hydroxyalkyl groups.

### Mean Molecular Weight MW (M_{w})

HAS and in particular HES compounds are present as polydisperse compositions, wherein each molecule differs from the other with respect to the polymerization degree, the number and pattern of branching sites, and the substitution pattern. HAS and in particular HES is therefore a mixture of compounds with different molecular weight. Consequently, a particular HAS and in particular a HES is determined by average molecular weight with the help of statistical means.

In this context the number average molecular weight is defined by equation 1:

$${\overline{M}}_{n} = \frac{{\sum }_{i} {n}_{i} \cdot {M}_{i}}{{\sum }_{i} {n}_{i}} \qquad ( 1 )$$

where nᵢ is the number of molecules of species i of molar mass Mᵢ. M̅ₙ indicates that the value is an average, but the line is normally omitted by convention. M_{w} is the weight average molecular weight, defined by equation 2:

$${\overline{M}}_{w} = \frac{{\sum }_{i} {n}_{i} \cdot {M}_{i}^{2}}{{\sum }_{i} {n}_{i} {M}_{i}} \qquad ( 2 )$$

where nᵢ is the number of molecules of species i of molar mass Mᵢ and M̅_{w} indicates that the value is an average, but the line is normally omitted by convention.

$${\overline{M}}_{n} = \frac{{\sum }_{i} {n}_{i} \cdot {M}_{i}}{{\sum }_{i} {n}_{i}}$$

Preferably, the hydroxyalkyl starch derivative, in particular the hydroxyethyl starch derivative comprised in the conjugate, as described above, has a mean molecular weight MW (weight mean) above the renal threshold.

The renal threshold is determined according to the method described by Waitzinger et al. (Clin. Drug Invest. 1998; 16: 151-160) and reviewed by Jungheinrich et al. (Clin. Pharmacokinet. 2006; 44(7): 681-699). Preferably, the renal threshold is denoted to mean a mean molecular weight MW above 40 kDa.

More preferably, the hydroxyalkyl starch derivative, in particular the hydroxyethyl starch derivative comprised in the conjugate, as described above, has a mean molecular weight MW above 45 kDa, more preferably above 50 kDa, more preferably above 60 kDa.

More preferably, the hydroxyalkyl starch derivative, in particular the hydroxyethyl starch derivative comprised in the conjugate, as described above, has a mean molecular weight MW in the range of from 60 to 800 kDa.

More preferably the hydroxyalkyl starch derivative, in particular the hydroxyethyl starch derivative, according to the invention, has a mean molecular weight MW (weight mean) in the range of from 80 to 800 kDa, more preferably in the range of from 80 to 500 kDa, more preferably in the range of from 85 to 450 kDa, more preferably in the range of from 90 to 400 kDa, more preferably in the range of from 95 to 350 kDa, more preferably in the range of from 95 to 300 kDa.

The term “mean molecular weight” as used in the context of the present invention relates to the weight as determined according to MALLS-GPC (multiple angle laser light scattering-GPC) method as described in example 1.4.16.

According to an especially preferred embodiment, the hydroxyalkyl starch derivative has a mean molecular weight MW in the range of from 95 to 150 kDa.

Therefore, the present invention also relates to a method as described above, for preparing a hydroxyalkyl starch derivative, as well as to a method for preparing a hydroxyalkyl starch conjugate, wherein the hydroxyalkyl starch derivative has a mean molecular weight MW above the renal threshold, preferably in the range of from 60 to 800 kDa, more preferably in the range of from 80 to 800 kDa, more preferably in the range of from 90 to 350 kDa, more preferably in the range of from 95 to 150 kDa. Likewise, the present invention relates to a hydroxyalkyl starch conjugate, as described above, comprising a hydroxyalkyl starch derivative, as well as to a hydroxyalkyl starch conjugate obtained or obtainable by the above-mentioned method, wherein the hydroxyalkyl starch derivative has a mean molecular weight MW in the range of from 90 to 350 kDa, preferably in the range of from 95 to 150 kDa.

According to an especially preferred embodiment, the hydroxyalkyl starch derivative has a MS in the range of from 0.70 to 1.45, more preferably in the range of from 0.80 to 1.40 and a mean molecular weight MW in the range of from 90 to 350 kDa, more preferably a mean molecular weight MW in the range of from 90 to 350 kDa and a molar substitution MS in the range of from 0.85 to 1.35, more preferably a mean molecular weight MW in the range of from 90 to 350 kDa and a molar substitution MS in the range of from 0.90 to 1.10, more preferably a mean molecular weight MW in the range of from 90 to 350 kDa and a MS in the range of from 0.95 to 1.05.

According to an especially preferred embodiment, the hydroxyalkyl starch derivative has a MS in the range of from 0.70 to 1.45, more preferably in the range of from 0.80 to 1.40 and a mean molecular weight MW in the range of from 95 to 150 kDa, more preferably a mean molecular weight MW in the range of from 95 to 150 kDa and a molar substitution MS in the range of from 0.85 to 1.35, more preferably a mean molecular weight MW in the range of from 95 to 150 kDa and a molar substitution in the range of from 0.90 to 1.10, more preferably a mean molecular weight MW in the range of from 95 to 150 kDa and a MS in the range of from 0.95 to 1.05.

### Integer n:

As regards the number of structural units of the Formula (I) present in the hydroxyalkyl starch derivative, according to a preferred embodiment of the present invention, the hydroxyalkyl starch derivative comprises at least one, preferably at least 2, more preferably 2 to 200, more preferably 3 to 200 structural units (-L-M).

### Drug Loading

The amount of M, present in the conjugates of the invention, can further be described by the drug loading (also: drug content). The “drug loading” as used in the context of the present invention is calculated as the mean molecular weight of the cytotoxic agent measured in mg drug, i.e. cytotoxic agent, per 1 g of the conjugate.

The drug loading is determined by measuring the absorbance of M (thus the cytotoxic agent bound to HAS) at a specific wavelength in a stock solution, and calculating the content using the following equation (Lambert Beer's law):

$${c}_{drag} [ µmol \text{/} {cm}^{2} ] = \frac{( A - {A}^{0} )}{ɛ * d}$$

where ε is the extinction coefficient of the cytotoxic agent at the specific wavelength, which is obtained from a calibration curve of the cytotoxic agent dissolved in the same solvent which is used as in the stock solution (given in cm²/μmol), at the specific wavelength, A is the absorption at this specific wavelength, measured in a UV-VIS spectrometer, A⁰ is the absorption of a blank sample and d the width of the cuvette (equals the slice of absorbing material in the path of the beam, usually 1 cm). The appropriate wavelength for the determination of drug loading is derived from a maximum in the UV-Vis-spectra, preferably at wavelengths above 230 nm.

With a known concentration of conjugate in the sample (c_{conjugate}) and the concentration of drug in the sample determined by Lambert Beer's law, the loading in micromol/g can be calculated according to the following equation:

$$Loading [ µmol \text{/} g ] = \frac{1000 * {c}_{drug} [ µmol \text{/} ml ]}{{c}_{conjugate} [ mg / ml ]}$$

The loading in mg/g can finally be determined taking into account the molecular weight of the drug M as shown in the following equation:

Loading[mg/g]=Loading[μmol/g]*MW_{drug}[μg/μmol]/1000

As regards the drug loading, according to a preferred embodiment of the present invention, the drug loading of the conjugates is preferably in the range of from 20 to 500 micromol drug/g conjugate, more preferably in the range of from 30 to 400 micromol drug/g conjugate, more preferably in the range of from 40 to 300 micromol drug/g conjugate and most preferably in the range of from 45 to 250 micromol drug/g conjugate (-L-M).

### The Cytotoxic Agent

The term “cytotoxic agent” as used in the context of the present invention refers to natural or synthetic substances, which inhibit the cell growth or the cell division in vivo. The term is intended to include chemotherapeutic agents, antibiotics and toxins such as enzymatically active toxins of bacterial, fungal, plant or animal origin, or fragments thereof.

Preferably, the term “cytotoxic agent” is a natural or synthetic substance which inhibits the cell growth or the cell division of a tumor in vivo. Most preferably, the cytotoxic agent is a chemotherapeutic agent. The therapeutic use of these preferred cytotoxic agents, most preferably of the chemotherapeutic agents, is based on this difference in the rate of cell division and cell growth of tumor cells compared to normal cells. Among others, tumor cells differ from normal cells in that tumor cells are no longer subject to physiological growth control and therefore have an increased rate of cell division. Since the toxic activity of cytotoxic agents is usually primarily directed against proliferating cells, such cytotoxic agents can be used for inhibiting a development or progression of a neoplasm in vivo, particularly a malignant (cancerous) lesion, such as a carcinoma, sarcoma, lymphoma, or leukemia. Inhibition of metastasis is frequently also a property of the cytotoxic agents encompassed by the present invention.

With respect to the chemistry used in the context of the present invention, any cytotoxic agent, preferably any chemotherapeutic agent, known to those skilled in the art can be incorporated into the conjugates according to the present invention provided that this cytotoxic agent, preferably the chemotherapeutic agent, comprises a secondary hydroxyl group. Preferably the cytotoxic agent is an agent for the treatment of cancer.

The following structures are mentioned by way of example:

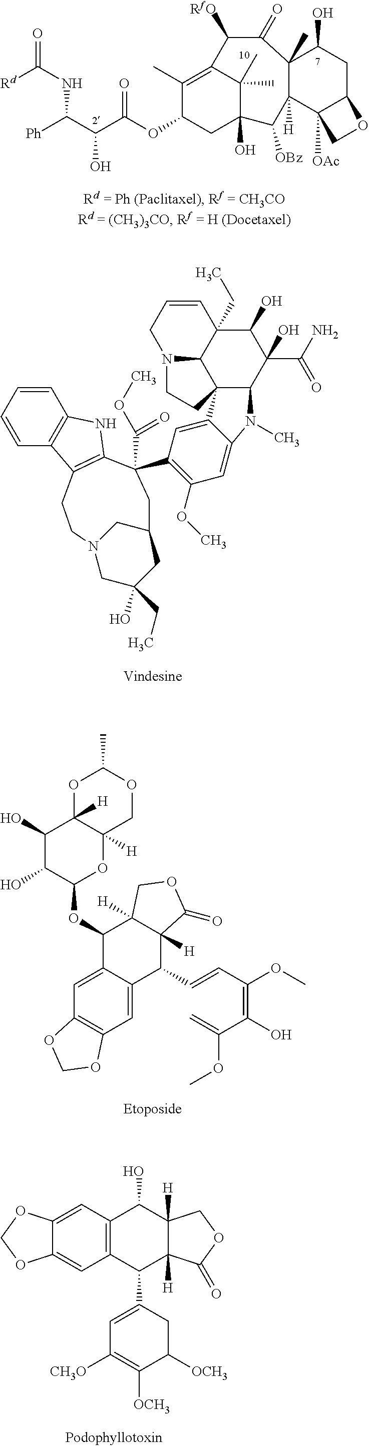

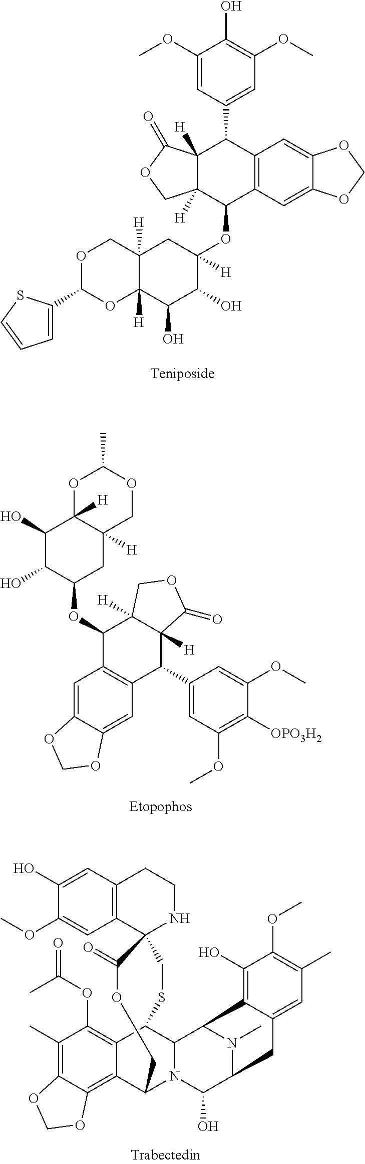

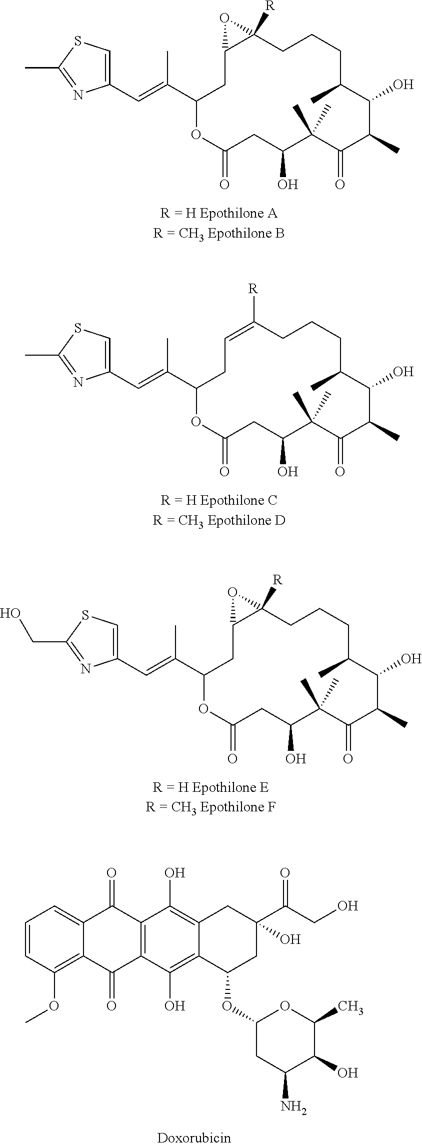

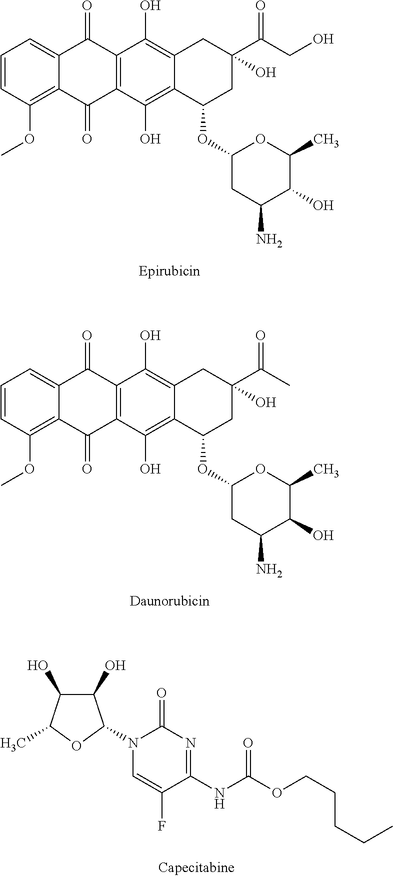 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1 ; COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1 ; CCCCCOC(=O)Nc1nc(=O)n([C@@H]2O[C@H](C)[C@@H](O)[C@H]2O)cc1F]

According to a preferred embodiment of the invention, the at least one secondary hydroxyl group containing cytotoxic agent is selected from the group consisting of tubulin interacting drugs, such as tubulin inhibitors (e.g. tubulysine U,) or tubulin stabilizers (such as peloruside A, the epothilone family, dictyostatin, discodermolide), topoisomerase I inhibitors (such as camptothecin, topotecan, irinotecan, silatecan (DB67), karenotecin (BNP 1350), exatecan, lurtotecan, gimatecan (ST 1481) and CKD 602), topoisomerase II inhibitors (such as etoposide and teniposide), DNA intercalators (such as mitoxantron), kinase inhibitors (such as rapamycin and analogues (temsirolimus, everolimus)), antimetabolites (such as capecitabine and gemcitabine), mitotic inhibitors (such as eribulin (E7389)), DNA damaging agents (such as trabectedin, bleomycin), anthracyclines (such as doxorubicin, epirubicin, daunorubicin), hormone analogues (such as fulvestrant), vinca alkaloids (such as vindesine, vinorelbine, vincristine, vinflunine and vinblastine), vascular disrupting agents (such as combretastatin (5-[(2R)-2-hydroxy-2-(3,4,5-trimethoxyphenyl)ethyl]-2-methoxyphenol and analogues) and HSP90-inhibitors (such as geldanamycin and analogues (e.g. 17-AAG)).

Preferably, the cytotoxic agent is selected from the group consisting of taxanes (wherein this term includes taxane derivatives), vindesine, etoposide, podophyllotoxin, teniposide, etopophos, trabectedin, epothilone A, epothilone B, epothilone C, epothilone D, epothilone E, epothilone F, ixabepilone, sagopilone, KOS-1584, capecitabine, epirubicin and daunorubicin.

A particularly preferred class of compounds according to the invention is the class of taxanes. For the purpose of the present invention, the term “taxane” refers to a class of compounds having the taxane ring system, shown by the core structure below

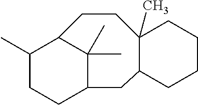 [SMILES: CC1CCC2CC3CCCCC3(C)CCC1C2(C)C]

derived from natural sources or which have been synthesized artificially.

It has to be understood, that any molecule comprising this core structure is, within the meaning of the present invention, encompassed by the term “taxane” provided that the core contains a secondary alcohol directly attached to the core structure or as part of a substituent. Apart from the hydroxyl group, the core structure may be further substituted in one or more positions and contain ethylenic unsaturation in the ring system thereof.

In this context also the so-called “second generation taxanes” should be mentioned which are meant to be encompassed by the term taxane used in the context of the present invention. A large variety of synthetic or semisynthetic paclitaxel analogues have been synthesized as so called “second generation taxanes” and identified as potential cytotoxic agents. By way of example larotaxel, carbitaxel, TPI-287, milataxel, tesetaxel, BMS-188797, BMS-184476, ortataxel, BMS-275183, simotaxel, TL-310 and the likes, should be mentioned (see following structures):

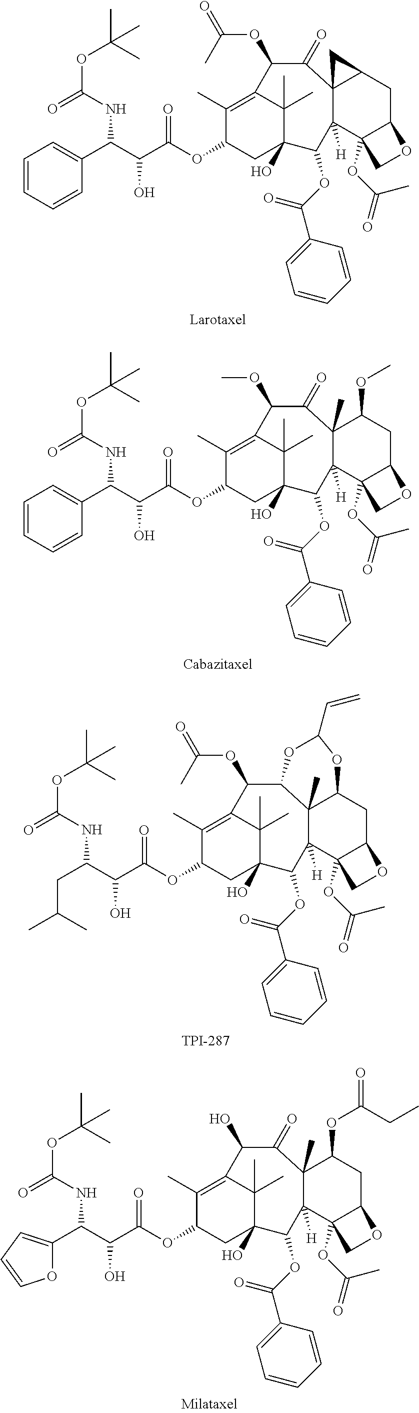

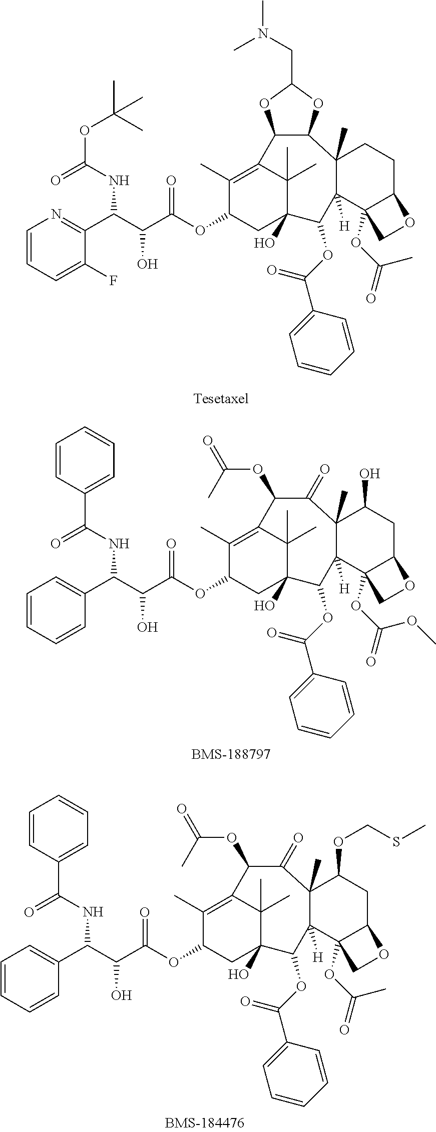

Most preferably, the cytotoxic agent according to the invention is a taxane having a structure according to the following formula, optionally being further substituted:

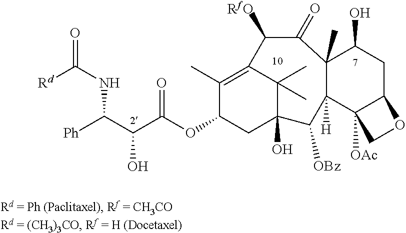

Most preferably, the cytotoxic agent is paclitaxel or docetaxel.

These compounds have been found to be effective anti-cancer agents. However, to date, their use is limited due to their poor water solubility. To date, this poor water solubility has to be overcome by complex formulation techniques. The standard formulation for paclitaxel (Taxol), for example, involves ethanol and the emulsifier Cremophor EL (polyethoxylated castor oil, an excipient infamous for its side effects which can be responsible for dose-limiting toxicities). This drawback can be overcome by the conjugates according to the present invention, wherein a hydroxyalkyl starch derivative, as described above, is linked via a linking moiety L to a secondary hydroxyl group of the cytotoxic agent, preferably to a secondary hydroxyl group of paclitaxel or docetaxel.

The present invention, thus, also relates to a method for preparing a hydroxyalkyl starch conjugate comprising a hydroxyalkyl starch derivative and a cytotoxic agent, wherein the cytotoxic agent is a cytotoxic agent selected from the group consisting of taxanes, taxane derivatives, vindesine, etoposide, podophyllotoxin, teniposide, etopophos, trabectedin, epothilone A, epothilone B, epothilone C, epothilone D, epothilone E, epothilone F, ixabepilone, sagopilone, KOS-1584, capecitabine, epirubicine and daunorubicine, more preferably the cytotoxic agent is a taxane, most preferably the cytotoxic agent is paclitaxel or docetaxel. Furthermore, the present invention also relates to a hydroxyalkyl starch conjugate, comprising a hydroxyalkyl starch derivative and a cytotoxic agent, as described above, as well as a hydroxyalkyl starch conjugate obtained or obtainable by the above-mentioned method, wherein the cytotoxic agent is selected from the group consisting of taxanes, taxane derivatives, vindesine, etoposide, podophyllotoxin, teniposide, etopophos, trabectedin, epothilone A, epothilone B, epothilone C, epothilone D, epothilone E, epothilone F, capecitabine, epirubicine and daunorubicine, more preferably the cytotoxic agent is a taxane, more preferably the cytotoxic agent is paclitaxel or docetaxel, most preferably the cytotoxic agent is docetaxel. Furthermore, the present invention also relates to a pharmaceutical composition comprising such hydroxyalkyl starch conjugates.

In case the cytotoxic agent is docetaxel or paclitaxel, the cytotoxic agent can be coupled via any secondary hydroxyl group present in these compounds. Thus, the coupling via the OH in 7-position as well as a coupling via the OH in 2′-position or in case R^{f} is H via the OH in 10-position is encompassed by the present invention. According to a preferred embodiment of the present invention, the linking moiety L is bound to the hydroxyl group present in 2′-position.

The term “is bound to the hydroxyl group” as used in the context of the present invention is denoted to mean that the cytotoxic agent is reacted via its secondary hydroxyl group, wherein the resulting conjugated residue of the cytotoxic agent M is thus linked via an —O— group to linking moiety -L- wherein the oxygen of this —O— group corresponds to the oxygen of the reacted secondary hydroxyl group of the cytotoxic agent.

Thus, the present invention also relates to a conjugate, as described above, as well as to a conjugate, obtained or obtainable by a method, as described above, the conjugate having a structure according to the following formula:

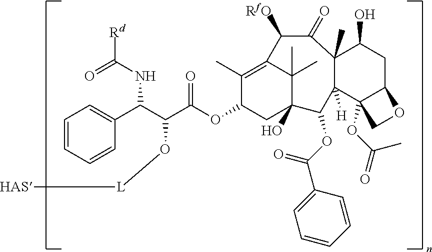

wherein R^{d} is preferably phenyl or O-t-butyl, and wherein R^{f} is preferably H or acetyl.

The following particular preferred structures are mentioned by way of example:

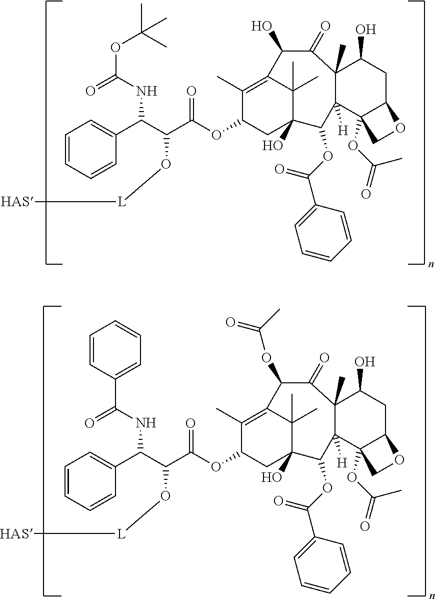

### The Linking Moiety L

According to the invention, the cytotoxic agent is preferably linked via a cleavable linker to the hydroxyalkyl starch derivative.

The expression “cleavable linker” refers to any linker which can be cleaved physically or chemically and preferably releases the cytotoxic agent in unmodified form. Examples for physical cleavage may be cleavage by light, radioactive emission or heat, while examples for chemical cleavage include cleavage by redox-reactions, hydrolysis, pH-dependent cleavage or cleavage by enzymes.

According to a preferred embodiment of the present invention, the cleavable linker comprises one or more cleavable bonds, preferably hydrolytically cleavable bonds, the cleavage, in particular the hydrolysis, of which releases the cytotoxic agent in vivo. Preferably the bond between the linking moiety L and the secondary hydroxyl group of the cytotoxic agent is a cleavable linkage.

Thus, the present invention also relates to a conjugate as described above, as well as to a conjugate obtained or obtainable by the above described method, wherein the linking moiety L and the residue of a cytotoxic agent M are linked via the secondary hydroxyl group of the cytotoxic agent via a linkage which hydrolyzes or is cleaved by an alternative mechanism, preferably which hydrolyzes, in vivo and allows for the release of the cytotoxic agent, preferably in unmodified form.

Preferably, the linking moiety L has a structure -L′-F³—, wherein F³ is the functional group linking L¹ with M, and wherein the linkage between F³ and the group —O— derived from the secondary hydroxyl group of the cytotoxic agent is cleaved in vivo and releases the (residue of the) cytotoxic agent. L¹ is a linking moiety linking the functional group F³ with the hydroxyalkyl starch derivative.

### The Functional Group F³

There are in principle no restrictions as to the nature of the functional group F³ provided that this group forms together with the secondary hydroxyl group of the cytotoxic agent a functional moiety capable of being cleaved in vivo.

Beside the —C(═Y)— function, in particular the —C(═O)— function, this accounts, inter alia, for groups F³ which form together with the group —O— of M (derived from the secondary hydroxyl group of the cytotoxic agent) the structural unit —F³—O—, with —F³—O— being a carbonate, thiocarbonate, xanthogenate, carbamate or thiocarbamate of the type —Y^{Y}—C(═Y)—O— with Y^{Y} being —O—, —S— or —NH— and Y being O, S or NH.

Preferably, the functional group F³ is —C(═Y)— or —Y^{Y}—C(═Y)—, with Y being O, NH or S and with Y^{Y} being —O—, —S— or —NH—. In particular, the functional group F³ is —C(═Y)—, with Y being O, NH or S. Together with the group —O— of M (derived from the secondary hydroxyl group of the cytotoxic agent), the functional group F³ therefore preferably forms a —C(═Y)—O— bond with Y being O, NH or S, in particular with Y being O or S, more preferably with Y being O, and wherein L¹ present in the above mentioned structure -L′-F³— is a linking moiety linking the functional group F³ with the hydroxyalkyl starch derivative.

Therefore, the present invention also relates to a hydroxyalkyl starch conjugate comprising a hydroxyalkyl starch derivative and a cytotoxic agent, said conjugate having a structure according to the following formula HAS′(-L-M)ₙ, wherein the linking moiety L has a structure -L′-F³—, wherein F³ is a functional group linking L¹ with M, preferably wherein F³ is a —C(═Y)— group, with Y being O, NH or S, and wherein F³ is linked to the secondary hydroxyl group of the cytotoxic agent, thereby forming a —C(═Y)—O— bond with Y being O, NH or S, in particular with Y being O or S, more preferably with Y being O, and wherein L′ is a linking moiety.

Likewise, the present invention relates to a method for preparing a conjugate having a structure HAS′(-L-M)ₙ, wherein L has a structure -L′-F³—, wherein F³ is a functional group linking L¹ with M, preferably wherein F³ is a —C(═Y)— group, with Y being O, NH or S, and wherein the structural unit —F³—O— is formed upon reaction of the crosslinking compound L with the secondary hydroxyl group of the cytotoxic agent. Likewise, the present invention relates to a conjugate obtained or obtainable by the method, as described above.

According to a particular preferred embodiment, the present invention relates to a conjugate, as described above, as well as to a conjugate, obtained or obtainable by a method, as described above, the conjugate having a structure according to the following formula:

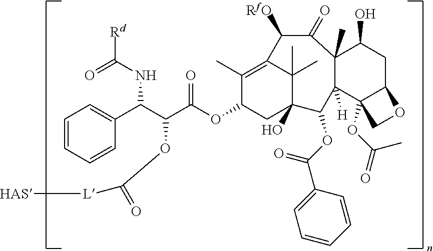

wherein R^{d} is preferably benzyl or O-t-butyl, and wherein R^{f} is preferably H or acetyl and n is greater than or equal to 1, preferably in the range of from 3 to 200.

### The Linking Moiety L′

According to a preferred embodiment of the present invention, the functional group F³ and the hydroxyalkyl starch derivative are separated by a suitable linking moiety L′, as described above. The term linking moiety L¹ as used in this context of the present invention relates to any suitable chemical moiety bridging F³ and the hydroxyalkyl starch derivative.

In general, there are no particular restrictions as to the chemical nature of the linking moiety L¹ with the proviso that L¹ provides suitable chemical properties for the novel conjugates for their intended use.

Preferably, L¹ is a linking moiety such as an alkyl, alkenyl, alkylaryl, arylalkyl, aryl, heteroaryl, alkylheteroaryl or heteroarylalkyl group.

Within the meaning of the present invention, the term “alkyl” relates to non-branched alkyl residues, branched alkyl residues, cycloalkyl residues, as well as residues comprising one or more heteroatoms or functional groups, such as, by way of example, —O—, —S—, —NH—, —NH—C(═O)—, —C(═O)—NH—, and the like. The term also encompasses alkyl groups which are further substituted by one or more suitable substituents. The term “substituted alkyl” as used in this context of the present invention preferably refers to alkyl groups being substituted in any position by one or more substituents, preferably by 1, 2, 3, 4, 5 or 6 substituents, more preferably by 1, 2, or 3 substituents. If two or more substituents are present, each substituent may be the same or may be different from the at least one other substituent. There are in general no limitations as to the substituent. The substituents may be, for example, selected from the group consisting of aryl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl, ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups.

The term “alkenyl” as used in the context of the present invention refers to unsaturated alkyl groups having at least one double bond. The term also encompasses alkenyl groups which are substituted by one or more suitable substituents.

The term “alkynyl” refers to unsaturated alkyl groups having at least one triple bond. The term also encompasses alkynyl groups which are substituted by one or more suitable substituents.

Within the meaning of the present invention, the term “aryl” refers to, but is not limited to, optionally suitably substituted 5- and 6-membered single-ring aromatic groups as well as optionally suitably substituted multicyclic groups, for example bicyclic or tricyclic aryl groups. The term “aryl” thus includes, for example, optionally substituted phenyl groups or optionally suitably substituted naphthyl groups. Aryl groups can also be fused or bridged with alicyclic or heterocycloalkyl rings which are not aromatic so as to form a polycycle, e.g., benzodioxolyl or tetraline.

The term “heteroaryl” as used within the meaning of the present invention includes optionally suitably substituted 5- and 6-membered single-ring aromatic groups as well as substituted or unsubstituted multicyclic aryl groups, for example tricyclic or bicyclic aryl groups, comprising one or more, preferably from 1 to 4 such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthyridinyl, quinolinyl, isoquinolinyl, indolyl, benzofuranyl, purinyl, deazapurinyl, or indolizinyl.

The term “optionally substituted aryl” and the term “optionally substituted heteroaryl” as used in the context of the present invention describes moieties having substituents replacing a hydrogen on one or more atoms, e.g. C or N, of an aryl or heteroaryl moiety. Again, there are in general no limitiations as to the substituent. The substituents may be, for example, selected from the group consisting of alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups.

The term “alkylaryl” as used in the context of any linking moiety described in the present invention is denoted to mean a linking moiety having the structure alkyl-aryl-, thus being linked on one side via the alkyl group and on the other side via the aryl group, wherein this term is meant to also encompass linking moieties such as alkyl-aryl-alkyl-linking moieties. The term “alkylaryl group”, when used in the context of any substituent described hereinunder and above, is denoted to mean a residue being linked via the alkyl portion, said alkyl portion being further substituted with an aryl moiety.

The term “arylalkyl” as used in the context of any linking moiety described in the present invention is denoted to mean a linking moiety having the structure aryl-alkyl-, thus being linked on one side via the aryl group and on the other side via the alkyl group, wherein this term is meant to also encompass linking moieties such as aryl-alkyl-aryl-linking moieties. The term “arylalkyl group”, when used in the context of any substituent described hereinunder and above, is denoted to mean a residue being linked via the aryl portion, said aryl portion being further substituted with an alkyl moiety.

The term “alkylheteroaryl” as used in the context of any linking moiety described in the present invention is denoted to mean a linking moiety having the structure alkyl-heteroaryl-, thus being linked on one side via the alkyl group and on the other side via the heteroaryl group, wherein this term is meant to also encompass linking moieties such as alkyl-heteroaryl-alkyl-linking moieties. The term “alkylheteroaryl group”, when used in the context of any substituent described hereinunder and above, is denoted to mean a residue being linked via the alkyl portion, said alkyl portion being further substituted with a heteroaryl moiety.

The term “heteroarylalkyl” as used in the context of any linking moiety described in the present invention is denoted to mean a linking moiety having the structure -heteroaryl-alkyl-, thus being linked on one side via the heteroaryl group and on the other side via the alkyl group, wherein this term is meant to also encompass linking moieties such as heteroaryl-alkyl-heteroaryl-linking moieties. The term “heteroarylalkyl group”, when used in the context of any substituent described hereinunder and above, is denoted to mean a residue being linked via the heteroaryl portion, said heteroaryl portion being further substituted with an alkyl moiety.

According to a preferred embodiment of the present invention, the hydroxyalkyl starch conjugate comprises an electron-withdrawing group in close proximity to the functional group F³. The term “electron-withdrawing group” is recognized in the art, and denotes the tendency of a functional group to attract valence electrons from neighboring atoms by means of a difference in electronegativity with respect to the neighboring atom (inductive effect) or by withdrawal of π-electrons via conjugation (mesomeric effect).

Preferably, the electron-withdrawing group is present in alpha, beta or gamma position to the functional group F³, more preferably in alpha or beta position, most preferably in alpha position. It was surprisingly found that conjugates comprising such linkages between the hydroxyalkyl starch and the cytotoxic agent show advantageous properties when used in mammals.

Without wanting to be bound to any theory, it is believed that a reason for the advantageous properties which are provided by the presence of these electron-withdrawing groups in close proximity to the functional group F³ may be an advantageous influence on the release rate of the cytotoxic agent comprised in the conjugate in the plasma of a mammal. The term “advantageous influence on the release rate” as used herein shall describe an influence allowing for a release rate which generates suitable amounts of the cytotoxic agent in a suitable time period so that therapeutic levels of the cytotoxic agent are delivered prior to excretion of the conjugate or conjugate fragments through the kidney or inactivation of the cytotoxic agent comprised in the conjugate by alternative mechanisms in the body. The term “suitable amounts” as used in this context of the present invention shall describe an amount with which the desired therapeutic effect of the cytotoxic agent is achieved, preferably together with a toxicity of the cytotoxic agent as low as possible. Without wanting to be bound to any theory, it is believed that the higher the tendency of the electron-withdrawing group to attract valence electrons, the faster the cytotoxic agent is released in vivo. Thus, it is assumed that the release rates can, inter alia, be tailored to specific needs by choosing a suitable electron-withdrawing group in alpha, beta or gamma position relative to the functional group F³.

Therefore, the present invention also relates to a conjugate, as described above, comprising an electron-withdrawing group in alpha, beta or gamma position, preferably in alpha or beta position, in particular in alpha position to each functional group F³. Further, the present invention also relates to a conjugate comprising an electron-withdrawing group in alpha, beta or gamma position, preferably in alpha or beta position, in particular in alpha position to each functional group F³, obtained or obtainable by the method as described above.

The electron-withdrawing group may be either part of the linking moiety L¹ or, according to an alternative embodiment, may be present in the hydroxyalkyl starch derivative, provided that the electron-withdrawing group is present in close proximity to the functional group F³, as described above. The term “present in close proximity to”, as used in the context of the present invention, is preferably denoted to mean a group which is present in alpha, beta, or gamma position to the functional group F³. More preferably the electron-withdrawing group is present in alpha, beta or gamma position, as described above.

Preferably, the electron-withdrawing group is a moiety selected from the group consisting of —O—, —S—, —SO—, —SO₂—, —NR^{e}—, —C(═Y^{e})—, —NR^{e}—C(═Y^{e})—, —C(═Y^{e})—NR^{e}—, —NO₂ comprising groups such as —CH(NO₂)—, —CN comprising groups such as —CH(CN)—, aryl groups, heteroaryl groups, cyclic imide groups and at least partially fluorinated alkyl moieties, wherein Y^{e} is either O, S or NR^{e}, and wherein R^{c} is one of hydrogen, alkyl, aryl, arylalkyl, heteroaryl, alkylaryl, alkylheteroaryl or heteroarylalkyl group, and the like.

Within the meaning of the present invention, the term “at least partially fluorinated alkyl moiety” refers to, optionally substituted, alkyl groups, such as non-branched alkyl residues, branched alkyl residues, cycloalkyl residues, as well as residues comprising one or more heteroatoms or functional groups, such as, by way of example, —O—, —S—, —NH—, —NH—C(═O), —C(═O)—NH, and the like, having at least one of the hydrogen atoms replaced with a fluorine atom. In some fluorinated alkyl groups, all the hydrogen atoms are replaced with fluorine atoms, i.e., the fluorinated alkyl group is a perfluoroalkyl group. The following groups are mentioned, by way of example: —CH₂F, CF₃, —CF₂—, —CHF—, —CH₂—CF₃, —CH₂—CHF₂ and —CH₂—CH₂F.

Within the context of the present invention, the term “cyclic imide groups” is denoted to mean a cyclic structural unit according to the general formula

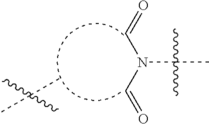

wherein the ring structure is preferably a 5-membered ring, 6-membered ring or 7-membered ring. Most preferably the cyclic imide is a -succinimide- having the following structure

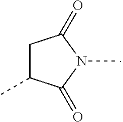

Preferably the electron-withdrawing group is selected from the group consisting of NH—C(═O)—, —C(═O)—NH—, —NH—, —O—, —S—, —SO—, —SO₂— and -succinimide-. More preferably the electron-withdrawing group is selected from the group consisting of —C(═O)—NH—, —NH—, —O—, —S—, —SO₂— and -succinimide-.

Thus, the present invention also relates to a conjugate, as described above, as well as a conjugate obtained or obtainable by the above-described method, wherein the conjugate comprises an electron-withdrawing group, preferably in alpha or beta position to each functional group F³, more particular in alpha position to each functional group F³, wherein the electron-withdrawing group is a group selected from the group consisting of —NH—C(═O)—, —C(═O)—NH—, —NH—, —O—, —S—, —SO—, —SO₂— and -succinimide-.

For certain preferred linker compounds incorporated in conjugates according to the present invention, it was clearly shown that the use of an electron-withdrawing group, preferably in alpha or beta position, more preferably in alpha position, has a significant influence on the release rates under physiological conditions (see example 1.4.15).

Surprisingly, in particular the groups —S— and —O— are believed to allow for a particularly advantageous influence on the release rate of the cytotoxic agent.

According to an alternative embodiment described by the present invention, the electron-withdrawing group is selected from the group consisting of —NH—C(═O), —C(═O)—NH—and —NH—.

According to a particularly preferred embodiment of the present invention, the linking moiety L¹ has a structure according to the following formula —[F²]_{q}-[L²]_{g}-[E]ₑ—[CR^{m}Rⁿ]_{f}—, wherein E is an electron-withdrawing group, L² is a linking moiety, F² is a functional group, f is 1, 2 or 3, g is 0 or 1, q is 0 or 1, e is 0 or 1, and wherein R^{m} and Rⁿ are, independently of each other, H or alkyl. Thus, the present invention also relates to a conjugate, as described above, wherein L¹ has a structure according to the following formula —[F²]_{q}-[L²]_{g}-[E]ₑ—[CR^{m}Rⁿ]_{f}—, the conjugate thus having the following formula HAS′(—[F²]_{q}-[L²]_{g}-[E]ₑ—[CR^{m}Rⁿ]_{f}—F³-M)ₙ.

According to the first preferred embodiment of the invention, an electron-withdrawing group E is present in the linking moiety L′. In this case, integer e is 1. Further, in this case, the electron-withdrawing group is preferably selected from the group as described above, most preferably E, is selected from the group consisting of —C(═O)—NH—, —NH—C(═O)—, —NH—, —O—, —S—, —SO—, —SO₂— and -succinimide-, more preferably E, is selected from the group consisting of —C(═O)—NH—, —O—, —S— and -succinimide-. According to this embodiment, the following conjugate structures are thus particularly preferred: HAS′(—[F²]_{q}-[L²]_{g}-C(═O)—NH—[CR^{m}Rⁿ]_{f}—F³-M)ₙ, HAS′(—[F²]_{q}-[L²]_{g}-NH—[CR^{m}Rⁿ]_{f}—F³-M)ₙ, HAS′(—[F²]_{q}-[L²]_{g}—O—[CR^{m}Rⁿ]_{f}—F³-M)ₙ, HAS′(—[F²]_{q}-[L²]_{g}—S—[CR^{m}Rⁿ]_{f}—F³-M)ₙ, HAS′(—[F²]_{q}-[L²]_{g}-succinimide-[CR^{m}Rⁿ]_{f}—F³-M)ₙ. More preferably, the electron-withdrawing group E is selected from the group consisting of —C(═O)—NH—, —NH—, —O—, —S—, and -succinimide- and the functional group F³ is a —C(═Y)— group, the hydroxyalkyl starch conjugate thus having preferably a structure selected from the group consisting of HAS′(—[F²]_{q}—[L²]_{g}—C(═O)—NH-[CR^{m}Rⁿ]_{f}—C(═Y)-M)ₙ, HAS′(—[F²]_{q}-[L²]_{g}—NH—[CR^{m}Rⁿ]_{f}—C(═Y)-Mₙ, HAS′(—[F²]_{q}-[L²]_{g}-O—[CR^{m}Rⁿ]_{f}—C(═Y)-M)ₙ, HAS′(—[F²]_{q}-[L²]_{g}—S—[CR^{m}Rⁿ]_{f}—C(═Y)-M)ₙ, HAS′(—[F²]_{q}-[L²]_{g}-succinimide-[CR^{m}Rⁿ]_{f}—C(═Y)-M)ₙ, wherein Y is preferably selected from O or S, in particular wherein Y is O.

Even more preferably E is —S— or —O—. Thus, the hydroxyalkyl starch conjugate more preferably has a structure selected from the group consisting of HAS′(—[F²]_{q}-[L²]_{g}-O—[CR^{m}Rⁿ]_{f}—C(═O)-M)ₙ, HAS′(—[F²]_{q}-[L²]_{g}—S—[CR^{m}Rⁿ]_{f}—C(═O)-M)ₙ, HAS′(—[F²]_{q}-[L²]_{g}-O—[CR^{m}Rⁿ]_{f}—C(═S)-M)ₙ, HAS′(—[F²]_{q}-[L²]_{g}—S—[CR^{m}Rⁿ]_{f}—C(═S)-M)ₙ, more preferably the structure HAS′(—[F²]_{q}-[L²]_{g}—O—[CR^{m}Rⁿ]_{f}—C(═O)-M)ₙ or the structure HAS′(—[F²]_{q}-[L²]_{g}—S—[CR^{m}Rⁿ]_{f}—C(═O)-M)ₙ.

According to an alternative preferred embodiment the functional group F² is an electron-withdrawing group present in close proximity to the functional group F³. In this case, F² may for example be a group such as a —C(═O)—NH—, —NH—, —O—, —S— or -succinimide- group.

In case F² is an electron-withdrawing group present in close proximity to the functional group F³, that is in alpha, beta or gamma position to the functional group F³, F² may be present instead of E or in addition to E.

According to this embodiment, the following conjugate structures are thus particularly preferred: HAS′(—C(═O)—NH-[L²]_{g}-[E]ₑ—[CR^{m}Rⁿ]_{f}—F³-M)ₙ, HAS′(—NH-[L²]_{g}-[E]ₑ-[CR^{m}Rⁿ]_{f}—F³-M)ₙ, HAS′(—O-[L²]_{g}-[E]ₑ—[CR^{m}Rⁿ]_{f}—F³-M)ₙ, HAS′(—S—[L²]_{g}-[E]ₑ—[CR^{m}Rⁿ]_{f} F³-M)ₙ and HAS′(-succinimide-[L²]_{g}-[E]ₑ—[CR^{m}Rⁿ]_{f}—F³-M)ₙ, more preferably a structure selected from the group consisting of HAS′ (—C(═O)—NH-[L²]_{g}-[E]ₑ—[CR^{m}Rⁿ]_{f}—C(═Y)-M)ₙ, HAS′(—NH-[L²]_{g}-[E]ₑ—[CR^{m}Rⁿ]_{f}—C(═Y)-M)ₙ, HAS′(—S—-[L²]_{g}-[E]ₑ—[CR^{m}Rⁿ]_{f}—C(═Y)-M)ₙ, HAS′(—O-[L²]_{g}-[E]ₑ[CR^{m}Rⁿ]_{f}—C(═Y)-M)ₙ and HAS′ (-succinimide-[L²]_{g}[E]ₑ—[CR^{m}Rⁿ]_{f}—C(═Y)-M)ₙ, wherein Y is preferably selected from O or S, in particular wherein Y is O.

According to an alternative embodiment, the electron-withdrawing group, if present in the linking moiety L¹ may also be present in the linking moiety L².

Further, the electron-withdrawing group, if present, may also be present in the structural unit [CR^{m}Rⁿ]. It is recalled that integer f of the structural unit [CR^{m}Rⁿ]_{f}, is preferably in the range of from 1 to 3 and R^{m} and Rⁿ are, independently of each other, H or alkyl. Since the term “alkyl” as used in the context of the present invention also encompasses alkyl groups which are further substituted, the electron withdrawing group may also be present in at least one of R^{m} or Rⁿ, such as, e.g. in the form of a —CH₂F, —CHF₂ or —CF₃ group or the like.

According to a further preferred embodiment of the present invention, the electron-withdrawing group, if present, is not present in the linking moiety L¹ but is instead part of the hydroxyalkyl starch derivative (HAS′). In this case e is 0 and the integer q, g and f are chosen so that the electron-withdrawing group is preferably present in the hydroxyalkyl starch derivative in a position being in close proximity to the functional group F³, as described above, preferably in alpha or beta position to the functional group F³.

### Linking Moiety L²

In general, there are no particular restrictions as to the chemical nature of the linking moiety L². The term “linking moiety L²” as used in the context of the present application, relates to any suitable chemical moiety bridging F² and E, in case q and e are 1, or bridging F² and the structural unit CR^{m}Rⁿ in case q is 1, e is 0 and f is 1, 2 or 3, or bridging E and the hydroxyalkyl starch derivative in case q is 0 and e is 1.

Preferably, L² is an alkyl group comprising 1 to 20, preferably 1 to 10, more preferably 1 to 8, more preferably 1 to 6, such as 1, 2, 3, 4, 5 or 6, more preferably 1 to 4, more preferably from 1 to 3, and most preferably from 2 to 3 carbon atoms. According to the definition of the term “alkyl”, the above mentioned alkyl groups may be substituted.

Preferably, L² comprises at least one structural unit according to the following formula

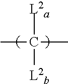

wherein L²ₐ and L²_{b} are independently from each other H or an organic residue selected from the group consisting of alkyl, alkenyl, alkylaryl, arylalkyl, aryl, heteroaryl, alkylheteroaryl, heteroarylalkyl, hydroxyl and halogen, such as fluorine, chlorine, bromine, or iodine.

More preferably, L² has a structure according to the following formula

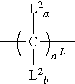

with L²ₐ and L²_{b} being selected from the group consisting of H, methyl or hydroxyl, with n^{L} being preferably in the range of from 1 to 8, more preferably in the range of from 1 to 6, more preferably in the range of from 1 to 4, more preferably in the range of from 1 to 3, and most preferably in the range of from 2 to 3. According to an even more preferred embodiment, the spacer L² consists of the structural unit according to the following formula

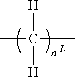

wherein integer n^{L} is in the range of from 1 to 8, more preferably in the range of from 1 to 6, more preferably in the range of from 1 to 4, more preferably in the range of from 1 to 3, and most preferably in the range of from 2 to 3. Therefore, according to a preferred embodiment of the present invention, L² has a structure selected from the group consisting of —CH₂—CH₂—CH₂—CH₂—CH₂—CH₂—, —CH₂—CH₂—CH₂—CH₂—CH₂—, —CH₂—CH₂—CH₂—CH₂—, —CH₂—CH₂—CH₂—, —CH₂—CH₂—, —CH₂—, more preferably L² is selected from the group consisting of —CH₂—, —CH₂—CH₂—, —CH₂—CH₂—CH₂—.

According to one preferred embodiment of the present invention, the present invention also relates to a conjugate, as described above, as well as a conjugate obtained or obtainable by the above-mentioned method, wherein the conjugate has a structure selected from the group consisting of the following formulas HAS′(—[F²]_{q}-[CH₂]_{g}-[E]ₑ—[CR^{m}Rⁿ]_{f}—F³-M)ₙ, HAS′(—[F²]_{q}-[CH₂—CH₂]_{g}-[E]ₑ—[CR^{m}Rⁿ]_{f}—F³-M)ₙ, HAS′(—[F²]_{q}-[CH₂—CH₂—CH₂]_{g}-[E]ₑ-[CR^{m}Rⁿ]_{f}—F³-M)ₙ, HAS′(—[F²]_{q}-[CH₂—CH₂—CH₂—CH₂]_{g}-[E]ₑ—[CR^{m}Rⁿ]_{f}—F³-M)ₙ, HAS′(—[F²]_{q}-[CH₂—CH₂—CH₂—CH₂—CH₂]_{g}-[E]ₑ—[CR^{m}Rⁿ]_{f}—F³-M)ₙ, HAS′(—[F²]_{q}-[CH₂—CH₂—CH₂—CH₂—CH₂—CH₂]_{g}-[E]ₑ—[CR^{m}Rⁿ]_{f}—F³-M)ₙ, more preferably the conjugate is selected from the following structures: HAS′(—[F²]_{q}-[CH₂]_{g}-[E]ₑ—[CR^{m}Rⁿ]_{f}—F³-M)ₙ, HAS′(—[F²]_{q}—[CH₂—CH₂]_{g}-[E]ₑ-[CR^{m}Rⁿ]_{f}—F³-M)ₙ and HAS′(—[F²]_{q}—[CH₂—CH₂—CH₂]_{g}-[E]ₑ—[CR^{m}Rⁿ]_{f}—F³-M), more preferably from the group consisting of HAS′(—[F²]_{q}—CH₂—[E]ₑ—[CR^{m}Rⁿ]_{f}—F³-M)ₙ, HAS′(—[F²]_{q}—CH₂—CH₂—[E]ₑ—[CR^{m}Rⁿ]_{f}—F³-M)ₙ and HAS′(—[F²]_{q}—CH₂—CH₂—CH₂—[E]ₑ—[CR^{m}Rⁿ]_{f}—F³-M)_{n.}

In case g is 1, the following most preferred combinations of group L² with the functional unit [E]ₑ, with e=1 are mentioned, by way of example: HAS′(—[F²]_{q}—CH₂—C(═O)—NH-[CR^{m}Rⁿ]_{f}—F³-M)ₙ, HAS′(—[F²]_{q}—CH₂—CH₂—C(═O)—NH—[CR^{m}R^{m}]_{f}—F³-M)ₙ and HAS′(—[F²]_{q}—CH₂—CH₂—CH₂—C(═O)—NH—[CR^{m}Rⁿ]_{f}—F³-M)ₙ, HAS′(—[F²]_{q}—CH₂—NH—[CR^{m}R^{n]}_{f}—F³-M)ₙ, HAS′(—[F²]_{q}—CH₂—CH₂—NH—[CR^{m}Rⁿ]_{f}—F³-M)ₙ and HAS′(—[F²]_{q}—CH₂—CH₂—CH₂—NH-[CR^{m}Rⁿ]_{f}—F³-M)ₙ, HAS′(—[F²]_{q}—CH₂—S—[CR^{m}Rⁿ]_{f}—F³-M)ₙ, HAS′(—[F²]_{q}—CH₂—CH₂—S-[CR^{m}Rⁿ]_{f}—F³-M)ₙ, HAS′(—[F²]_{q}—CH₂—CH₂—CH₂—S—[CR^{m}Rⁿ]_{f}—F³-M)ₙ, HAS′(—[F²]_{q}—CH₂—O—[CR^{m}Rⁿ]_{f}—F³-M)ₙ, HAS′(—[F²]_{q}—CH₂—CH₂—O—[CR^{m}Rⁿ]_{f}—F³-M)ₙ, HAS′(—[F²]_{q}—CH₂—CH₂—CH₂—O—[CR^{m}Rⁿ]_{f}—F³-M)ₙ, HAS′(—[F²]_{q}—CH₂-succinimide-[CR^{m}Rⁿ]ᵣ—F³-M)ₙ, HAS′(—[F²]_{q}—CH₂—CH₂-succinimide-[CR^{m}Rⁿ]_{f}—F³-M)ₙ, HAS′(—[F²]_{q}—CH₂—CH₂—CH₂-succinimide-[CR^{m}Rⁿ]_{f}—F³-M).

Most preferably g is 1, i.e. L² is present, and L² is —CH₂—CH₂— or —CH₂—CH₂—CH₂—.

### The Functional Group F²

The functional group F² is, if present, a functional group linking the hydroxyalkyl starch derivative with the linking moiety L², in case g is 1, or with the electron-withdrawing group E in case g is 0 and e is 1, or with the structure unit CR^{m}Rⁿ, in case g and e are 0.

There are, in general, no particular restrictions as regards the chemical nature of the functional group F² provided that a stable bond is formed linking the hydroxyalkyl starch derivative with L², E or the structural unit CR^{m}Rⁿ, respectively. The stable bond may also be a bond which is eventually cleaved in vivo. As described above, the functional group F² may serve as electron-withdrawing group in close proximity to the functional group F³ to provide an optimized hydrolysis rate of the linkage between F³ and the cytotoxic agent.

Preferably, F² is a group consisting of —Y¹—, —C(═Y²)—, —C(═Y²)—NR^{F2}—,

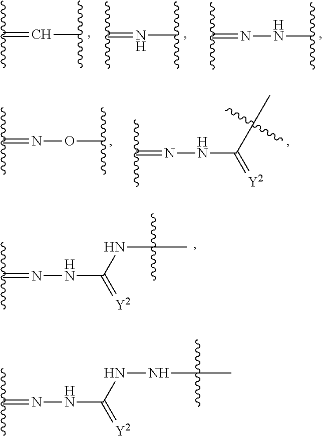

### and —CH₂—CH₂—C(═Y²)—NR^{F2}—,

wherein Y¹ is selected from the group consisting of —S—, —O—, —NH—, —NH—NH—, —CH₂—CH₂—SO₂—NR^{F2}—, —CH₂—CHOH—, and cyclic imides, such as succinimide, and wherein Y² is selected from the group consisting of NH, S and O, and wherein R^{F2} is selected from the group consisting of hydrogen, alkyl, alkylaryl, arylalkyl, aryl, heteroaryl, alkylheteroaryl or heteroarylalkyl group.

More preferably, F² is a group consisting of —Y¹—, —C(═Y²)—, —C(═Y²)—NR^{F2}—,

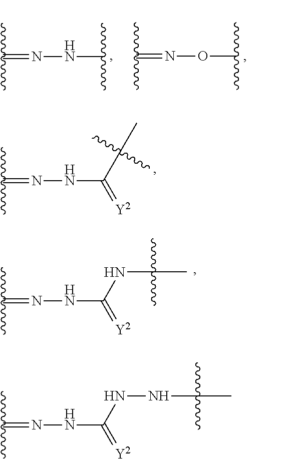

### and —CH₂—CH₂—C(═Y²)—NR^{F2}—.

Preferably, F² is selected from the group consisting of —S—, —NH—NH—, succinimide- and

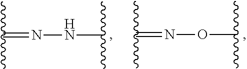

more preferably F² is succinimide- or —S—, most preferably succinimide-.

Thus, the present invention also relates to the conjugate as described above, the conjugate having a structure selected from the group consisting of HAS′(-[succinimide]_{q}-[L²]_{g}-[E]ₑ-[CR^{m}Rⁿ]_{f}—F³-M)ₙ and HAS′(—[S]_{q}-[L²]_{g}-[E]ₑ—[CR^{m}Rⁿ]_{f}—F³-M)ₙ, in particular, according to one preferred embodiment, according to which F² is present, i.e. integer q is 1, the conjugate has a structure selected from the group consisting of HAS′(-succinimide-[L²]_{g}-[E]ₑ[CR^{m}Rⁿ]_{f}—F³-M)ₙ and HAS′(—S[L²]_{g}-[E]ₑ—[CR^{m}Rⁿ]_{f}—F³-M)ₙ, more preferably HAS′(-succinimide-[L²]_{g}-[E]ₑ—[CR^{m}Rⁿ]_{f}—F³-M)ₙ.

Furthermore, the functional group F² may form together with a functional group of the hydroxyalkyl starch a 1,2,3-triazole ring. In the event that the functional F² forms together with a functional group of the hydroxyalkyl starch derivative a 1,2,3-triazole, inter alia, the following structures are conceivable for this structural building block

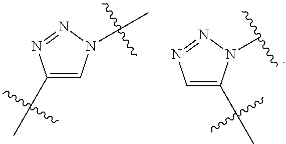 [SMILES: *c1cn(*)nn1 ; *c1cnnn1*]

In case the conjugate comprises a triazole linking group, preferably the functional group F² forms together with the functional group X present in the residue of the hydroxyalkyl starch derivative a 1,2,3-triazole. Preferably such a triazole group is formed via a 1,3-dipolar cycloaddition between an azide and a terminal or internal alkynyl group to give a 1,2,3-triazole. For example in case Z¹ is an alkynyl group or azide and the crosslinking compound L bears a functional group K² being the respective azide or alkynyl, a triazole linkage may be formed when linking L to the hydroxyalkyl starch derivative.

### The Structural Unit [CR^{m}Rⁿ]_{f}

As regards the structural unit [CR^{m}Rⁿ]_{f}, integer f is preferably in the range of from 1 to 3 and R^{m} and Rⁿ are, independently of each other, H, alkyl or aryl, more preferably H or alkyl. In case integer f is greater than 1, each repeating unit [CR^{m}Rⁿ] may be the same or may be different from each other.

More preferably, integer f is 1 or 2, most preferably 1.

As described above the term “alkyl” relates to non-branched alkyl residues, branched alkyl residues, cycloalkyl residues, as well as residues comprising one or more heteroatoms or functional groups, such as, by way of example, —O—, —S—, —NH—, —NH—C(═O), —C(═O)—NH, and the like. These residues may be further substituted by one or more suitable substituents. Preferably, R^{m} and Rⁿ are, independently of each other, H or an unsubstituted alkyl group.

In case integer f is 2 or 3, each repeating unit [CR^{m}Rⁿ] may be the same or may be different from each other.

Preferably, R^{m} and Rⁿ are, independently of each other, selected from H or branched or linear alkyl chains, comprising 1 to 10, preferably 1 to 8, more preferably 1 to 5, most preferably 1 to 3 carbon atoms. More preferably R^{m} and Rⁿ are, independently of each other, selected from the group consisting of H, methyl, ethyl, propyl, butyl, sec-butyl and tert-butyl, more preferably R^{m} and Rⁿ are, independently of each other, H or methyl.

By way of example, the following preferred structures for the structural unit [CR^{m}Rⁿ]_{f} are mentioned: CH₂—CH₂—CH₂—, CH₂—CH₂—, CH₂—, —CH(CH₃)—, —C(CH₃)₂—, —CH(CH₂—CH₃)—, —CH(CH(CH₃)₂)—, —CH(CH₃)—CH₂—, —CH₂—CH(CH₃)—, —CH(CH₃)—CH₂—CH₂—, —CH₂—CH(CH₃)—CH₂—, —CH₂—CH₂—CH(CH₃)—, —CH(CH₃)—CH(CH₃)—, CH(CH₃)—CH(CH₃)—CH₂—, —CH₂—CH(CH₃)—CH(CH₃)—, —CH(CH₃)—CH₂—CH(CH₃)—.

According to a particularly preferred embodiment of the present invention, R^{m} and Rⁿ are both H. The structural unit [CR^{m}Rⁿ]_{f} is thus preferably —CH₂—CH₂—CH₂—, CH₂—CH₂— or CH₂—, more preferably f is 1 or 2, the structural unit [CR^{m}Rⁿ]_{f} thus preferably having the structure CH₂—CH₂— or CH₂—.

Thus, the present invention also relates to the conjugate as described above, the conjugate having a structure selected from the group consisting of HAS′(—[F²]_{q}-[L²]_{g}-[E]ₑ—CH₂—CH₂—CH₂—F³-M)ₙ, HAS′(—[F²]_{q}[L²]_{g}-[E]ₑ—CH₂—CH₂—F³-M)ₙ and HAS′(—[F²]_{q}-[L²]_{g}-[E]ₑ—CH₂—F³-M)ₙ, more preferably HAS′(—[F²]_{q}-[L²]_{g}-[E]ₑ—CH₂—CH₂—F³-M)ₙ and HAS′(—[F²]_{q}-[L²]_{g}-[E]ₑ—CH₂—F³-M)ₙ, more preferably HAS′(—[F²]_{q}-[L²]_{g}-[E]ₑ—CH₂—F³-M)ₙ.

### Examples of Preferred Linking Moieties L

By way of example, the following linking moieties L are mentioned:

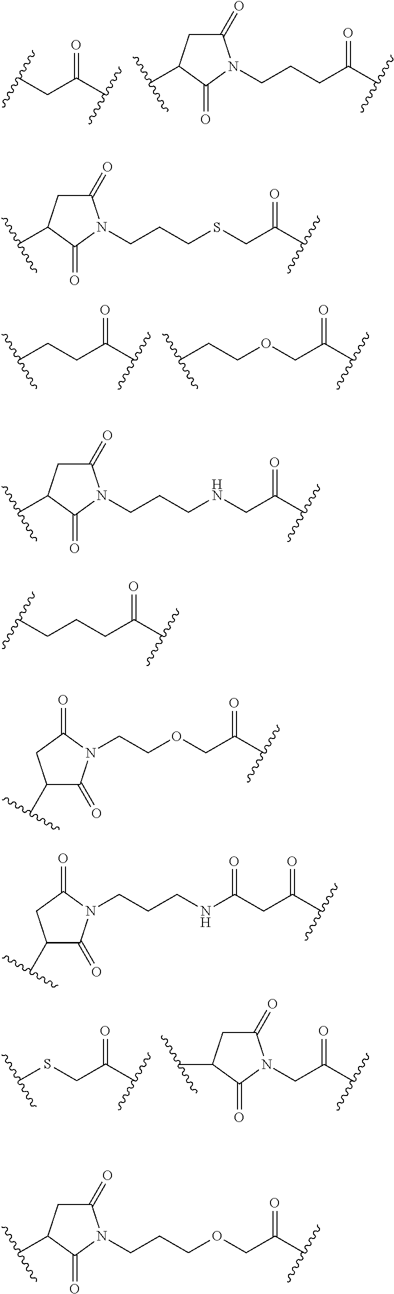

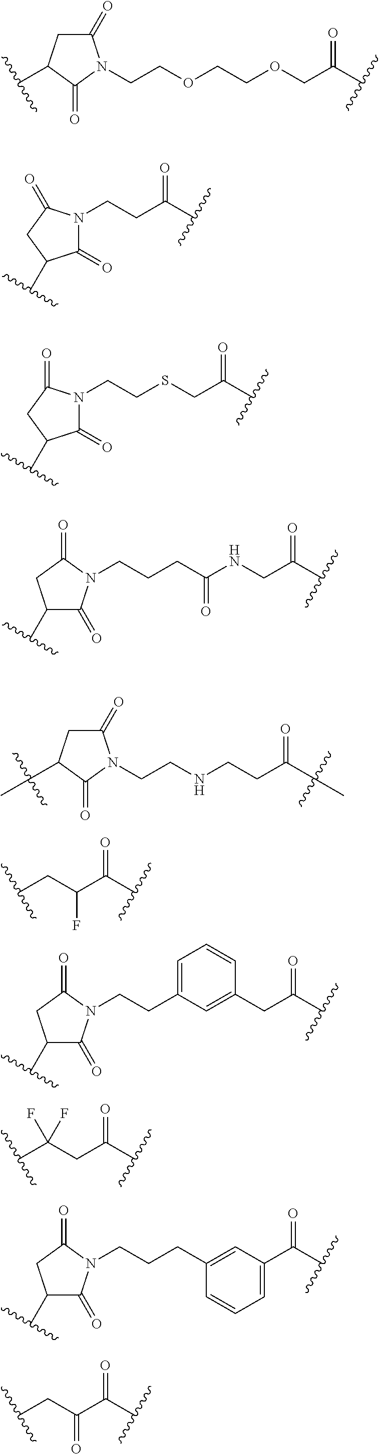

According to the invention the at least one structural unit -L-M is linked via L to a hydroxyalkyl starch derivative thereby forming a linkage between HAS′ and the at least one structural unit L-M.

### The Residue of the Hydroxyalkyl Starch Derivative Comprised in the Conjugate

In accordance with the above-mentioned definition of HAS, the residue of the hydroxyalkyl starch derivative preferably comprises at least one structural unit according to the following formula (I)

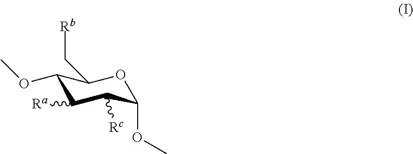

wherein at least one of R^{a}, R^{b} or R^{c} comprises the functional group —X— and wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—X—, —[O—(CR^{w}R^{x})—(CR^{y}R^{z})]_{y}-[F¹]ₚ-L¹-X—, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety and —X— is a functional group linking the hydroxyalkyl starch derivative and the linking moiety L. Preferably X is formed upon reaction of Z¹ with the crosslinking compound L, HAS″ is a remainder of the hydroxyalkyl starch derivative, as described above. According to a preferred embodiment of the present invention, the hydroxyalkyl starch derivative is a hydroxyethyl starch derivative.

The amount of functional groups X present in the residue of the hydroxyalkyl starch derivative being incorporated into the conjugate of the invention corresponds to the amount of functional groups Z¹ present in the corresponding hydroxyalkyl starch derivative prior to the conjugation of said derivative to the crosslinking compound L or the structural unit L-M. Thus, preferably 0.15% to 2% of all residues R^{a}, R^{b} and R^{c} present in the hydroxyalkyl starch derivative contain the functional group X. More preferably, 0.15% to 2% of all residues R^{a}, R^{b} and R^{c} present in the hydroxyalkyl starch derivative have the structure —[O—(CR^{w}R^{x})—(CR^{y}R^{z})]_{y}—X— or —[O—(CR^{w}R^{x})—(CR^{y}R^{z})]_{y}-[F¹]ₚ-L¹-X—. According to a particularly preferred embodiment, R^{a}, R^{b} and R^{c} are selected from the group consisting of —O—HAS″, —[O—(CR^{w}R^{x})—(CR^{y}R^{z})]ₓ—OH and —[O—(CR^{w}R^{x})—(CR^{y}R^{z}]_{y}—X—, wherein 0.15% to 2% of all residues R^{a}, R^{b} and R^{c} present in the hydroxyalkyl starch derivative have the structure —[O—(CR^{w}R^{x})—(CR^{y}R^{z})]_{y}—X—. According to an alternative preferred embodiment, R^{a}, R^{b} and R^{c} are selected from the group consisting of —O—HAS″, —[O—(CR^{w}R^{x})—(CR^{y}R^{z})]ₓ—OH and —[O—(CR^{w}R^{x})—(CR^{y}R^{z})]_{y}-[F¹]ₚ-L¹-X—, wherein 0.15% to 2% of all residues R^{a}, R^{b} and R^{c} present in the hydroxyalkyl starch derivative have the structure —[O—(CR^{w}R^{x})—(CR^{y}R^{z})]_{y}-[F¹]ₚ-L¹-X—.

Preferably, the present invention also describes a conjugate, comprising a residue of a hydroxyalkyl starch derivative, as described above, as well as a conjugate obtained or obtainable by the above-mentioned method, wherein the conjugate comprises a residue of a hydroxyethyl starch derivative and a cytotoxic agent, the residue of the HES derivative preferably comprises at least one structural unit, preferably 3 to 200 structural units, according to the following formula (I)

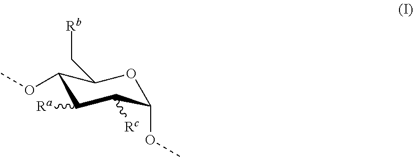

wherein R^{a}, R^{b} and R^{c} are independently of each other selected from the group consisting of —O—HAS″, —[O—CH₂—CH₂]_{S}—OH, —[O—CH₂—CH₂]ₜ—X— and —[O—CH₂—CH₂]ₜ-[F¹]ₚ-L¹-X—, wherein t is in the range of from 0 to 4, and wherein s is in the range of from 0 to 4, p being 0 or 1, and wherein at least one of R^{a}, R^{b} and R^{c} comprises the functional group X, and wherein preferably 0.15% to 2% of all residues R^{a}, R^{b} and R^{c} present in the hydroxyalkyl starch derivative comprise the functional group —X— and wherein X is linked to the linking moiety L comprised in the conjugate of the present invention.

According to a preferred embodiment of the present invention, this linkage between X and L is obtained by coupling a hydroxyalkyl starch derivative being functionalized with at least one functional group Z¹, as described above, to the crosslinking compound L, thereby obtaining a covalent linkage between HAS′ and L, wherein the residue of the hydroxyalkyl starch is linked via the functional group X to the linking moiety L. Further preferred embodiments as to this method are described below.

### The Functional Group X

X is a functional group linking the hydroxyalkyl starch derivative with the linking moiety L, wherein L is preferably -L′-F³—, and wherein more preferably L¹ is —[F²]_{q}-[L²]_{g}-[E]ₑ-[CR^{m}Rⁿ]_{f}—. Thus, X is a linking group preferably linking the hydroxyalkyl starch derivative with the functional group F² in case q is 1, or with the linking moiety L² in case q is 0 and g is 1, or with the electron-withdrawing group E in case q and g are 0 and e is 1, or with the structural unit —[CR^{m}Rⁿ]_{f}— in case q, g, e are 0 and f is 1.

In general, there exists no limitation regarding the functional group X provided that the functional group X is able to link the hydroxyalkyl starch derivative with the linking moiety L. According to a preferred embodiment of the present invention, and depending on the respective group of the linking moiety L being linked to X, X is selected from the group consisting of —Y^{xx}—, —C(═Y^{x})—, C(═Y^{x})—NR^{xx}—, —CH₂—CH₂—C(═Y^{x})—NR^{xx}—,

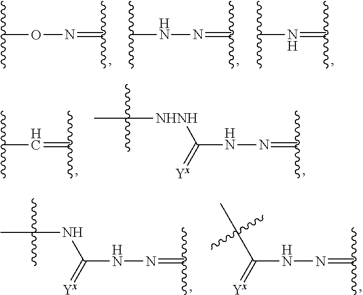

wherein Y^{xx} is selected from the group consisting of —S—, —O—, —NH—, —NH—NH—, —CH₂—CH₂—SO₂—NR^{xx}—, and cyclic imides, such as succinimide, and wherein Y^{x} is selected from the group consisting of NH, S and O, and wherein R^{xx} is selected from the group consisting of hydrogen, alkyl, alkylaryl, arylalkyl, aryl, heteroaryl, alkylheteroaryl or heteroarylalkyl group.

Furthermore, the functional group X may form together with a functional group of the linking moiety L, such as with the functional group F², a 1,2,3-triazole ring, as described hereinabove.

More preferably X is selected from the group consisting of —Y^{xx}—, —C(═Y^{x})—, —C(═Y^{x})—NR^{xx}—, —CH₂—CH₂—C(═Y^{x})—NR^{xx}—,

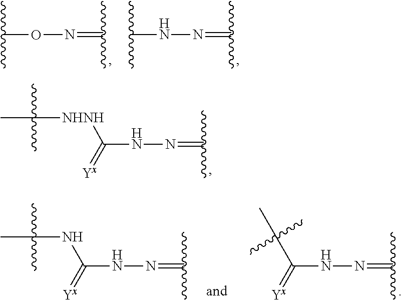

Most preferably X is selected from the group consisting of —O—, —S—, —NH— and —NH—NH—, more preferably —O—, —S— or —NH—. Most preferably X is —S—.

Therefore, the present invention also describes a conjugate, comprising a residue of a hydroxyalkyl starch derivative, as described above, as well as a conjugate obtained or obtainable by the above-mentioned method, wherein the conjugate comprises a residue of a hydroxyalkyl starch derivative and a cytotoxic agent, the residue of the hydroxyalkyl starch derivative preferably comprises at least one structural unit, preferably 3 to 200 structural units, according to the following formula (I)

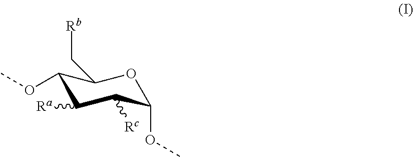

wherein at least one of R^{a}, R^{b} and R^{c} is —[O—(CR^{w}R^{x})—(CR^{y}R^{z})]_{y}—S— or —[O—(CR^{w}R^{x})—(CR^{y}R^{z})]_{y}-[F¹]ₚ-L¹-S—, preferably wherein at least one of R^{a}, R^{b} and R^{c} is —[O—CH₂—CH₂]ₜ—S— or —[O—CH₂—CH₂]ₜ-[F¹]ₚ-L¹-S—.

According to one preferred embodiment of the present invention, at least one of R^{a}, R^{b} and R^{c} is —[O—CH₂—CH₂]ₜ—S—. Thus, the following hydroxyalkyl starch derivatives may be mentioned as preferred embodiments of the invention:

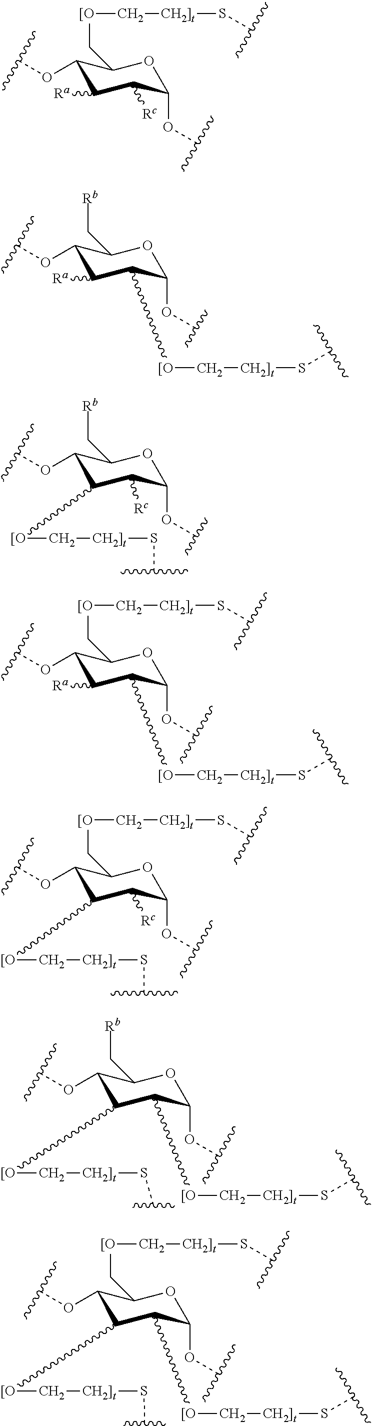

According to another preferred embodiment of the present invention, at least one of R^{a}, R^{b} and R^{c} is —[O—CH₂—CH₂]ₜ-[F¹]ₚ-L¹-S—. Thus, the following hydroxyalkyl starch derivatives may be mentioned as preferred embodiments of the invention:

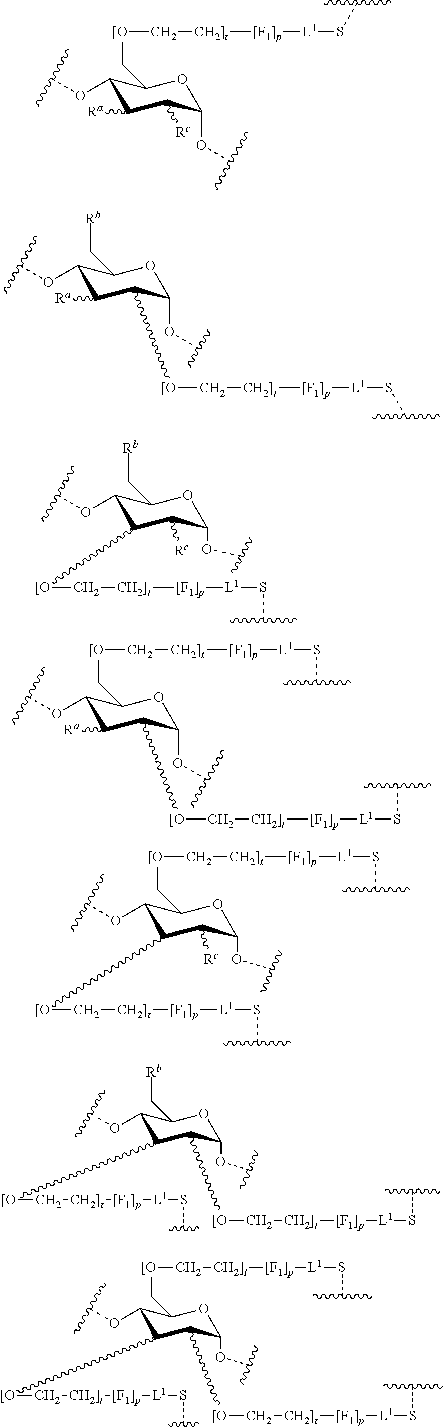

According to a preferred embodiment of the invention, the linking moiety L is directly linked to the functional group X of the hydroxyalkyl starch derivative and, on the other side, directly linked to a secondary hydroxyl group of the cytotoxic agent. According to a preferred embodiment wherein the cytotoxic agent is docetaxel or paclitaxel, the conjugate of the invention has a structure according to the following formula:

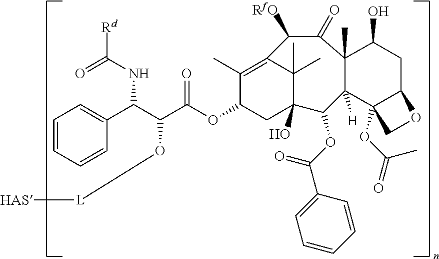

wherein R^{d} is preferably phenyl or O-t-butyl, and wherein R^{f} is preferably H or acetyl and wherein HAS′ comprises at least one structural unit according to the following formula (I)

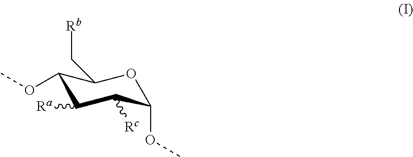

wherein at least one of R^{a}, R^{b} and R^{c} is —[O(CR^{w}R^{x})—(CR^{y}R^{z})]_{y}—S— or —[O—(CR^{w}R^{x})—(CR^{y}R^{z})]_{y}-[F¹]ₚ-L¹-S—, preferably wherein at least one of R^{a}, R^{b} and R^{c} is —[O—CH₂—CH₂]ₜ—S— or —-[O—CH₂—CH₂]ₜ-[F¹]ₚ-L¹-S— and wherein L is linked to the functional group —S—.

### The Functional Group F¹

F¹ is a functional group, which, if present, is preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, —NH—NH—, —NH—O—, —CH═N—O—, —O—N═CH—, —CH═N—, —N═CH— and cyclic imides, such as -succinimide, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H.

According to a preferred embodiment of the present invention the functional group F¹ is, if present, selected from the group consisting of —NH—, —O—, —S—, —NH—C(═O)—, —NH—C(═S)—, —O—C(═O)—NH—, —O—C(═O)—, —C(═O)—, —NH—C(═O)—NH—, —NH—NH—C(═O)—, —C(═O)—NH—NH—, —NH—C(═O)—NH—NH—, more preferably F¹ is, if present, —O— or —O—C(═O)—NH—.

Therefore, the present invention also describes a conjugate, comprising a hydroxyalkyl starch derivative, as described above, as well as a conjugate obtained or obtainable by the above-mentioned method, the hydroxyalkyl starch derivative preferably comprising at least one structural unit, preferably 3 to 200 structural units, according to the following formula (I)

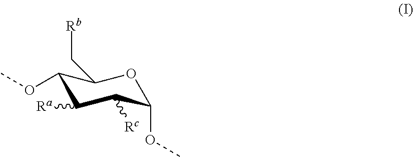

wherein at least one of R^{a}, R^{b} and R^{c} is —[O—(CR^{w}R^{x})—(CR^{y}R^{z})]_{y}—X— or —[O—(CR^{w}R^{x})—(CR^{y}R^{z})]_{y}-[F¹]ₚ-L¹-X—, preferably wherein at least one of R^{a}, R^{b} and R^{c} is —[O—CH₂—CH₂]ₜ—X— or —[O—CH₂—CH₂]ₜ-[F¹]ₚ-L¹-X—, more preferably wherein at least one of R^{a}, R^{b} and R^{c} is —[O—CH₂—CH₂]ₜ—S— or —[O—CH₂—CH₂]ₜ-[F¹]ₚ-L¹-S—, wherein F¹, if present, is preferably O— or —O—C(═O)—NH—.

Thus, the following preferred conjugates are described, which comprise a hydroxyalkyl starch derivative, as described above, wherein the hydroxyalkyl starch derivative comprises at least 1, preferably at least 3 to 200, structural units according to the following formula (I)

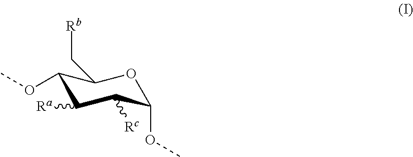

wherein at least one of R^{a}, R^{b} and R^{c} is
(i) —[O—CH₂—CH₂]ₜ—X— or
(ii) —[O—CH₂—CH₂]ₜ-[F¹]ₚ-L¹-X—, preferably with p being 1 and F¹ being —O—, or
(iii) —[O—CH₂—CH₂]ₜ-[F¹]ₚ-L¹-X—, preferably with p being 1 and F¹ being —O—C(═O)—NH—,
   wherein —X— is —S—, and wherein t is in the range of from 0 to 4, and wherein the linking moiety L of the structural unit -L-M is directly linked to at least one group X, preferably wherein all groups X present in the hydroxyalkyl starch derivative are linked to the structural unit -L-M, and wherein the linking moiety L is being attached to the group —O— of M derived from the secondary hydroxyl group of the cytotoxic agent.

### The Linking Moiety L¹

The term “linking moiety L¹” as used in this context of the present invention relates to any suitable chemical moiety bridging X with the functional group F¹ or the building block —[O—(CR^{w}R^{x})—(CR^{y}R^{z})]_{y}- or the sugar backbone of the hydroxyalkyl starch derivative.

In general, there are no particular restrictions as to the chemical nature of the spacer L¹ with the proviso that L¹ provides for a stable linkage between the functional group —X— and the hydroxyalkyl starch building block. Preferably, L¹ is an alkyl, alkenyl, alkylaryl, arylalkyl, aryl, heteroaryl, alkylheteroaryl or heteroarylalkyl group. As described above, the terms alkyl, alkenyl alkylaryl, arylalkyl, aryl, heteroaryl, alkylheteroaryl or heteroarylalkyl group also encompass groups which are substituted by one or more suitable substituents.

According to a preferred embodiment of the present invention, the linking moiety L¹ is a spacer comprising at least one structural unit according to the following formula —{[CR^{d}R^{f}]ₕ-[F⁴]ᵤ—[CR^{dd}R^{ff}]_{z}}ₐₗₚₕₐ—, wherein F⁴ is a functional group, preferably selected from the group consisting of —S—, —O— and —NH—, preferably wherein F⁴ is —O— or —S—, more preferably wherein F⁴ is —S—. The integer h is preferably in the range of from 1 to 20, more preferably 1 to 10, such as 1, 2, 3, 4, 5, 6, 7, 8, 9 or 10, more preferably 1 to 5, most preferably 1 to 3. Integer z is in the range of from 0 to 20, more preferably from 0 to 10, such as 0, 1, 2, 3, 4, 5, 6, 7, 8, 9 or 10, more preferably 0 to 3, most preferably 0 to 2, such as 0, 1 or 2. Integer u is 0 or 1. Integer alpha is in the range of from 1 to 10, preferably 1 to 5, such as 1, 2, 3, 4, 5, more preferably 1 or 2. As regards residues R^{d}, R^{f}, R^{dd} and R^{ff}, these residues are, independently of each other, preferably selected from the group consisting of halogens, alkyl groups, H or hydroxyl groups. The repeating units of —[CR^{d}R^{f}]ₕ— may be the same or may be different. Likewise, the repeating units of —[CR^{dd}R^{ff}]_{Z}— may be the same or may be different. Likewise in case integer alpha is greater than 1, the groups F⁴ in each repeating unit may be the same or may be different. Further, in case alpha is greater than 1, integer h in each repeating unit may be the same or may be different, integer z in each repeating unit may be the same or may be different and integer u in each repeating unit may be the same or may be different. Thus, in case alpha is greater than 1, each repeating unit of [CR^{d}R^{f}]ₕ-[F⁴]ᵤ—[CR^{dd}R^{ff}]_{z}, may be the same or may be different. Most preferably, R^{d}, R^{f}, R^{dd} and R^{ff} are independently from each other H, alkyl or hydroxyl.

According to one embodiment of the present invention, u and z are 0, the linking moiety L¹ thus corresponds to the structural unit —[CR^{d}R^{f}]ₕ—.

According to an alternative embodiment of the present invention u is 1. According to this embodiment z is preferably greater than 0, preferably 1 or 2.

Thus, the following preferred structures for the linking moiety L¹ are mentioned, by way of example: —{[CR^{d}R^{f}]ₕ-[F⁴]ᵤ—[CR^{dd}R^{ff}]_{Z}}ₐₗₚₕₐ- and —[CR^{d}R^{f}]ₕ—.

Thus, by way of the example, the following linking moieties L¹ are mentioned:

### —CH₂—,

### —CH₂—CH₂—,

### —CH₂—CH₂—CH₂—,

### —CH₂—CH₂—CH₂—CH₂—,

### —CH₂—CH₂—CH₂—CH₂—CH₂—,

### —CH₂—CH₂—CH₂—S—CH₂—CH₂—,

### —CH₂—CH₂—S—CH₂—CH₂—,

### —CH₂—CH₂—O—CH₂—CH₂—,

### —CH₂—CH₂—O—CH₂—CH₂—O—CH₂—CH₂—,

### —CH₂—CHOH—CH₂—,

### —CH₂—CHOH—CH₂—S—CH₂—CH₂—,

### —CH₂—CHOH—CH₂—S—CH₂—CH₂—CH₂—,

### —CH₂—CHOH—CH₂—NH—CH₂—CH₂—,

### —CH₂—CHOH—CH₂—NH—CH₂—CH₂—CH₂—,

### —CH₂—CHOH—CH₂—O—CH₂—CHOH—CH₂—,

### —CH₂—CHOH—CH₂—O—CH₂—CHOH—CH₂—S—CH₂—CH₂—,

### —CH₂—CH(CH₂OH)— and

### —CH₂—CH(CH₂OH)—S—CH₂—CH₂—.

According to one preferred embodiment, R^{d}, R^{f} and, if present, R^{dd} and R^{ff} are preferably H or hydroxyl, more preferably, at least one of R^{d} and R^{f} of at least one repeating unit of —[CR^{d}R^{f}]ₕ— is —OH, wherein even more preferably, in this case, both R^{dd} and R^{ff} are H, if present. In particular, in this case, L¹ is selected from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂—, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—.

According to an alternative preferred embodiment, both residues R^{d} and R^{f} are H, and R^{dd} and R^{ff} are, if present, H as well. In particular, in this case, L¹ is selected from the group consisting of: —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—CH₂—O—CH₂—CH₂— and —CH₂—CH₂—O—CH₂—CH₂—.

Therefore, the present invention also describes a hydroxyalkyl starch derivative, and a hydroxyalkyl starch derivative obtained or obtainable by the above-described method, the hydroxyalkyl starch derivative comprising at least one structural unit, preferably 3 to 200 structural units, according to the following formula (I)

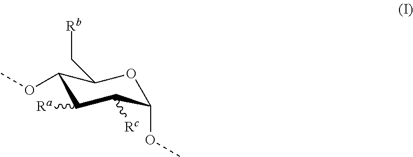

wherein at least one of R^{a}, R^{b} and R^{c} have a structure according to the following formula —[O—CH₂—CH₂]ₜ-[F¹]ₚ-L¹-X—, wherein L¹ is selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—O—CH₂—CH₂—, —CH₂—CH₂—O—CH₂—CH₂—O—CH₂—CH₂—, —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, —CH₂—CH(CH₂OH)— and —CH₂—CH(CH₂OH)—S—CH₂—CH₂—, more preferably from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—.

Further, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the hydroxyalkyl starch derivative preferably comprises at least one structural unit, preferably 3 to 200 structural units, according to the following formula (I)

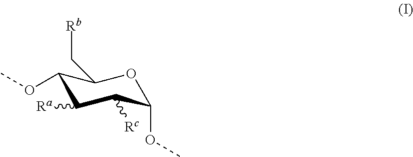

wherein at least one of R^{a}, R^{b} and R^{c} have a structure according to the following formula —[O—CH₂—CH₂]ₜ-[F¹]ₚ-L¹-X—, wherein L¹ is selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—O—CH₂—CH₂—, —CH₂—CH₂—O—CH₂—CH₂—O—CH₂—CH₂—, —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, —CH₂—CH(CH₂OH)— and —CH₂—CH(CH₂OH)—S—CH₂—CH₂—, more preferably from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂—, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—.

### Especially Preferred Conjugates According to the Present Invention

In the following, conjugate structures are mentioned, which comprise a particularly preferred combination of HAS′ and different structural units -L-M.

According to a first especially preferred embodiment of the present invention, a residue of hydroxyalkyl starch derivative comprising at least one structural unit, preferably 3 to 200 structural units, according to the following formula (I)

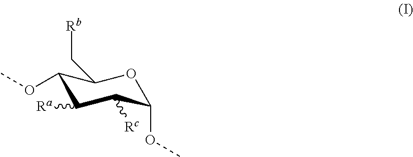

wherein at least one of R^{a}, R^{b} and R^{c} is —[O—CH₂—CH₂]ₜ—X— and X is —S—. This hydroxyalkyl starch derivative is according to this preferred embodiment of the invention, combined with the structural unit -L-M having the structure —[F²]_{q}-[L²]_{g}-[E]ₑ—[CR^{m}Rⁿ]_{f}—F³-M, wherein q is 0, g is 0 and e is 0.

Accordingly, in this preferred embodiment, the functional group X represents an electron-withdrawing group in close proximity to the functional group F³, and X is directly linked to the structural unit —[CR^{m}Rⁿ]_{f}—. Depending on integer f, which is 1, 2 or 3, the electron-withdrawing group is either present in alpha, beta or gamma position to the functional group F³.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(—[F²]_{q}-[L²]_{g}-[E]ₑ—[CR^{m}Rⁿ]_{f}—F³-M)ₙ

wherein q is 0, g is 0, e is 0, and wherein HAS′ preferably comprises at least one structural unit, preferably 3 to 200 structural units, according to the following formula (I)

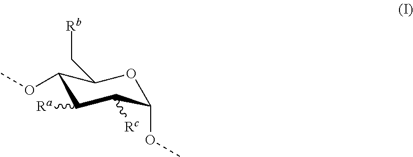

wherein, independently of each other, at least one of R^{a}, R^{b} and R^{c} is —[O—CH₂—CH₂]ₜ—X— and X is —S— and the functional group X is directly linked to the —[CR^{m}Rⁿ]_{f}— group, and wherein the hydroxyalkyl starch derivative comprises at least n functional groups X.

In case the electron-withdrawing group is —S—, this electron-withdrawing group is preferably present in alpha position to the functional group F³. Thus, according to this first preferred embodiment, according to which the functional group X represents the electron-withdrawing group, the integer f is preferably 1, so that X is present in alpha position to the functional group F³.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(—[F²]_{q}-[L²]_{g}-[E]ₑ—[CR^{m}Rⁿ]_{f}—F³-M)ₙ

wherein q is 0, g is 0, e is 0, wherein HAS′ preferably comprises at least one structural unit, preferably 3 to 200 structural units, according to the following formula (I)

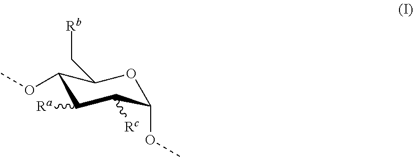

wherein at least one of R^{a}, R^{b} and R^{c} is —[O—CH₂—CH₂]ₜ—X— and X is —S— and the functional group X is directly linked to the —[CR^{m}Rⁿ]_{f}— group, and wherein the hydroxyalkyl starch derivative comprises at least n functional groups X, and wherein f is 1. R^{m} and Rⁿ are, independently of each other, H or alkyl. Most preferably R^{m} and Rⁿ are H.

Thus, according to this embodiment, the conjugate, or the conjugate obtained or obtainable by the above-mentioned method, preferably has a structure according to the following formula

HAS′(—CH₂—F³-M)ₙ

wherein HAS′ comprises at least one structural unit, preferably 3 to 200 structural units, according to the following formula (I)

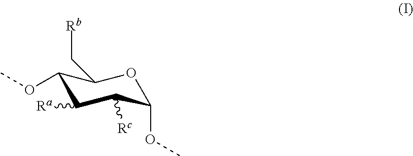

wherein at least one of R^{a}, R^{b} and R^{c} is —-[O—CH₂—CH₂]ₜ—X— and X is —S— and wherein the CH₂ group of the structural unit —(CH₂—F³-M) is directly linked to X. Particularly preferably F³ in the above mentioned formula is C(═O)—, as described above.

Most preferably the cytotoxic agent is docetaxel or paclitaxel, as described above. The present invention thus also relates to a conjugate, comprising a hydroxyalkyl starch derivative, as described above, as well as a conjugate obtained or obtainable by the above-mentioned method, the conjugate having a structure according to the following formula

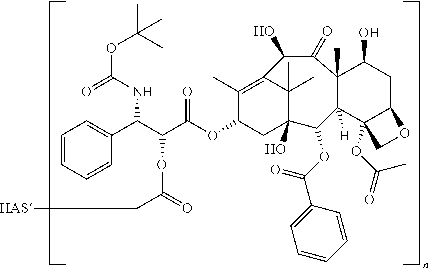

or the following formula

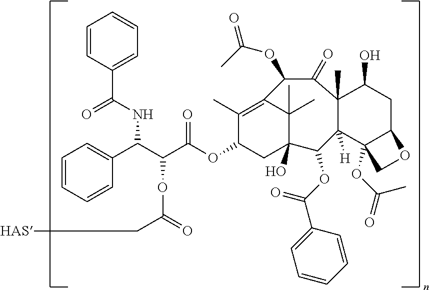

and wherein HAS′ comprises at least one structural unit, preferably 3 to 200 structural units, according to the following formula (I)

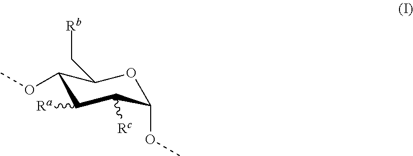

wherein at least one of R^{a}, R^{b} and R^{c} is —[O—CH₂—CH₂]ₜ—X— and X is —S— and the functional group X is directly linked to the —CH₂—C(═O)— group, shown in the formulas above.

According to a second especially preferred embodiment of the present invention, HAS′ comprises at least one structural unit, preferably 3 to 200 structural units, according to the following formula (I)

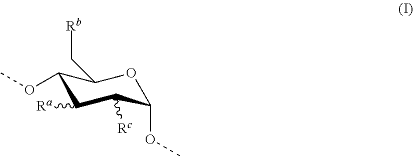

wherein at least one of R^{a}, R^{b} and R^{c} is —-[O—CH₂—CH₂]ₜ-—X— and X is —S—, thus at least one of R^{a}, R^{b} and R^{c} is —[O—CH₂—CH₂]ₜ—S—. This hydroxyalkyl starch derivative is according to this preferred embodiment of the invention, combined with a moiety -L-M having the structure (—[F²]_{q}-[L²]_{g}-[E]ₑ—[CR^{m}Rⁿ]_{f}—F³-M)ₙ, wherein e is 1 and E is preferably —S— or —O—.

Accordingly, in this preferred embodiment, again, an electron-withdrawing group is present in close proximity to the functional group F³, the electron-withdrawing group being represented by the group E.

According to this embodiment, X is directly linked to the functional group F² with q and g preferably both being 1.

As described above, the functional group F² is, if present, preferably selected from —S— and -succinimide-, preferably succinimide-.

Thus, according to this embodiment, the conjugate, or the conjugate obtained or obtainable by the above-mentioned method, preferably has a structure according to the following formulas

HAS′(-succinimide-L²-O—[CR^{m}Rⁿ]_{f}—F³-M)ₙ

or

HAS′(-succinimide-L²-S—[CR^{m}Rⁿ]_{f}—F³-M)ₙ

wherein HAS′ comprises at least one structural unit, preferably 3 to 200 structural units, according to the following formula (I)

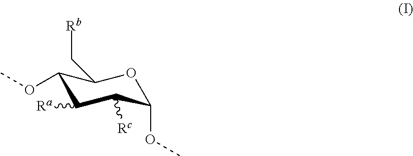

wherein at least one of R^{a}, R^{b} and R^{c} is —-[O—CH₂—CH₂]ₜ—X— and X is —S— and wherein the succinimide is directly linked to X, thereby forming a

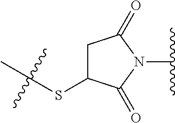 [SMILES: *SC1CC(=O)N(*)C1=O]

bond.

Particularly preferably F³ in the above mentioned formula is —C(═O)—, as described above.

As regards, the linking moiety L² according to this preferred embodiment, L² is preferably an alkyl group, as described above. More preferably L² is selected from the group consisting of —CH₂—CH₂—CH₂—CH₂—CH₂—CH₂—, —CH₂—CH₂—CH₂—CH₂—CH₂—, —CH₂—CH₂—CH₂—CH₂—, —CH₂—CH₂—CH₂—, —CH₂—CH₂—, —CH₂—, more preferably L² is selected from the group consisting of —CH₂—, —CH₂—CH₂—, —CH₂—CH₂—CH₂—, most preferably L² is —CH₂—CH₂—.

Accordingly, the present invention also relates to a conjugate, comprising a residue of a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(-succinimide-CH₂—CH₂-E-[CR^{m}Rⁿ]_{f}—C(═O)-M)ₙ

more preferably a structure according to one of the following formulas

HAS′(-succinimide-CH₂—CH₂—O—[CR^{m}Rⁿ]_{f}—C(═O)-M)ₙ and

HAS′(-succinimide-CH₂—CH₂—S—[CR^{m}Rⁿ]_{f}—C(═O)-M)ₙ

wherein HAS′ preferably comprises at least one structural unit, preferably 3 to 200 structural units, according to the following formula (I)

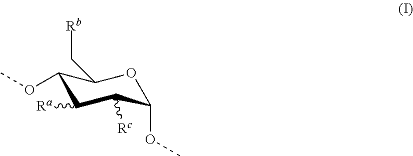

wherein at least one of R^{a}, R^{b} and R^{c} is
—[O—CH₂—CH₂], —X— and X is —S— and the functional group X is directly linked to the succinimide group, thereby forming a

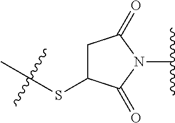 [SMILES: *SC1CC(=O)N(*)C1=O]

bond and wherein the hydroxyalkyl starch derivative comprises at least n functional groups X.

Most preferably, according to this embodiment of the present invention, R^{m} and Rⁿ are both H and f is 1.

The present invention thus also relates to a conjugate, comprising a hydroxyalkyl starch derivative, as described above, as well as a conjugate obtained or obtainable by the above-mentioned method, the conjugate having a structure according to one of the following formulas:

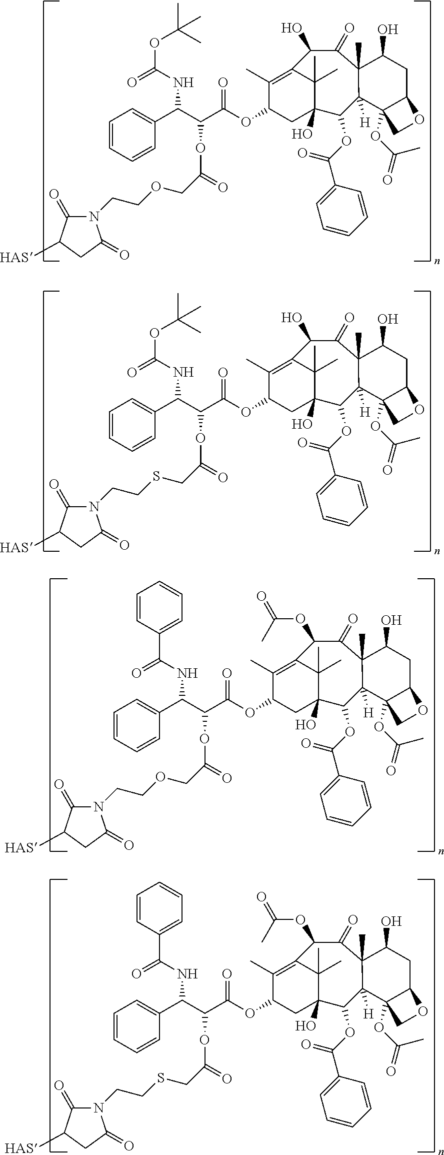

wherein HAS′ comprises at least one structural unit, preferably 3 to 200 structural units, according to the following formula (I)

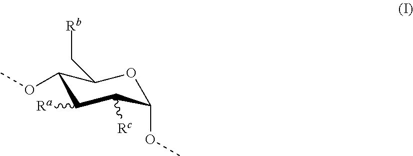

wherein at least one of R^{a}, R^{b} and R^{c} is —[O—CH₂—CH₂]ₜ—X— and X is —S— thereby forming a

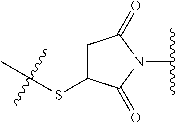 [SMILES: *SC1CC(=O)N(*)C1=O]

bond and wherein the hydroxyalkyl starch derivative comprises at least n functional groups X.

According to a third especially preferred embodiment of the present invention, the residue of hydroxyalkyl starch derivative comprises at least one structural unit, preferably 3 to 200 structural units, according to the following formula (Ib)

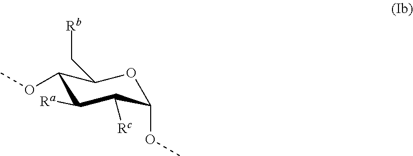

wherein at least one of R^{a}, R^{b} and R^{c} is —[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —[O—CH₂—CH₂]ₜ—O-L¹-S—, and wherein t is in the range of from 0 to 4.

As regards, the structural moiety L¹, L¹ is preferably an alkyl group. Reference is made to the definition of the term “alkyl” presented above. The term also encompasses substituted alkyl groups, as mentioned above.

According to a preferred embodiment of the present invention, the linking moiety L¹ is a spacer comprising at least one structural unit according to the formula —{[CR^{d}R^{f}]ₕ-[F⁴]ᵤ-[CR^{dd}R^{ff}]_{z}}ₐₗₚₕₐ—, as described above, wherein F⁴ is preferably selected from the group consisting of —S—, —O— and NH—, more preferably wherein F⁴, if present, is O— or —S—, more preferably wherein F⁴ is —S—. Reference is made to the discussion of the linking moiety L¹ above.

According to this third especially preferred embodiment of the present invention, preferably at least one of R^{d} and R^{f} of at least one repeating unit of —[CR^{d}R^{f}]ₕ— is OH. More preferably, R^{d} and R^{f} are either H or OH, wherein at least one of R^{d} and R^{f} of at least one repeating unit of —[CR^{d}R^{f}]ₕ— is —OH, wherein the repeating units may be the same or may be different. Most preferably R^{dd} and R^{ff} are, if present, H as well.

Particularly preferably, L¹ has a structure selected from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NE-CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—, most preferably L¹ is —CH₂—CHOH—CH₂—S CH₂—CH₂—.

The hydroxyalkyl starch derivative according to this third preferred embodiment, is preferably combined with a moiety -L-M having the structure

—[F²]_{q}-[L²]_{g}-[E]ₑ—[CR^{m}Rⁿ]_{f}—F³-M

wherein q, g and e are 0.

Most preferably f is 1 and R^{m} and Rⁿ are both H.

Thus, the present invention also relates to a conjugate, comprising a residue of a hydroxyalkyl starch derivative, as described above, as well as a conjugate obtained or obtainable by the above-mentioned method, wherein the conjugate comprises a residue of a hydroxyalkyl starch derivative and a cytotoxic agent, the conjugate having a structure according to the following formula

HAS′(—[F²]_{q}-[L²]_{g}-[E]ₑ—[CR^{m}Rⁿ]_{f}—F³-M)ₙ

wherein f is 1 and wherein R^{m} and Rⁿ are both H, and wherein q, g and e are 0 and wherein the residue of the hydroxyalkyl starch derivative preferably comprises at least one structural unit, preferably 3 to 200 structural units, according to the following formula (Ib)

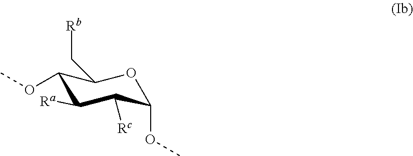

wherein at least one of R^{a}, R^{b} and R^{c} is —-[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —[O—CH₂—CH₂]-O-L¹-S—, wherein t is in the range of from 0 to 4, and wherein L¹ is preferably —CH₂—CHOH—CH₂—S—CH₂—CH₂—. Most preferably F³ is C(═O)— and M is a residue of a cytotoxic agent, said cytotoxic agent being docetaxel or paclitaxel.

According to an especially preferred embodiment, the conjugate has a structure according to the following formula

HAS′(—CH₂—C(═O)-M)ₙ

and wherein HAS′ comprises at least one structural unit, preferably 3 to 200 structural units, according to the following formula (Ib)

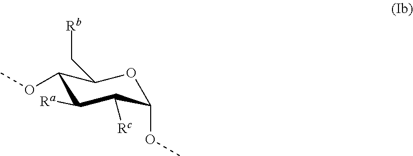

wherein at least one of R^{a}, R^{b} and R^{c} is —O—CH₂—CH₂—O—CH₂—CHOH—CH₂—S—CH₂—CH₂—S—.

The present invention in particular relates to a conjugate, comprising a residue of a hydroxyalkyl starch derivative, as described above, as well as a conjugate obtained or obtainable by the above-mentioned method, the conjugate having a structure according to the following formula

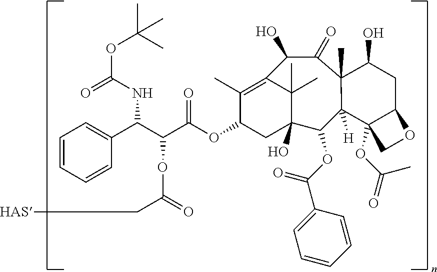

or the following formula

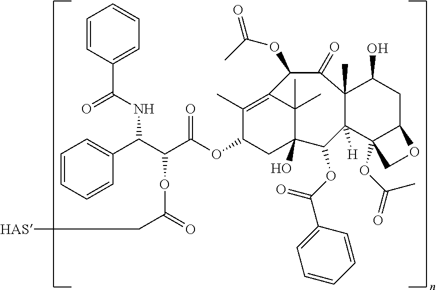

and wherein HAS′ comprises at least one structural unit, preferably 3 to 200 structural units, according to the following formula (Ib)

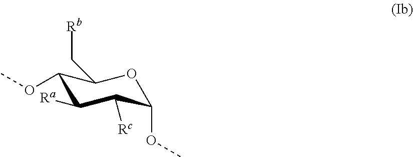

wherein at least one of R^{a}, R^{b} and R^{c} is —[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —-[O—CH₂—CH₂], —O-L¹-S—, wherein t is in the range of from 0 to 4, and wherein L¹ is preferably —CH₂—CHOH—CH₂—S—CH₂—CH₂—.

According to an alternative embodiment, the hydroxyalkyl starch derivative according to this third preferred embodiment, is combined with a moiety -L-M having the structure

-[F²]_{q}-[L²]_{g}-[E]ₑ—[CR^{m}Rⁿ]_{f}—F³-M

wherein q is 1 and F² is succinimide. More preferably F³ is —C(═O)—. Further preferably, e is 1, and E is O— or —S—.

Accordingly, the present invention also relates to a conjugate, comprising a residue of a hydroxyalkyl starch derivative, as described above, as well as a conjugate obtained or obtainable by the above-mentioned method, wherein the conjugate comprises a residue of a hydroxyalkyl starch derivative and a cytotoxic agent, the conjugate having a structure according to the following formula

HAS′(-succinimide-[L²]_{g}-E-[CR^{m}Rⁿ]_{f}—C(═O)-M)ₙ

more preferably a structure according to one of the following formulas

HAS′(-succinimide-[L²]_{g}—O—[CR^{m}Rⁿ]_{f}—C(═O)-M)ₙ

and

HAS′(succinimide-[L²]_{g}—S—[CR^{m}Rⁿ]_{f}—C(═O)-M)ₙ

wherein HAS′ preferably comprises at least one structural unit, preferably 3 to 200 structural units, according to the following formula (Ib)

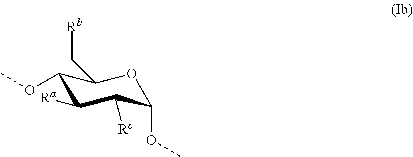

wherein at least one of R^{a}, R^{b} and R^{c} is —-[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —[O—CH₂—CH₂]ₜ—O-L¹-S—, wherein t is in the range of from 0 to 4, and wherein L¹ is preferably —CH₂—CHOH—CH₂—S—CH₂—CH₂—.

Depending on integer f, which is 1, 2 or 3, the electron-withdrawing group E is either present in alpha, beta or gamma position to the functional group F³. As regards, the position of the functional group E to the functional group F³, E is preferably present in alpha position to the functional group F³. Thus, according to this preferred embodiment, according to which the functional group E is present as electron-withdrawing group, the integer f is preferably 1, so that E is present in alpha position to the functional group F³.

Most preferably f is 1 and R^{m} and Rⁿ are both H.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(succinimide-[L²]_{g}-E-CH₂—C(═O)-M)ₙ

more preferably a structure according to one of the following formulas

HAS′(-succinimide-[L²]_{g}-O—CH₂—C(═O)-M)ₙ

and

HAS′(-succinimide-[L²]_{g}—S—CH₂—C(═O)-M)ₙ

wherein HAS′ preferably comprises at least one structural unit, preferably 3 to 200 structural units, according to the following formula (Ib)

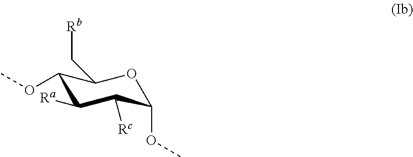

wherein at least one of R^{a}, R^{b} and R^{c} is —-[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —-[O—CH₂—CH₂], —O-L¹-S—, wherein t is in the range of from 0 to 4, and wherein L¹ is preferably —CH₂—CHOH—CH₂—S—CH₂—CH₂—. L² is preferably an alkyl group, most preferably g is 1 and L² has a structure selected from the group consisting of CH₂—CH₂—, CH₂—CH₂—CH₂— and CH₂—CH₂—CH₂—CH₂—.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(-succinimide-CH₂—CH₂—S—CH₂—C(═O)-M)ₙ

or the following formula

HAS′(succinimide-CH₂—CH₂—O—CH₂—C(═O)-M)ₙ

wherein HAS′ preferably comprises at least one structural unit, preferably 3 to 200 structural units, according to the following formula (Ib)

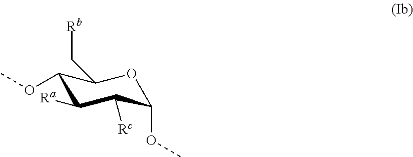

wherein at least one of R^{a}, R^{b} and R^{c} is —-[O—CH₂—CH₂]ₜ-[F¹]ₚ-L¹-X— with X being —S—, preferably with p being 1 and F¹ being —O—, thus at least one of R^{a}, R^{b} and R^{c} has preferably the structure —-[O—CH₂—CH₂]ₜ—O-L¹-S—, wherein t is in the range of from 0 to 4, and wherein L¹ is preferably —CH₂—CHOH—CH₂—S—CH₂—CH₂—, wherein the succinimide is linked to the functional group X.

In particular, the present invention thus also relates to a conjugate, comprising a hydroxyalkyl starch derivative, as described above, as well as a conjugate obtained or obtainable by the above-mentioned method, the conjugate having a structure according to one of the following formulas:

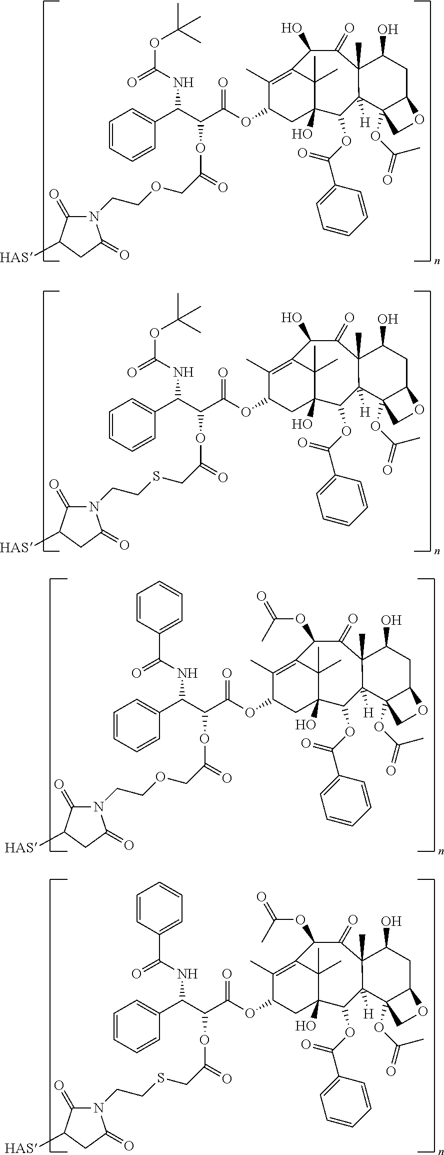

wherein HAS′ comprises at least one structural unit, preferably 3 to 200 structural units, according to the following formula (Ib)

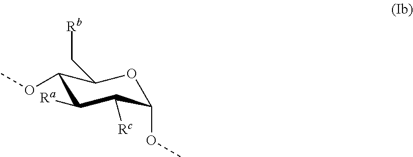

wherein at least one of R^{a}, R^{b} and R^{c} is —[O—CH₂—CH₂]ₜ—[F¹]ₚ-L¹-X— with X being —S—, preferably with p being 1 and F¹ being —O—, preferably the structure —[O—CH₂—CH₂]ₜ—O-L¹-S—, wherein t is in the range of from 0 to 4, and wherein L¹ is preferably —CH₂—CHOH—CH₂—S—CH₂—CH₂—, wherein —X— is attached to the succinimide, thereby forming a

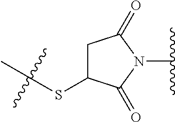 [SMILES: *SC1CC(=O)N(*)C1=O]

bond and wherein the hydroxyalkyl starch derivative comprises at least n functional groups X.

According to a fourth especially preferred embodiment of the present invention, the residue of hydroxyalkyl starch derivative comprises at least one structural unit, preferably 3 to 200 structural units, according to the following formula (Ib)

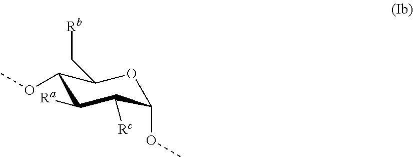

wherein at least one of R^{a}, R^{b} and R^{c} is —-[O—CH₂—CH₂]ₜ-[F¹]ₚ-L¹-X— with X being —S—, preferably with p being 1 and with F¹ being —Y⁷—C(═Y⁶)—, —C(═Y⁶)—, —Y⁷—C(═Y⁶)—Y⁸—, wherein Y⁷ is selected from the group consisting of —NR^{Y7}—, —O— or —S—, -succinimide, —NH—NH—, —HN—O—, —CH═N—O—, —O—N═CH—, —CH═N—, —N═CH—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H. More preferably F¹ has the structure —Y⁷—C(═Y⁶)—Y⁸—, wherein Y⁶ is selected from the group consisting of NR^{Y6}, O and S, with R^{Y6} being H or alkyl, preferably H, and wherein —Y⁸— is selected from the group consisting of —NR^{Y8}—, —S—, —O—, —NH—NH—, with R^{Y8} being H or alkyl, preferably H, and wherein Y⁷ is O— or —S—, preferably —O—. More preferably F¹ has the structure O—C(═O)—NH—.

As regards, the structural moiety L¹, L¹ is preferably an alkyl group, as described above. According to a preferred embodiment of the present invention, the linking moiety L¹ is a spacer comprising at least one structural unit according to the formula —{[CR^{d}R^{f}]ₙ-[F⁴]ᵤ-[CR^{dd}R^{ff}]_{z}}ₐₗₚₕₐ—, as described above, wherein F⁴ is preferably selected from the group consisting of —S—, —O— and NH—, more preferably wherein F⁴, if present, is O— or —S—, more preferably wherein F⁴ is —S—. Reference is made to the discussion of linking moiety L¹ above. As described above, residues R^{d}, R^{f}, R^{dd} and R^{ff} are, independently of each other, preferably selected from the group consisting of halogens, alkyl groups, H or hydroxyl groups. More preferably, these residues are independently from each other H, alkyl or hydroxyl.

Preferably, in case p is 1 and F¹ has the structure —Y⁷—C(═Y⁶)—Y⁸—, such as the structure O—C(═O)—NH—, integer u and integer z of the formula —{[CR^{d}R^{f}]ₕ-[F⁴]ᵤ—[CR^{dd}R^{ff}]_{z}}ₐₗₚₕₐ— are 0, alpha is 1, the linking moiety L¹ thus corresponds to the structural unit —[CR^{d}R^{f}]ₕ—.

As described above, the integer h is preferably in the range of from 1 to 20, more preferably 1 to 10, such as 1, 2, 3, 4, 5, 6, 7, 8, 9 or 10, more preferably 1 to 5, most preferably 1 to 3. More preferably R^{d} and R^{f} are both H. Thus, by way of example, the following preferred linking moieties L¹ are mentioned: —CH₂—, —CH₂—CH₂—, —CH₂—CH₂—CH₂—, —CH₂—CH₂—CH₂—CH₂—, —CH₂—CH₂—CH₂—CH₂—CH₂—, more preferably —CH₂—CH₂—, in the context of the fourth preferred embodiment.

The hydroxyalkyl starch derivative according to this fourth preferred embodiment, is preferably combined with a moiety -L-M having the structure

-[F²]_{q}-[L²]_{g}-[E]ₑ—[CR^{m}Rⁿ]_{f}—F³-M

wherein q, g and e are 0.

Accordingly, in this preferred embodiment, the functional group X (which in this case is —S—) represents an electron-withdrawing group in close proximity to the functional group F³ since X is directly linked to the structural unit —[CR^{m}Rⁿ]_{f}—, thereby forming the structural unit —X—[CR^{m}Rⁿ]_{f}. Depending on integer f, which is 1, 2 or 3, the electron-withdrawing group is either present in alpha, beta or gamma position to the functional group F³. As regards, the position of the functional group X to the functional group F³, X is preferably present in alpha position to the functional group F³. Thus, according to this preferred embodiment, according to which the functional group X represents the electron-withdrawing group, the integer f is preferably 1, so that X is present in alpha position to the functional group F³.

Most preferably f is 1 and R^{m} and Rⁿ are both H.

Thus, the present invention also relates to a conjugate, comprising a hydroxyalkyl starch derivative, as described above, as well as a conjugate obtained or obtainable by the above-mentioned method, wherein the conjugate comprises a residue of a hydroxyalkyl starch derivative and a cytotoxic agent, the conjugate having a structure according to the following formula

HAS′(—[F²]_{q}-[L²]_{g}-[E]ₑ—[CR^{m}Rⁿ]_{f}—F³-M)ₙ

wherein f is 1 and wherein R^{m} and Rⁿ are both H, and wherein q, g and e are 0 and wherein HAS′ preferably comprises at least one structural unit, preferably 3 to 200 structural units, according to the following formula (Ib)

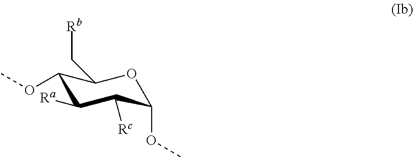

wherein at least one of R^{a}, R^{b} and R^{c} is —[O—CH₂—CH₂]ₜ-[F¹]ₚ-L¹-X— with X being —S—, preferably with p being 1 and F¹ being O—C(═O)—NH—, wherein t is in the range of from 0 to 4. Most preferably the functional group F³ is C(═O)—. According to an especially preferred embodiment, the conjugate has a structure according to the following formula

HAS′(—CH₂—C(═O)-Mₙ

and wherein HAS′ comprises at least one structural unit, preferably 3 to 200 structural units, according to the following formula (Ib)

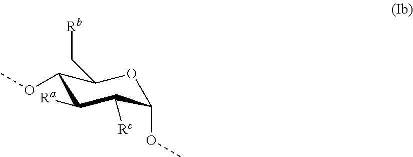

wherein at least one of R^{a}, R^{b} and R^{c} is —-[O—CH₂—CH₂]ₜ—O—C(═O)—NH—CH₂—CH₂—S—.

The present invention in particular relates to a conjugate, comprising a hydroxyalkyl starch derivative, as described above, as well as a conjugate obtained or obtainable by the above-mentioned method, the conjugate having a structure according to the following formula

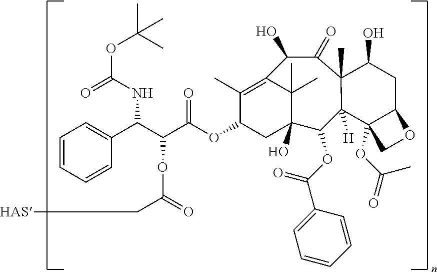

or the following formula

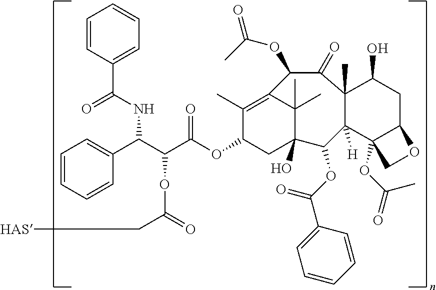

and wherein HAS′ comprises at least one structural unit, preferably 3 to 200 structural units, according to the following formula (I)

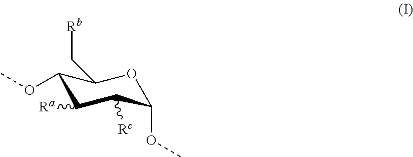

wherein at least one of R^{a}, R^{b} and R′ is —[O—CH₂—CH₂]ₜ-[F¹]ₚ-L¹-X— with X being —S—, with p being 1 and F¹ being O—C(═O)—NH—, and wherein t is in the range of from 0 to 4.

According to an alternative embodiment, the hydroxyalkyl starch derivative according to the fourth preferred embodiment, is combined with a moiety -L-M having the structure

-[F²]_{q}-[L²]_{g}-[E]ₑ—[CR^{m}Rⁿ]_{f}—F³-M

wherein q is 1 and F² is succinimide. More preferably F³ is —C(═O)—. Further preferably, e is 1, and E is O— or —S—.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(-succinimide-[L²]_{g}-E-[CR^{m}Rⁿ]_{f}—C(═O)-M)ₙ,

more preferably a structure according to one of the following formulas

HAS′(succinimide-[L²]_{g}-O—[CR^{m}Rⁿ]_{f}—C(═O)-M)ₙ

and

HAS′(-succinimide-[L²]_{g}—S—[CR^{m}Rⁿ]_{f}—C(═O)-M)ₙ

wherein HAS′ preferably comprises at least one structural unit, preferably 3 to 200 structural units, according to the following formula (I)

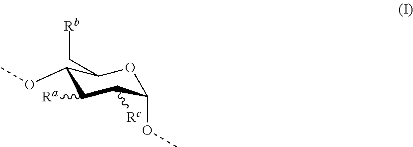

wherein at least one of R^{a}, R^{b} and R^{c} is —[O—CH₂—CH₂]ₜ-[F¹]ₚ-L¹-X— with X being —S—, with p being 1 and F¹ being O—C(═O)—NH—, wherein t is in the range of from 0 to 4.

Depending on integer f, which is 1, 2 or 3, the electron-withdrawing group E is either present in alpha, beta or gamma position to the functional group F³. As regards, the position of the functional group E to the functional group F³, E is preferably present in alpha position to the functional group F³. Thus, according to this preferred embodiment, according to which the functional group E is present as electron-withdrawing group, the integer f is preferably 1, so that E is present in alpha position to the functional group F³. Most preferably f is 1 and R^{m} and Rⁿ are both H.

Accordingly, the present invention also relates to a conjugate, comprising a hydroxyalkyl starch derivative, as described above, as well as a conjugate obtained or obtainable by the above-mentioned method, wherein the conjugate comprises a hydroxyalkyl starch derivative and a cytotoxic agent, the conjugate having a structure according to the following formula

HAS′(succinimide-[L²]_{g}-E-CH₂—C(═O)-M)ₙ,

more preferably a structure according to one of the following formulas

HAS′(succinimide-[L²]_{g}—O—CH₂—C(═O)-M)ₙ

and

HAS′(-succinimide-[L²]_{g}—S—CH₂—C(═O)-M)

wherein the residue of the hydroxyalkyl starch derivative preferably comprises at least one structural unit, preferably 3 to 200 structural units, according to the following formula (I)

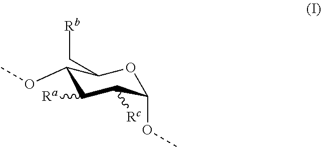

wherein at least one of R^{a}, R^{b} and R^{c} is —[O—CH₂—CH₂]ₜ-[F¹]ₚ-L¹-X— with X being —S—, with p being 1 and F¹ being O—C(═O)—NH—, wherein t is in the range of from 0 to 4. Preferably, g is 1 and L² has a structure selected from the group consisting of CH₂—CH₂—, —CH₂—CH₂—CH₂— and CH₂—CH₂—CH₂—CH₂—.

Thus, the present invention also relates to a hydroxyalkyl starch conjugate, as described above, as well as a conjugate obtained or obtainable by the above-mentioned method, wherein the conjugate comprises a residue of a hydroxyalkyl starch derivative and a cytotoxic agent, the conjugate having a structure according to the following formula

HAS′(-succinimide-CH₂—CH₂—S—CH₂—C(═O)-M)ₙ

or the following formula

HAS′(-succinimide-CH₂—CH₂—O—CH₂—C(═O)-M)ₙ.

The residue of the hydroxyalkyl starch derivative preferably comprises at least one structural unit, preferably 3 to 200 structural units, according to the following formula (I)

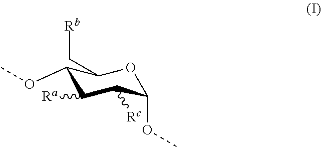

wherein at least one of R^{a}, R^{b} and R^{c} is —[O—CH₂—CH₂]ₜ-[F¹]ₚ-L¹-X— with X being —S—, with p being 1 and F¹ being O—C(═O)—NH—, wherein t is in the range of from 0 to 4, wherein the succinimide is linked to the functional group X.

In particular, the present invention thus also relates to a conjugate, comprising a residue of a hydroxyalkyl starch derivative, as described above, as well as a conjugate obtained or obtainable by the above-mentioned method, the conjugate having a structure according to one of the following formulas:

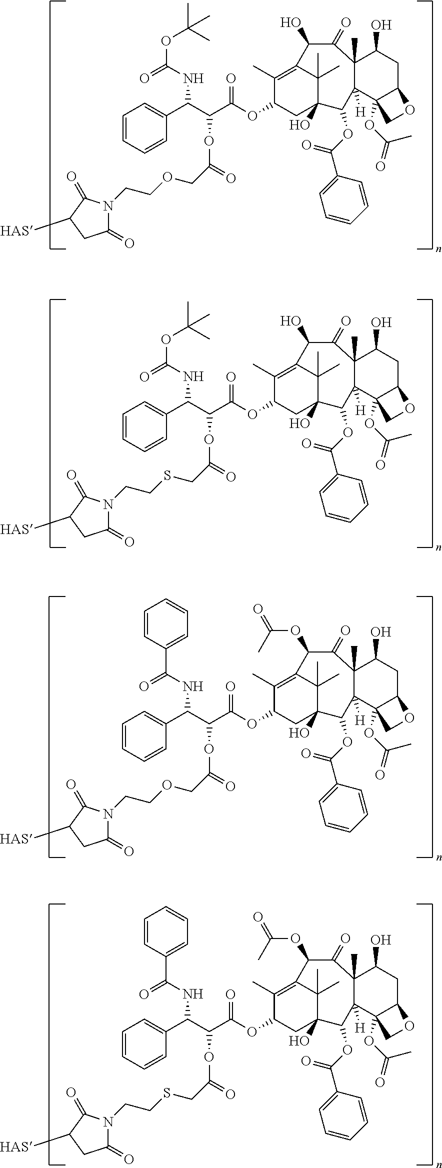

wherein HAS′ comprises at least one structural unit, preferably 3 to 200 structural units, according to the following formula (I)

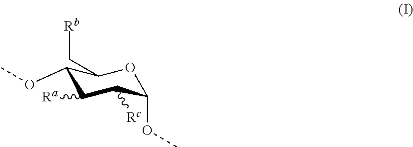

wherein at least one of R^{a}, R^{b} and R^{c} is —-[O—CH₂—CH₂]ₜ-[F¹]-L¹-X— with X being —S—, with p being 1 and F¹ being O—C(═O)—NH—, wherein t is in the range of from 0 to 4, wherein X is attached to the succinimide, thereby forming a

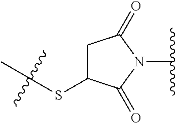 [SMILES: *SC1CC(=O)N(*)C1=O]

bond and wherein the hydroxyalkyl starch derivative comprises at least n functional groups X.

### Synthesis of HAS Conjugates

As described above, the present invention also relates to a method for preparing a hydroxyalkyl starch conjugate comprising a hydroxyalkyl starch derivative and a cytotoxic agent, said conjugate having a structure according to the following formula HAS′(-L-M)ₙ, wherein M is a residue of a cytotoxic agent, said cytotoxic agent comprising a secondary hydroxyl group, L is a linking moiety, HAS′ is a residue of the hydroxyalkyl starch derivative, and n is greater than or equal to 1, preferably wherein n is in the range of from 3 to 200,

said method comprising the steps
(a) providing a hydroxyalkyl starch derivative having a mean molecular weight MW above the renal threshold, preferably in the range of from 60 to 800 kDa, more preferably of from 80 to 800 kDa, and a molar substitution MS in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent.

### The at Least Bifunctional Crosslinking Compound L

The term “at least bifunctional crosslinking compound L” as used in the context of the present invention refers to an at least bifunctional compound comprising the functional groups K¹ and K².

Besides the functional group K¹ and the functional group K² the at least bifunctional crosslinking may optionally contain further functional groups, which may be used, for example, for the attachment of radiolabels, or the like. Hereinunder and above, the “at least bifunctional crosslinking compound L” is also referred to as “crosslinking compound L”

The crosslinking compound L is reacted via its functional group K¹ with the secondary hydroxyl group of the cytotoxic agent, thereby forming a covalent linkage. On the other side, the at least bifunctional crosslinking compound L is reacted via its functional group K² with the functional group Z¹ of the hydroxyalkyl starch derivative, thereby forming a covalent linkage as well.

The crosslinking compound L can be reacted with a cytotoxic agent prior to the reaction with the hydroxyalkyl starch derivative or subsequent to the reaction with the hydroxyalkyl starch derivative. Preferably the crosslinking compound L is coupled to the cytotoxic agent prior to the reaction with the hydroxyalkyl starch derivative.

Thus, the present invention also relates to a method for preparing a hydroxyalkyl starch conjugate comprising a hydroxyalkyl starch derivative and a cytotoxic agent, said conjugate having a structure according to the following formula HAS′(-L-M)ₙ, wherein M is a residue of a cytotoxic agent, wherein the cytotoxic agent comprises a secondary hydroxyl group, L is a linking moiety, HAS′ is a residue of the hydroxyalkyl starch derivative, and n is greater than or equal to 1, preferably wherein n is in the range of from 3 to 200, said method comprising the steps
(a) providing a hydroxyalkyl starch derivative having a mean molecular weight MW above the renal threshold, preferably in the range of from 60 to 800 kDa, more preferably of from 80 to 800 kDa, and a molar substitution in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent, wherein L is coupled to Z¹ via the functional group K² comprised in L, and wherein each cytotoxic agent is coupled via the secondary hydroxyl group to the HAS derivative via the functional group K¹ comprised in L, and wherein the cytotoxic agent is preferably reacted with at least one crosslinking compound L prior to the reaction with the hydroxyalkyl starch derivative, thereby forming a cytotoxic agent derivative comprising the functional group K², and wherein said cytotoxic agent derivative is coupled in a subsequent step to the hydroxyalkyl starch derivative according to step (a).

Further, the present invention relates to a hydroxyalkyl starch conjugate obtained or obtainable by said method.

Upon reaction of the at least bifunctional crosslinking compound L with the hydroxyalkyl starch derivative and the cytotoxic agent the hydroxyalkyl starch conjugate HAS′(-L-M)ₙ is formed. In said conjugate, HAS′ and M are linked via the linking moiety L, wherein said linking moiety L is the linking moiety derived from the at least bifunctional crosslinking compound L.

Preferably, the at least bifunctional crosslinking compound L has a structure according to the following formula

K²-L′-K¹

wherein L¹ is a linking moiety, K² is the functional group capable of being reacted with the functional group Z¹ of the hydroxyalkyl starch derivative and K¹ is the group capable of being reacted with the cytotoxic agent M, as described above.

### The Functional Group K¹

Accordingly, the functional group K¹ is a group capable of being coupled to a secondary hydroxyl group of the cytotoxic agent. Upon reaction of the functional group K¹ with the hydroxyl group, preferably the linking unit —F³—O—, as described above, is formed. Preferably, K¹ is a functional group with which (upon reaction with the hydroxyl group) a covalent linkage between L, preferably L′ and M, is formed which is cleavable in vivo as described above.

The crosslinking compound L may be reacted with either the cytotoxic agent or the hydroxyalkyl starch derivative in an initial step. Preferably, the crosslinking compound L is reacted with the cytotoxic agent prior to the reaction with the hydroxyalkyl starch derivative, a derivative of the cytotoxic agent is formed, the derivative of the cytotoxic agent preferably having the structure K²-L′-F³-M.

Thus, the present invention also describes a method for preparing a hydroxyalkyl starch conjugate, as described above, wherein step (b) comprises the steps
(b1) coupling the cytotoxic agent to the crosslinking compound L having the structure K²-L′-K¹, thereby forming a derivative of the cytotoxic agent having the structure K²-L′-F³-M, wherein M is the residue of the cytotoxic agent,
(b2) coupling the derivative of the cytotoxic agent having the structure K²-L′-F³-M to the hydroxyalkyl starch derivative according to step (a), thereby forming the hydroxyalkyl starch conjugate.

Further, the present invention relates to a hydroxyalkyl starch conjugate obtained or obtainable by said method.

Preferably K¹ comprises the structural unit C(═Y)—, with Y being O, NH or S. Thus, the present invention also relates to a method for preparing a hydroxyalkyl starch conjugate, as described above, wherein the cytotoxic agent is reacted with the at least one crosslinking compound L via the functional group K¹ comprised in said crosslinking compound L, wherein said functional group K¹ comprises the structural unit C(═Y)—, with Y being O, NH or S, more preferably Y is O. Further, the present invention relates to a hydroxyalkyl starch conjugate obtained or obtainable by said method.

According to a particular preferred embodiment K¹ is a carboxylic acid group or a reactive carboxy group.

The term “reactive carboxy group” as used in this context of the present invention is intended to mean an activated carboxylic acid derivative that reacts readily with electrophilic groups, such as the —OH group of the cytotoxic agent, optionally in the presence of a suitable base, in contrast to those groups that require a further catalyst, such as a coupling reagent, in order to react. The term “activated carboxylic acid derivative” as used herein preferably refers to acid halides such as acid chlorides and also refers to activated ester derivatives including, but not limited to, formic and acetic acid derived anhydrides, anhydrides derived from alkoxycarbonyl halides such as isobutyloxycarbonylchloride and the like, isothiocyanates or isocyanates, anhydrides derived from reaction of the carboxylic acid with N,N′-carbonyldiimidazole and the like, and esters derived from activation of the corresponding carboxylic acid with a coupling reagent. Such coupling reagents include, but are not limited to, HATU (0-(7-azabenzotriazol-1-yl)-N,N,N′,N′-tetramethyluronium hexafluorophosphate); HOAt, HBTU (O-benzotriazol-1-yl)-N,N,N′,N′-tetramethyluronium hexafluorophosphate); TBTU (2-(1H-benzotriazol-1-yl)-1,1,3,3-tetramethyluronium hexafluorophosphate); TFFH(N,N,N″,N″-tetramethyluronium-2-fluoro-hexafluorophosphate); BOP (benzotriazol-1-yloxytris(dimethylamino)phosphonium hexafluorophosphate); PyBOP (benzotriazol-1-yl-oxy-trispyrrolidino-phosphonium hexafluorophosphate; EEDQ (2-ethoxy-1-ethoxycarbonyl-1,2-dihydro-quinoline); DCC (dicyclohexylcarbodiimide); DIPCDI (diisopropylcarbodiimide); HOBt (1-hydroxybenzotriazole); NHS(N-hydroxysuccinimide); MSNT (1-(mesitylene-2-sulfonyl)-3-nitro-1H-1,2,4-triazole); aryl sulfonyl halides, e.g. triisopropylbenzenesulfonyl chloride, EDC (1-ethyl-3-(3-dimethylaminopropyl) carbodiimide hydrochloride, CDC (1-cyclohexyl-3-(2-morpholinoethyl)carbodiimide), Pyclop, T3P, CDI, Mukayama's reagent, HODhbt, HAPyU, TAPipU, TPTU, TSTU, TNTU, TOTU, BroP, PyBroP, BOI, TOO, NEPIS, BBC, BDMP, BOMI, AOP, BDP, PyAOP, TDBTU, BOP-Cl, CIP, DEPBT, Dpp-Cl, EEDQ, FDPP, HOTT, TOTT, PyCloP.

In case, K¹ is a carboxylic acid group, the coupling between the cytotoxic agent and the crosslinking compound L is preferably carried out in the presence of at least one coupling reagent, wherein the coupling reagent is preferably selected from the group of coupling reagents mentioned above. In case a coupling reagent is used, most preferably EDC (1-ethyl-3-(3-dimethylaminopropyl)carbodiimide) is used. Additionally additives promoting the activation of the carboxylic acid, such as DMAP (4-(dimethylamino)-pyridine), may be added.

The coupling between the cytotoxic agent and the crosslinking compound is preferably carried out in the presence of a suitable base, preferably an organic base, most preferably an amino group comprising base, most preferably a base selected from the group consisting of diisopropylamine (DIEA), triethylamine (TEA), N-methylmorpholine, N-methyl imidazole, 1,4-diazabicyclo[2.2.2]octane (DABCO), N-methylpiperidine, N-methylpyrrolidine, 2,6-lutidine, collidine, pyridine, 4-dimethylaminopyridine, 1,8-diazabicyclo[5.4.0]undec-7-ene (DBU). As regards the reaction conditions used in this coupling step, preferably, the reaction is carried out in an organic solvent, such as N-methylpyrrolidone (NMP), dimethyl sulfoxide (DMSO), acetonitrile, acetone, dimethyl acetamide (DMA), dimethyl formamide (DMF), formamide, tetrahydrofuran (THF), 1,4-dioxane, diethyl ether, tert.-butyl methyl ether (MTBE), dichloromethane (DCM), chloroform, tetrachloromethane and mixtures of two or more thereof. More preferably, the reaction is carried out in dichloromethane.

The temperature of the coupling reaction is preferably in the range of from 0 to 100° C., more preferably in the range of from 5 to 50° C., and especially preferably in the range of from 15 to 30° C. During the course of the reaction, the temperature may be varied, preferably in the above given ranges, or held essentially constant.

The derivative of the cytotoxic agent, which in particular has the following structure

K²-L′-F³-M,

may be subjected to at least one isolation and/or purification step prior to the reaction with the hydroxyalkyl starch derivative.

### The Functional Group K² and the Functional Group Z¹

In the context of the present invention, K² is a functional group capable of being reacted with a functional group Z¹ of the hydroxyalkyl starch derivative, and Z¹ is the respective functional group capable of being reacted with the functional group K². Upon reaction of K² with Z¹ the unit —X—[F²]_{q}— is formed, with X and —[F²]_{q}— being as described above in the context of the conjugate structures.

Such functional groups Z¹ and K² may be suitably chosen. By way of example, one of the groups Z¹ and K², i.e. Z¹ or K², may be chosen from the group consisting of the functional groups according to the following list while the other group, K² or Z¹, is suitably selected and capable of forming a chemical linkage with Z¹ or K², wherein K² or Z¹ is also preferably selected from the following list:
C—C-double bonds or C—C-triple bonds, such as alkenyl groups, alkynyl groups or aromatic C—C-bonds, in particular alkynyl groups, in particular-C≡C—H;
alkyl sulfonic acid hydrazides, aryl sulfonic acid hydrazides;
the thiol group or the hydroxy group;
thiol reactive groups such as
   a disulfide group comprising the structure —S—S—; such as pyridyl disulfides,
   maleimide group,
   haloacetyl groups,
   haloacetamides,
   vinyl sulfones,
   vinyl pyridines,
   haloalkanes;

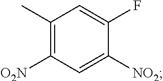

the group
dienes or dienophiles;
azides;
1,2-aminoalcohols;
amino groups comprising the structure —NR′R″, wherein R′ and R″ are independently of each other selected from the group consisting of H, alkyl groups, aryl groups, arylalkyl groups and alkylaryl groups; preferably —NH₂;
hydroxylamino groups comprising the structure O—NR′R″, wherein R′ and R″ are independently of each other selected from the group consisting of H, alkyl groups, aryl groups, arylalkyl groups and alkylaryl groups; preferably O—NH₂;
oxyamino groups comprising the structure NR'—O—, with R′ being selected from the group consisting of alkyl groups, aryl groups, arylalkyl groups and alkylaryl groups; preferably NH—O—;
residues having a carbonyl group -Q-C(=G)-M′, wherein G is O or S, and M′ is, for example,
   —OH or —SH;
   an alkoxy group, an aryloxy group, an arylalkyloxy group, or an alkylaryloxy group;
   an alkylthio group, an arylthio group, an arylalkylthio group, or an alkylarylthio group;
   an alkylcarbonyloxy group, an arylcarbonyloxy group, an arylalkylcarbonyloxy group, an alkylarylcarbonyloxy group;
   activated esters such as esters of hydroxylamines having an imide structure such as N-hydroxysuccinimide;
—NR′—NH₂, wherein R′ is selected from the group consisting of H, alkyl, aryl, arylalkyl and alkylaryl groups; preferably wherein R′ is H;
carbonyl groups such as aldehyde groups, keto groups, hemiacetal groups or acetal groups;
the carboxy groups;
the —N═C═O group or the —N═C═S group;
vinyl halide groups such as vinyl iodide or vinyl bromide, or triflate;
—(C═NH₂Cl)-OAlkyl;
epoxide;
residues comprising a leaving group such as e.g. halogens or sulfonates.

Preferably, Z¹ is selected from the group consisting of aldehyde, keto, hemiacetal, acetal, alkynyl, azide, carboxy groups, alkenyl, thiol reactive groups, such as maleimide, halogen acetyl, pyridyl disulfides, haloacetamides, vinyl sulfones and vinyl pyridines, —SH, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, where G is O or S and, if G is present twice, it is independently O or S.

Thus, the present invention also relates to a method for preparing a hydroxyalkyl starch conjugate, as described above, wherein K² is reacted with the functional group Z¹, wherein Z¹ is selected from the group consisting of aldehyde groups, keto groups, hemiacetal groups, acetal groups, alkynyl groups, azide groups, carboxy groups, alkenyl groups, thiol reactive groups, —SH, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, where G is O or S and, if G is present twice, it is independently O or S. Further, the present invention also relates to the conjugate obtained or obtainable by said method.

By way of example, in the following Table 1, suitable combinations of Z¹ and K² are mentioned:

TABLE 1
Examples for K² and Z¹

| K² | Z¹ |
|---|---|
| —SH | thiol reactive group |
| —NH₂ | aldehyde group, keto group, |
| | hemiacetal group, acetal group or |
| | carboxy group |
| —O—NH₂ | aldehyde group, keto group, |
| | hemiacetal group, acetal group or |
| | carboxy group |
| —(C═G)—NH—NH₂ | aldehyde group, keto group, |
| | hemiacetal group, acetal group or |
| | carboxy group |
| —G—(C═G)—NH—NH₂ | aldehyde group, keto group, |
| | hemiacetal group, acetal group or |
| | carboxy group |
| —SO₂—NH—NH₂ | aldehyde group, keto group, |
| | hemiacetal group, acetal group or |
| | carboxy group |
| alkynyl or | azide |
| diphenylphosphinomethylthioester | |
| azide | alkynyl or |
| | diphenylphosphinomethylthioester |
| aldehyde group, keto group, | —NH₂ |
| hemiacetal group, acetal group or | |
| carboxy group | |
| aldehyde group, keto group, | —O—NH₂ |
| hemiacetal group, acetal group or | |
| carboxy group | |
| aldehyde group, keto group, | —(C═G)—NH—NH₂ |
| hemiacetal group, acetal group or | |
| carboxy group | |
| aldehyde group, keto group, | —G—(C═G)—NH—NH₂ |
| hemiacetal group, acetal group or | |
| carboxy group | |
| aldehyde group, keto group, | —SO₂—NH—NH₂ |
| hemiacetal group, acetal group or | |
| carboxy group | |
| thiol reactive group | —SH |
| thioester | alpha-thiol-beta-amino group |
| alpha-thiol-beta-amino group | thioester |

It has to be understood that the groups Z¹ are statistically distributed throughout the hydroxyalkyl starch derivative. Thus, the hydroxyalkyl starch derivative formed in step (a) of the method of the present invention comprises at least one structural unit, preferably 3 to 200 structural units, according to the following formula (I)

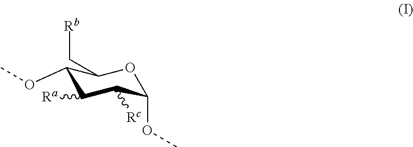

with Z¹ being comprised in at least one of R^{a}, R^{b} or R^{c} and preferably being comprised in multiple repeating units of the structural unit according to the formula (I).

According to a preferred embodiment of the present invention, the functional group Z¹ is a thiol group. Thus, the present invention also relates to a method for preparing a hydroxyalkyl starch conjugate, as described above, wherein in step (a) a derivative is formed comprising at least one thiol group, preferably comprising multiple thiol groups, the derivative having a mean molecular weight MW above the renal threshold, preferably in the range of from 60 to 800 kDa, more preferably of from 80 to 800 kDa, and a molar substitution in the range of from 0.6 to 1.5. The present invention further relates to the conjugate obtained or obtainable by said method.

In case Z¹ is a thiol group, K² is preferably a thiol reactive group, preferably a group selected from the group consisting of pyridyl disulfides, maleimide group, haloacetyl groups, haloacetamides, vinyl sulfones and vinyl pyridines. Preferably, K² is a thiol-reactive group selected from the group consisting of the following structures:

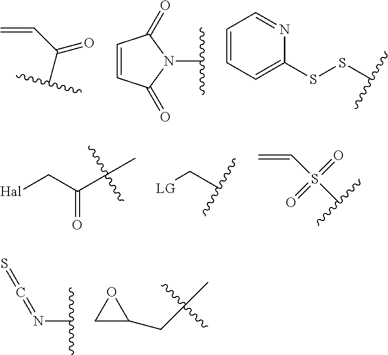

wherein Hal is a halogen, such as Cl, Br, or I, and LG is a leaving group (or nucleofuge). The term “leaving group”, as used in this context of the present invention, is denoted to mean a molecular fragment that departs with a pair of electrons in heterolytic bond cleavage upon reaction with the functional group Z¹. Examples are, inter alia, halogens or sulfonic esters. Examples for sulfonic esters are, inter alia, the mesyl and tosyl group.

More preferably, K² is a thiol-reactive group selected from the group consisting of the following structures:

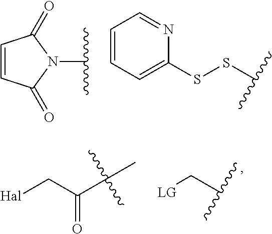

more preferably from the following structures:

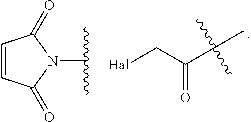

Thus, the present invention also describes a method for preparing a hydroxyalkyl starch conjugate comprising a hydroxyalkyl starch derivative and a cytotoxic agent comprising a secondary hydroxyl group said conjugate having a structure according to the following formula HAS′(-L-M)ₙ, wherein M is a residue of a cytotoxic agent, L is a linking moiety, HAS′ is a residue of the hydroxyalkyl starch derivative, and n is greater than or equal to 1, preferably wherein n is in the range of from 3 to 200,

said method comprising the steps
(a) providing a hydroxyalkyl starch derivative having a mean molecular weight MW above the renal threshold, preferably in the range of from 60 to 800 kDa, more preferably of from 80 to 800 kDa, and a molar substitution in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent, and wherein L is coupled to Z¹ via the functional group K² comprised in L, and wherein each cytotoxic agent is coupled via the secondary hydroxyl group to the hydroxyalkyl starch derivative via the functional group K¹ comprised in L,
   and wherein Z¹ is —SH, and wherein K² is a thiol reactive group, preferably a group selected from the group consisting of the following structures:

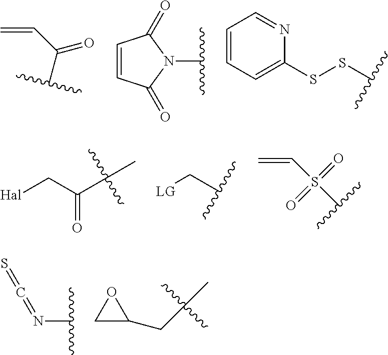

and wherein K¹ comprises the structural unit C(═Y)—, with Y being O, NH or S, more preferably Y is O, preferably, wherein K¹ is a carboxylic acid group or a reactive carboxy group. Further, the present invention also relates to the respective conjugate obtained or obtainable by said method.

Preferably, the at least bifunctional crosslinking compound L has a structure according to the following formula, K²-[L²]_{g}-[E]ₑ—[CR^{m}Rⁿ]_{f}—K¹, wherein L² is a linking moiety, E is an electron-withdrawing group, and R^{m} and Rⁿ are, independently of each other H or alkyl, and g is 0 or 1, e is 0 or 1, and f is in the range of from 1 to 3, as described above.

Thus, in step (b) of the present invention, the hydroxyalkyl starch derivative according to step (a) is preferably reacted with a crosslinking compound L, with L having the structure

K²-[L²]_{g}-[E]ₑ—[CR^{m}Rⁿ]_{f}—K¹,

wherein the crosslinking compound L is coupled to Z¹ comprised in the hydroxyalkyl starch derivative via the functional group K², and wherein each cytotoxic agent is coupled via the secondary hydroxyl group to the hydroxyalkyl starch derivative via the functional group K¹ thereby forming a conjugate having the structure

HAS′(—[F²]_{q}-[L²]_{g}-[E]ₑ—[CR^{m}Rⁿ]_{f}—F³-M)ₙ

with F², L², E, q, g, e and —[CR^{m}Rⁿ]_{f}-being as described hereinabove, preferably wherein E is an electron-withdrawing group selected from the group consisting of —O—, —S—, —SO—, —SO₂—, —NR^{e}—, succinimide, —C(═Y^{e})—, —NR^{e}—C(═Y^{e})—, —C(═Y^{c})—, —CH(NO₂)—, —CH(CN)—, aryl moieties or an at least partially fluorinated alkyl moiety, wherein Y^{e} is either O, S or NR^{e}, and R^{e} is hydrogen or alkyl, more preferably wherein E is selected from the group consisting of —NH—C(═O)—, —C(═O)—NH—, —NH—, —O—, —S—, —SO—, —SO₂ and -succinimide-, L² is a linking moiety, preferably an alkyl, alkenyl, alkylaryl, arylalkyl, heteroaryl, alkylheteroaryl, heteroarylalkyl or aryl group, f is in the range of from 1 to 3, g is 0 or 1, e is 0 or 1, and wherein R^{m} and Rⁿ are, independently of each other, H or alkyl, more preferably H or methyl.

By way of example, the following preferred crosslinking compounds L are mentioned in table 1a:

TABLE 1a
Preferred crosslinking compounds L, by way of example
K₂—[L₂]_{g}—[E]ₑ—[CR^{m}Rⁿ]_{f}—K¹

| | K² | L²/g | [E]ₑ | [CR^{m}Rⁿ]_{f} | K¹ |
|---|---|---|---|---|---|
| 1 | maleimide- | g is 0 | e is 0 | —CH₂—CH₂— | —COOH |
| 2 | Hal- | g is 0 | e is 0 | —CH₂— | —COOH |
| 3 | maleimide- | g is 1 | e is 1 | —CH₂— | —COOH |
| | | L² is selected from the group: | E is —S— | | |
| | | —CH₂—CH₂—CH₂—CH₂—CH₂—CH₂—, | | | |
| | | —CH₂—CH₂—CH₂—CH₂—CH₂—, | | | |
| | | —CH₂—CH₂—CH₂—CH₂—, | | | |
| | | —CH₂—CH₂—CH₂—, | | | |
| | | —CH₂—CH₂—, | | | |
| | | —CH₂— | | | |
| 4 | selected from | g is 1 | e is 1 | —CH₂— | —COOH |
| | group A | L² is -ethyl- | E is —S— | | |
| | (see entry 9) | | | | |
| 5 | selected from | g is 1 | e is 1 | —CH₂— | —COOH |
| | group A | L² is -butyl- | E is —S— | | |
| | (see entry 9) | | | | |
| 6 | selected from | g is 1 | e is 1 | —CH₂— | —COOH |
| | group A | L² is -propyl- | E is —O— | | |
| | (see entry 9) | | | | |
| 7 | selected from | g is 1 | e is 1 | —CH₂— | —COOH |
| | group A | L² is -ethyl- | E is —O— | | |
| | (see entry 9) | | | | |
| 8 | selected from | g is 1 | e is 1 | —CH₂— | —COOH |
| | group A | L² is -butyl- | E is —O— | | |
| | (see entry 9) | | | | |

| | |
|---|---|
| 9 | 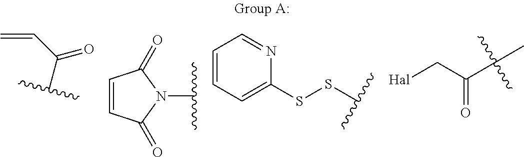 |
| | 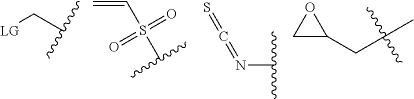 |

### Step (a)

As regards, the provision of the hydroxyalkyl starch derivative according to step (a) preferably step (a) comprises the introduction of at least one functional group Z¹ into the hydroxyalkyl starch by
(i) coupling hydroxyalkyl starch via at least one hydroxyl group to at least one suitable linker comprising the functional group Z¹ or a precursor of the functional group Z¹, or
(ii) displacing a hydroxyl group present in the hydroxyalkyl starch in a substitution reaction with a precursor of the functional group Z¹ or with a bifunctional linker comprising the functional group Z¹ or a precursor of the functional group Z¹.

According to a preferred embodiment of the present invention, the present invention relates to a method for preparing a hydroxyalkyl starch conjugate, as described above, wherein the hydroxyalkyl starch derivative comprises at least one structural unit, preferably 3 to 200 structural units, according to the following formula (I)

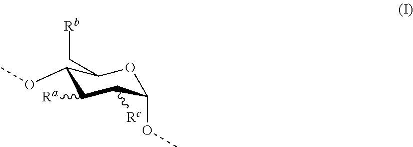

wherein at least one of R^{a}, R^{b} or R^{c} comprises the functional group Z¹, wherein R^{a}, R^{b} and R^{c} are, independently of each other, selected from the group consisting of —O—HAS″, —[O—(CR^{w}R^{x})—(CR^{y}R^{z})]ₓ—OH, —-[O—(CR^{x}R^{x})—(CR^{y}R^{z})]_{y}—Z¹, —-[O—(CR^{w}R^{x})—(CR^{y}R^{z})]_{y}—[F¹]ₚ-L¹-Z¹, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, and L¹ is a linking moiety,
and wherein step (a) comprises the steps
(a1) providing a hydroxyalkyl starch having a mean molecular weight MW above the renal threshold, preferably in the range of from 60 to 800 kDa, more preferably of from 80 to 800 kDa, and a molar substitution in the range of from 0.6 to 1.5, comprising the structural unit according to the following formula (II)

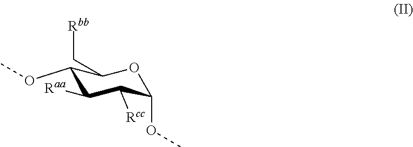

wherein R^{aa}, R^{bb} and R^{cc} are independently of each other selected from the group consisting of —[O(CR^{w}R^{x})—(CR^{y}R^{z})—(CR^{y}R^{z})]ₓ—OH and —O—HAS″, wherein HAS″ is a remainder of the hydroxyalkyl starch,
(a2) introducing at least one functional group Z¹ by
   (i) coupling the hydroxyalkyl starch via at least one hydroxyl group to at least one suitable linker comprising the functional group Z¹ or a precursor of the functional group Z¹, or
   (ii) displacing a hydroxyl group present in the hydroxyalkyl starch in a substitution reaction with a precursor of the functional group Z¹ or with a bifunctional linker comprising the functional group Z¹ or a precursor of the functional group Z¹.

Furthermore, the present invention relates to a conjugate obtained or obtainable by said method.

According to a preferred embodiment of the present invention, the present invention relates to a method for preparing a hydroxyalkyl starch conjugate, as described above, as well as to a conjugate obtained or obtainable by said method, wherein the hydroxyalkyl starch derivative provided in step (a2) comprises at least one structural unit, preferably 3 to 200 structural units, according to the following formula (I)

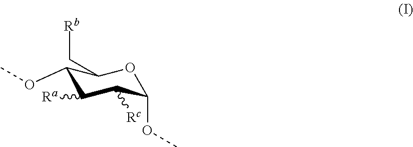

wherein R^{a}, R^{b} and R^{c} are independently of each other selected from the group consisting of —O—HAS″, —[O—CH₂—CH₂]ₓ—OH, —-[O—CH₂—CH₂]ₜ—Z¹ and —-[O—CH₂—CH₂]ₜ-[F¹]ₚ-L¹-Z¹, with p being 0 or 1, and wherein at least one of R^{a}, R^{b} and R^{c} comprises the functional group Z¹, and wherein t is in the range of from 0 to 4, wherein s is in the range of from 0 to 4.

Hydroxyalkyl starches having the desired properties are preferably produced from waxy maize starch or potato starch by acidic hydrolysis and reaction with ethylene oxide and purification by ultrafiltration.

Step (a2)-(i)

According to a first preferred embodiment of the present invention, the functional group Z¹ is introduced by coupling the hydroxyalkyl starch via at least one hydroxyl group to at least one suitable linker comprising the functional group Z¹ or a precursor of the functional group Z¹.

Organic chemistry offers a wide range of reactions to modify hydroxyl groups with linker constructs bearing functionalities such as aldehyde, keto, hemiacetal, acetal, alkynyl, azide, carboxy, alkenyl and thiol reactive groups, such as maleimide, halogens, pyridyl disulfides, haloacetamides, vinyl sulfones, vinyl pyridines, —SH, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, wherein G is O, NH or S, preferably O or S, and if present twice may be the same or may be different from each other. However, the hydroxyalkyl starch's polymeric nature and the abundance of hydroxyl groups present in the hydroxyalkyl starch usually strongly promote the number of possible side reactions such as inter- and intramolecular crosslinking. Therefore, a method was needed to functionalize the polymer under maximum retention of its molecular characteristics such as solubility, molecular weight and polydispersity. It was surprisingly found that when using the method according to this preferred embodiment, possible side reactions such as inter- and intramolecular crosslinking can be significantly diminished.

According to a preferred embodiment of the present invention, in step (a2)-(i), the hydroxyalkyl starch is coupled to a linker comprising a functional group Z², said functional group Z² being capable of being coupled to a hydroxyl group of the hydroxyalkyl starch, thereby forming a covalent linkage between the first linker and the hydroxyalkyl starch. Further, the linker preferably comprises the functional group Z¹ or a precursor thereof. According to a particularly preferred embodiment, the linker comprises a precursor of the functional group Z¹ which is transformed in at least one further step to give the functional group Z¹.

### The Functional Group Z²

The functional group Z² is a functional group capable of being coupled to at least one hydroxyl function of the hydroxyalkyl starch or to an activated hydroxyl function of hydroxyalkyl starch, thereby forming a covalent linkage F¹.

According to a preferred embodiment, the functional group Z² is a leaving group or a nucleophilic group. According to an alternative embodiment, the functional group Z² is an epoxide.

According to a first preferred embodiment, Z² is a leaving group, preferably a leaving group being attached to a CH₂— group comprised in the at least one suitable linker which is reacted in step (a2)-(ii) with the hydroxyalkyl starch. The term “leaving group” as used in this context of the present invention is denoted to mean a molecular fragment that departs with a pair of electrons in heterolytic bond cleavage upon reaction with the hydroxyl group of the hydroxyalkyl starch, thereby forming a covalent bond between the oxygen atom of the hydroxyl group and the carbon atom formerly bearing the leaving group. Common leaving groups are, for example, halides such as chloride, bromide and iodide, and sulfonates such as tosylates, mesylates, fluorosulfonates, triflates and the like. According to a preferred embodiment of the present invention, the functional group Z² is a halide leaving group. Thus, upon reaction of the hydroxyl group with the functional group Z², preferably a functional group F¹ is formed, which is preferably an —O— group.

Alternatively, Z² may also be an epoxide group, which reacts with a hydroxyl group of HAS in a ring opening reaction, thereby forming a covalent bond.

According to another embodiment, Z² is a nucleophile, thus a group capable of forming a covalent bond with an electrophile by donating both bonding electrons. In case Z² is a nucleophile, the method preferably comprises an initial step, in which at least one hydroxyl function of hydroxyalkyl starch is activated, thereby forming an electrophilic group. For example, the hydroxyl group may be activated by reacting at least one hydroxyl function with a reactive carbonyl compound, as described in detail below. Thus, the present invention also describes a method, as described above, wherein the functional group Z² is a nucleophile, said nucleophile being capable of being reacted with at least one activated hydroxyl function of hydroxyalkyl starch, as described above, wherein the hydroxyl group is initially activated with a reactive carbonyl compound prior to coupling the hydroxyalkyl starch in step (a2)-(ii) to the at least one suitable linker comprising the functional group Z² and the functional group Z¹ or a precursor of the functional group Z¹.

The term “reactive carbonyl compound” as used in this context of the present invention, refers to carbonyl di-cation synthons having a structure R**—(C═O)—R*, wherein R* and R** may be the same or different, and wherein R* and R** are both leaving groups. As leaving groups halides, such as chloride, and/or residues derived from alcohols, may be used. The term “residue derived from alcohols” refers to R* and/or R** being a unit —O—R^{ff} or O—R^{gg}, with —O—R^{ff} and O—R^{gg} preferably being residues derived from alcohols such as N-hydroxy succinimide or sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, heterocycles such as imidazol or hydroxyazoles such as hydroxybenzotriazole may be mentioned. Reactive carbonyl compounds containing halides are phosgene, related compounds such as diphosgene or triphosgene, chloroformic esters and other phosgene substitutes known in the art. Especially preferred are carbonyldiimidazol (CDI), N,N′-disuccinimidyl carbonate and sulfo-N,N′-disuccinimidyl carbonate, or mixed compounds such as p-nitrophenyl chloroformate.

Preferably, the reactive carbonyl compound having the structure R** (C═O)—R* is selected from the group consisting of phosgene, diphosgene, triphosgene, chloroformates and carbonic acid esters, more preferably from the group consisting of p-nitrophenylchloroformate, pentafluorophenylchloroformate, N,N′-disuccinimidyl carbonate, sulfo-N,N′-disuccinimidyl carbonate, dibenzotriazol-1-yl carbonate and carbonyldiimidazol.

Preferably upon reaction of at least one hydroxyl group with the reactive carbonyl compound R**-(C═O)—R′ prior to the coupling step according to step (a2)-(ii) an activated hydroxyalkyl starch derivative is formed, which comprises at least one structural unit, preferably 3 to 200 structural units, according to the following formula (Ib)

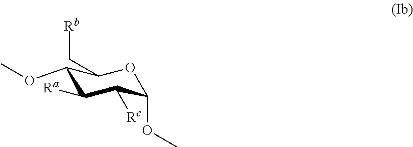

wherein R^{a}, R^{b} and R^{c} are independently of each other selected from the group consisting of —O—HAS″, —[O—CH₂—CH₂]ₛ—OH and —[O—CH₂—CH₂]ₜ—O—C(═O)—R*, wherein t is in the range of from 0 to 4, and wherein s is in the range of from 0 to 4, and wherein at least one of R^{a}, R^{b} and R^{c} comprises the group —[O—CH₂—CH₂]ₜ—O—C(═O)—R*, and wherein R* is a leaving group, preferably a group selected from the group consisting of p-nitrophenoxy-, 2,4-dichlorophenoxy, 2,4,6-trichlorophenoxy, trichloromethoxy, imidazolyl, azide and halides, such as chloride or bromide.

According to this embodiment, according to which the hydroxyalkyl starch is activated to give a hydroxyalkyl starch derivative comprising a reactive —O—C(═O)—R* group, Z² is preferably a nucleophilic group, such as a group comprising an amino group.

Possible groups are, for example, NHR^{Z2}, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂ wherein G is O or S, and if present twice in one structural unit, may be the same or may be different, and wherein R^{z2} is an alkyl group, preferably methyl. More preferably, Z² is —NH₂ or —NHR^{z2}, most preferably —NH₂.

As described above, besides the functional group Z², the linker comprises either the functional group Z¹ or a precursor thereof.

Preferably, the linker further comprises the functional group W, this functional group being a group capable of being transformed in at least one further step to give the functional group Z¹. Preferably W is an epoxide or a functional group which is transformed in a further step to give an epoxide or W has the structure Z¹*-PG, with PG being a suitable protecting group, and wherein Z¹* is the protected form of Z¹.

### Synthesis of the Hydroxyalkyl Starch Derivative Via an Epoxide Modified Hydroxyalkyl Starch Derivative

According to a first preferred embodiment, in step (a2)-(i), a first linker is used comprising the functional group W, wherein W is an epoxide or a functional group which is transformed in a further step to give an epoxide.

Thus, the present invention also relates to a method for preparing a hydroxyalkyl starch conjugate, as described above, and a hydroxyalkyl starch conjugate obtained or obtainable by said method, wherein step (a2)-(i) comprises the step (I)
(1) coupling the hydroxyalkyl starch (HAS) via at least one hydroxyl group comprised in HAS to a first linker comprising a functional group Z² capable of being reacted with the at least one hydroxyl group of the hydroxyalkyl starch, thereby forming a covalent linkage between the first linker and the hydroxyalkyl starch, the first linker further comprising a functional group W, wherein the functional group W is an epoxide or a group which is transformed in a further step to give an epoxide.

Preferably, the first linker has the structure Z²-L^{w}-W, wherein Z² is a functional group capable of being reacted with at least one hydroxyl group of hydroxyalkyl starch, as described above, and wherein L^{W} is a linking moiety.

Thus, the present invention also relates to a method for preparing a hydroxyalkyl starch conjugate, as described above, and a hydroxyalkyl starch conjugate obtained or obtainable by said method, wherein step (a2)-(i) comprises the step (1)
(I) coupling the hydroxyalkyl starch via at least one hydroxyl group comprised in HAS to a first linker having a structure according to the following formula Z²-L^{w}-W, wherein Z² is a functional group capable of being reacted with at least one hydroxyl group of hydroxyalkyl starch, as described above, and wherein L^{W} is a linking moiety, and wherein, upon reaction of the hydroxyalkyl starch, a hydroxyalkyl starch derivative is formed comprising at least one structural unit, preferably 3 to 200 structural units, according to the following formula (Ib)

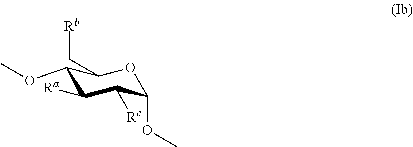

wherein R^{a}, R^{b} and R^{c} are independently of each other selected from the group consisting of —O—HAS″, —[O(CR^{w}R^{x})—(CR^{y}R^{z})]ₓ—OH and —-[O—(CR^{w}R^{x})—(CR^{y}R^{z})]_{y}-[F¹]ₚ-L^{w}-W, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{c} comprises the group —-[O—(CR^{w}R^{x})—(CR^{y}R^{z})]_{y}-[F¹]ₚ-L^{w}-W, and wherein [F¹]ₚ is the functional group being formed upon reaction of Z² with the at least one hydroxyl group of the hydroxyalkyl starch, more preferably, wherein R^{a}, R^{b} and R^{c} are independently of each other selected from the group consisting of —O—HAS″, —[O—CH₂—CH₂]ₛ—OH and —[O—CH₂—CH₂]ₜ-[F¹]ₚ-L^{w}-W, and wherein t is in the range of from 0 to 4 and wherein s is in the range of from 0 to 4, and p is 1, and wherein at least one of R^{a}, R^{b} and R^{c} comprises the group —[O—CH₂—CH₂]ₜ-[F¹]ₚ-L^{W}-W, and wherein [F¹]ₚ is the functional group being formed upon reaction of Z² with the at least one hydroxyl group of the hydroxyalkyl starch.

According to one embodiment of the present invention, the functionalization of at least one hydroxyl group of hydroxyalkyl starch to give the epoxide comprising hydroxyalkyl starch, is carried out in a one-step procedure, wherein at least one hydroxyl group is reacted with a first linker, as described above, wherein the first linker comprises the functional group W, and wherein W is an epoxide.

Therefore, the present invention also describes a method for preparing a hydroxyalkyl starch conjugate, as described above, as well as to a hydroxyalkyl starch conjugate obtained or obtainable by said method, wherein in step (a2)-(i)-(I) the hydroxyalkyl starch is reacted with a linker comprising a functional group Z² capable of being reacted with a hydroxyl group of the hydroxyalkyl starch, thereby forming a covalent linkage, the linker further comprising a functional group W, wherein the functional group W is an epoxide.

This linker has in this case a structure according to the following formula

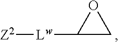

such as, for example, epichlorohydrine.

Upon reaction of this linker with at least one hydroxyl group of hydroxyalkyl starch, a hydroxyalkyl starch derivative is formed comprising at least one structural unit, preferably 3 to 200 structural units, according to the following formula (Ib)

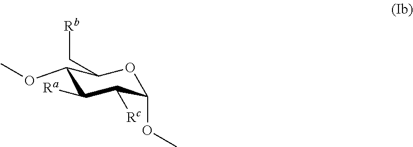

wherein R^{a}, R^{b} and R^{c} are independently of each other selected from the group consisting of —O—HAS″, —[O—(CR^{w}R^{x})—(CR^{y}R^{z})]ₓ—OH and

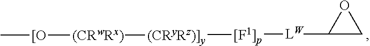

and wherein at least one of R^{a}, R^{b} and R^{c} comprises the group

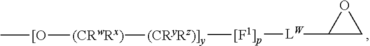

preferably wherein R^{a}, R^{b} and R^{c} are independently of each other selected from the group consisting of —O—HAS″, —[O—CH₂—CH₂]ₛ—OH and

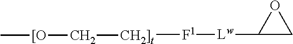

(i.e. p is 1), and wherein t is in the range of from 0 to 4 and wherein s is in the range of from 0 to 4, and wherein at least one of R^{a}, R^{b} and R^{c} comprises the group

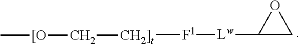

According to a preferred embodiment of the invention, the epoxide is generated in a two-step procedure, comprising the steps (I) and (II)
(I) coupling at least one hydroxyl group of the hydroxyalkyl starch, preferably of hydroxyethyl starch, to a first linker, comprising a functional group Z² capable of being reacted with a hydroxyl group of the hydroxyalkyl starch, thereby forming a covalent linkage between the first linker and the hydroxyalkyl starch, the linker further comprising a functional group W, wherein the functional group W is a functional group which is capable of being transformed in a further step to give an epoxide, such as an alkenyl group,
(II) transforming the functional group W to give an epoxide.

It was surprisingly found that this two-step procedure is superior to the one-step procedure in that higher loadings of the hydroxyalkyl starch with epoxide groups can be achieved and/or undesired side reactions such as inter- and intra-molecular crosslinking can be substantially avoided.

Preferably, the functional group W is an alkenyl group. In this case, step (II) preferably comprises the oxidation of the alkenyl group to give an epoxide and transforming the epoxide to give the functional group Z¹.

According to a preferred embodiment, the present invention also relates to a method for preparing a hydroxyalkyl starch conjugate, as described above, wherein the hydroxyalkyl starch, preferably the hydroxyethyl starch, is coupled in step (a2)-(i) via at least one hydroxyl group to at least one suitable linker, the linker having the structure Z²-L^{w}-W, wherein upon reaction of a hydroxyl group of the hydroxyalkyl starch with the linker, the leaving group Z² departs, thereby forming a covalent linkage between the hydroxyalkyl starch and the linking moiety L^{w}, and wherein the functional group F¹ which links the hydroxyalkyl starch and the linking moiety L^{w}, is an —O— bond. Likewise, the present invention also relates to the respective hydroxyalkyl starch conjugates obtained or obtainable by said method.

According to the present invention, the term “linking moiety L^{w}” as used in the context of the present invention relates to any suitable chemical moiety bridging the functional group Z² and the functional group W.

In general, there are no particular restrictions as to the chemical nature of the linking moiety L^{W} with the proviso that L^{W} has particular chemical properties enabling carrying out the inventive method for the preparation of the novel derivatives comprising the functional group Z¹, i.e. in particular, in case W is a functional group to be transformed to an epoxide, the linking moiety L^{W} has suitable chemical properties enabling the transformation of the chemical moiety W to the functional group Z¹. According to a preferred embodiment of the present invention, L^{W} bridging W and HAS′ comprises at least one structural unit according to the following formula

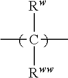

wherein R^{vv} and R^{ww} are independently of each other H or an organic residue selected from the group consisting of alkyl, alkenyl, alkylaryl, arylalkyl, aryl, heteroaryl, alkylheteroaryl and heteroarylalkyl groups.

Preferably, L^{W} is an optionally substituted, non-branched alkyl residue such as a group selected from the following groups:

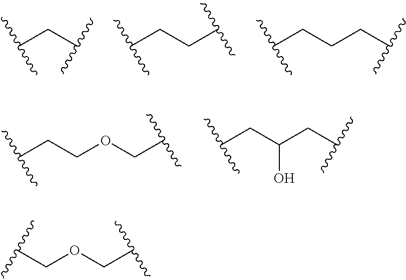

According to a first preferred embodiment of the present invention, the functional group W is an alkenyl group, wherein the first linker Z²-L^{w}-W has a structure according to the following formula

Z²-L^{W}-CH═CH₂

preferably with Z² being a leaving group or an epoxide.

Thus, preferred structures of the first linker are by way of example, the following structures:

Hal-CH₂—CH═CH₂ such as Cl-CH₂—CH═CH₂ or Br—CH₂—CH═CH₂ or I—CH₂—CH═CH₂, sulfonic esters, such as TsO—CH₂—CH═CH₂ or MsO—CH₂—CH═CH₂, epoxides such as

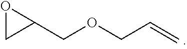

More preferably, Z² in the first linker Z²-L^{W}-W is a leaving group, most preferably, the first linker Z²-L^{W}-W has a structure according to the following formula

Hal-L^{W}-CH═CH₂.

According to an especially preferred embodiment of the present invention, the linker Z²-L^{W}-W has a structure according to the following formula

Hal-CH₂—CH═CH₂

with Hal being a halogen, preferably the halogen being iodine, bromine or chlorine, more preferably bromine.

Thus, the present invention also relates to a method for preparing a hydroxyalkyl starch conjugate, as described above, wherein in step (a2)-(i) the hydroxyalkyl starch, preferably the hydroxyethyl starch, is coupled via at least one hydroxyl group to at least one suitable linker having the structure Hal-CH₂—CH═CH₂, wherein upon reaction of the hydroxyalkyl starch with the linker, a hydroxyalkyl starch derivative is formed, comprising at least one structural unit according to the following formula (Ib)

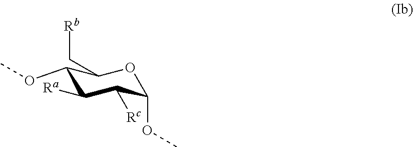

wherein R^{a}, R^{b} and R^{c} are independently of each other selected from the group consisting of —O—HAS″, —[O(CR^{w}R^{x})—(CR^{y}R^{z})]ₓ—OH and —[O—(CR^{w}R^{x})—(CR^{Y}R^{z})]_{y}—O—CH₂—CH═CH₂, and wherein at least one of R^{a}, R^{b} and R^{c} comprises the group —[O—(CR^{w}R^{x})—(CR^{y}R^{z})]_{y}—O—CH₂—CH═CH₂, preferably wherein R^{a}, R^{b} and R^{c} are independently of each other selected form the group consisting of —OH, —O—HAS″, —[O—CH₂—CH₂]ₛ—OH and —[O—CH₂—CH₂]ₜ—O—CH₂—CH═CH₂, wherein t is in the range of from 0 to 4, wherein s is in the range of from 0 to 4, and wherein at least one of R^{a}, R^{b} and R^{c} comprises the group —[O—CH₂—CH₂]ₜ—O—CH₂—CH═CH₂, and wherein the functional group —O— linking the —CH₂—CH═CH₂ group to the hydroxyalkyl starch is formed upon reaction of the linker Hal-CH₂—CH═CH₂ with the hydroxyl group of the hydroxyalkyl starch. Likewise, the present invention also relates to a hydroxyalkyl starch conjugate obtained or obtainable by the above-mentioned method.

As regards, the reaction conditions used in this step (I), wherein the hydroxyalkyl starch is reacted with the first linker, in particular wherein the first linker comprises the functional group W with W being an alkenyl, in principle any reaction conditions known to those skilled in the art can be used. Preferably, the reaction is carried out in an organic solvent, such as N-methylpyrrolidone, dimethyl acetamide (DMA), dimethyl formamide (DMF), formamide, dimethyl sulfoxide (DMSO) or mixtures of two or more thereof. More preferably, the reaction is carried out in anhydrous solvents or solvent mixtures.

Preferably, the hydroxyalkyl starch is dried prior to use, by means of heating to constant weight at a temperature range from 50 to 80° C. in a drying oven or with related techniques.

The temperature of the reaction is preferably in the range of from 5 to 55° C., more preferably in the range of from 10 to 30° C., and especially preferably in the range of from 15 to 25° C. During the course of the reaction, the temperature may be varied, preferably in the above given ranges, or held essentially constant.

The reaction time for the reaction of HAS with the linker Z²-L^{W}-W may be adapted to the specific needs and is generally in the range of from 1 h to 7 days, preferably 2 hours to 24 hours, more preferably 3 hours to 18 hours.

More preferably, the reaction is carried out in the presence of a base. The base may be added together with the linker Z²-L^{W}-W, or may be added prior to the addition of the linker, to pre-activate the hydroxyl groups of the hydroxyalkyl starch. Preferably, a base, such as alkali metal hydrides, alkali metal hydroxides, alkali metal carbonates, amine bases such as diisopropylethyl amine (DIEA) and the like, amidine bases such as 1,8-diazabicyclo[5.4.0]undec-7-ene (DBU), amide bases such as lithium diisopropylamide (LDA) or alkali metal hexamethyldisilazyl bases (e.g. LiHMDS) may be used. Most preferably the hydroxyalkyl starch is pre-activated with sodium hydride prior to the addition of the first linker Z²-L^{W}-W.

The derivative comprising the functional group W, preferably the alkenyl group, may be isolated prior to transforming this group in at least one further step to give an epoxide comprising hydroxyalkyl starch derivative. Isolation of this polymer derivative comprising the functional group W may be carried out by a suitable process which may comprise one or more steps. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated polymer derivative is first precipitated, subjected to centrifugation, re-dissolved and finally subjected to ultrafiltration.

Preferably, the precipitation is carried out with an organic solvent such as ethanol, isopropanol, acetone or tetrahydrofurane (THF). The precipitated derivative is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/I such as about 20 mmol/l, a pH value preferably in the range of from 3 to 10, more preferably of from 4 to 8, such as about 7. The number of exchange cycles preferably is in the range of from 5 to 50, more preferably of from 10 to 30, and even more preferably of from 15 to 25, such as about 20. Most preferably, the obtained derivative comprising the functional group W is further lyophilized until the solvent content of the reaction product is sufficiently low according to the desired specifications of the product.

In case W is an alkenyl, the method preferably further comprises step (II), that is the oxidation of the alkenyl group to give an epoxide group. As to the reaction conditions used in the epoxidation (oxidation) step (II), in principle, any known method to those skilled in the art can be applied to oxidize an alkenyl group to yield an epoxide.

The following oxidizing reagents are mentioned, by way of example, metal peroxysulfates such as potassium peroxymonosulfate (Oxone®) or ammonium peroxydisulfate, peroxides such as hydrogen peroxide, tert.-butyl peroxide, acetone peroxide (dimethyldioxirane), sodium percarbonate, sodium perborate, peroxy acids such as peroxoacetic acid, meta-chloroperbenzoic acid (MCPBA) or salts like sodium hypochlorite or hypobromite.

According to a particularly preferred embodiment of the present invention, the epoxidation is carried out with potassium peroxymonosulfate (Oxone®) as oxidizing agent.

Thus, the present invention also relates to a method for preparing a hydroxyalkyl starch conjugate, as described above, wherein step (a2)-(i) comprises
(1) coupling at least one hydroxyl group of the hydroxyalkyl starch, preferably of hydroxyethyl starch, to a first linker, comprising a functional group Z² capable of being reacted with a hydroxyl group of the hydroxyalkyl starch, thereby forming a covalent linkage between the first linker and the hydroxyalkyl starch, the linker further comprising a functional group W, wherein the functional group W is an alkenyl group,
(II) oxidizing the alkenyl group to give an epoxide, wherein as oxidizing agent, preferably potassium peroxymonosulfate (Oxone®) is employed.

Further, the present invention also relates to a hydroxyalkyl starch conjugate obtained or obtainable by said method.

According to an even more preferred embodiment of the present invention, the reaction with potassium peroxymonosulfate (Oxone®) is carried out in the presence of a suitable catalyst. Catalysts may consist of transition metals and their complexes, such as manganese (Mn-salene complexes are known as Jacobsen catalysts), vanadium, molybdenium, titanium (Ti-dialkyltartrate complexes are known as Sharpless catalysts), rare earth metals and the like. Additionally, metal free systems can be used as catalysts. Acids such as acetic acid may form peracids in situ and epoxidize alkenes. The same accounts for ketones such as acetone or tetrahydrothiopyran-4-one, which react with peroxide donors under formation of dioxiranes, which are powerful epoxidation agents. In case of non-metal catalysts, traces of transition metals from solvents may lead to unwanted side reactions, which can be excluded by metal chelation with EDTA.

Preferably, said suitable catalyst is tetrahydrothiopyran-4-one.

Upon epoxidation, in step (II) a hydroxyalkyl starch derivative is formed comprising at least one structural unit, preferably 3 to 200 structural units, according to the following formula (Ib)

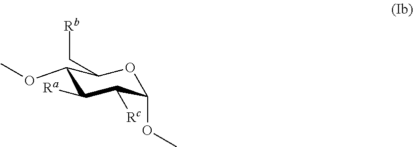

wherein R^{a}, R^{b} and R^{c} are independently of each other selected from the group consisting of —O—HAS″, —[O(CR^{w}R^{x})—(CR^{y}R^{z})]ₓ—OH and

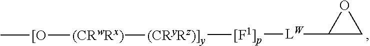

and wherein at least one of R^{a}, R^{b} and R^{c} comprises the group

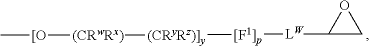

preferably wherein R^{a}, R^{b} and R^{c} are independently of each other selected from the group consisting of —O—HAS″, —[O—CH₂—CH₂]ₛ—OH and

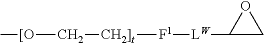

(i.e. p is 1), and wherein t is in the range of from 0 to 4 and wherein s is in the range of from 0 to 4 and wherein at least one of R^{a}, R^{b} and R^{c} comprises the group

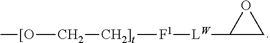

According to a preferred embodiment, the epoxidation of the alkenyl-modified hydroxyalkyl starch derivatives is carried out in aqueous medium, preferably at a temperature in the range of from 0 to 80° C., more preferably in the range of from 0 to 50° C. and especially preferably in the range of from 10 to 30° C.

During the course of the epoxidation reaction, the temperature may be varied, preferably in the above-given ranges, or held essentially constant. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably from 0.01 to 1 mM, most preferably from 0.1 to 0.5 mM, such as about 0.4 mM.

The pH value for the reaction of the HAS derivative with potassium peroxymonosulfate (Oxone®) may be adapted to the specific needs of the reactants. Preferably, the reaction is carried out in buffered solution, at a pH value in the range of from 3 to 10, more preferably of from 5 to 9, and even more preferably of from 7 to 8. Among the preferred buffers, carbonate, phosphate, borate and acetate buffers as well as tris(hydroxymethyl)aminomethane (TRIS) may be mentioned. Among the preferred bases, alkali metal bicarbonates may be mentioned.

According to the invention, the epoxide-modified HAS derivative may be purified or isolated in a further step prior to the transformation of the epoxide group to the functional group Z¹.

The separated derivative is optionally lyophilized.

After the purification step, the HAS derivative is preferably obtained as a solid. According to a further conceivable embodiment of the present invention, the HAS derivative solutions or frozen HAS derivative solutions may be mentioned.

The epoxide comprising HAS derivative is preferably reacted in a subsequent step (III) with at least one suitable reagent to yield the HAS derivative comprising the functional group Z¹. Preferably, the epoxide is reacted with a nucleophile comprising the functional group Z¹ or a precursor thereof. Preferably, the nucleophile reacts with the epoxide in a ring opening reaction and yields a HAS derivative comprising at least one structural unit, preferably 3 to 200 structural units according to the following formula (Ib)

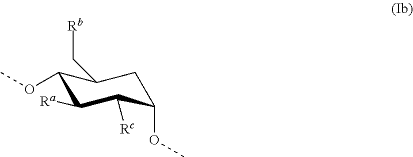

wherein at least one of R^{a}, R^{b} and R^{c} is —[O—(CR^{w}R^{x})—(CR^{y}R^{z})]_{y}-[F¹]ₚ-L^{w}-CHOH—CH₂-Nuc, preferably wherein at least one of R^{a}, R^{b} and R^{c} is —[O—CH₂—CH₂]ₜ-[F¹]ₚ-L^{W}-CHOH—CH₂—Nuc, wherein the residue Nuc is the remaining part of the nucleophile covalently linked to the hydroxyalkyl starch after being reacted with the epoxide.

Any nucleophile capable of reacting with the epoxide thereby forming a covalent linkage and comprising the functional group Z¹ or a precursor thereof may be used. As nucleophile, for example, linker compounds comprising at least one nucleophilic functional group capable of reacting with the epoxide and at least one functional group W′ capable of being transformed to the functional group Z¹, such as, for example, a group —Z¹—PG can be used. Alternatively, a linker such as an at least bifunctional linker comprising a nucleophilic group such as a thiol group and further comprising the functional group Z¹ may be used.

As described above, according to a particularly preferred embodiment of the present invention, Z¹ is a thiol group.

According to a further preferred embodiment of the present invention, the nucleophilic group reacting with the epoxide is a thiol group.

Thus, the present invention also relates to a method as described above, wherein step (a2)-(i) comprises
(III) reacting the epoxide with a nucleophile comprising the functional group Z¹ or a precursor of the functional group Z¹, the nucleophile additionally comprising a nucleophilic group, preferably wherein Z¹ and the nucleophilic group are both —SH groups.

According to an especially preferred embodiment of the present invention, the present invention also relates to a method for preparing a hydroxyalkyl starch conjugate, as well as to a hydroxyalkyl starch conjugate obtained or obtainable by said method, as described above, wherein the epoxide is reacted with a nucleophile comprising the functional group Z¹, with Z¹ being a thiol group, and comprising a nucleophilic group, this group being a thiol. Thus, according to a preferred embodiment, the nucleophile is a dithiol.

The invention also relates to the respective derivative obtained or obtainable by said method, said derivative preferably comprising at least one structural unit, preferably 3 to 200 structural units according to the following formula (Ib)

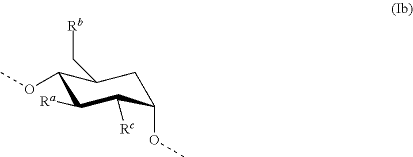

wherein at least one of R^{a}, R^{b} and R^{c} is —-[O—(CR^{w}R^{x})—(CR^{y}R^{z})]_{y}-[F¹]ₚ-L¹-SH, preferably wherein at least one of R^{a}, R^{b} and R^{c} is —[O—CH²—CH²)]ₜ-[F¹]ₚ-L¹-SH, wherein L¹ is a linking moiety which is obtained when reacting the structural unit

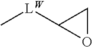

with the nucleophile and which links the functional group F¹ to the functional group Z¹. According to the preferred embodiment, the linking moiety L¹ has a structure selected from the groups below:

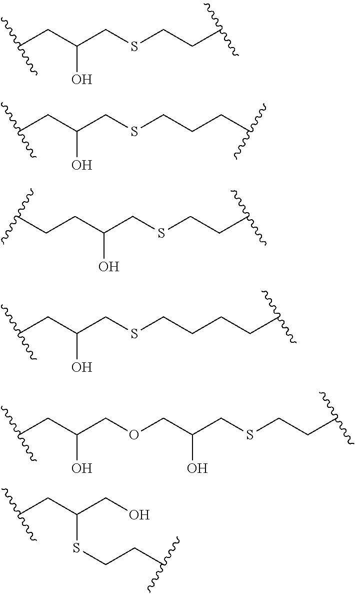

more preferably L¹ has a structure according to the following formula

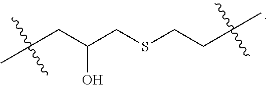 [SMILES: *CCSCC(O)C*]

According to an alternative embodiment of the present method, the epoxide is reacted with a nucleophile suitable for the introduction of thiol groups such as thiosulfate, alkyl or aryl thiosulfonates or thiourea, preferably sodium thiosulfate. Thus, the present invention also relates to a method as described above as well as to a hydroxyalkyl starch derivative obtained or obtainable by said method, wherein the epoxide-modified hydroxyalkyl starch is reacted with a nucleophile, said nucleophile being thiosulfate, alkyl or aryl thiosulfonates or thiourea, preferably sodium thiosulfate.

Upon reaction of the thiosulfate with the epoxide in a ring opening reaction, preferably a hydroxyalkyl starch derivative is formed comprising at least one structural unit, preferably 3 to 200 structural units, according to the following formula (Ib)

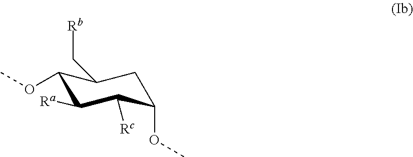

wherein at least one of R^{a}, R^{b} and R^{c} is —[O(CR^{w}R^{x})—(CR^{y}R^{z})]ₓ-[F¹]ₚ-L^{W}-CHOH—CH₂—SSO₃Na, preferably wherein at least one of R^{a}, R^{b} and R^{c} is —-[O—CH₂—CH₂]ₜ-[F¹]ₚ-L^{W}-CHOH—CH₂—SSO₃Na.

Preferably, this derivative is reduced in a subsequent step to yield the HAS derivative comprising the functional group Z¹ with Z¹ being —SH. Any suitable methods known to those skilled in the art can be used to reduce the respective intermediate shown above. Preferably, the thiosulfonate is reduced with sodium borohydride in aqueous solution.

According to a preferred embodiment of the present invention, the hydroxyalkyl starch derivative comprising the functional group Z¹, obtained by the above-described method, is purified in a further step. Again, the purification of the HAS derivative from step (III) can be carried out by any suitable method such as ultrafiltration, dialysis or precipitation or a combined method using for example precipitation and afterwards ultrafiltration. Furthermore, the HAS derivative may be lyophilized, as described above, using conventional methods, prior to step (b).

Synthesis of the Hydroxyalkyl Starch Derivative Via the Reaction of the Carboxy Activated Hydroxyalkyl Starch with a Linker Compound

According to a second embodiment, in step (a2)-(i), a linker is used, comprising the functional group Z¹ or the functional group W, wherein W has the structure —Z¹-PG, with PG being a suitable protecting group. Preferably, in case this linker is used, the hydroxyalkyl starch is activated prior to the reaction using a reactive carbonate as described above.

Thus, the present invention also relates to a method, as described above, wherein step (a2)-(i) comprises
(aa) activating at least one hydroxyl group comprised in the hydroxyalkyl starch with a reactive carbonyl compound having the structure R**—(C═O)—R*, wherein R* and R** may be the same or different, and wherein R* and R** are both leaving groups, wherein upon activation an activated hydroxyalkyl starch derivative comprising at least one structural unit according to the following formula (Ib)

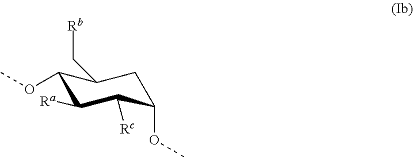

is formed, in which R^{a}, R^{b} and R^{c} are independently of each other selected from the group consisting of —O—HAS″, —[O—CH₂—CH₂]ₛ—OH, —[O—CH₂—CH₂]ₜ—O—C(═O)—R*, wherein s is in the range of from 0 to 4, wherein t is in the range of from 0 to 4, wherein at least one of R^{a}, R^{b} and R^{c} comprises the group —[O—CH₂—CH₂]ₜ—O—C(═O)—R*, and
(bb) reacting the activated hydroxyalkyl starch derivative according to step (aa) with the suitable linker comprising the functional group Z¹ or a precursor of the functional group Z¹.

The invention further relates to a conjugate obtained or obtainable by said method.

In particular, in step (a2)-(i) the hydroxyalkyl starch is reacted with a linker comprising the functional group Z¹ or a precursor thereof and a functional group Z², the linker preferably having the structure Z²-L¹-Z¹ or Z²-L¹-Z¹*-PG, with Z² being a functional group capable of being reacted with the hydroxyalkyl starch or an activated hydroxyalkyl starch, preferably with an activated hydroxyalkyl starch, the method further comprising activating the hydroxyalkyl starch prior to the reaction with the linker using a reactive carbonate, and with Z¹* being the protected form of the functional group Z¹.

As described above, the linker preferably comprises a functional group Z², which in this case, is preferably a nucleophile, such as a group comprising an amino group, more preferably a group selected from the group consisting of —NHR^{Z2}, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, wherein G is O or S, and if present twice in one structural unit, may be the same or may be different, and wherein R^{Z2} is an alkyl group, preferably methyl. More preferably Z² is —NH₂ or —NHR^{Z2}, most preferably —NH₂.

The linker has preferably a structure Z²-L¹-Z¹*-PG, wherein Z¹* is in particular —S-(and the respective unprotected functional group Z¹ a thiol group). According to this embodiment, the linking moiety L¹ is preferably an alkyl group. More preferably, the linking moiety L¹ is a spacer comprising at least one structural unit according to the formula —{[CR^{d}R^{f}]ₕ-[F⁴]ᵤ—[CR^{dd}R^{ff}]_{z}}ₐₗₚₕₐ—, as described above, wherein integer alpha is in the range of from 1 to 10, and wherein F⁴ is preferably selected from the group consisting of —S—, —O— and —NH—, more preferably wherein F⁴, if present, is —O— or —S—, more preferably wherein F⁴ is —S—. As described above, in the context of the preferred conjugates, residues R^{d}, R^{f}, R^{dd} and R^{ff} are, independently of each other, preferably selected from the group consisting of halogens, alkyl groups, H or hydroxyl groups. More preferably, these residues are independently from each other H, alkyl or hydroxyl groups. Preferably, integer u and integer z of the formula —{[CR^{d}R^{f}]ₕ-[F⁴]ᵤ—[CR^{dd}R^{ff}]_{z}}ₐₗₚₕₐ— are 0, and alpha is 1, the linking moiety L¹ thus corresponds to the structural unit —[CR^{d}R^{f}]ₕ—. The integer h is preferably in the range of from 1 to 20, more preferably of from 1 to 10, such as 1, 2, 3, 4, 5, 6, 7, 8, 9 or 10, more preferably of from 1 to 5, most preferably of from 1 to 3. More preferably R^{d} and R^{f} are both H. Thus, by way of example, the following preferred linker moieties L¹ are mentioned: —CH₂—, —CH₂—CH₂—, CH₂—CH₂—CH₂—, —CH₂—CH₂—CH₂—CH₂—, —CH₂—CH₂—CH₂—CH₂—CH₂—, more preferably —CH₂—CH₂—.

In case Z¹ is a thiol group, and Z¹* is —S—, the group PG is preferably a thiol protecting group, more preferably a protecting group forming together with Z¹* a thioether (e.g. trityl, benzyl, allyl), a disulfide (e.g. S-sulfonates, S-tert.-butyl, S-(2-aminoethyl)) or a thioester (e.g. thioacetyl). In case the linker comprises a protecting group, the method further comprises a deprotection step.

In case the group —Z¹*-PG is a disulfide, and Z¹* is —S—, the linker Z²-L¹-S-PG is preferably a symmetrical disulfide, with PG having the structure S-L¹-Z². As preferred linker compound, thus cystamine and the like, may be mentioned.

In the context of this embodiment, the following linker compounds having the structure Z²-L¹-Z¹—PG are mentioned by way of example: H₂N—CH₂—S-Trt, H₂N—CH₂—CH₂—S-Trt, H₂N—CH₂—CH₂—CH₂—S-Trt, H₂N—CH₂—CH₂—CH₂—CH₂—S-Trt, H₂N—CH₂—CH₂—CH₂—CH₂—CH₂—S-Trt, H₂N—CH₂—CH₂—S—S—CH₂—CH₂—NH₂, H₂N—CH₂—CH₂—S—S-tBu, wherein Trt is a trityl group.

Subsequent to the activation, the hydroxyalkyl starch is preferably reacted with the linker Z²-L¹-Z¹*-PG, thereby most preferably forming a derivative, comprising the functional group Z¹*-PG, more preferably this derivative comprises at least one structural unit, preferably 3 to 200 structural units, according to the following formula (Ib)

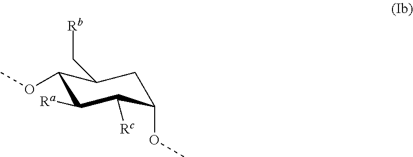

wherein at least one of R^{a}, R^{b} and R^{c} is —[O(CR^{w}R^{x})—(CR^{y}R^{z})]ₓ—F¹-L¹-Z¹*-PG, more preferably wherein R^{a}, R^{b} and R^{c} are independently of each other selected from the group consisting of —O—HAS″, —-[O—CH₂—CH₂]-OH, and —-[O—CH₂—CH₂]ₜ—F¹-L¹-Z¹*-PG, wherein t is in the range of from 0 to 4, and wherein s is in the range of from 0 to 4, and wherein at least one of R^{a}, R^{b} and R^{c} comprises the group —[O—CH₂—CH₂]t-F¹-L¹-Z¹*-PG, and wherein F¹ is the functional group being formed upon reaction of the group —O—C(═O)—R* with the functional group Z². According to a preferred embodiment, the functional group Z² is —NH₂, thus F¹ preferably has the structure O—C(═O)—NH—.

The coupling reaction between the activated hydroxyalkyl starch and the linker, comprising the functional group Z¹ or the functional group W, wherein W has preferably the structure —Z¹*-PG, with PG being a suitable protecting group, in principle any reaction conditions known to those skilled in the art can be used. Preferably, the reaction is carried out in an organic solvent, such as N-methylpyrrolidone, dimethyl acetamide (DMA), dimethyl formamide (DMF), formamide, dimethyl sulfoxide (DMSO), or mixtures of two or more thereof, preferably at a temperature in the range of from 5 to 80° C., more preferably in the range of from 5 to 50° C. and especially preferably in the range of from 15 to 30° C. The temperature may be held essentially constant or may be varied during the reaction procedure.

The pH value for this reaction may be adapted to the specific needs of the reactants. Preferably, the reaction is carried out in the presence of a base. Among the preferred bases pyridine, substituted pyridines, such as 4-(dimethylamino)-pyridine, 2,6-lutidine or collidine, tertiary amine bases such as triethyl amine, diisopropyl ethyl amine (DIEA), N-methyl morpholine, amidine bases such as 1,8-diazabicyclo[5.4.0]undec-7-ene or inorganic bases such as alkali metal carbonates may be mentioned.

The reaction time for the reaction of activated hydroxyalkyl starch with the linker Z²-L¹-Z¹*-PG or Z²-L¹-Z¹ may be adapted to the specific needs and is generally in the range of from 1 h to 7 days, preferably 2 hours to 48 hours, more preferably 4 hours to 24 hours.

The derivative comprising the functional group Z¹*-PG or Z¹, may be subjected to at least one further isolation and/or purification step. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated polymer derivative is first precipitated, subjected to centrifugation, re-dissolved and finally subjected to ultrafiltration.

Preferably, the precipitation is carried out with an organic solvent such as ethanol, isopropanol, acetone or tetrahydrofurane (THF). The precipitated conjugate is subsequently subjected to centrifugation and subsequent ultrafiltration using water or an aqueous buffer solution having a concentration preferably from 1 to 1000 mmol/l, more preferably from 1 to 100 mmol/I, and more preferably from 10 to 50 mmol/l, such as about 20 mmol/I, a pH value preferably in the range of from 3 to 10, more preferably of from 4 to 8, such as about 7. The number of exchange cycles preferably is in the range of from 5 to 50, more preferably of from 10 to 30, and even more preferably of from 15 to 25, such as about 20.

Most preferably the obtained derivative is further lyophilized until the solvent content of the reaction product is sufficiently low according to the desired specifications of the product.

In case the linker comprises a protecting group (PG), the method preferably further comprises a deprotection step. The reaction conditions used are adapted to the respective protecting group used. According to a preferred embodiment of the invention, Z¹ is a thiol group, and the group Z¹*-PG is a disulfide, as described above. In this case, the deprotection step comprises the reduction of this disulfide bond to give the respective thiol group. This deprotection step is preferably carried out using specific reducing agents. As possible reducing agents, complex hydrides such as borohydrides, especially sodium borohydride, and thiols, especially dithiothreitol (DTT) and dithioerythritol (DTE) or phosphines such as tris-(2-carboxyethyl)phosphine (TCEP) are mentioned. The reduction is preferably carried out using DTT.

The deprotection step is preferably carried out at a temperature in the range of from 0 to 80° C., more preferably in the range of from 10 to 50° C. and especially preferably in the range of from 20 to 40° C. During the course of the reaction, the temperature may be varied, preferably in the above-given ranges, or held essentially constant.

Preferably, the reaction is carried out in aqueous medium. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably from 0.01 to 1 mM, most preferably from 0.1 to 0.5 mM, such as about 0.4 mM.

The pH value in the deprotection step may be adapted to the specific needs of the reactants. Preferably, the reaction is carried out in buffered solution, at a pH value in the range of from 3 to 14, more preferably of from 5 to 11, and even more preferably of from 7.5 to 8.5. Among the preferred buffers, carbonate, phosphate, borate and acetate buffers as well as tris(hydroxymethyl)aminomethane (TRIS) may be mentioned.

Again, at least one isolation step/and or purification step, as described above, may be carried out subsequent to the deprotection step. Most preferably the obtained derivative is further lyophilized prior to step (b) until the solvent content of the reaction product is sufficiently low according to the desired specifications of the derivative.

Step (a2)-(ii)

As regards step (a2)-(ii) of the method according to the present invention, in this step, the functional group Z¹ is introduced by displacing a hydroxyl group present in the hydroxyalkyl starch in a substitution reaction with a precursor of the functional group Z¹ or with a bifunctional linker comprising the functional group Z¹ or a precursor thereof.

Preferably, prior to the replacement of the hydroxyl group with the functional group Z¹, the at least one hydroxyl group of the hydroxyalkyl starch is activated to generate a suitable leaving group. Preferably, a group R^{L} is added to the at least one hydroxyl group thereby generating a group —O—R^{L}, wherein the structural unit —O—R^{L} is the leaving group.

Thus, the present invention also relates to a method for preparing a hydroxyalkyl starch conjugate, as described above, as well as to a hydroxyalkyl starch conjugate obtained or obtainable by said method wherein in step (a2)-(ii), prior to the substitution (displacement) of the hydroxyl group with the group comprising the functional group Z¹ or a precursor thereof, a group R^{L} is added to at least one hydroxyl group thereby generating a group —O—R^{L}, wherein —O—R^{L} is the leaving group.

The term “leaving group” as used in this context of the present invention is denoted to mean that the molecular fragment O—R^{L} departs when reacting the hydroxyalkyl starch derivative with a reagent, such as a crosslinking compound, comprising the functional group Z¹ or a precursor thereof.

As regards, preferred leaving groups used in this context of the present invention, according to a preferred embodiment, the hydroxyl group is transformed to a sulfonic ester, such as a mesylic ester (—OMs), tosylic ester (—OTs), imsyl ester (imidazylsulfonyl ester) or a carboxylic ester such as trifluoroacetic ester.

Preferably, the at least one leaving group is generated by reacting at least one hydroxyl group of hydroxyalkyl starch, preferably in the presence of a base, with the respective sulfonyl chloride to give the sulfonic ester, preferably the mesylic ester.

Thus, the present invention also relates to a method for preparing a hydroxyalkyl starch conjugate as described above, as well as to a hydroxyalkyl starch conjugate obtained or obtainable by said method, wherein in step (a2)-(ii), prior to the substitution (displacement) of the hydroxyl group with the group comprising the functional group Z¹ or a precursor thereof, a group R^{L} is added to at least one hydroxyl group, thereby generating a group —O—R^{L}, wherein —O—R^{L} is —O-Ms or —OTs (i.e. R^{L} is Ms or Ts), and wherein the —O-Ms group is preferably introduced by reacting at least one hydroxyl group of hydroxyalkyl starch with methanesulfonyl chloride, and —OTs is introduced by reacting at least one hydroxyl group with toluenesulfonyl chloride.

The addition of the group R^{L} to at least one hydroxyl group of hydroxyalkyl starch, whereupon a group —O—R^{L} is formed, is preferably carried out in an organic solvent, such as N-methylpyrrolidone, dimethyl acetamide (DMA), dimethyl formamide (DMF), formamide, dimethylsulfoxide (DMSO) and mixtures of two or more thereof, preferably at a temperature in the range of from −60 to 80° C., more preferably in the range of from −30 to 50° C. and especially preferably in the range of from −30 to 30° C. The temperature may be held essentially constant or may be varied during the reaction procedure. The pH value for this reaction may be adapted to the specific needs of the reactants. Preferably, the reaction is carried out in the presence of a base. Among the preferred bases pyridine, substituted pyridines such as collidine or 2,6-lutidine, tertiary amine bases such as triethylamine, diisopropyl ethyl amine (DIEA), N-methylmorpholine, N-methyl imidazole or amidine bases such as 1,8-diazabicyclo[5.4.0]undec-7-ene (DBU) and inorganic bases such as metal hydrides and carbonates may be mentioned. Especially preferred are substituted pyridines (collidine) and tertiary amine bases (DIEA, N-methylmorpholine). The reaction time for this reaction step may be adapted to the specific needs and is generally in the range of from 5 min to 24 hours, preferably 15 min to 10 hours, more preferably 30 min to 5 hours.

The derivative comprising the group —O—R^{L}, may be subjected to at least one further isolation and/or purification step such as precipitation and/or centrifugation and/or filtration prior to the substitution reaction according to step (a2)-(ii). Likewise, instead or additionally, the derivative comprising the —O—R^{L} group may be subjected to an after-treatment like ultrafiltration, dialysis, centrifugal filtration or pressure filtration, ion exchange chromatography, reversed phase chromatography, HPLC, MPLC, gel filtration and/or lyophilization. According to a preferred embodiment, the derivative comprising the —O—R^{L} group is in situ reacted with the precursor of the functional group Z¹ or with the bifunctional linker, comprising the functional group Z¹ or a precursor thereof.

As described above, the at least one hydroxyl group, preferably the at least one —O—R^{L} group, more preferably the O-Ms group, is displaced, in a substitution reaction, with the precursor of the functional group Z¹ or with a bifunctional linker comprising the functional group Z¹ or a precursor thereof.

According to a preferred embodiment of the present invention, the activated hydroxyl group, preferably the —O—R^{L} group, more preferably the O-Ms group, is reacted with the precursor of the functional group Z¹. The term “a precursor” as used in this context of the present invention is denoted to mean a reagent which is capable of displacing the group, thereby forming a functional group Z¹ or a group, which can be modified in at least one further step to give the functional group Z¹.

Thus, the present invention also relates to a method for preparing a hydroxyalkyl starch conjugate, as described above, as well as to a hydroxyalkyl starch conjugate obtained or obtainable by said method, wherein in step (a2)-(ii), prior to the substitution (displacement) of the hydroxyl group with the group comprising the functional group Z¹ or a precursor thereof, a group R^{L} is added to at least one hydroxyl group, thereby generating a group —O—R^{L}, wherein —O—R^{L} is a leaving group, and subsequently —O—R^{L} is replaced by a precursor of the functional group Z¹, the method further comprising converting the precursor after the substitution reaction to the functional group Z¹, and wherein Z¹ is preferably a thiol group.

In case Z¹ is an amine, reagents such as ammonia, hydrazine, acyl hydrazides, such as carbohydrazide, potassium phthalimide, azides, such as sodium azide, and the like, can be employed to introduce the functional group Z¹

In case Z¹ is a thiol group, reagents such as thioacetic acid, alkyl or aryl thiosulfonates such as sodium benzenethiosulfonate, thiourea, thiosulfate or hydrogen sulfide can be employed as precursor to introduce the functional group Z¹.

According to an especially preferred embodiment of the present invention, the hydroxyl group present in the hydroxyalkyl starch is first activated and then reacted with thioacetate, thereby replacing the hydroxyl group with the structure —S—C(═O)—CH₃. A particularly preferred reagent is potassium thioacetate. Thus, the present invention also relates to a method, as described above, wherein in step (a2)-(ii) the hydroxyl group present in the hydroxyalkyl starch is reacted with thioacetate giving a functional group having the structure —S—C(═O)—CH₃.

In this substitution step, in principle any reaction conditions known to those skilled in the art can be used. Preferably, the reaction is carried out in organic solvents, such as N-methyl pyrrolidone, dimethyl acetamide (DMA), dimethyl formamide (DMF), formamide, dimethyl sulfoxide (DMSO) and mixtures of two or more thereof. Preferably this step is carried out at a temperature in the range of from 0 to 80° C., more preferably in the range of from 20 to 70° C. and especially preferably in the range of from 40 to 60° C. The temperature may be held essentially constant or may be varied during the reaction procedure.

The pH value for this reaction may be adapted to the specific needs of the reactants. Optionally, the reaction is carried out in the presence of a scavenger, which reacts with the leaving group —O—R^{L}, such as mercaptoethanol or the like.

The reaction time for the substitution step is generally in the range of from 1 hour to 7 days, preferably 3 to 48 hours, more preferably 4 to 18 hours.

The derivative obtained may be subjected to at least one further isolation and/or purification step, as described above.

Preferably, the derivative is subjected to at least one further step. In particular, in case the hydroxyl group present in the hydroxyalkyl starch is reacted with thioacetate, thereby replacing the hydroxyl group with the structure —S—C(═O)—CH₃, the derivative is preferably saponified in a subsequent step to give the functional group Z¹ with Z¹ being an —SH group. Thus, the present invention also relates to a method as described above as well as to a conjugate obtained or obtainable by said method, wherein in step (a2)-(ii), the hydroxyl group present in the hydroxyalkyl starch is reacted with thioacetate giving a functional group having the structure —S—C(═O)—CH₃, wherein the method further comprises saponification of the group —S—C(═O)—CH₃ to give the functional group Z¹.

It has to be understood, that in case at least one hydroxyl group present in hydroxyalkyl starch, comprising the structural unit according to the following formula (II)

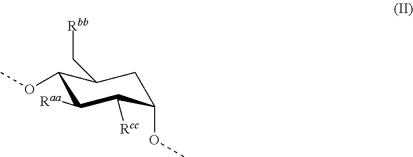

with R^{aa}, R^{bb} and R^{cc} being independently of each other selected from the group consisting of —[O—(CR^{w}R^{x})—(CR^{y}R^{z})]ₓ—OH and —O—HAS″, is displaced in a substitution reaction, the stereochemistry of the carbon atom which bears the respective hydroxyl function, which is displaced, may be inverted.

Thus, in case at least one of R^{aa} and R^{bb} in the above shown structural unit is OH (i.e. integer x is 0), and in case, this at least one group is displaced by a precursor of the functional group Z¹, thereby yielding in a hydroxyalkyl starch derivative comprising the functional group Z¹ in this structural unit, the stereochemistry of the carbon atoms bearing this functional group Z¹ may be inverted.

Since, it cannot be excluded that such a substitution of secondary hydroxyl groups occur, in the method of the invention according to step (a2)-(ii), the stereochemistry of the carbon atoms bearing the functional group R^{a} and R^{c} is not further defined, as shown in the structure with the formula (I)

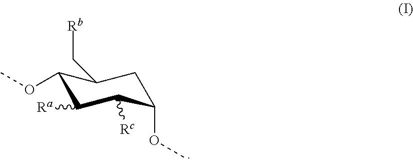

However, without wanting to be bound to any theory, it is believed that mainly primary hydroxyl groups will be displaced in the substitution reaction according to step (a2)-(ii). Thus, according to this theory, the stereochemistry of most carbon atoms bearing the residues R^{a} or R^{c} will not be inverted but the respective structural unit of the hydroxyalkyl starch will comprise the stereochemistry as shown in the formula (Ib)

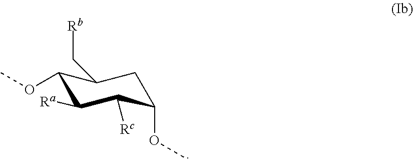

The thioacetate is preferably saponified in at least one further step to give the thiol comprising hydroxyalkyl starch derivatives. As regards the saponification of the functional group —S—C(═O)—CH₃, all methods known to those skilled in the art are encompassed by the present invention. This includes the use of bases (such as metal hydroxides) and strong nucleophiles (such as ammonia, amines, thiols or hydroxides) in order to saponify the present thioesters to give thiols. Preferred reagents are sodium hydroxide and ammonia.

Since thiols are well known to oxidize via the formation of disulfides, especially under basic conditions present in most saponification protocols, the molecular weight of the hydroxyalkyl starch derivative obtained may vary due to unspecific crosslinking. To prevent the formation of disulfides, preferably a reducing agent is added prior, during or after the saponification step. According to a preferred embodiment of the invention, a reducing agent is directly added to the saponification mixture in order to keep the forming thiol groups in their low oxidation state. Regarding the reduction of the thiol groups, all reduction methods known to those skilled in the art such as borohydrides, especially sodium borohydride, and thiols, especially dithiothreitol (DTT) and dithioerythritol (DTE) or phosphines such as tris-(2-carboxyethyl)phosphine (TCEP) are encompassed by the present invention. According to preferred embodiments of the present invention, dithiothreitol (DTT), dithioerythritol (DTE) or sodium borohydride are employed.

In an alternative embodiment of the reaction, aqueous sodium hydroxide is used as saponification agent together with sodium borohydride as reducing agent.

Optionally, mercaptoethanol can be used as an additive in this reaction.

Thus, the present invention also relates to a method, as described above, wherein in step (a2)-(ii) the at least one activated hydroxyl group present in the hydroxyalkyl starch is reacted with thioacetate giving a functional group having the structure —S—C(═O)—CH₃, wherein the method further comprises saponfying the group S—C(═O)—CH₃ to give the functional group Z¹, wherein the hydroxyalkyl starch derivative comprises at least one structural unit, preferably 3 to 200 structural units, according to the following formula (I)

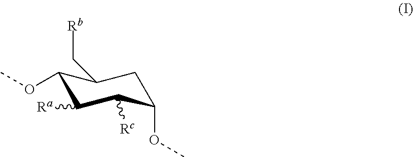

wherein R^{a}, R^{b} and R^{c} are independently of each other selected from the group consisting of —O—HAS″, —-[O—CH₂—CH₂]ₛ—OH, —-[O—CH₂—CH₂]ₜ—SH and wherein at least one R^{a}, R^{b} and R^{c} is —-[O—CH₂—CH₂]ₜ—SH and wherein t is in the range of from 0 to 4, and wherein s is in the range of from 0 to 4.

Again, the hydroxyalkyl starch derivative, comprising the functional group —SH, obtained by the above-described preferred embodiment, may be isolated/and or purified prior to step (b) in a further step. Again, the purification/isolation of the HAS derivative from step (a2)-(ii) can be carried out by any suitable method such as ultrafiltration, dialysis or precipitation or a combined method using for example precipitation and afterwards ultrafiltration.

Furthermore, the hydroxyalkyl starch derivative may be lyophilized, as described above, using conventional methods.

According to an especially preferred embodiment, the hydroxyalkyl starch derivative, obtained in step (a2)-(ii), comprises at least one structural unit according to the following formula (I)

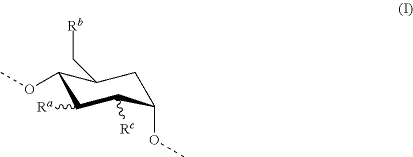

wherein R^{a}, R^{b} and R^{c} are independently of each other selected from the group consisting of —O—HAS″, —-[O—CH₂—CH₂]ₛ—OH, —[O—CH₂—CH₂]ₜ—Z¹, wherein t is in the range of from 0 to 4, and wherein s is in the range of from 0 to 4, and wherein at least one of R^{a}, R^{b} and R^{c} is —[O—CH₂—CH₂]ₜ—Z¹, with Z¹ being —SH. This derivative is preferably reacted in step (b) with a crosslinking compound L having a structure according to the following formula K²-[L³]_{g}-[E]ₑ—[CR^{m}Rⁿ]_{f}—K¹ with g and e being 0, and wherein K² is a halogen.

According to an especially preferred embodiment the hydroxyalkyl starch derivative obtained in step (a2)-(ii) comprises at least one structural unit according to the following formula (I)

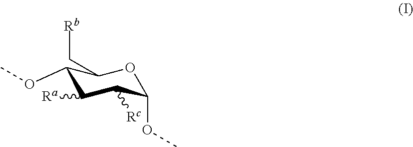

wherein R^{a}, R^{b} and R^{c} are independently of each other selected from the group consisting of —O—HAS″, —[O—CH₂—CH₂]ₛ—OH, and —[O—CH₂—CH₂]ₜ—Z¹, wherein t is in the range of from 0 to 4, and wherein s is in the range of from 0 to 4, and wherein at least one of R^{a}, R^{b} and R^{c} is —[O—CH₂—CH₂]ₜ—Z¹, with Z¹ being —SH. This derivative is preferably reacted in step (b) with a crosslinking compound L having a structure according to the formula K²-[L²]_{g}-[E]ₑ—[CR^{m}Rⁿ]_{f}—K¹, wherein K² is maleimide, and wherein upon reaction of Z¹ with K², a functional group —X—F²— is formed.

### Step (b)

As already described above, the hydroxyalkyl starch derivative obtained according to step (a) is, optionally after at least one purification and/or isolation step, further reacted in step (b).

In step (b) the HAS derivative is coupled via the functional group Z¹ to at least one cytotoxic agent via the at least bifunctional crosslinking compound L, wherein L comprises the functional groups K¹ and K², wherein L is coupled to Z¹ via a functional group K² comprised in L, and wherein each cytotoxic agent is coupled via the secondary hydroxyl group to the HAS derivative via the functional group K¹, comprised in L.

Thus, step (b) preferably comprises the steps (b 1) and (b2):
(b1) coupling the cytotoxic agent to the crosslinking compound L, thereby forming a derivative of the cytotoxic agent having the structure -L-M, wherein M is the residue of the cytotoxic agent;
(b2) coupling the derivative of the cytotoxic agent having the structure -L-M to the hydroxyalkyl starch derivative according to step (a), thereby forming the hydroxyalkyl starch conjugate.

As to the preferred reaction conditions used in step (b1), reference is made to the details given above.

As regards to the reaction conditions used in step (b2), in principle any reaction conditions known to those skilled in the art can be used. Preferably, the reaction is carried out in an aqueous reaction medium, preferably in a mixture comprising water and at least one organic solvent, preferably at least one water miscible solvent, in particular a solvent selected from the group such as N-methylpyrrolidone, dimethyl acetamide (DMA), dimethyl formamide (DMF), formamide, dimethyl sulfoxide (DMSO), acetonitrile, tetrahydrofurane (THF), dioxane, alcohols such as methanol, ethanol, isopropanol and mixtures of two or more thereof. More preferably, the reaction is carried out in DMF.

The temperature of the reaction is preferably in the range of from 5 to 55° C., more preferably in the range of from 10 to 30° C., and especially preferably in the range of from 15 to 25° C. During the course of the reaction, the temperature may be varied, preferably in the above given ranges, or held essentially constant.

The reaction time for the reaction of step (b2) may be adapted to the specific needs and is generally in the range of from 30 min to 2 days, preferably 1 hour to 18 hours, more preferably 2 hours to 6 hours.

The pH value for the reaction of step (b) may be adapted to the specific needs of the reactants. Preferably, the reaction is carried out in a buffered solution, at a pH value in the range of from 3 to 10, more preferably of from 5 to 9, and even more preferably of from 6 to 8. Among the preferred buffers, citrate buffer (pH 6.4), phosphate buffers (pH 7.5) and bicarbonate buffers (pH 8) may be mentioned.

As described above, the hydroxyalkyl starch derivative may comprise multiple functional groups Z¹, such as multiple thiol groups. Preferably, all groups Z¹ present in the hydroxyalkyl starch derivative participate in the coupling reaction in step (b2). However, it is also possible that in step (b2) not all of the functional groups Z¹ are coupled to the at least bifunctional crosslinking compound L, or preferably with the derivative of the cytotoxic agent having the structure -L-M. Thus, in this case, the hydroxyalkyl starch conjugate according to step (b2) may comprise at least one unreacted functional group Z¹.

To avoid side effects due to the presence of such unreacted functional groups Z¹, the hydroxyalkyl starch conjugate may be further reacted, as described above, in a subsequent step (c) with a suitable capping reagent D*. In case Z¹ is a thiol group, possible free thiol groups present in the conjugate, which may lead to unwanted side effects such as oxidative disulfide formation and consequently crosslinking, may be reacted, for example, with small molecules comprising a thiol-reactive group. Examples of thiol reactive groups are given above.

Preferred capping reagents D* thus in particular comprise a group selected from the group consisting of pyridyl disulfides, maleimide group, haloacetyl groups, haloacetamides, vinyl sulfones and vinyl pyridines. Preferably, the capping reagent D* comprises a thiol-reactive group selected from the group consisting of the following structures:

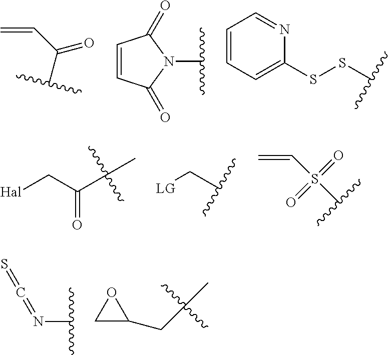

wherein Hal is a halogen, such as Cl, Br, or I, and LG is a leaving group (or nucleofuge).

In particular D* is iodoacetic acid and/or ethylbromoacetate.

Optionally, a reducing agent such as tris-(2-carboxyethyl)phosphine (TCEP) may be added prior to the capping step in order to break existing disulfides and to keep thiols in their low oxidation state.

Thus, the present invention also describes a method, as described above, the method further comprises

(c) reacting the hydroxyalkyl starch conjugate with a capping reagent D.

Likewise, in case the crosslinking compound L is reacted with the hydroxyalkyl starch derivative prior to the coupling to the cytotoxic agent, and only in a subsequent step with the cytotoxic agent, the hydroxyalkyl starch conjugate may comprise at least one unreacted functional group Z¹ and/or at least one unreacted group K¹.

In this case, the present invention may comprise a further capping step

(c1) reacting the hydroxyalkyl starch conjugate with a further capping reagent D**, wherein D** may be the same or may differ from D*, depending on the nature of the functional group to be capped.

Most preferably the hydroxyalkyl starch conjugate according to step (b) comprises no unreacted functional groups Z¹ and/or no unreacted group K¹.

Preferably, the hydroxyalkyl starch conjugate obtained according to step (b), optionally according to step (c) and/or (c1), is subjected to at least one isolation and/or purification step. Isolation of the conjugate may be carried out by a suitable process which may comprise one or more steps.

According to a preferred embodiment of the present invention, the conjugate is first separated from the reaction mixture by a suitable method such as precipitation and subsequent centrifugation or filtration. In a second step, the separated conjugate may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated polymer derivative is first precipitated, subjected to centrifugation, re-dissolved and finally subjected to ultrafiltration.

Preferably, the precipitation is carried out with an organic solvent such as ethanol or isopropanol. The precipitated conjugate is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/l, a pH value in the range of preferably from 3 to 10, more preferably from 4 to 8, such as about 5. The number of exchange cycles preferably is from 5 to 50, more preferably from 10 to 30, and even more preferably from 15 to 25, such as about 20.

Most preferably, the obtained conjugate is further lyophilized until the solvent content of the reaction product is sufficiently low according to the desired specifications of the product.

### Hydroxyalkyl Starch Derivative:

Further, the present invention also relates to a method for preparing a hydroxyalkyl starch derivative as such, said hydroxyalkyl starch derivative comprising a functional group Z¹ being capable of being linked to a further compound, preferably capable of being coupled to a functional group of a crosslinking compound L, more preferably to a derivative of a cytotoxic agent having the structure K²-L′-F³-M as described above.

Preferably, the present invention relates to a method for preparing a hydroxyalkyl starch derivative, preferably having a mean molecular weight MW above the renal threshold, preferably of from 60 to 800 kDa, more preferably of from 80 to 800 kDa, and preferably having a molar substitution MS in the range of from 0.6 to 1.5, the hydroxyalkyl starch derivative comprising at least one structural unit, preferably 3 to 200 structural units, according to the following formula (I)

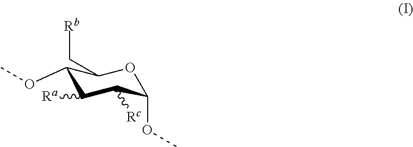

wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—Z¹, and —[O—(CR^{w}R^{x})—(CR^{y}R^{z})]_{y}-[F¹]ₚ-L¹-Z¹, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety and Z¹ is a functional group capable of being reacted with a functional group of a further compound and wherein at least one of R^{a}, R^{b} and R^{c} comprises the functional group Z¹, said method comprising
(a1) providing a hydroxyalkyl starch, preferably having a mean molecular weight MW above the renal threshold, preferably from 60 to 800 kDa, more preferably of from 80 to 800 kDa, and preferably having a molar substitution MS in the range of from 0.6 to 1.5, comprising the structural unit according to the following formula (II)

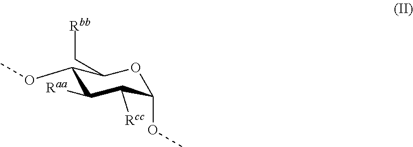

wherein R^{aa}, R^{bb} and R^{cc} are independently of each other selected from the group consisting of —-[O—(CR^{w}R^{x})—(CR^{y}R^{z})]ₓ—OH and —O—HAS″,
(a2) introducing the at least one functional group Z¹ by
   (i) coupling the hydroxyalkyl starch via at least one hydroxyl group to at least one suitable linker comprising the functional group Z¹ or a precursor of the functional group Z¹, or
   (ii) displacing a hydroxyl group present in the hydroxyalkyl starch in a substitution reaction with a precursor of the functional group Z¹ or with a bifunctional linker comprising the functional group Z¹ or a precursor of the functional group Z¹.

Further the present invention also relates to a hydroxyalkyl starch derivative obtained or obtainable by said method.

As regards step (a1), hydroxyalkyl starches having the desired properties are preferably produced from waxy maize starch or potato starch by acidic hydrolysis and reaction with ethylene oxide and purification by ultrafiltration.

The term “functional group Z¹ or a precursor of the functional group Z¹” as used in the context of the present invention is denoted to mean a functional group Z¹ or a functional group being transformed in one or more synthesis step(s) to give a hydroxyalkyl starch derivative comprising the functional group Z¹.

Preferably R^{a}, R^{b} and R^{c} are independently of each other selected from the group consisting of —O—HAS″, —-[O—CH₂—CH₂]ₛ—OH, —[O—CH₂—CH₂]ₜ—Z¹ and —[O—CH₂—CH₂]ₜ[F¹]ₚ-L¹-Z¹ wherein t is in the range of from 0 to 4, and wherein s is in the range of from 0 to 4, with p being 0 or 1, and wherein F¹ is a functional group, and L¹ is a linking moiety.

Z¹ is preferably selected from the group consisting of aldehyde, keto, hemiacetal, acetal groups, alkynyl, azide, carboxy groups, alkenyl, thiol reactive groups, such as maleimide, halogen acetyl, pyridyl disulfides, haloacetamides, vinyl sulfones and vinyl pyridines, —SH, —NH₂, —O—NH₂, —NH—O-alkyl, —(C=G)-NH—NH₂, -G-(C=G)-NH—NH₂, —NH—(C=G)—NH—NH₂, and —SO₂—NH—NH₂ where G is O or S and, if G is present twice, it is independently O or S.

It has to be understood that the one or several groups defined as Z¹ are statistically distributed throughout the hydroxyalkyl starch derivative. Thus, the hydroxyalkyl starch derivative comprises at least one structural unit, preferably 3 to 200 structural units, according to the following formula (I)

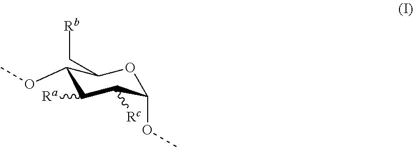

with Z¹ being comprised in at least one of R^{a}, R^{b} or R^{c} and preferably being comprised in multiple repeating units of the structural unit of the formula (I).

Most preferably, the functional group Z¹ is a thiol group (—SH).

Thus, the present invention also relates to a method for a hydroxyalkyl starch derivative comprising at least one thiol group, preferably comprising multiple thiol groups, the derivative having a mean molecular weight MW above the renal threshold, preferably of from 60 to 800 kDa, more preferably of from 80 to 800 kDa, and preferably a molar substitution MS in the range of from 0.6 to 1.5. Further, the present invention also relates to a hydroxyalkyl starch derivative comprising at least one thiol group, preferably comprising multiple thiol groups, obtained or obtainable by the above-mentioned method. More preferably the hydroxyalkyl starch comprises multiple thiol groups, such as 2 to 200 thiol groups, more preferably 3 to 100 thiol groups.

Likewise, the present invention also describes a hydroxyalkyl starch derivative preferably having a mean molecular weight MW above the renal threshold, preferably in the range of from 60 to 800 kDa, more preferably of from 80 to 800 kDa, and preferably having a molar substitution in the range of from 0.6 to 1.5, said hydroxyalkyl starch derivative comprising at least one structural unit, preferably 3 to 200 structural units, according to the following formula (I)

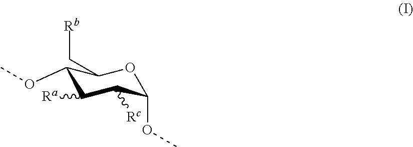

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹, and wherein at least one R^{a}, R^{b} and R^{c} is —[O—CH₂—CH₂]ₜ—Z¹ or —[O—CH₂—CH₂]ₜ-[F₁]ₚ-L¹-Z¹, and wherein t is in the range of from 0 to 4, and wherein s is in the range of from 0 to 4, and wherein p is 0 or 1, and wherein Z¹ is SH.
Step (a2)-(i)

According to a first preferred embodiment of the present invention, the functional group Z¹ is introduced by coupling the hydroxyalkyl starch via at least one hydroxyl group to at least one suitable linker comprising the functional group Z¹ or a precursor of the functional group Z¹.

Organic chemistry offers a wide range of reactions to modify hydroxyl groups with linking constructs bearing functionalities such as aldehyde, keto, hemiacetal, acetal, alkynyl, azide, carboxy, alkaline and thiol reactive groups, such as maleimide, halogens, pyridyl disulfides, haloacetamides, vinyl sulfones, vinyl pyridines, —SH, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂, where G is O or S and, if G is present twice, it is independently O or S, preferably a thiol functionality. However, the hydroxyalkyl starch polymeric nature and the abundance of hydroxyl groups present in the hydroxyalkyl starch usually strongly promotes the number of possible side reactions such as inter- and intramolecular crosslinking. Therefore, a method was needed to functionalize the polymer under maximum retention of its molecular characteristics such as solubility, molecular weight and polydispersity. It was surprisingly found that when using the method according to this preferred embodiment, possible side reactions such as inter- and intramolecular crosslinking can be significantly diminished.

According to a preferred embodiment of the present invention in step (a2)-(i), the hydroxyalkyl starch is coupled to a linker comprising a functional group Z², said functional group Z² being capable of being coupled to a hydroxyl group of the hydroxyalkyl starch, thereby forming a covalent linkage between the first linker and the hydroxyalkyl starch. Further, the linker preferably comprises the functional group Z¹ or a precursor thereof. According to a particularly preferred embodiment, the linker comprises a precursor of the functional group Z¹ which is transformed in at least one further step to give the functional group Z¹.

### The Functional Group Z²

The “functional group Z²” is a functional group capable of being reacted with at least one hydroxyl function of the hydroxyalkyl starch or activated hydroxyl function of hydroxyalkyl starch, thereby forming a covalent linkage F′.

According to a preferred embodiment, the functional group Z² is a leaving group or a nucleophilic group.

According to an alternative embodiment, the functional group Z² is an epoxide.

According to a first preferred embodiment, Z² is a leaving group, preferably a leaving group being attached to a CH₂— group comprised in the at least one suitable linker which is reacted in step (a2)-(ii) with the hydroxyalkyl starch. The term “leaving group” as used in this context of the present invention is denoted to mean a molecular fragment that departs with a pair of electrons in heterolytic bond cleavage upon reaction with the hydroxyl group of the hydroxyalkyl starch, thereby forming a covalent bond between the oxygen atom of the hydroxyl group and the carbon atom formerly bearing the leaving group. Common leaving groups are, for example, halides such as chloride, bromide and iodide, and sulfonates such as tosylates, mesylates, fluorosulfonates, triflates and the like. According to a preferred embodiment of the present invention, the functional group Z² is a halide leaving group. Thus, upon reaction of the hydroxyl group with the functional group Z², preferably a functional group F¹ is formed, which is preferably an —O— group.

Alternatively, Z² may also be an epoxide group, which reacts with a hydroxyl group in a ring opening reaction, thereby forming a covalent bond.

According to another embodiment, Z² is a nucleophile, thus a group capable of forming a covalent bond with an electrophile by donating both bonding electrons. In case Z² is a nucleophile, the method preferably comprises an initial step, in which at least one hydroxyl function of hydroxyalkyl starch is activated, thereby forming an electrophilic group. For example, the hydroxyl group may be activated by reacting at least one hydroxyl function with a reactive carbonyl compound, as described in detail below. Thus, the present invention also describes a method, wherein the functional group Z² is a nucleophile, said nucleophile being capable of being reacted with at least one activated hydroxyl function of hydroxyalkyl starch, as described above, wherein the hydroxyl group is initially activated with a reactive carbonyl compound prior to coupling the hydroxyalkyl starch in step (a2)-(ii) to the at least one suitable linker comprising the functional group Z² and the functional group Z¹ or a precursor of the functional group Z¹.

The term “reactive carbonyl compound” as used in this context of the present invention, refers to carbonyl di-cation synthons having a structure R**—(C═O)—R*, wherein R* and R** may be the same or different, and wherein R* and R** are both leaving groups. As leaving groups halides, such as chloride, and/or residues derived from alcohols, may be used. The term “residue derived from alcohols”, refers to R* and/or R** being a unit —O—R^{ff} or —O—R^{gg}, with —O—R^{ff} and —O—R^{gg} preferably being residues derived from alcohols such as N-hydroxy succinimide or sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, heterocycles such as imidazol or hydroxyazoles such as hydroxybenzotriazole may be mentioned. Reactive carbonyl compounds containing halides are phosgene, related compounds such as diphosgene or triphosgene, chloroformic esters and other phosgene substitutes known in the art. Especially preferred are carbonyldiimidazol (CDI), N,N′-disuccinimidyl carbonate and sulfo-N,N′-disuccinimidyl carbonate, or mixed compounds such as p-nitrophenyl chloroformate.

Preferably, the reactive carbonyl compound having the structure R**—(C═O)—R* is selected from the group consisting of phosgene, diphosgene, triphosgene, chloroformates and carbonic acid esters, more preferably from the group consisting of p-nitrophenylchloroformate, pentafluorophenylchloroformate, N,N′-disuccinimidyl carbonate, sulfo-N,N′-disuccinimidyl carbonate, dibenzotriazol-1-yl carbonate and carbonyldiimidazol.

Preferably, upon reaction of at least one hydroxyl group with the reactive carbonyl compound R^{dd}—(C═O)—R^{d} prior to the coupling step according to step (a2)-(ii), an activated hydroxyalkyl starch derivative is formed, which comprises at least one structural unit, preferably 3 to 200 structural units, according to the following formula (Ib)

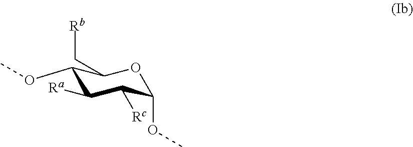

wherein R^{a}, R^{b} and R^{c} are independently of each other selected from the group consisting of —O—HAS″, —-[O—CH₂—CH₂]_{S}—OH, and —[O—CH₂—CH₂]ₜ—O—C(═O)—R*, wherein t is in the range of from 0 to 4, and wherein s is in the range of from 0 to 4, and wherein at least one of R^{a}, R^{b} and R^{c} comprises the group —-[O—CH₂—CH₂]ₜ—O—C(═O)—R*, and wherein R* is a leaving group, preferably a group selected from the group consisting of p-nitrophenoxy-, 2,4-dichlorophenoxy, 2,4,6-trichlorophenoxy, trichloromethoxy, imidazolyl, azide and halides, such as chloride or bromide.

According to this embodiment, according to which the hydroxyalkyl starch is activated to give a hydroxyalkyl starch derivative comprising a reactive —O—C(═O)—R* group, Z² is preferably a nucleophilic group, such as a group comprising an amino group. Possible groups are, for example, —NHR^{Z2}—NH₂, —O—NH₂, —NH—O-alkyl, —(C=G)-NH—NH₂, -G-(C=G)-NH—NH₂, —NH—(C=G)-NH—NH₂, and —SO₂—NH—NH₂ wherein G is O or S, and, if present twice in one structural unit, may be the same or may be different, and wherein R^{Z2} is an alkyl group, preferably methyl. More preferably Z² is —NH₂ or —NHR^{Z2}, most preferably —NH₂.

As described above, besides the functional group Z², the linker comprises either the functional group Z¹ or a precursor thereof.

Preferably, the linker further comprises the functional group W, this functional group being a group capable of being transformed in at least one further step to give the functional group Z¹. Preferably W is an epoxide or a functional group which is transformed in a further step to give an epoxide, or W has the structure Z¹-PG, with PG being a suitable protecting group.

According to a first preferred embodiment, in step (a2)-(i), a first linker is used comprising the functional group W, wherein W is an epoxide or a functional group which is transformed in a further step to give an epoxide.

Thus, the present invention also relates to a method for preparing a hydroxyalkyl starch derivative, as described above, and a hydroxyalkyl starch derivative obtained or obtainable by said method, wherein step (a2)-(i) comprises the step (I):
(1) coupling the hydroxyalkyl starch (HAS) via at least one hydroxyl group comprised in HAS to a first linker comprising a functional group Z² capable of being reacted with the at least one hydroxyl group of the hydroxyalkyl starch, thereby forming a covalent linkage between the first linker and the hydroxyalkyl starch, the first linker further comprising a functional group W, wherein the functional group W is an epoxide or a group which is transformed in a further step to give an epoxide.

Preferably, the first linker has the structure Z²-L″-W, wherein Z² is a functional group capable of being reacted with at least one hydroxyl group of hydroxyalkyl starch, as described above, and wherein L^{W} is a linking moiety.

Thus, the present invention also relates to a method for preparing a hydroxyalkyl starch derivative, as described above, and a hydroxyalkyl starch derivative obtained or obtainable by said method, wherein step (a2)-(i) comprises the step (I):
(I) coupling the hydroxyalkyl starch via at least one hydroxyl group comprised in HAS to a first linker having a structure according to the following formula Z²-L^{w}-W, wherein Z² is a functional group capable of being reacted with at least one hydroxyl group of hydroxyalkyl starch, as described above, and wherein L^{W} is a linking moiety, and wherein, upon reaction of the hydroxyalkyl starch, a hydroxyalkyl starch derivative is formed comprising at least one structural unit, preferably 3 to 200 structural units, according to the following formula (Ib)

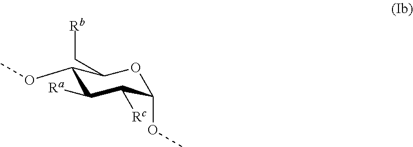

wherein R^{a}, R^{b} and R^{c} are independently of each other selected from the group consisting of —O—HAS″, —[O—(CR^{w}R^{x})—(CR^{y}R^{z})]ₓ—OH and —[O—(CR^{w}R^{x})—(CR^{y}R^{z})]_{y}-[F¹]ₚ-L^{w}-W, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{c} comprises the group —[O(CR^{w}R^{x})—(CR^{y}R^{z})]_{y}-[F¹]ₚ-L^{w}-W, and wherein [F¹]ₚ is the functional group being formed upon reaction of Z² with the at least one hydroxyl group of the hydroxyalkyl starch, more preferably, wherein R^{a}, R^{b} and R^{c} are independently of each other selected from the group consisting of —O—HAS″, —[O—CH₂—CH₂]ₛ—OH and —[O—CH₂—CH₂]ₜ-[F¹]ₚ-L^{W}-W, wherein t is in the range of from 0 to 4, and wherein s is in the range of from 0 to 4, and wherein p is 1, and wherein at least one of R^{a}, R^{b} and R^{c} comprises the group —-[O—CH₂—CH₂]ₜ-[F¹]ₚ-L^{W}-W, and wherein [F¹]ₚ is the functional group being formed upon reaction of Z² with the at least one hydroxyl group of the hydroxyalkyl starch.

According to one embodiment of the present invention, the functionalization of at least one hydroxyl group of hydroxyalkyl starch to give the epoxide comprising hydroxyalkyl starch, is carried out in a one-step procedure, wherein at least one hydroxyl group is reacted with a first linker, as described above, wherein the first linker comprises the functional group W, and wherein W is an epoxide.

Therefore, the present invention also describes a method for preparing a hydroxyalkyl starch derivative, as described above, as well as to a hydroxyalkyl starch derivative obtained or obtainable by said method, wherein in step (a2)-(i)-(1) the hydroxyalkyl starch is reacted with a linker comprising a functional group Z² capable of being reacted with a hydroxyl group of the hydroxyalkyl starch, thereby forming a covalent linkage, the linker further comprising a functional group W, wherein the functional group W is an epoxide.

This linker has in this case a structure according to the following formula

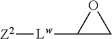

such as, for example, epichlorohydrine.

Upon reaction of this linker with at least one hydroxyl group of hydroxyalkyl starch, a hydroxyalkyl starch derivative is formed comprising at least one structural unit, preferably 3 to 200 structural units, according to the following formula (Ib)

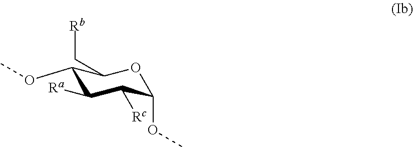

wherein R^{a}, R^{b} and R^{c} are independently of each other selected from the group consisting of —O—HAS″, —[O—(CR^{w}R^{x})—(CR^{y}R^{z})]ₓ—OH and

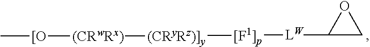

and wherein at least one of R^{a}, R^{b} and R^{c} comprises the group

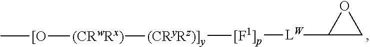

preferably wherein R^{a}, R^{b} and R^{c} are independently of each other selected from the group consisting of —O—HAS″, —[O—CH₂—CH₂]ₛ—OH and

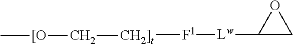

(i.e. p is 1), and wherein t is in the range of from 0 to 4 and wherein s is in the range of from 0 to 4, and wherein at least one of R^{a}, R^{b} and R^{c} comprises the group

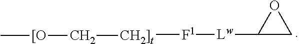

According to a preferred embodiment of the invention, the epoxide is generated in a two-step procedure, comprising the steps (I) and (II)
(I) coupling at least one hydroxyl group of the hydroxyalkyl starch, preferably of hydroxyethyl starch, to a first linker, comprising a functional group Z² capable of being reacted with a hydroxyl group of the hydroxyalkyl starch, thereby forming a covalent linkage between the first linker and the hydroxyalkyl starch, the linker further comprising a functional group W, wherein the functional group W is a functional group which is capable of being transformed in a further step to give an epoxide, such as an alkenyl group,
(II) transforming the functional group W to give an epoxide.

It was surprisingly found that this two-step procedure is superior to the one-step procedure in that higher loadings of the hydroxyalkyl starch with epoxide groups can be achieved and/or undesired side reactions such as inter- and intramolecular crosslinking can be substantially avoided.

Preferably, the functional group W is an alkenyl group. In this case, step (II) preferably comprises the oxidation of the alkenyl group to give an epoxide and transforming the epoxide to give the functional group Z¹.

According to a preferred embodiment, the present invention also relates to a method for preparing a hydroxyalkyl starch derivative, as described above, wherein the hydroxyalkyl starch, preferably the hydroxyethyl starch, is coupled in step (a2)-(i) via at least one hydroxyl group to at least one suitable linker, the linker having the structure Z²-L^{w}-W, wherein upon reaction of a hydroxyl group of the hydroxyalkyl starch with the linker, the leaving group Z² departs, thereby forming a covalent linkage between the hydroxyalkyl starch and the linking moiety L^{w}, and wherein the functional group F¹ which links the hydroxyalkyl starch and the linking moiety L^{w}, is an —O— bond. Likewise, the present invention also relates to the respective hydroxyalkyl starch derivatives obtained or obtainable by said method.

According to the present invention, the term “linking moiety L^{w}” as used in the context of the present invention relates to any suitable chemical moiety bridging the functional group Z² and the functional group W.

In general, there are no particular restrictions as to the chemical nature of the linking moiety L^{W} with the proviso that L^{W} has particular chemical properties enabling carrying out the inventive method for the preparation of the novel derivatives comprising the functional group Z¹, i.e. in particular, in case W is a functional group to be transformed to an epoxide, the linking moiety L^{W} has suitable chemical properties enabling the transformation of the chemical moiety W to the functional group Z¹. According to a preferred embodiment of the present invention, L^{W} bridging W and HAS′ comprises at least one structural unit according to the following formula

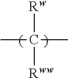

wherein R^{vv} and R^{ww} are independently of each other H or an organic residue selected from the group consisting of alkyl, alkenyl, alkylaryl, arylalkyl, aryl, heteroaryl, alkylheteroaryl and heteroarylalkyl groups.

Preferably, L^{W} is an optionally substituted, non-branched alkyl residue such as a group selected from the following groups:

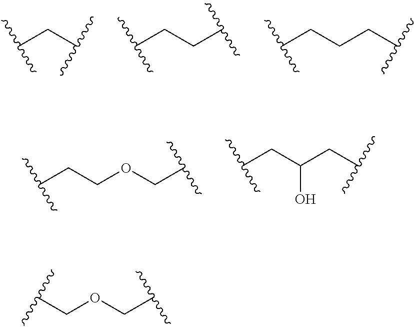

According to a first preferred embodiment of the present invention, the functional group W is an alkenyl group, wherein the first linker Z²-L^{W}-W has a structure according to the following formula

Z²-L^{w}-CH═CH₂

preferably with Z² being a leaving group or an epoxide.

Thus preferred structures of the first linker are by way of example, the following structures:

Hal-CH₂—CH═CH₂ such as Cl-CH₂—CH═CH₂ or Br—CH₂—CH═CH₂ or I—CH₂—CH═CH₂, sulfonic esters, such as TsO—CH₂—CH═CH₂ or MsO—CH₂—CH═CH₂, epoxides such as

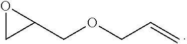

More preferably, Z² in the first linker Z²-L^{W}-W is a leaving group, most preferably the first linker Z²-L^{W}-W has a structure according to the following formula

Hal-L^{w}-CH═CH₂.

According to an especially preferred embodiment of the present invention, the linker Z²-L^{W}-W has a structure according to the following formula

Hal-CH₂—CH═CH₂

with Hal being a halogen, preferably the halogen being iodine, bromine or chlorine, more preferably bromine.

Thus, the present invention also relates to a method for preparing a hydroxyalkyl starch derivative, as described above, wherein in step (a2)-(i) the hydroxyalkyl starch, preferably the hydroxyethyl starch, is coupled via at least one hydroxyl group to at least one suitable linker having the structure Hal-CH₂—CH═CH₂, wherein upon reaction of the hydroxyalkyl starch with the linker, a hydroxyalkyl starch derivative is formed, comprising at least one structural unit according to the following formula (Ib)

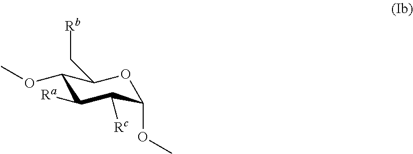

wherein R^{a}, R^{b} and R^{c} are independently of each other selected from the group consisting of —O—HAS″, —[O—(CR^{w}R^{x})—(CR^{y}R^{z})]ₓ—OH and —[O—(CR^{w}R^{x})—(CR^{y}R^{z})]_{y}—O—CH₂—CH═CH₂, and wherein at least one of R^{a}, R^{b} and R^{c} comprises the group —[O—(CR^{w}R^{x})—(CR^{y}R^{z})]_{y}—O—CH₂—CH═CH₂, preferably wherein R^{a}, R^{b} and R^{c} are independently of each other selected form the group consisting of —O—HAS″, —[O—CH₂—CH₂]ₛ—OH and —[O—CH₂—CH₂]ₜ—O—CH₂—CH═CH₂, wherein t is in the range of from 0 to 4 and wherein s in the range of from 0 to 4, and wherein at least one of R^{a}, R^{b} and R^{c} comprises the group —[O—CH₂—CH₂]ₜ—O—CH₂—CH═CH₂, and wherein the functional group —O— linking the —CH₂—CH═CH₂ group to the hydroxyalkyl starch is formed upon reaction of the linker Hal-CH₂—CH═CH₂ with the hydroxyl group of the hydroxyalkyl starch. Likewise, the present invention also relates to a hydroxyalkyl starch derivative obtained or obtainable by the above-mentioned method.

As regards, the reaction conditions used in this step (I), wherein the hydroxyalkyl starch is reacted with the first linker, in particular wherein the first linker comprises the functional group W with W being an alkenyl, in principle any reaction conditions known to those skilled in the art can be used. Preferably, the reaction is carried out in an organic solvent, such as N-methylpyrrolidone, dimethyl acetamide (DMA), dimethyl formamide (DMF), formamide, dimethyl sulfoxide (DMSO) or mixtures of two or more thereof. More preferably, the reaction is carried out in anhydrous solvents or solvent mixtures.

Preferably, the hydroxyalkyl starch is dried prior to use, by means of heating to constant weight at a temperature range from 50 to 80° C. in a drying oven or with related techniques.

The temperature of the reaction is preferably in the range of from 5 to 55° C., more preferably in the range of from 10 to 30° C., and especially preferably in the range of from 15 to 25° C. During the course of the reaction, the temperature may be varied, preferably in the above given ranges, or held essentially constant.

The reaction time for the reaction of HAS with the linker Z²-L^{w}-W may be adapted to the specific needs and is generally in the range of from 1 h to 7 days, preferably of from 2 hours to 24 hours, more preferably of from 3 hours to 18 hours.

More preferably, the reaction is carried out in the presence of a base. The base may be added together with the linker Z²-L^{W}-W, or may be added prior to the addition of the linker, to pre-activate the hydroxyl groups of the hydroxyalkyl starch. Preferably, a base, such as alkali metal hydrides, alkali metal hydroxides, alkali metal carbonates, amine bases such as diisopropylethyl amine (DIEA) and the like, amidine bases such as 1,8-diazabicyclo[5.4.0]undec-7-ene (DBU), amide bases such as lithium diisopropylamide (LDA) or alkali metal hexamethyldisilazyl bases (e.g. LiHMDS) may be used. Most preferably the hydroxyalkyl starch is pre-activated with sodium hydride prior to the addition of the first linker Z²-L^{W}-W.

The derivative comprising the functional group W, preferably the alkenyl group, may be isolated prior to transforming this group in at least one further step to give an epoxide comprising hydroxyalkyl starch derivative. Isolation of this polymer derivative comprising the functional group W may be carried out by a suitable process which may comprise one or more steps. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated polymer derivative is first precipitated, subjected to centrifugation, re-dissolved and finally subjected to ultrafiltration.

Preferably, the precipitation is carried out with an organic solvent such as ethanol, isopropanol, acetone or tetrahydrofurane (THF). The precipitated derivative is subsequently subjected to centrifugation and subsequent ultrafiltration using water or an aqueous buffer solution having a concentration preferably from 1 to 1000 mmol/l, more preferably from 1 to 100 mmol/I, and more preferably from 10 to 50 mmol/l, such as about 20 mmol/l, a pH value preferably in the range of from 3 to 10, more preferably of from 4 to 8, such as about 7. The number of exchange cycles preferably is in the range of from 5 to 50, more preferably of from 10 to 30, and even more preferably of from 15 to 25, such as about 20. Most preferably, the obtained derivative comprising the functional group W is further lyophilized until the solvent content of the reaction product is sufficiently low according to the desired specifications of the product.

In case W is an alkenyl, the method preferably further comprises step (II), that is the oxidation of the alkenyl group to give an epoxide group. As to the reaction conditions used in the epoxidation (oxidation) step (II), in principle, any known method to those skilled in the art can be applied to oxidize an alkenyl group to yield an epoxide.

The following oxidizing reagents are mentioned, by way of example, metal peroxysulfates such as potassium peroxymonosulfate (Oxone®) or ammonium peroxydisulfate, peroxides such as hydrogen peroxide, tert.-butyl peroxide, acetone peroxide (dimethyldioxirane), sodium percarbonate, sodium perborate, peroxy acids such as peroxyacetic acid, meta-chloroperbenzoic acid (MCPBA) or salts like sodium hypochlorite or hypobromite.

According to a particularly preferred embodiment of the present invention, the epoxidation is carried out with potassium peroxymonosulfate (Oxone®) as oxidizing agent.

Thus, the present invention also relates to a method for preparing a hydroxyalkyl starch derivative, as described above, wherein step (a2)-(i) comprises
(I) coupling at least one hydroxyl group of the hydroxyalkyl starch, preferably of hydroxyethyl starch, to a first linker, comprising a functional group Z² capable of being reacted with a hydroxyl group of the hydroxyalkyl starch, thereby forming a covalent linkage between the first linker and the hydroxyalkyl starch, the linker further comprising a functional group W, wherein the functional group W is an alkenyl group,
(II) oxidizing the alkenyl group to give an epoxide, wherein as oxidizing agent, preferably potassium peroxymonosulfate (Oxone®) is employed.

Further, the present invention also relates to a hydroxyalkyl starch derivative obtained or obtainable by said method.

According to an even more preferred embodiment of the present invention, the reaction with potassium peroxymonosulfate (Oxone®) is carried out in the presence of a suitable catalyst. Catalysts may consist of transition metals and their complexes, such as manganese (Mn-salene complexes are known as Jacobsen catalysts), vanadium, molybdenium, titanium (Ti-dialkyltartrate complexes are known as Sharpless catalysts), rare earth metals and the like. Additionally, metal free systems can be used as catalysts. Acids such as acetic acid may form peracids in situ and epoxidize alkenes. The same accounts for ketones such as acetone or tetrahydrothiopyran-4-one, which react with peroxide donors under formation of dioxiranes, which are powerful epoxidation agents. In case of non-metal catalysts, traces of transition metals from solvents may lead to unwanted side reactions, which can be excluded by metal chelation with EDTA.

Preferably, said suitable catalyst is tetrahydrothiopyran-4-one.

Upon epoxidation, in step (II) a hydroxyalkyl starch derivative is formed comprising at least one structural unit, preferably 3 to 200 structural units, according to the following formula (Ib)

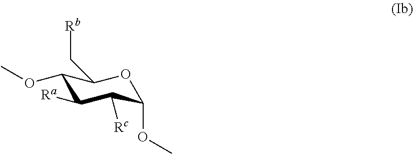

wherein R^{a}, R^{b} and R^{c} are independently of each other selected from the group consisting of —O—HAS″, —[O(CR^{w}R^{x})—(CR^{y}R^{z})]ₓ—OH and

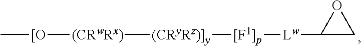

and wherein at least one of R^{a}, R^{b} and R^{c} comprises the group

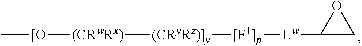

preferably wherein R^{a}, R^{b} and R^{c} are independently of each other selected from the group consisting of —O—HAS″, —[O—CH₂—CH₂]ₛ—OH and

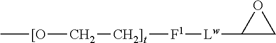

(i.e. p is 1), and wherein t is in the range of from 0 to 4 and wherein s is in the range of from 0 to 4, and wherein at least one of R^{a}, R^{b} and R^{c} comprises the group

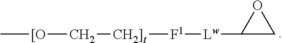

According to a preferred embodiment, the epoxidation of the alkenyl-modified hydroxyalkyl starch derivatives is carried out in aqueous medium, preferably at a temperature in the range of from 0 to 80° C., more preferably in the range of from 0 to 50° C. and especially preferably in the range of from 10 to 30° C.

During the course of the epoxidation reaction, the temperature may be varied, preferably in the above-given ranges, or held essentially constant. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminotetraacetate, EDTA, or the like) in the concentration ranging from 0.01 to 100 mM, preferably from 0.01 to 1 mM, most preferably from 0.1 to 0.5 mM, such as about 0.4 mM.

The pH value for the reaction of the HAS derivative with potassium peroxymonosulfate (Oxone®) may be adapted to the specific needs of the reactants. Preferably, the reaction is carried out in buffered solution, at a pH value in the range of from 3 to 10, more preferably of from 5 to 9, and even more preferably of from 7 to 8. Among the preferred buffers, carbonate, phosphate, borate and acetate buffers as well as tris(hydroxymethyl)aminomethane (TRIS) may be mentioned. Among the preferred bases, alkali metal bicarbonates may be mentioned.

According to the invention, the epoxide-modified HAS derivative may be purified or isolated in a further step prior to the transformation of the epoxide group to the functional group Z¹.

The separated derivative is optionally lyophilized.

After the purification step, the HAS derivative is preferably obtained as a solid. According to a further conceivable embodiment of the present invention, the HAS derivative solutions or frozen HAS derivative solutions may be mentioned.

The epoxide comprising HAS derivative is preferably reacted in a subsequent step (III) with at least one suitable reagent to yield the HAS derivative comprising the functional group Z¹. Preferably, the epoxide is reacted with a nucleophile comprising the functional group Z¹ or a precursor thereof. Preferably, the nucleophile reacts with the epoxide in a ring opening reaction and yields a HAS derivative comprising at least one structural unit, preferably 3 to 200 structural units according to the following formula (Ib)

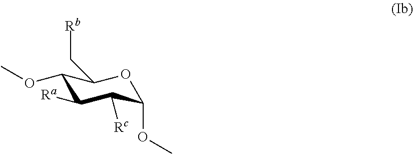

wherein at least one of R^{a}, R^{b} and R^{c} is —-[O—(CR^{w}R^{x})—(CR^{y}R^{z})]_{y}-[F¹]ₚ-L^{W}-CHOH—CH₂-Nuc, preferably wherein at least one of R^{a}, R^{b} and R^{c} is —[O—CH₂—CH₂]ₜ-[F¹]ₚ-L^{W}-CHOH—CH₂—Nuc, wherein the residue Nuc is the remaining part of the nucleophile covalently linked to the hydroxyalkyl starch after being reacted with the epoxide.

Any nucleophile capable of reacting with the epoxide thereby forming a covalent linkage and comprising the functional group Z¹ or a precursor thereof may be used. As nucleophile, for example, linker compounds comprising at least one nucleophilic functional group capable of reacting with the epoxide and at least one functional group W′, such as a group Z¹*-PG (with Z¹* being the protected form of the functional group Z¹), capable of being transformed to the functional group Z¹ can be used. Alternatively, a linker such as an at least bifunctional linker comprising a nucleophilic group such as a thiol group and further comprising the functional group Z¹ may be used.

As described above, according to a particularly preferred embodiment of the present invention, Z¹ is a thiol group.

According to a further preferred embodiment of the present invention, the nucleophilic group reacting with the epoxide is a thiol group.

Thus, the present invention also relates to a method as described above, wherein step (a2)-(i) comprises
(III) reacting the epoxide with a nucleophile comprising the functional group Z¹ or a precursor of the functional group Z¹, the nucleophile additionally comprising a nucleophilic group, preferably wherein Z¹ and the nucleophilic group are both —SH groups.

According to an especially preferred embodiment of the present invention, the present invention also relates to a method for preparing a hydroxyalkyl starch derivative, as well as to a hydroxyalkyl starch derivative obtained or obtainable by said method, as described above, wherein the epoxide is reacted with a nucleophile comprising the functional group Z¹, with Z¹ being a thiol group, and comprising a nucleophilic group, this group being a thiol. Thus, according to a preferred embodiment, the nucleophile is a dithiol.

The invention also relates to the respective derivative obtained or obtainable by said method, said derivative preferably comprising at least one structural unit, preferably 3 to 200 structural units according to the following formula (Ib)

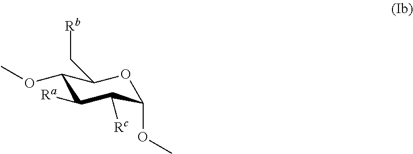

wherein at least one of R^{a}, R^{b} and R^{c} is —[O(CR^{w}R^{x})—(CR^{y}R^{z})]_{y}-[F¹]ₚ-L¹-SH, preferably wherein at least one of R^{a}, R^{b} and R^{c} is —[O—CH₂—CH₂]ₜ—[F¹]ₚ-L¹-SH, wherein L¹ is a linking moiety which is obtained when reacting the structural unit

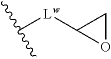

with the nucleophile and which links the functional group F¹ to the functional group Z¹. According to the preferred embodiment, the linking moiety L¹ has a structure selected from the groups below:

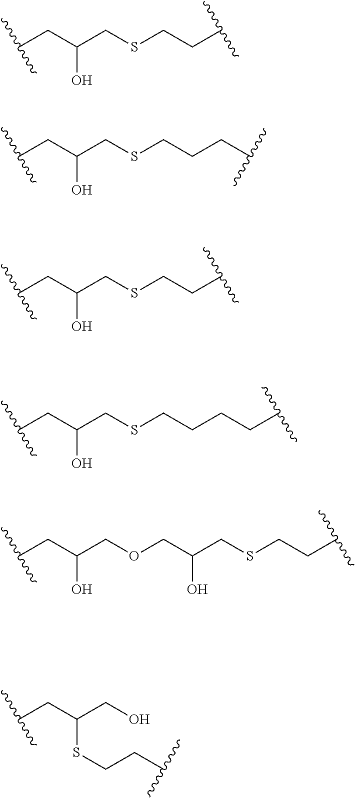 [SMILES: *CCSCC(O)C* ; *CCCSCC(O)C* ; *CCSCC(O)C* ; *CCCCSCC(O)C* ; *CCSCC(O)COCC(O)C* ; *CCSC(C*)CO]

more preferably L¹ has a structure according to the following formula

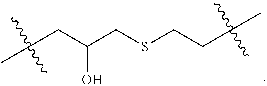 [SMILES: *CCSCC(O)C*]

According to an alternative embodiment of the present method, the epoxide is reacted with a nucleophile suitable for the introduction of thiol groups such as thiosulfate, alkyl or aryl thiosulfonates or thiourea, preferably sodium thiosulfate. Thus, the present invention also relates to a method as described above as well as to a hydroxyalkyl starch derivative obtained or obtainable by said method, wherein the epoxide-modified hydroxyalkyl starch is reacted with a nucleophile, said nucleophile being thiosulfate, alkyl or aryl thiosulfonates or thiourea, preferably sodium thiosulfate.

Upon reaction of the thiosulfate with the epoxide in a ring opening reaction, preferably a hydroxyalkyl starch derivative is formed comprising at least one structural unit, preferably 3 to 200 structural units, according to the following formula (Ib)

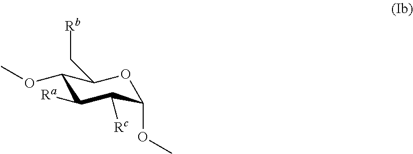

wherein at least one of R^{a}, R^{b} and R^{c} is —[O(CR^{w}R^{x})—(CR^{y}R^{z})]ₓ-[F¹]ₚ-L^{W}-CHOH—CH₂—SSO₃Na, preferably wherein at least one of R^{a}, R^{b} and R^{c} is —[O—CH₂—CH₂]ₜ-[F¹]ₚ-L^{W}-CHOH—CH₂—SSO₃Na.

Preferably, this derivative is reduced in a subsequent step to yield the HAS derivative comprising the functional group Z¹ with Z¹ being —SH. Any suitable methods known to those skilled in the art can be used to reduce the respective intermediate shown above. Preferably, the thiosulfonate is reduced with sodium borohydride in aqueous solution.

According to a preferred embodiment of the present invention, the hydroxyalkyl starch derivative comprising the functional group Z¹, obtained by the above-described method, is purified in a further step. Again, the purification of the HAS derivative from step (III) can be carried out by any suitable method such as ultrafiltration, dialysis or precipitation or a combined method using for example precipitation and afterwards ultrafiltration. Furthermore, the HAS derivative may be lyophilized, as described above, using conventional methods.

Synthesis of the Hydroxyalkyl Starch Derivative Via the Reaction of the Carboxy Activated Hydroxyalkyl Starch with a Linker Compound

According to a second embodiment, in step (a2)-(i), a linker is used, comprising the functional group Z¹ or the functional group W, wherein W has the structure —Z¹-PG, with PG being a suitable protecting group. Preferably, in case this linker is used, the hydroxyalkyl starch is activated prior to the reaction using a reactive carbonate as described above.

Thus, the present invention also relates to a method, as described above, wherein in step (a2)-(i) the hydroxyalkyl starch is reacted with a linker comprising the functional group Z¹ or a precursor thereof and a functional group Z², the linker preferably having the structure Z²-L¹-Z¹ or Z²-L¹-Z¹*-PG, with Z² being a functional group capable of being reacted with the hydroxyalkyl starch or an activated hydroxyalkyl starch, preferably with an activated hydroxyalkyl starch, the method further comprising activating the hydroxyalkyl starch prior to the reaction with the linker using a reactive carbonate.

As described above, the linker preferably comprises a functional group Z², which in this case, is preferably a nucleophile, such as a group comprising an amino group, more preferably a group selected from the group consisting of NHR^{Z2}, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂ wherein G is O or S, and if present twice in one structural unit, may be the same or may be different, and wherein R^{Z2} is an alkyl group, preferably methyl. More preferably Z² is —NH₂ or —NHR^{Z2}, most preferably NH₂.

The linker has preferably a structure Z²-L¹-Z¹*—PG, wherein Z¹* is in particular —S-(and the respective corresponding unprotected functional group Z¹ a thiol group). According to this embodiment, the linking moiety L¹ is preferably an alkyl group. More preferably, the linking moiety L¹ is a spacer comprising at least one structural unit according to the formula —{[CR^{d}R^{f}]ₕ-[F⁴]ᵤ—[CR^{dd}R^{ff}]_{z}}ₐₗₚₕₐ—, as described above, wherein integer alpha is in the range of from 1 to 10, and wherein F⁴ is preferably selected from the group consisting of —S—, —O— and —NH—, more preferably wherein F⁴, if present, is —O— or —S—, more preferably wherein F⁴ is —S—. As described above, in the context of the preferred conjugates, residues R^{d}, R^{f}, R^{dd} and R^{ff} are, independently of each other, preferably selected from the group consisting of halogens, alkyl groups, H or hydroxyl groups. More preferably, these residues are independently from each other H, alkyl or hydroxyl groups. Preferably, integer u and integer z of the formula —{[CR^{d}R^{f}]ₕ-[F⁴]—[CR^{dd}R^{ff}]_{z}}ₐₗₚₕₐ— are 0, and alpha is 1, the linking moiety L¹ thus corresponds to the structural unit —[CR^{d}R^{f}]ₕ—. The integer h is preferably in the range of from 1 to 20, more preferably of from 1 to 10, such as 1, 2, 3, 4, 5, 6, 7, 8, 9 or 10, more preferably of from 1 to 5, most preferably of from 1 to 3. More preferably R^{d} and R^{f} are both H. Thus, by way of example, the following preferred linker moieties L¹ are mentioned: —CH₂—, —CH₂—CH₂, —CH₂—CH₂—CH₂—, —CH₂—CH₂—CH₂—CH₂—, —CH₂—CH₂—CH₂—C₂—CH₂—, more preferably —CH₂—CH₂—, in the context of this second embodiment.

In case Z¹ is a thiol group, and Z¹* is —S—, the group PG is preferably a thiol protecting group, more preferably a protecting group forming together with Z¹* a thioether (e.g. trityl, benzyl, allyl), a disulfide (e.g. S-sulfonates, S-tert.-butyl, S-(2-aminoethyl)) or a thioester (e.g. thioacetyl). In case the linker comprises a protecting group, the method further comprises a deprotection step.

In case the group —Z¹*-PG is a disulfide, and Z¹* is —S—, the linker Z²-L¹-S-PG is preferably a symmetrical disulfide, with PG having the structure S-L¹-Z². As preferred linker compound, thus cystamine and the like may be mentioned.

In the context of this embodiment, the following linker compounds having the structure Z²-L¹-Z¹*-PG are mentioned by way of example: H₂N—CH₂—S-Trt, H₂N—CH₂—CH₂—S-Trt, H₂N—CH₂—CH₂—CH₂—S-Trt, H₂N—CH₂—CH₂—CH₂—CH₂—S-Trt, H₂N—CH₂—CH₂—CH₂—CH₂—CH₂—S-Trt, H₂N—CH₂—CH₂—S—S—CH₂—CH₂—NH₂, H₂N—CH₂—CH₂—S—S-tBu, wherein Trt is a trityl group.

Subsequent to the activation, the hydroxyalkyl starch is preferably reacted with the linker Z²-L¹-Z¹*-PG, thereby most preferably forming a derivative, comprising the functional group Z¹*-PG, more preferably this derivative comprises at least one structural unit, preferably 3 to 200 structural units, according to the following formula (Ib)

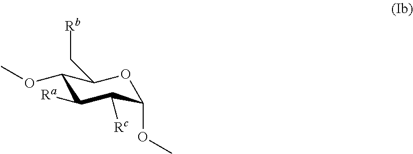

wherein at least one of R^{a}, R^{b} and R^{c} is —[O—(CR^{w}R^{x})—(CR^{y}R^{z})]ₓ—F¹-L¹-Z¹*-PG, more preferably wherein R^{a}, R^{b} and R^{c} are independently of each other selected from the group consisting of —O—HAS″, —[O—CH₂—CH₂]ₛ—OH, and —[O—CH₂—CH₂]ₜ—F¹-L¹-Z¹*-PG, wherein t is in the range of from 0 to 4, and wherein s is in the range of from 0 to 4, and wherein at least one of R^{a}, R^{b} and R^{c} comprises the group —[O—CH₂—CH₂]ₜ—F¹-L¹-Z¹*-PG, and wherein F¹ is the functional group being formed upon reaction of the group —O—C(═O)—R^{d} with the functional group Z². According to a preferred embodiment, the functional group Z² is —NH₂, thus F¹ preferably has the structure O—C(═O)—NH—.

The coupling reaction between the activated hydroxyalkyl starch and the linker, comprising the functional group Z¹ or the functional group W, wherein W has preferably the structure —Z¹*-PG, with PG being a suitable protecting group, in principle any reaction conditions known to those skilled in the art can be used. Preferably, the reaction is carried out in an organic solvent, such as N-methylpyrrolidone, dimethyl acetamide (DMA), dimethyl formamide (DMF), formamide, dimethyl sulfoxide (DMSO), or mixtures of two or more thereof, preferably at a temperature in the range of from 5 to 80° C., more preferably in the range of from 5 to 50° C. and especially preferably in the range of from 15 to 30° C. The temperature may be held essentially constant or may be varied during the reaction procedure.

The pH value for this reaction may be adapted to the specific needs of the reactants. Preferably, the reaction is carried out in the presence of a base. Among the preferred bases pyridine, substituted pyridines, such as 4-(dimethylamino)-pyridine, 2,6-lutidine or collidine, tertiary amine bases such as triethyl amine, diisopropyl ethyl amine (DIEA), N-methyl morpholine, amidine bases such as 1,8-diazabicyclo[5.4.0]undec-7-ene or inorganic bases such as alkali metal carbonates may be mentioned.

The reaction time for the reaction of activated hydroxyalkyl starch with the linker Z²-L¹-Z¹*-PG or Z²-L¹-Z¹ may be adapted to the specific needs and is generally in the range of from 1 h to 7 days, preferably 2 hours to 48 hours, more preferably 4 hours to 24 hours.

The derivative comprising the functional group Z¹*-PG or Z¹, may be subjected to at least one further isolation and/or purification step. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilization. According to an even more preferred embodiment, the separated polymer derivative is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated derivative is subsequently subjected to centrifugation and subsequent ultrafiltration using water or an aqueous buffer solution having a concentration preferably from 1 to 1000 mmol/I, more preferably from 1 to 100 mmol/l, and more preferably from 10 to 50 mmol/l, such as about 20 mmol/l, a pH value preferably in the range of from 3 to 10, more preferably of from 4 to 8, such as about 7. The number of exchange cycles preferably is in the range of from 5 to 50, more preferably of from 10 to 30, and even more preferably of from 15 to 25, such as about 20.

Most preferably, the obtained derivative is further lyophilized until the solvent content of the reaction product is sufficiently low according to the desired specifications of the product.

In case the linker comprises a protecting group (PG), the method preferably further comprises a deprotection step.

The reaction conditions used are adapted to the respective protecting group used. According to a preferred embodiment of the invention, Z¹ is a thiol group, and the group Z¹*-PG is a disulfide, as described above. In this case, the deprotection step comprises the reduction of this disulfide bond to give the respective thiol group. This deprotection step is preferably carried out using specific reducing agents. As possible reducing agents, complex hydrides such as borohydrides, especially sodium borohydride, and thiols, especially dithiothreitol (DTT) and dithioerythritol (DTE) are mentioned. The reduction is preferably carried out using DTT.

The deprotection step is preferably carried out at a temperature in the range of from 0 to 80° C., more preferably in the range of from 10 to 50° C. and especially preferably in the range of from 20 to 40° C. During the course of the reaction, the temperature may be varied, preferably in the above-given ranges, or held essentially constant.

Preferably, the reaction is carried out in aqueous medium. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably from 0.01 to 1 mM, most preferably from 0.1 to 0.5 mM, such as about 0.4 mM.

The pH value in the deprotection step may be adapted to the specific needs of the reactants. Preferably, the reaction is carried out in buffered solution, at a pH value in the range of from 3 to 14, more preferably of from 5 to 11, and even more preferably of from 7.5 to 8.5. Among the preferred buffers, carbonate, phosphate, borate and acetate buffers as well as tris(hydroxymethyl)aminomethane (TRIS) may be mentioned.

Again, at least one of the isolation steps/and or purification steps, as described above, may be carried out subsequent to the deprotection step. Most preferably the obtained derivative is further lyophilized until the solvent content of the reaction product is sufficiently low according to the desired specifications of the derivative.

Step (a2)-(ii)

As regards step (a2)-(ii) of the method according to the present invention, in this step, the functional group Z¹ is introduced by displacing a hydroxyl group present in the hydroxyalkyl starch in a substitution reaction with a precursor of the functional group Z¹ or with a bifunctional linker comprising the functional group Z¹ or a precursor thereof.

Preferably, prior to the replacement of the hydroxyl group with the functional group Z¹, the at least one hydroxyl group of the hydroxyalkyl starch is activated to generate a suitable leaving group. Preferably, a group R^{L} is added to the at least one hydroxyl group thereby generating a group —O—R^{L}, wherein the structural unit —O—R^{L} is the leaving group.

Thus, the present invention also relates to a method for preparing a hydroxyalkyl starch derivative, as described above, as well as to a hydroxyalkyl starch derivative obtained or obtainable by said method wherein in step (a2)-(ii), prior to the substitution (displacement) of the hydroxyl group with the group comprising the functional group Z¹ or a precursor thereof, a group R^{L} is added to at least one hydroxyl group thereby generating a group —O—R^{L}, wherein —O—R^{L} is the leaving group.

The term “leaving group” as used in this context of the present invention is denoted to mean that the molecular fragment —O—R^{L} departs when reacting the hydroxyalkyl starch derivative with a reagent, such as a crosslinking compound, comprising the functional group Z¹ or a precursor thereof.

As regards, preferred leaving groups used in this context of the present invention, according to a preferred embodiment, the hydroxyl group is transformed to a sulfonic ester, such as a mesylic ester (—OMs), tosylic ester (—OTs), imsyl ester (imidazylsulfonyl ester) or a carboxylic ester such as trifluoroacetic ester.

Preferably, the at least one leaving group is generated by reacting at least one hydroxyl group of hydroxyalkyl starch, preferably in the presence of a base, with the respective sulfonyl chloride to give the sulfonic ester, preferably the mesylic ester.

Thus, the present invention also relates to a method for preparing a hydroxyalkyl starch derivative as described above, as well as to a hydroxyalkyl starch derivative obtained or obtainable by said method, wherein in step (a2)-(ii), prior to the substitution (displacement) of the hydroxyl group with the group comprising the functional group Z¹ or a precursor thereof, a group R^{L} is added to at least one hydroxyl group, thereby generating a group —O—R^{L}, wherein —O—R^{L} is —O-Ms or —OTs (i.e. R^{L} is Ms or Ts), and wherein the OMs group is preferably introduced by reacting at least one hydroxyl group of hydroxyalkyl starch with methanesulfonyl chloride, and —OTs is introduced by reacting at least one hydroxyl group with toluenesulfonyl chloride.

The addition of the group R^{L} to at least one hydroxyl group of hydroxyalkyl starch, whereupon a group —O—R^{L} is formed, is preferably carried out in an organic solvent, such as N-methylpyrrolidone, dimethyl acetamide (DMA), dimethyl formamide (DMF), formamide, dimethylsulfoxide (DMSO) and mixtures of two or more thereof, preferably at a temperature in the range of from −60 to 80° C., more preferably in the range of from −30 to 50° C. and especially preferably in the range of from −30 to 30° C. The temperature may be held essentially constant or may be varied during the reaction procedure. The pH value for this reaction may be adapted to the specific needs of the reactants. Preferably, the reaction is carried out in the presence of a base. Among the preferred bases pyridine, substituted pyridines such as collidine or 2,6-lutidine, tertiary amine bases such as triethylamine, diisopropyl ethyl amine (DIEA), N-methylmorpholine, N-methylimidazole or amidine bases such as 1,8-diazabicyclo[5.4.0]undec-7-ene (DBU) and inorganic bases such as metal hydrides and carbonates may be mentioned. Especially preferred are substituted pyridines (collidine) and tertiary amine bases (DIEA, N-methylmorpholine). The reaction time for this reaction step may be adapted to the specific needs and is generally in the range of from 5 min to 24 hours, preferably of from 15 min to 10 hours, more preferably of from 30 min to 5 hours.

The derivative comprising the group O—R^{L}, may be subjected to at least one further isolation and/or purification step such as precipitation and/or centrifugation and/or filtration prior to the substitution reaction according to step (a2)-(ii). Likewise, instead or additionally, the derivative comprising the O—R^{L} group may be subjected to an after-treatment like ultrafiltration, dialysis, centrifugal filtration or pressure filtration, ion exchange chromatography, reversed phase chromatography, HPLC, MPLC, gel filtration and/or lyophilisation. According to a preferred embodiment, the derivative comprising the O—R^{L} group is in situ reacted with the precursor of the functional group Z¹ or with the bifunctional linker, comprising the functional group Z¹ or a precursor thereof.

As described above, the at least one hydroxyl group, preferably the at least one —O—R^{L} group, more preferably the O-Ms group, is displaced, in a substitution reaction, with the precursor of the functional group Z¹ or with a bifunctional linker comprising the functional group Z¹ or a precursor thereof.

According to a preferred embodiment of the present invention, the activated hydroxyl group, preferably the —O—R^{L} group, more preferably the O-Ms group, is reacted with the precursor of the functional group Z¹. The term “a precursor” as used in this context of the present invention is denoted to mean a reagent which is capable of displacing the group, thereby forming a functional group Z¹ or a group, which can be modified in at least one further step to give the functional group Z¹.

Thus, the present invention also relates to a method for preparing a hydroxyalkyl starch derivative, as described above, as well as to a hydroxyalkyl starch derivative obtained or obtainable by said method, wherein in step (a2)-(ii), prior to the substitution (displacement) of the hydroxyl group with the group comprising the functional group Z¹ or a precursor thereof, a group R^{L} is added to at least one hydroxyl group, thereby generating a group —O—R^{L}, wherein —O—R^{L} is a leaving group, and subsequently —O—R^{L} is replaced by a precursor of the functional group Z¹, the method further comprising converting the precursor after the substitution reaction to the functional group Z¹, and wherein Z¹ is preferably a thiol group.

In case Z¹ is an amine, reagents such as ammonia, hydrazine, acyl hydrazides, such as carbohydrazide, potassium phthalimide, azides, such as sodium azide, and the like, can be employed to introduce the functional group Z¹.

In case Z¹ is a thiol group, reagents such as thioacetic acid, alkyl or aryl thiosulfonates such as sodium benzenethiosulfonate, thiourea, thiosulfate or hydrogen sulfide can be employed as precursor to introduce the functional group Z¹.

According to an especially preferred embodiment of the present invention, the hydroxyl group present in the hydroxyalkyl starch is first activated and then reacted with thioacetate, thereby replacing the hydroxyl group with the structure —S—C(═O)—CH₃. A particularly preferred reagent is potassium thioacetate. Thus, the present invention also relates to a method, as described above, wherein in step (a2)-(ii) the hydroxyl group present in the hydroxyalkyl starch is reacted with thioacetate giving a functional group having the structure —S—C(═O)—CH₃.

In this substitution step, in principle any reaction conditions known to those skilled in the art can be used. Preferably, the reaction is carried out in organic solvents, such as N-methyl pyrrolidone, dimethyl acetamide (DMA), dimethyl formamide (DMF), formamide, dimethyl sulfoxide (DMSO) and mixtures of two or more thereof. Preferably this step is carried out at a temperature in the range of from 0 to 80° C., more preferably in the range of from 20 to 70° C. and especially preferably in the range of from 40 to 60° C. The temperature may be held essentially constant or may be varied during the reaction procedure.

The pH value for this reaction may be adapted to the specific needs of the reactants. Optionally, the reaction is carried out in the presence of a scavenger, which reacts with the leaving group —O—R^{L}, such as mercaptoethanol or the like.

The reaction time for the substitution step is generally in the range of from 1 hour to 7 days, preferably of from 3 to 48 hours, more preferably of from 4 to 18 hours.

The derivative obtained may be subjected to at least one further isolation and/or purification step, as described above.

Preferably, the derivative is subjected to at least one further step. In particular, in case the hydroxyl group present in the hydroxyalkyl starch is reacted with thioacetate, thereby replacing the hydroxyl group with the structure —S—C(═O)—CH₃, the derivative is preferably saponified in a subsequent step to give the functional group Z¹ with Z¹ being an —SH group.

Thus, the present invention also relates to a method as described above as well as to a derivative obtained or obtainable by said method, wherein in step (a2)-(ii), the hydroxyl group present in the hydroxyalkyl starch is reacted with thioacetate giving a functional group having the structure —S—C(═O)—CH₃, wherein the method further comprises saponification of the group —S—C(═O)—CH₃ to give the functional group Z¹.

It has to be understood, that in case at least one hydroxyl group present in hydroxyalkyl starch, comprising the structural unit according to the following formula (II)

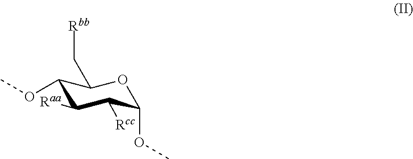

with R^{aa}, R^{bb} and R^{cc} being independently of each other selected from the group consisting of —O—HAS″ and —[O—(CR^{w}R^{x})—(CR^{y}R^{z})]ₓ—OH, is displaced in a substitution reaction, the stereochemistry of the carbon atom which bears the respective hydroxyl function, which is displaced, may be inverted.

Thus, in case at least one of R^{aa} and R^{bb} in the above shown structural unit is —OH (i.e. integer x is 0), and in case, this at least one group is displaced by a precursor of the functional group Z¹, thereby yielding in a hydroxyalkyl starch derivative comprising the functional group Z¹ in this structural unit, the stereochemistry of the carbon atoms bearing this functional group Z¹ may be inverted.

Since, it cannot be excluded that such a substitution of secondary hydroxyl groups occur, in the method of the invention according to step (a2)-(ii), the stereochemistry of the carbon atoms bearing the functional group R^{a} and R^{c} is not further defined, as shown in the structure with the formula (I)

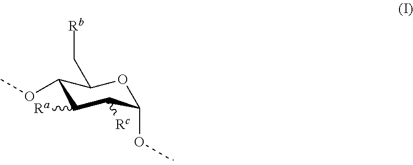

However, without wanting to be bound to any theory, it is believed that mainly primary hydroxyl groups will be displaced in the substitution reaction according to step (a2)-(ii). Thus, according to this theory, the stereochemistry of most carbon atoms bearing the residues R^{a} or R^{c} will not be inverted but the respective structural unit of the hydroxyalkyl starch will comprise the stereochemistry as shown in the formula (Ib)

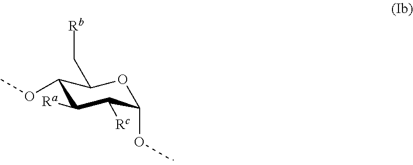

The thioacetate is preferably saponified in at least one further step to give the thiol comprising hydroxyalkyl starch derivatives. As regards the saponification of the functional group —S—C(═O)—CH₃, all methods known to those skilled in the art are encompassed by the present invention. This includes the use of bases (such as metal hydroxides) and strong nucleophiles (such as ammonia, amines, thiols or hydroxides) in order to saponify the present thioesters to give thiols. Preferred reagents are sodium hydroxide and ammonia.

Since thiols are well known to oxidize via the formation of disulfides, especially under basic conditions present in most saponification protocols, the molecular weight of the hydroxyalkyl starch derivative obtained may vary due to unspecific crosslinking. To prevent the formation of disulfides, preferably a reducing agent is added prior, during or after the saponification step. According to a preferred embodiment of the invention, a reducing agent is directly added to the saponification mixture in order to keep the forming thiol groups in their low oxidation state. Regarding the reduction of the thiol groups, all reduction methods known to those skilled in the art are encompassed by the present invention. According to preferred embodiments of the present invention, dithiothreitol (DTT), dithioerythritol (DTE) or sodium borohydride are employed.

In an alternative embodiment of the reaction, aqueous sodium hydroxide is used as saponification agent together with sodium borohydride as reducing agent.

Optionally, mercaptoethanol can be used as an additive in this reaction.

Thus, the present invention also relates to a method, as described above, wherein in step (a2)-(ii) the at least one activated hydroxyl group present in the hydroxyalkyl starch is reacted with thioacetate giving a functional group having the structure —S—C(═O)—CH₃, wherein the method further comprises saponification of the group —S—C(═O)—CH₃ to give the functional group Z¹, wherein the hydroxyalkyl starch derivative comprises at least one structural unit, preferably 3 to 200 structural units, according to the following formula (I)

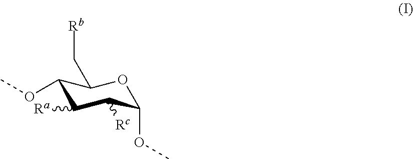

wherein R^{a}, R^{b} and R^{c} are independently of each other selected from the group consisting of —O—HAS″, —[O—CH₂—CH₂]ₛ—OH and —[O—CH₂—CH₂]ₜ—SH and wherein at least one R^{a}, R^{b} and R^{c} is —[O—CH₂—CH₂]ₜ—SH and wherein t is in the range of from 0 to 4, and wherein s is in the range of from 0 to 4.

Again, the hydroxyalkyl starch derivative, comprising the functional group —SH, obtained by the above-described preferred embodiment, may be isolated/and or purified in a further step. Again, the purification/isolation of the HAS derivative from step (a2)-(ii) can be carried out by any suitable method such as ultrafiltration, dialysis or precipitation or a combined method using for example precipitation and afterwards ultrafiltration.

Furthermore, the hydroxyalkyl starch derivative may be lyophilized, as described above, using conventional methods.

The following preferred embodiments directed to hydroxyalkyl starch derivatives are described:
1a. A hydroxyalkyl starch derivative preferably having a mean molecular weight MW above the renal threshold, preferably in the range of from 60 to 800 kDa, more preferably of from 80 to 800 kDa, and preferably having a molar substitution MS in the range of from 0.6 to 1.5, said hydroxyalkyl starch derivative comprising at least one structural unit, preferably 3 to 200 structural units, comprising at least one structural unit according to the following formula (I)

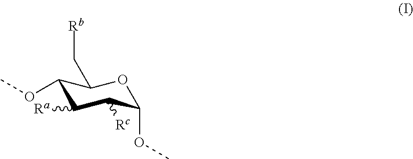

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹, and wherein at least one R^{a}, R^{b} and R^{c} is —[O—CH₂—CH₂]ₜ—Z¹ or —[O—CH₂—CH₂]ₜ-[F¹]ₚ-L¹-Z¹, and wherein t is in the range of from 0 to 4, and wherein s is in the range of from 0 to 4, p is 0 or 1, and wherein Z¹ is —SH, and

F¹ is a functional group, preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O— or —S—, -succinimide, —NH—NH—, —NH—O—, —CH═N—O—, —O—N═CH—, —CH═N—, —N═CH—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,

L¹ is a linking moiety, preferably selected from the group consisting of alkyl, alkylaryl, arylalkyl, aryl, heteroaryl, alkylheteroaryl and heteroarylalkyl.

and wherein HAS″ is a remainder of HAS.
2a. The hydroxyalkyl starch derivative according to embodiment 1a, said derivative comprising at least one structural unit according to the following formula (I)

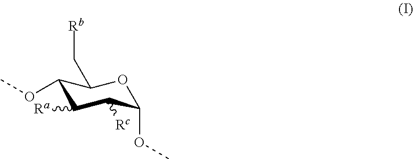

wherein R^{a}, R^{b} and R^{c} are
(i) independently of each other selected from the group consisting of —O—HAS″, —[O—CH₂—CH₂]ₛ—OH and —[O—CH₂—CH₂]ₜ—Z¹, with Z¹ being —SH wherein at least one of R^{a}, R^{b} and R^{c} is —[O—CH₂—CH₂]ₜ—Z¹, or
(ii) independently of each other selected from the group consisting of —O—HAS″, —[CH₂—CH₂]ₛ—Z¹ and —[O—CH₂—CH₂]ₜ-[F¹]ₚ-L¹-Z¹, with p being 1, and with Z¹ being —SH, wherein at least one of R^{a}, R^{b} and R^{c} is —[O—CH₂—CH₂]ₜ-[F¹]ₚ-L¹-Z¹, and wherein t is in the range of from 0 to 4, and wherein s is in the range of from 0 to 4 and wherein F¹ is preferably —O—.
3a. The hydroxyalkyl starch derivative according to embodiment 1a or 2a, said derivative comprising at least one structural unit according to the following formula (Ib)

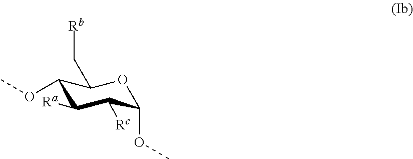

wherein at least one of R^{a}, R^{b} and R^{c} is
—[O—CH₂—CH₂]ₜ-[F¹]ₚ-L¹-Z¹ with Z¹ being —S—, preferably with p being 1 and F¹ being —O—,
wherein L¹ is preferably an alkyl chain, more preferably L¹ has a structure according to the following formula —{[CR^{d}R^{f}]ₕ-[F⁴]ᵤ—[CR^{dd}R^{ff}]_{z}}ₐₗₚₕₐ, wherein F⁴ is a functional group, preferably a group selected from the group consisting of —S—, —O— and —NH—, in particular —S—,
and wherein z is in the range of from 1 to 5, preferably in the range of from 1 to 3, more preferably 2,
and wherein h is in the range of from 1 to 5, preferably in the range of from Ito 3, more preferably 3,
and wherein u is 0 or 1,
integer alpha is in the range of from 1 to 10,
and wherein R^{d}, R^{f}, R^{dd} and R^{ff} are, independently of each other, selected from the group consisting of H, alkyl, hydroxyl, and halogen, preferably selected from the group consisting of H, methyl and hydroxyl,
and wherein each repeating unit of —[CR^{d}R^{f}]ₕ—F⁴—[CR^{dd}R^{ff}]_{z}— may be the same or may be different,
more preferably wherein L¹ has a structure selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—O—CH₂—CH₂—, —CH₂—CH₂—O—CH₂—CH₂—O—CH₂—CH₂—, —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, —CH₂—CH(CH₂OH)— and —CH₂—CH(CH₂OH)—S—CH₂—CH₂—, more preferably from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂—, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—.
4a. The hydroxyalkyl starch derivative according to embodiment 3a, wherein R^{a}, R^{b} and R^{c} are independently of each other selected from the group consisting of —O—HAS″, —[O—CH₂—CH₂]-OH and —[O—CH₂—CH₂]ₜ—O—CH₂—CHOH—CH₂—S—CH₂—CH₂—S— and wherein at least one of R^{a}, R^{b} and R^{c} is —[O—CH₂—CH₂]ₜ—O—CH₂—CHOH—CH₂—S—CH₂—CH₂—S—, wherein t is in the range of from 0 to 4, and wherein s is in the range of from 0 to 4.

### Pharmaceutical Composition

Furthermore, the present invention relates to a pharmaceutical composition comprising in a therapeutically effective amount a HAS conjugate, as described above, or a HAS conjugate obtained or obtainable by the above described method.

As far as the pharmaceutical compositions according to the present invention comprising the hydroxyalkyl starch conjugate, as described above, are concerned, the hydroxyalkyl starch conjugate may be used in combination with a pharmaceutical excipient. Generally, the hydroxyalkyl starch conjugate will be in a solid form which can be combined with a suitable pharmaceutical excipient that can be in either solid or liquid form. As excipients, carbohydrates, inorganic salts, antimicrobial agents, antioxidants, surfactants, buffers, acids, bases, and combinations thereof may be mentioned. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like. The excipient may also include an inorganic salt or buffer such as citric acid, sodium chloride, potassium chloride, sodium sulfate, potassium nitrate, sodium phosphate monobasic, sodium phosphate dibasic, and combinations thereof. The pharmaceutical composition according to the present invention may also comprise an antimicrobial agent for preventing or determining microbial growth, such as, e.g., benzalkonium chloride, benzethonium chloride, benzyl alcohol, cetylpyridinium chloride, chlorobutanol, phenol, phenylethyl alcohol, phenylmercuric nitrate, thimersol, and combinations thereof.

The pharmaceutical composition according to the present invention may also comprise an antioxidant, such as, e.g., ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The pharmaceutical composition according to the present invention may also comprise a surfactant, such as, e.g., polysorbates, or pluronics sorbitan esters; lipids, such as phospholipids and lecithin and other phosphatidylcholines, phosphatidylethanolamines, acids and fatty esters; steroids, such as cholesterol; and chelating agents, such as EDTA or zinc.

The pharmaceutical composition according to the present invention may also comprise acids or bases such as, e.g., hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof, and/or sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof. Generally, the excipient will be present in a pharmaceutical composition according to the present invention in an amount of 0.001 to 99.999 wt.-%, preferably from 0.01 to 99.99 wt.-%, more preferably from 0.1 to 99.9 wt.-%, in each case based on the total weight of the pharmaceutical composition.

Preferably the pharmaceutical composition contains no surfactants such as cremophore EL, polysorbates, in particular no Tween 80®, and/or no ethanol.

The present invention also relates to a method of treating cancer, comprising administering to a patient suffering from cancer a therapeutically effective amount of the hydroxyalkyl starch conjugate as defined herein, or the hydroxyalkyl starch conjugate obtained or obtainable by the method according to the present invention, or the pharmaceutical composition according to the present invention.

The term “patient”, as used herein, relates to animals and, preferably, to mammals. More preferably, the patient is a rodent such as a mouse or a rat. Even more preferably, the patient is a primate. Most preferably, the patient is a human. It is, however, envisaged by the method of the present invention that the patient shall suffer from cancer.

The term “cancer”, as used herein, preferably refers to a proliferative disorder or disease caused or characterized by the proliferation of cells which have lost susceptibility to normal growth control. Preferably, the term encompasses tumors and any other proliferative disorders. Thus, the term is meant to include all pathological conditions involving malignant cells, irrespective of stage or of invasiveness. The term, preferably, includes solid tumors arising in solid tissues or organs as well as hematopoietic tumors (e.g. leukemias and lymphomas).

The cancer may be localized to a specific tissue or organ (e.g. in the breast, the prostate or the lung), and, thus, may not have spread beyond the tissue of origin. Furthermore the cancer may be invasive, and, thus may have spread beyond the layer of tissue in which it originated into the normal surrounding tissues (frequently also referred to as locally advanced cancer). Invasive cancers may or may not be metastatic. Thus, the cancer may be also metastatic. A cancer is metastatic, if it has spread from its original location to distant parts of the body. E.g., it is well known in the art that breast cancer cells may spread to another organ or body part, such as the lymph nodes.

Preferred cancers are breast cancer (particularly, locally advanced or metastatic breast cancer), colorectal cancer, lung cancer (particularly, locally advanced or metastatic non-small cell lung cancer), prostate cancer (preferably, hormone-refractory prostate cancer), ovarian cancer, liver cancer, renal cancer, gastric cancer (e.g., including adenocarcinoma such as adenocarcinoma of the gastroesophageal junction), head and neck cancers (particularly locally advanced squamous cell carcinoma of the head and neck), Kaposi's sarcoma and melanoma.

Moreover, it is also envisaged that the cancer is selected from the group consisting of neuroblastoma, intestine carcinoma such as rectum carcinoma, familiary adenomatous polyposis carcinoma and hereditary non-polyposis, esophageal carcinoma, labial carcinoma, larynx carcinoma, hypopharynx carcinoma, tong carcinoma, salivary gland carcinoma, adenocarcinoma, medullary thyroidea carcinoma, papillary thyroidea carcinoma, renal carcinoma, kidney parenchym carcinoma, ovarian carcinoma, cervical carcinoma, uterine corpus carcinoma, endometriuni carcinoma, chorion carcinoma, pancreatic carcinoma, testis carcinoma, urinary carcinoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, small cell lung carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

The terms “treating cancer” and “treatment of cancer”, preferably, refer to therapeutic measures, wherein the object is to prevent or to slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. It is to be understood that a treatment can also mean prolonging survival as compared to expected survival if not receiving treatment.

The term “administering” as used herein, preferably, refers to the introduction of the hydroxyalkyl starch conjugate as defined herein, the hydroxyalkyl starch conjugate obtained or obtainable by the method according to the present invention, or the pharmaceutical composition according to the present invention into cancer patients. Methods for administering a particular compound are well known in the art and include parenteral, intravascular, paracanceral, transmucosal, transdermal, intramuscular (i.m.), intravenous (i.v.), intradermal, subcutaneous (s.c.), sublingual, intraperitoneal (i.p.), intraventricular, intracranial, intravaginal, intratumoral, and oral administration. It is to be understood that the route of administration may depend on the cancer to be treated. Preferably, the hydroxyalkyl starch conjugate as defined herein, the hydroxyalkyl starch conjugate obtained or obtainable by the method according to the present invention, or the pharmaceutical composition according to the present invention are administered parenterally. More preferably, it is administered intravenously. Preferably, the administration of a single dose of a therapeutically effective amount of the aforementioned compounds is over a period of 5 min to 5 h.

Preferably, the conjugates are administered together with a suitable carrier, and/or a suitable diluent, such as preferably a sterile solution for i.v., i.m., i.p. or s.c. application.

The term “therapeutically effective amount”, as used herein, preferably refers to an amount of the hydroxyalkyl starch conjugate as defined herein, the hydroxyalkyl starch conjugate obtained or obtainable by the method according to the present invention, or the pharmaceutical composition according to the present invention that (a) treats the cancer, (b) attenuates, ameliorates, or eliminates the cancer. More preferably, the term refers to the amount of the cytotoxic agent present in the hydroxyalkyl starch conjugate as defined herein, the hydroxyalkyl starch conjugate obtained or obtainable by the method according to the present invention, or the pharmaceutical composition according to the present invention that (a) treats the cancer, (b) attenuates, ameliorates, or eliminates the cancer. How to calculate the amount of a cytotoxic agent present in the aforementioned conjugates or pharmaceutical composition is described elsewhere herein. It is particularly envisaged that the therapeutically effective amount of the aforementioned compounds shall reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, at least to some extent, tumor growth; and/or relieve to some extent one or more of the symptoms associated with the cancer. Whether a particular amount of the aforementioned compounds exerts these effects (and, thus is pharmaceutically effective) can be determined by well known measures. Particularly, it can be determined by assessing cancer therapy efficacy. Cancer therapy efficacy, e.g., can be assessed by determining the time to disease progression and/or by determining the response rate. Thus, the required dosage will depend on the severity of the condition being treated, the patient's individual response, the method of administration used, and the like. The skilled person is able to establish a correct dosage based on his general knowledge.

Advantageously, it has been shown in the studies carried out in the context of the present invention that

i) the cytotoxic agent is less toxic when present in the conjugates described herein as compared to an agent not being present in a conjugate and/or that
ii) the use of said conjugate, or of a pharmaceutical composition comprising said conjugate allows for a more efficient treatment of cancer in a subject (see Examples 2.4 and 2.5).

Moreover, the present invention relates to the hydroxyalkyl starch conjugate as defined above, or the hydroxyalkyl starch conjugate obtained or obtainable by the method according to the present invention, or the pharmaceutical composition according to the present invention for use as a medicament.

Moreover, the present invention relates to the hydroxyalkyl starch conjugate as defined above, or the hydroxyalkyl starch conjugate obtained or obtainable by the method according to the present invention, or the pharmaceutical composition according to the present invention for the treatment of cancer.

Also envisaged by the present invention is the hydroxyalkyl starch conjugate as defined above, or the hydroxyalkyl starch conjugate obtained or obtainable by the method according to the present invention, or the pharmaceutical composition according to the present invention for the treatment of cancer selected from the group consisting of breast cancer, colorectal cancer, lung cancer, prostate cancer, ovarian cancer, liver cancer, renal cancer, gastric cancer, head and neck cancers, Kaposi's sarcoma and melanoma.

Finally, the present invention pertains to the use of the hydroxyalkyl starch conjugate as defined above, or the hydroxyalkyl starch conjugate obtained or obtainable by the method according to the present invention, or the pharmaceutical composition according to the present invention for the manufacture of a medicament for the treatment of cancer. Preferably, the cancer is selected from the group consisting of breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.

How to administer the conjugates, compositions or medicaments has been explained elsewhere herein.

In the following especially preferred embodiments of the present invention are described:
1. A hydroxyalkyl starch (HAS) conjugate comprising a hydroxyalkyl starch derivative and a cytotoxic agent, said conjugate having a structure according to the following formula

HAS′(-L-M)ₙ
wherein
M is a residue of a cytotoxic agent, wherein the cytotoxic agent comprises a secondary hydroxyl group,
L is a linking moiety,
HAS′ is a residue of the hydroxyalkyl starch derivative,
n is greater than or equal to 1, preferably in the range of from 3 to 200,
and wherein the hydroxyalkyl starch derivative has a mean molecular weight MW above the renal threshold, preferably in the range of from 60 to 800 kDa, more preferably of from 80 to 800 kDa,
and a molar substitution MS in the range of from 0.6 to 1.5,
and wherein the linking moiety L is linked to the secondary hydroxyl group of the cytotoxic agent, and wherein the cytotoxic agent is preferably a taxane.
2. The conjugate according to embodiment 1, wherein the hydroxyalkyl starch derivative is a hydroxyethyl starch derivative (HES′).
3. The conjugate according to embodiment 1 or 2, wherein the hydroxyalkyl starch derivative has a mean molecular weight MW in the range of from 90 to 350 kDa, preferably in the range of from 95 to 150 kDa.
4. The conjugate according to any of embodiments 1 to 3, wherein the hydroxyalkyl starch derivative has a molar substitution MS in the range of from 0.70 to 1.45, more preferably in the range of 0.80 to 1.40, more preferably in the range of from 0.85 to 1.35, more preferably in the range of from 0.90 to 1.10, most preferably in the range of from 0.95 to 1.05.
5. The conjugate according to any of embodiments 1 to 4, wherein the linking moiety L has a structure -L′-F³—, wherein F³ is a functional group linking L¹ with the secondary hydroxyl group of the cytotoxic agent thereby forming a —F³—O— bond, preferably wherein F³ is a —C(═Y)— group, with Y being O, NH or S, with Y being in particular O or S, and wherein L¹ is a linking moiety.
6. The conjugate according to embodiment 5, wherein the conjugate comprises an electron-withdrawing group in alpha or beta position to each F³ group.
7. The conjugate according to embodiment 6, wherein the electron-withdrawing group is a group selected from the group consisting of —NH—C(═O)—, —C(═O)—NH—, —NH—, —O—, —S—, —SO—, —SO₂— and succinimide-.
8. The conjugate according to embodiment 5, wherein L¹ has a structure according to the following formula

-[F²]_{q}-[L²]_{g}-[E]ₑ—[CR^{m}Rⁿ]_{f}-
wherein E is an electron-withdrawing group, preferably selected from the group consisting of —C(═O)—NH—, —NH—, —O—, —S—, —SO—, -succinimide- and —SO₂—,
L² is a linking moiety, preferably selected from the group consisting of alkyl, alkenyl, alkylaryl, arylalkyl, aryl, heteroaryl, alkylheteroaryl and heteroarylalkyl,
F² is a group consisting of —Y¹, C(═Y²), C(═Y²)—NR^{F2},

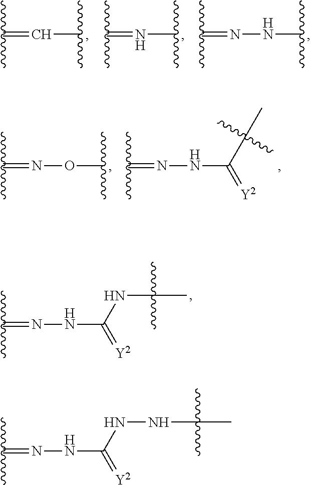

and —CH₂—CH₂—C(═Y²)—NR^{F2}—,
wherein Y¹ is selected from the group consisting of —S—, —O—, —NH—, —NH—NH—, —CH₂—CH₂—SO₂—NR^{F2}_, —CH₂—CHOH—, and cyclic imides, and wherein Y² is selected from the group consisting of NH, S and O, and wherein R^{F2} is selected from the group consisting of hydrogen, alkyl, alkylaryl, arylalkyl, aryl, heteroaryl, alkylheteroaryl or heteroarylalkyl group,
f is 1, 2 or 3, preferably 1 or 2, most preferably 1, g is 0 or 1, q is 0 or 1, e is 0 or 1,
and wherein R^{m} and Rⁿ are, independently of each other, H or alkyl, preferably H or methyl, in particular H.
9. The conjugate according to any of embodiments 1 to 8, wherein the hydroxyalkyl starch derivative comprises at least one structural unit according to the following formula, preferably at least 3 to 200 structural units according to the following formula (I)

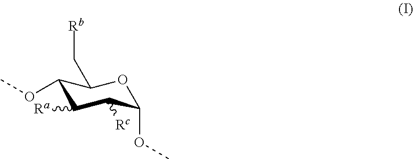

wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—X—, —[O—(CR^{w}R^{x})—(CR^{y}R^{z})]_{y}—[F¹]ₚ-L¹-X—, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{c} is —[O(CR^{w}R^{x})—(CR^{y}R^{z})]_{y}—X— or —[O—(CR^{w}R^{x})—(CR^{y}R^{z})]_{y}—[F¹]ₚ-L¹-X—,
preferably wherein R^{a}, R^{b} and R^{c} are independently of each other selected from the group consisting of —O—HAS″, —[O—CH₂—CH₂]ₛ—OH, —[O—CH₂—CH₂], —X— and —[O—CH₂—CH₂]ₜ-[F¹]ₚ-L¹-X—, wherein t is in the range of from 0 to 4, and wherein s is in the range of from 0 to 4 and wherein at least one of R^{a}, R^{b} and R^{c} is —[O—CH₂—CH₂]ₜ—X— or —[O—CH₂—CH₂]ₜ-[F¹]ₚ-L¹-X—,
and wherein X is selected from the group consisting of —Y^{xx}—, —C(═Y^{x})—, —C(═Y^{x})—NR^{xx}—,

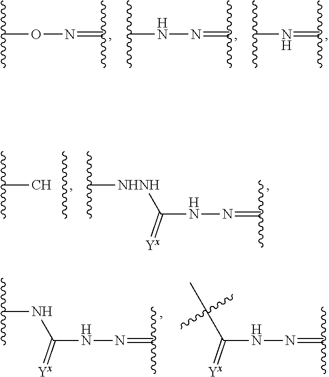

and —CH₂—CH₂—C(═Y^{x})—NR^{xx}—, wherein Y^{xx} is selected from the group consisting of —S—, —O—, —NH—, —NH—NH—, —CH₂—CH₂—SO₂—NR^{xx}—, and cyclic imides, such as succinimide, and wherein r is selected from the group consisting of NH, S and O, and wherein R^{xx} is selected from the group consisting of hydrogen, alkyl, alkylaryl, arylalkyl, aryl, heteroaryl, alkylheteroaryl or heteroarylalkyl group,
preferably wherein X is —S—,
F¹ is a functional group, preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, -succinimide, —NH—NH—, —NH—O—, —CH═N—O—, —O—N═CH—, —CH═N—, —N═CH—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,
L¹ is a linking moiety, preferably selected from the group consisting of alkyl, alkenyl, alkylaryl, arylalkyl, aryl, heteroaryl, alkylheteroaryl and heteroarylalkyl,
and wherein HAS″ is a remainder of HAS.
10. The conjugate according to embodiment 9 or 10, wherein the linking moiety L, preferably L′, is covalently linked to the —[O—CH₂—CH₂]ₜ—X— group or —[O—CH₂—CH₂]ₜ-[F¹]ₚ-L¹-X— group.
11. The conjugate according to embodiment 10, wherein at least one of R^{a}, R^{b} and R^{c} is
   (i) —[O—CH₂—CH₂]ₜ—X— and X is —S—, or
   (ii) —[O—CH₂—CH₂]ₜ-[F¹]ₚ-L¹-X— with X being —S—, preferably with p being 1 and F¹ being —O—,
   and wherein the structural unit -L-M is linked directly to the group X via the linking moiety L.
12. The conjugates according to any of embodiments 1 to 11, wherein the cytotoxic agent is a taxane.
13. The conjugates according to any of embodiments 1 to 12, wherein the cytotoxic agent is docetaxel or paclitaxel, most preferably docetaxel.
14. The conjugate according to embodiments 1 to 13, wherein the conjugate has a structure according to the following formula:

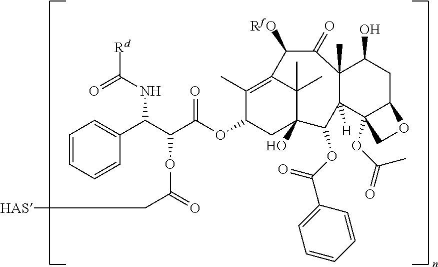

wherein R^{d} is preferably phenyl or O-t-butyl, and wherein R^{f} is preferably H or acetyl.
15. The conjugate according to embodiment 8, wherein L′ has a structure according to the following formula

-[F²]_{q}-[L²]_{g}-[E]ₑ—[CR^{m}Rⁿ]_{f}-
wherein e is 1, and wherein E is O— or —S—.
16. The conjugate according to embodiment 9, wherein HAS′ comprises at least one structural unit, preferably 3 to 200 structural units, according to the following formula (I)

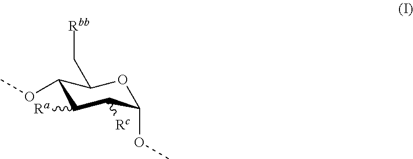

wherein R^{a}, R^{b} and R^{c} are
(i) independently of each other selected from the group consisting of —O—HAS″, —[O—CH₂—CH₂]ₛ—OH and —[O—CH₂—CH₂]ₜ—X—, with X being —S— wherein at least one of R^{a}, R^{b} and R^{c} is —[O—CH₂—CH₂]ₜ—X, or
(ii) independently of each other selected from the group consisting of —O—HAS″, —[O—CH₂—CH₂]ₛ—OH and —[O—CH₂—CH₂]ₜ-[F¹]ₚ-L¹-X, with p being 1, and with X being —S—, wherein at least one of R^{a}, R^{b} and R^{c} is —[O—CH₂—CH₂]ₜ-[F¹]ₚ-L¹-X—,
and wherein t is in the range of from 0 to 4, and wherein s is in the range of from 0 to 4 and wherein L¹ is linked directly to the group X, and wherein F³ is C(═O)—, and wherein F³ is linked to a group O— derived from the secondary hydroxyl group of the cytotoxic agent, thereby forming a C(═O)—O— bond.
17. The conjugate according to any of embodiments 9 to 11 and 16, wherein v is 1 and t is 1.
18. The conjugate according to embodiment 8 having a structure according to the following formula

HAS′(—[F²]_{q}-[L²]_{g}-[E]ₑ—[CR^{m}Rⁿ]_{f}—F³-M)ₙ
wherein q is 0, g is 0, e is 0, and wherein HAS′ comprises at least one structural unit, preferably 3 to 200 structural units, according to the following formula (I)

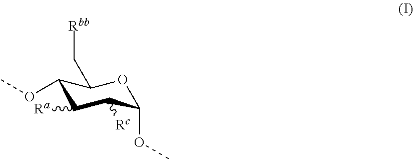

wherein at least one of R^{a}, R^{b} and R^{c} is —[O—CH₂—CH₂]ₜ—X— and X is —S— and the functional group X is directly linked to the
—[CR^{m}Rⁿ]_{f}— group, and wherein the hydroxyalkyl starch derivative comprises at least n functional groups X.
19. The conjugate according to embodiment 18, wherein f is 1, preferably wherein f is 1 and R^{m} and Ware H.
20. The conjugate according to embodiment 18 or 19, the conjugate having a structure according to the following formula

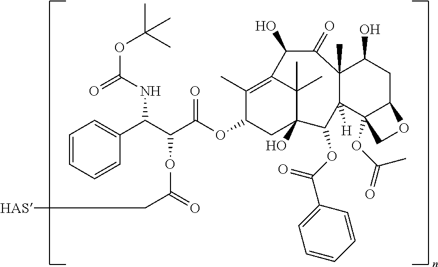

or the following formula

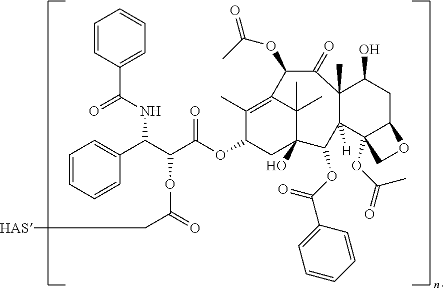

21. The conjugate according to embodiment 8, the conjugate having a structure according to the following formula

HAS′(—[F²]_{q}-L[L²]_{g}-[E]ₑ—[CR^{m}Rⁿ]_{f}—F³-M)ₙ
wherein HAS′ comprises at least one structural unit, preferably 3 to 200 structural units, according to the following formula (I)

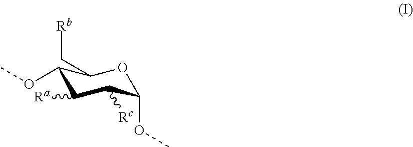

wherein at least one of R^{a}, R^{b} and R^{c} is —[O—CH₂—CH₂]ₜ—X— with X being —S—, wherein e is 1 and E is —S— or —O—, and wherein g and q are both 1.
22. The conjugate according to embodiment 21, wherein F² is —S— or -succinimide-, in particular succinimide-.
23. The conjugate according to embodiment 21 or 22, wherein L² is CH₂—CH₂—, the conjugate preferably having the structure

HAS′(-succinimide-CH₂—CH₂-E-[CR^{m}Rⁿ]_{f}—C(═O)-M)ₙ,
most preferably wherein R^{m} and Rⁿ are both H and f is 1.
24. The conjugate according to embodiment 8, the conjugate having a structure according to the following formula,

HAS′(—[F²]_{q}-[L²]_{g}-[E]ₑ—[CR^{m}Rⁿ]_{f}—F³-M)ₙ
wherein HAS′ comprises at least one structural unit, preferably 3 to 200 structural units, according to the following formula (Ib)

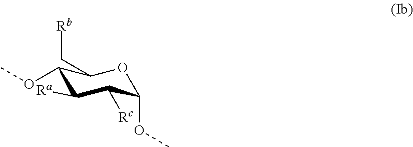

wherein at least one of R^{a}, R^{b} and R^{c} is
—[O—CH₂—CH₂]ₜ-[F¹]ₚ-L¹-X— with X being —S—, preferably with p being 1 and F¹ being —O—,
wherein L¹ is preferably an alkyl chain, more preferably L¹ has a structure according to the following formula —{[CR^{d}R^{f}]ₕ-[F⁴]ᵤ—[CR^{dd}R^{ff}]z}ₐₗₚₕₐ, wherein F⁴ is a functional group, preferably a group selected from the group consisting of —S—, —O— and —NH—, in particular —S—,
and wherein z is in the range of from 0 to 20, more preferably of from 0 to 10, more preferably 0 to 3, most preferably 0 to 2,
or and wherein z is in the range of from 1 to 5, preferably in the range of from 1 to 3, more preferably 2,
and wherein h is in the range of from 1 to 5, preferably in the range of from 1 to 3, more preferably 3,
and wherein u is 0 or 1,
integer alpha is in the range of from 1 to 10,
and wherein R^{d}, R^{f}, R^{dd} and R^{ff} are, independently of each other, selected from the group consisting of H, alkyl, hydroxyl, and halogen, preferably selected from the group consisting of H, methyl and hydroxyl,
and wherein each repeating unit of —[CR^{d}R^{f}]ₕ-[F⁴]ᵤ—[CR^{dd}R^{ff}]_{z}— may be the same or may be different,
more preferably wherein L¹ has a structure selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—O—CH₂—CH₂—, —CH₂—CH₂—O—CH₂—CH₂—O—CH₂—CH₂—, —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, —CH₂—CH(CH₂OH)— and —CH₂—CH(CH₂OH)—S—CH₂—CH₂—, more preferably from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂—, more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—.
25. The conjugate according to embodiment 24, wherein f is 1 and wherein R^{m} and Rⁿ are both H, and wherein q, g and e are 0 and wherein L¹ is preferably —CH₂—CHOH—CH₂—S—CH₂—CH₂—.
26. The conjugate according to embodiment 24 or 25, wherein F³ is C(═O)— and M is the residue of docetaxel or of paclitaxel.
27. The conjugate according to any of embodiments 24 to 26, having the structure

HAS′(—CH₂—C(═O)-M)ₙ
wherein HAS′ comprises at least one structural unit, preferably 3 to 200 structural units, according to the following formula (Ib)

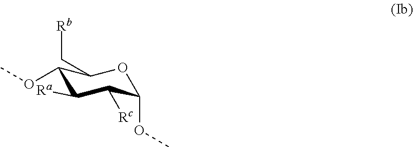

wherein R^{a}, R^{b} and R^{c} are independently of each other selected from the group consisting of —O—HAS″, —[O—CH₂—CH₂]ₛ—OH and —[O—CH₂—CH₂]—O—CH₂—CHOH—CH₂—S—CH₂—CH₂—S— and wherein at least one of R^{a}, R^{b} and R^{c} is —[O—CH₂—CH₂]—O—CH₂—CHOH—CH₂—S—CH₂—CH₂—S—, wherein t is in the range of from 0 to 4, and wherein s is in the range of from 0 to 4.
28. The conjugate according to embodiment 27 having a structure according to the following formula:

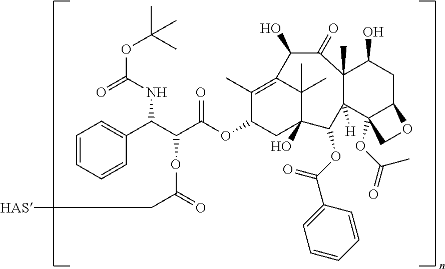

or the following formula

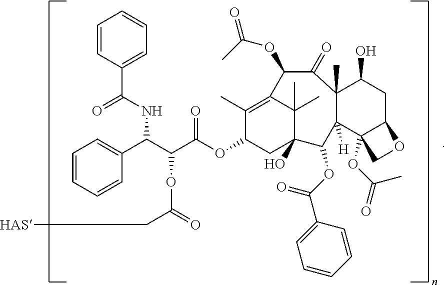

29. The conjugate according to embodiment 24, wherein q is 1 and F² is succinimide.
30. The conjugate according to embodiment 24 or 29, wherein e is 1, and E is O— or —S—.
31. The conjugate according to embodiment 29 or 30, wherein f is 1 and wherein R^{m} and Rⁿ are preferably both H, the conjugate more preferably having the formula

HAS′(succinimide-[L²]_{g}—S—CH₂—C(═O)-M)ₙ.
32. The conjugate according to any of embodiments 29 to 31, wherein g is 1 and L² has a structure selected from the group consisting of CH₂—CH₂—, CH₂—CH₂—CH₂— and CH₂—CH₂—CH₂—CH₂—.
33. The conjugate according to any of the embodiments 30 to 32, having the structure

HAS′(succinimide-CH₂—CH₂—S—CH₂—C(═O)-M)ₙ
wherein the succinimide is linked to the functional group —X— and —X— is —S—.
34. The conjugate according to embodiment 8, the conjugate having a structure according to the following formula,

HAS′(—[F²]_{q}-[L²]_{g}-[E]ₑ—[CR^{m}Rⁿ]_{f}—F³-M)ₙ
wherein HAS′ comprises at least one structural unit, preferably 3 to 200 structural units, according to the following formula (I)

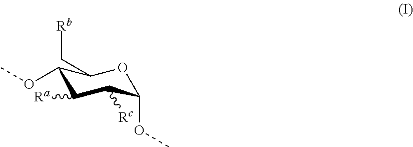

wherein at least one of R^{a}, R^{b} and R^{c} is with —[O—CH₂—CH₂]ₜ—[F¹]ₚ-L¹-X— with —X— being —S—,
with p being 1 and
F¹ being selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, —NH—NH—, —NH—O—, —CH═N—O—, —O—N═CH—, —CH═N—, —N═CH and cyclic imides, such as -succinimide, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,
preferably with F¹ being —Y⁷—C(═Y⁶)—Y⁸—, more preferably O—C(═O)—NH—,
and wherein L¹ is preferably an alkyl group.
35. The conjugate according to embodiment 34, having a structure according to the following formula

HAS′(—[F²]_{q}-[L²]_{g}-[E]ₑ—[CR^{m}Rⁿ]_{f}—F³-M)ₙ
wherein f is 1 and wherein R^{m} and Rⁿ are both H, and wherein q, g and e are 0.
36. The conjugate according to embodiment 34 or 35, wherein F³ is —C(═O)— and M is docetaxel or paclitaxel.
37. The conjugate according to embodiment 34 to 36, having the structure

HAS′(—CH₂—C(═O)-M)ₙ
and wherein HAS′ comprises at least one structural unit, preferably 3 to 200 structural units, according to the following formula (Ib)

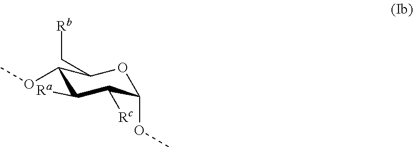

wherein R^{a}, R^{b} and R^{c} are independently of each other selected from the group consisting of —O—HAS″, —[O—CH₂—CH₂], —OH and —[O—CH₂—CH₂]ₜ—O—C(═O)—NH—CH₂—CH₂—S—, wherein t is in the range of from 0 to 4 and wherein s is in the range of from 0 to 4, and wherein at least one of R^{a}, R^{b} and R^{c} is —[O—CH₂—CH₂]ₜ—O—C(═O)—NH—CH₂—CH₂—S—.
38. The conjugate according to embodiment 34, wherein q is 1 and F² is succinimide.
39. The conjugate according to embodiment 38, wherein e is 1 and E is O— or —S—.
40. The conjugate according to embodiment 38 or 39, wherein f is 1 and wherein R^{m} and Rⁿ are preferably both H, the conjugate more preferably having the formula

HAS′(-succinimide-[L²]_{g}-E-CH₂—C(═O)-M)ₙ
with E being —O— or —S—.
41. The conjugate according to any of embodiments 38 to 40, wherein g is 1 and L² has a structure selected from the group consisting of —CH₂—CH₂—, —CH₂—CH₂—CH₂— and —CH₂—CH₂—CH₂—CH₂—.
42. The conjugate according to any of embodiments 38 to 41, having the structure

HAS′(-succinimide-CH₂—CH₂-E-CH₂—C(═O)-M)ₙ
with E being —O— or —S—, and wherein the succinimide is linked to the functional group —X— and —X— is S.
43. A method for preparing a hydroxyalkyl starch (HAS) conjugate comprising a hydroxyalkyl starch derivative and a cytotoxic agent, said conjugate having a structure according to the following formula

HAS′(-L-M)ₙ
wherein
M is a residue of a cytotoxic agent, wherein the cytotoxic agent comprises a secondary hydroxyl group,
L is a linking moiety,
HAS′ is a residue of the hydroxyalkyl starch derivative,
and n is greater than or equal to 1, preferably wherein n is in the range of from 3 to 200,
said method comprising
(a) providing a hydroxyalkyl starch derivative having a mean molecular weight MW above the renal threshold, preferably in the range of from 60 to 800 kDa, more preferably of from 80 to 800 kDa, and a molar substitution MS in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent.
44. The method according to embodiment 43, wherein the cytotoxic agent is reacted with the at least one crosslinking compound L via the functional group K¹ comprised in the crosslinking compound L, wherein said functional group K¹ comprises the structural unit C(═Y)—, with Y being O, NH or S, preferably, wherein K¹ is a carboxylic acid group or a reactive carboxy group.
45. The method according to embodiment 43 to 44, wherein the cytotoxic agent is reacted with the crosslinking compound L prior to the reaction with the HAS derivative.
46. The method according to any of embodiments 43 to 45, wherein the crosslinking compound L has a structure according to the following formula

K²-L′-K¹
wherein K¹ is a functional group comprising the structural unit C(═Y)— and L¹ is a linking moiety.
47. The method according to embodiment 46, wherein K² is reacted with the functional group Z¹ comprised in the HAS derivative, wherein Z¹ is selected from the group consisting of aldehyde groups, keto groups, hemiacetal groups, acetal groups, alkynyl groups, azides, carboxy groups, alkenyl groups, thiol reactive groups, —SH, —NH₂, —O—NH₂, —NH—O-alkyl, —(C=G)-NH—NH₂, -G-(C=G)-NH—NH₂, —NH—(C=G)-NH—NH₂, and —SO₂—NH—NH₂ where G is O or S and, if G is present twice, it is independently O or S, more preferably wherein Z¹ is a thiol group (—SH).
48. The method according to embodiment 47, wherein the cytotoxic agent is reacted via a secondary hydroxyl group with the functional group K′, thereby forming a functional group —F³—O—, wherein F³ is a C(═Y)— group, with Y being O, NH or S, in particular with Y being O or S.
49. The method according to any of embodiments 43 to 48, wherein the at least one crosslinking compound L has a structure according to the following formula

K²-[L²]_{g}-[E]ₑ—[CR^{m}Rⁿ]_{f}—K¹
wherein E is an electron-withdrawing group, preferably selected from the group consisting of —NH—C(═O)—, —C(═O)—NH—, —NH—, —O—, —S—, —SO—, —SO₂— and succinimide
   L² is a linking moiety, preferably selected from the group consisting of alkyl, alkylaryl, arylalkyl, aryl, heteroaryl, alkylheteroaryl and heteroarylalkyl,
g is 0 or 1,
e is 0 or 1,
and f is 1, 2 or 3, preferably 1 or 2, most preferably 1
and wherein R^{m} and Rⁿ are; independently of each other, H or alkyl, more preferably H or methyl, in particular H.
50. The method according to embodiment 43, wherein the derivative provided in step (a) comprises at least one structural unit, preferably 3 to 200 structural units, according to the following formula (I)

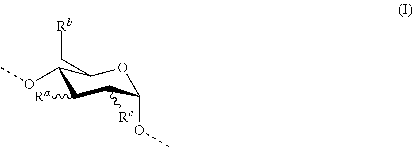

wherein at least one of R^{a}, R^{b} or R^{c} comprises the functional group Z¹,
preferably wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—Z¹ and —[O—(CR^{w}R^{x})—(CR^{y}R^{z})]_{y}-[F¹]ₚ-L¹-Z¹, and wherein
R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
F¹ is a functional group,
p is 0 or 1,
HAS″ is a remainder of the hydroxyalkyl starch
and L¹ is a linking moiety,
and wherein step (a) comprises the steps
(a1) providing a hydroxyalkyl starch (HAS) having a mean molecular weight MW above the renal threshold, preferably in the range of from 60 to 800 kDa, more preferably of from 80 to 800 kDa, and a molar substitution MS in the range of from 0.6 to 1.5, comprising the structural unit according to the following formula (II)

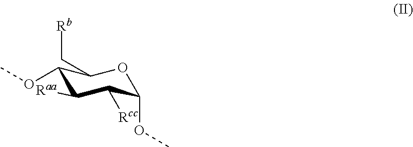

wherein R^{aa}, R^{bb} and R^{cc} are independently of each other selected from the group consisting —O—HAS″ and —[O—(CR^{w}R^{x})—(CR^{y}R^{z})]ₓ—OH,
wherein HAS″ is a remainder of the hydroxyalkyl starch,
R^{w}, R^{x}, R^{y} and R^{z} are independently of each other selected from the group consisting of hydrogen and alkyl,
x is an integer in the range of from 0 to 20, preferably in the range of from 0 to 4,
(a2) introducing at least one functional group Z¹ into the hydroxyalkyl starch by
   (i) coupling the hydroxyalkyl starch via at least one hydroxyl group to at least one suitable linker comprising the functional group Z¹ or a precursor of the functional group Z¹, or
   (ii) displacing a hydroxyl group present in the hydroxyalkyl starch in a substitution reaction with a precursor of the functional group Z¹ or with a bifunctional linker comprising the functional group Z¹ or a precursor thereof.
51. The method according to embodiment 50, wherein the HAS derivative formed in step (a2) comprises at least one structural unit, preferably 3 to 200 structural units, according to the following formula (I)

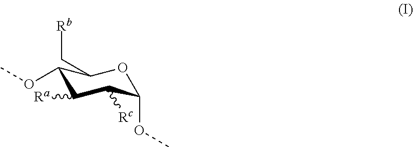

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹,
with t being in the range of from 0 to 4,
with s being in the range of from 0 to 4,
p being 0 or 1,
and wherein at least one of R^{a}, R^{b} and R^{c} comprises the functional group Z¹,
and wherein HAS″ is a remainder of HAS.
52. The method according to embodiment 50 or 51, wherein in (a2)-(i) the hydroxyalkyl starch is reacted with a suitable linker comprising the functional group Z¹ or a precursor of the functional group Z¹, and a functional group Z², the linker preferably having the structure Z²-L¹-Z¹ or Z²-L¹-Z¹*-PG, with Z² being a functional group capable of being reacted with the hydroxyalkyl starch or an activated hydroxyalkyl starch, thereby forming a hydroxyalkyl starch derivative comprising at least one structural unit, preferably 3 to 200 structural units, according to the following formula (I)

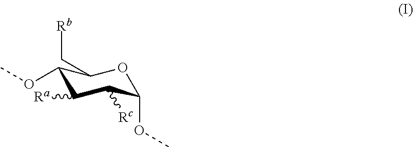

wherein at least one of R^{a}, R^{b} and R^{c} is —[O—CH₂—CH₂]ₜ-[F¹]ₚ-L¹-Z¹*-PG or —[O—CH₂—CH₂]ₜ-[F¹]ₚ-L¹-Z¹, and wherein PG is a suitable protecting group, more preferably Z¹ is —SH, Z¹* is —S—, and the group PG is a thiol protecting group, more preferably a protecting group forming together with Z¹* a thioether (e.g. trityl, benzyl, allyl); a disulfide (e.g. S-sulfonates, S-tert.-butyl, S-(2-aminoethyl)), or a thioester, and wherein in case the linker comprises a protecting group, the method further comprises a deprotection step.
53. The method according to any of embodiments 50 to 52, wherein step (a2)-(i) comprises
   (aa) activating at least one hydroxyl group of the hydroxyalkyl starch with a reactive carbonyl compound having the structure R**—(C═O)—R*, wherein R* and R** may be the same or different, and wherein R* and R** are both leaving groups, wherein upon activation an activated hydroxyalkyl starch derivative comprising at least one structural unit, preferably 3 to 200 structural units, according to the following formula (Ib)

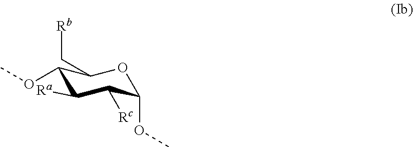

is formed, and wherein R^{a}, R^{b} and R^{c} are independently of each other selected from the group consisting of —O—HAS″, —[O—CH₂—CH₂]ₛ—OH and —[O—CH₂—CH₂]ₜ—O—C(═O)—R*, wherein t is in the range of from 0 to 4, and wherein s is in the range of from 0 to 4, and wherein at least one of R^{a}, R^{b} and R^{c} comprises the group —[O—CH₂—CH₂]ₜ—O—C(═O)—R*, and
(bb) reacting the activated hydroxyalkyl starch according to step (aa) with the at least one suitable linker comprising the functional group Z¹ or a precursor of the functional group Z¹.
54. The method according to embodiment 53, wherein the reactive carbonyl compound R**—(C═O)—R* is selected from the group consisting of phosgene, diphosgene, triphosgene, chloroformates and carbonic acid esters, preferably wherein the reactive carbonyl compound is selected from the group consisting of p-nitrophenylchloroformate, pentafluorophenylchloroformate, N,N′-disuccinimidyl carbonate, sulfo-N,N′-disuccinimidyl carbonate, dibenzotriazol-1-yl carbonate and carbonyldiimidazol.
55. The method according to embodiment 53 or 54, wherein in (bb) the activated hydroxyalkyl starch derivative is reacted with a linker comprising the functional group Z¹ or a precursor thereof and a functional group Z², the linker preferably having the structure Z²-L¹-Z¹* or Z²-L¹-Z¹*-PG, with Z² being a functional group capable of being reacted with the —[O—CH₂—CH₂]ₜ—O—C(═O)—R* group, and wherein L¹ being an alkyl group, and wherein upon reaction of the —O—C(═O)—R* group with the functional group Z², the functional group F¹ is formed, and wherein Z² is preferably —NH₂.
56. The method according to embodiment 55, wherein Z¹ is a thiol group and the linker has the structure Z²-L¹-Z¹*-PG, wherein PG is a suitable protecting group, more preferably wherein Z¹* is —S—, and the group PG is a thiol protecting group, more preferably a protecting group forming together with Z¹* a thioether (e.g. trityl, benzyl, allyl), a disulfide (e.g. S-sulfonates, S-tert.-butyl, S-(2-aminoethyl)), or a thioester, and wherein the method further comprises a deprotection step.
57. The method according to embodiment 50, wherein step (a2)-(i) comprises
   (I) coupling the hydroxyalkyl starch via at least one hydroxyl group comprised in the hydroxyalkyl starch to a first linker comprising a functional group Z², Z² being capable of being reacted with a hydroxyl group of the hydroxyalkyl starch, thereby forming a covalent linkage, the first linker further comprising a functional group W, wherein the functional group W is an epoxide or a group which is transformed in a further step to give an epoxide.
58. The method according to embodiment 57, wherein the first linker has a structure according to the formula Z²-L^{W}-W, wherein Z² is a functional group capable of being reacted with a hydroxyl group of the hydroxyalkyl starch and wherein L^{W} is a linking moiety, and wherein upon reaction of the hydroxyalkyl starch a hydroxyalkyl starch derivative is formed comprising at least one structural unit, preferably 3 to 200 structural units, according to the following formula (Ib)

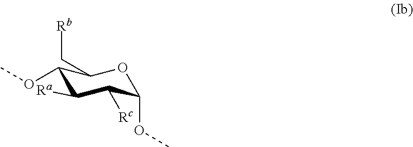

wherein R^{a}, R^{b} and R^{c} are independently of each other selected from the group consisting of —O—HAS″, —[O—CH₂—CH₂]ₛ—OH and is —[O—CH₂—CH₂]ₜ-[F¹]ₚ-L^{W}-W, wherein t is in the range of from 0 to 4, and s is in the range of from 0 to 4, and p is 1, and wherein at least one of R^{a}, R^{b} and R^{c} comprises the group —[O—CH₂—CH₂]ₜ-[F¹]ₚ-L^{W}-W, and wherein [F¹]ₚ is the functional group being formed upon reaction of Z² with the at least one hydroxyl group of the hydroxyalkyl starch.
59. The method according to embodiment 57 or 58, wherein W is an alkenyl group and the method further comprises
   (II) oxidizing the alkenyl group to give the epoxide, wherein potassium peroxymonosulfate is preferably employed as oxidizing agent.
60. The method according to any of embodiments 57 to 59, wherein Z² is a halogen (Hal) and wherein the functional group F¹ is —O—, preferably wherein the linker Z²-L^{w}-W has the structure Hal-CH₂—CH═CH₂.
61. The method according to any of embodiments 57 to 60, the method comprising
   (III) reacting the epoxide with a nucleophile comprising the functional group Z¹ or a precursor of the functional group Z¹, wherein the nucleophile is preferably a dithiol or a thiosulfate, thereby forming a hydroxyalkyl starch derivative comprising at least one structural unit, preferably 3 to 200 structural units, according to the following formula (Ib)

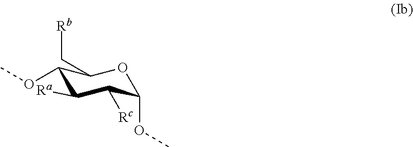

wherein R^{a}, R^{b} and R^{c} are independently of each other selected from the group consisting of —O—HAS″, —[O—CH₂—CH₂]ₛ—OH and —[O—CH₂—CH₂]ₜ-[F¹]ₚ-L¹-Z¹, wherein t is in the range of from 0 to 4, and s is in the range of from 0 to 4, and p is 1, and wherein at least one of R^{a}, R^{b} and R^{c} comprises the group —[O—CH₂—CH₂]ₜ-[F¹]ₚ-L¹-Z¹, and wherein L¹ is a linking moiety and wherein Z¹ is —SH.
62. The method according to embodiment 61, wherein the nucleophile is ethanedithiol or sodium thiosulfate.
63. The method according to embodiment 50, wherein in step (a2)-(ii), prior to the displacement of the hydroxyl group, a group R^{L} is added to at least one hydroxyl group thereby generating a group wherein —O—R^{L}, wherein —O—R^{L} is the leaving group, in particular a O-Mesyl (—OMs) or O-Tosyl (—OTs) group.
64. The method according to embodiment 63, wherein Z¹ is a thiol group, and wherein in step (a2)-(ii) the hydroxyl group present in the hydroxyalkyl starch is displaced by a suitable precursor, the method further comprising converting the precursor after the substitution reaction to the functional group Z¹.
65. The method according to embodiment 64, wherein in step (a2)-(ii) the hydroxyl group present in the hydroxyalkyl starch is displaced with thioacetate giving a functional group having the structure —S—C(═O)—CH₃, wherein the method further comprises conversion of the group —S—C(═O)—CH₃ to give the functional group Z¹, preferably wherein the conversion is carried out using sodium hydroxide and sodium borohydride.
66. The method according to any of embodiments 64 or 65, wherein the hydroxyalkyl starch derivative obtained according to step (a2)-(ii) comprises at least one structural unit according to the following formula (I)

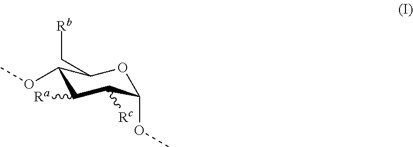

wherein R^{a}, R^{b} and R^{c} are independently of each other selected from the group consisting of —O—HAS″, —[O—CH₂—CH₂]ₛ—OH and —[O—CH₂—CH₂]ₜ—Z¹, wherein t is in the range of from 0 to 4, and s is in the range of from 0 to 4, and wherein at least one of R^{a}, R^{b} and R^{c} is —[O—CH₂—CH₂]ₜ—Z¹, with Z¹ being —SH, and wherein HAS″ is a remainder of HAS,
the method preferably further comprising reacting the hydroxyalkyl starch derivative in step (b) with a crosslinking compound L having a structure according to the formula K²—[L²]^{g}-[E]ₑ—[CR^{m}Rⁿ]_{f}—K¹ with g and e being 0, f is 1, 2 or 3, preferably 1 or 2, most preferably 1, wherein R^{m} and Rⁿ are, independently of each other H or alkyl, preferably H or methyl, in particular H, and wherein K² is a halogen.
67. The method according to any of embodiments 64 or 65, wherein HAS′ comprises at least one structural unit according to the following formula (I)

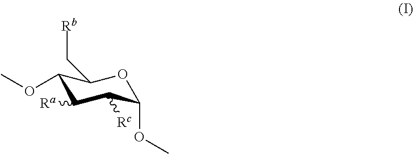

wherein R^{a}, R^{b} and R^{c} are independently of each other selected from the group consisting of —O—HAS″, —[O—CH₂—CH₂]ₛ—OH and —[O—CH₂—CH₂]ₜ—Z¹, wherein t is in the range of from 0 to 4, and s is in the range of from 0 to 4, and wherein at least one of R^{a}, R^{b} and R^{c} is —[O—CH₂—CH₂]-Z¹, with Z¹ being —SH,
and wherein the hydroxyalkyl starch derivative is preferably reacted in step (b) with a crosslinking compound L having a structure according to the formula K²-[L²]_{g}-[E]ₑ-[CR^{m}Rⁿ]_{f}—K¹,
and wherein K² is maleimide,
and wherein upon reaction of Z¹ with K², a functional group —X—F²— is formed,
wherein E is an electron-withdrawing group, preferably selected from the group consisting of NH—C(═O)—, —C(═O)—NH—, NH—, —O—, —SO—, —S—, -succinimide, and —SO₂
L² is a linking moiety, preferably selected from the group consisting of alkyl, alkylaryl, arylalkyl, aryl, heteroaryl, alkylheteroaryl and heteroarylalkyl group, g is 0 or 1,
e is 0 or 1,
and f is 1, 2 or 3, preferably 1 or 2, most preferably 1, and wherein R^{m} and Rⁿ are, independently of each other H or alkyl, preferably H or methyl.
68. The method according to embodiment 67, wherein g, e and f are 1 and E is O— or —S—, preferably —S—.
69. A hydroxyalkyl starch conjugate obtained or obtainable by a method according to any of embodiments 43 to 68.
70. A method for preparing a hydroxyalkyl starch derivative, preferably having a mean molecular weight MW above the renal threshold, preferably in the range of from 60 to 800 kDa, more preferably of from 80 to 800 kDa, and preferably having a molar substitution MS in the range of from 0.6 to 1.5, the hydroxyalkyl starch derivative comprising at least one structural unit, preferably 3 to 200 structural units, according to the following formula (I)

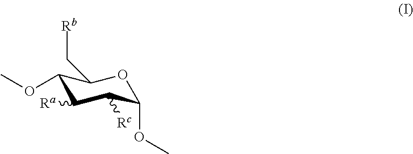

wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—Z¹, —[O—(CR^{w}R^{x})—(CR^{y}R^{z})]_{y}—[F¹]ₚ-L¹-Z¹, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, p is 0 or 1, L¹ is a linking moiety, HAS″ is the remainder of HAS and wherein Z¹ is a functional group capable of being reacted with a functional group of a further compound and wherein at least one of R^{a}, R^{b} and R^{c} comprises the functional group Z¹, and wherein Z¹ is preferably —SH,
said method comprising
(a1) providing a hydroxyalkyl starch, preferably having a mean molecular weight MW above the renal threshold, preferably from 60 to 800 kDa, preferably of from 80 to 800 kDa, and preferably having a molar substitution MS in the range of from 0.6 to 1.5, comprising the structural unit according to the following formula (II)

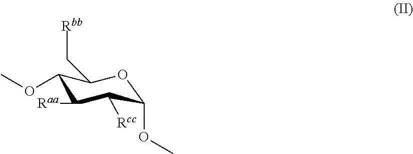

wherein R^{aa}, R^{bb} and R^{cc} are independently of each other selected from the group consisting of —O—HAS″ and —[O—(CR^{w}R^{x})—(CR^{y}R^{z})]ₓ—OH,
wherein HAS″ is a remainder of the hydroxyalkyl starch,
R^{w}, R^{x}, R^{y} and R^{z} are independently of each other selected from the group consisting of hydrogen and alkyl,
and x is an integer in the range of from 0 to 20, preferably in the range of from 0 to 4,
(a2) introducing at least one functional group Z¹ into the hydroxyalkyl starch by
   (i) coupling the hydroxyalkyl starch via at least one hydroxyl group to at least one suitable linker comprising the functional group Z¹ or a precursor of the functional group Z¹, or
   (ii) displacing a hydroxyl group present in the hydroxyalkyl starch in a substitution reaction with a precursor of the functional group Z¹ or with a bifunctional linker comprising the functional group Z¹ or a precursor thereof.
71. The method according to embodiment 70, wherein the HAS derivative formed in step (a2) comprises at least one structural unit, preferably 3 to 200 structural units, according to the following formula (I)

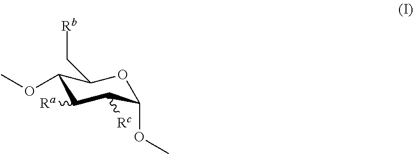

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹, wherein t is in the range of from 0 to 4, and wherein s is in the range of from 0 to 4, p being 0 or 1, and wherein at least one of R^{a}, R^{b} and R^{c} comprises the functional group Z¹, and wherein HAS″ is a remainder of HAS.
72. The method according to embodiment 70 or 71, wherein step (a2)-(i) comprises
   (I) coupling the hydroxyalkyl starch via at least one hydroxyl group comprised in the hydroxyalkyl starch to a first linker comprising a functional group Z², Z² being capable of being reacted with a hydroxyl group of the hydroxyalkyl starch, thereby forming a covalent linkage, the first linker further comprising a functional group W, wherein the functional group W is an epoxide or a group which is transformed in a further step to give an epoxide.
73. The method according to embodiment 72, wherein the first linker has a structure according to the following formula Z²-L^{w}-W, wherein Z² is a functional group capable of being reacted with at least one hydroxyl group of hydroxyalkyl starch and wherein L^{W} is a linking moiety, and wherein upon reaction of the hydroxyalkyl starch a hydroxyalkyl starch derivative is formed comprising at least one structural unit, preferably 3 to 200 structural units, according to the following formula (Ib)

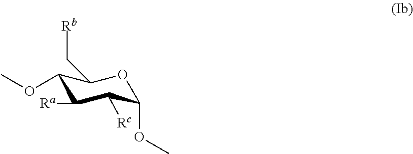

wherein R^{a}, R^{b} and R′ are independently of each other selected from the group consisting of —O—HAS″, —[O—CH₂CH₂]ₛ—OH and —[O—CH₂—CH₂]ₜ-[F¹]ₚ-L^{w}-W, wherein t is in the range of from 0 to 4, and wherein s is in the range of from 0 to 4, and p is 1, and wherein at least one of R^{a}, R^{b} and R^{c} comprises the group [—O—CH₂—CH₂]ₜ-[F¹]ₚ-L^{w}-W, and wherein [F¹]ₚ is the functional group being formed upon reaction of Z² with the at least one hydroxyl group of the hydroxyalkyl starch.
74. The method according to embodiment 72 or 73, wherein W is an alkenyl group and the method further comprises
   (II) oxidizing the alkenyl to give the epoxide, wherein potassium peroxymonosulfate is preferably employed as oxidizing agent.
75. The method according to embodiment 73 or 74, wherein Z² is a halogen (Hal) and wherein the functional group F¹ is —O—, preferably wherein the linker has the structure Hal-CH₂—CH═CH₂.
76. The method according to any of embodiments 72 to 75, the method comprising
   (III) reacting the epoxide with a nucleophile comprising the functional group Z¹ or a precursor of the functional group Z¹, wherein the nucleophile is preferably a dithiol or a thiosulfate, thereby forming a hydroxyalkyl starch derivative comprising at least one structural unit, preferably 3 to 200 structural units, according to the following formula (Ib)

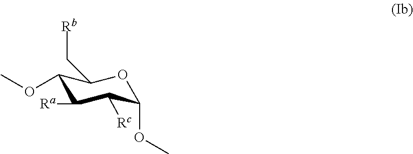

wherein R^{a}, R^{b} and R^{c} are independently of each other selected from the group consisting of —O—HAS″, —[O—CH₂—CH₂]ₛ—OH and —[O—CH₂—CH₂]ₜ-[F¹]ₚ-L¹-Z¹, wherein t is in the range of from 0 to 4, and wherein s is in the range of from 0 to 4, and p is 1, and wherein at least one of R^{a}, R^{b} and R^{c} comprises the group —[O—CH₂—CH₂]ₜ-[F¹]ₚ-L 1-Z¹, and wherein L¹ is a linking moiety and wherein Z¹ is —SH.
77. The method according to embodiment 76, wherein the nucleophile is ethanedithiol or sodium thiosulfate.
78. The method according to embodiment 70 or 71, wherein in step (a2)-(ii), prior to the displacement of the hydroxyl group with the group comprising the functional group Z¹ or a precursor thereof, a group R^{L} is added to at least one hydroxyl group thereby generating a group —O—R^{L}, wherein —O—R^{L} is a leaving group, in particular a —O-Ms or —O-Ts group.
79. The method according to embodiment 70 or 71 or 78, wherein in step (a2)-(ii) the hydroxyl group present in the hydroxyalkyl starch is displaced by a suitable precursor, the method further comprising converting the precursor after the substitution reaction to the functional group Z¹.
80. The method according to embodiment 79, wherein in step (a2)-(ii) the hydroxyl group present in the hydroxyalkyl starch is reacted with thioacetate as precursor giving a functional group having the structure —S—C(═O)—CH₃, wherein the method further comprises converting the group —S—C(═O)—CH₃ to give the functional group Z¹,
   preferably wherein the conversion is carried out using sodium hydroxide and sodium borohydride.
81. A hydroxyalkyl starch derivative, preferably having a mean molecular weight MW above the renal threshold, preferably in the range of from 60 to 800 kDa, more preferably of from 80 to 800 kDa, and preferably having a molar substitution MS in the range of from 0.6 to 1.5, said hydroxyalkyl starch derivative comprising at least one structural unit, preferably 3 to 200 structural units, according to the following formula (I)

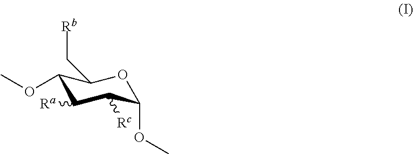

wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹, and wherein at least one R^{a}, R^{b} and R^{c} is —[O—CH₂—CH₂]ₜ—Z¹ or —[O—CH₂—CH₂]ₜ[F¹]ₚ-L¹-Z¹, and wherein t is in the range of from 0 to 4, and wherein s is in the range of from 0 to 4, p is 0 or 1, and wherein Z¹ is —SH, and
F¹ is a functional group, preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O— or —S—, -succinimide, —NH—NH—, —NH—O—, —CH═N—O—, —O—N═CH—, —CH═N—, —N═CH—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,
L¹ is a linking moiety, preferably selected from the group consisting of alkyl, alkylaryl, arylalkyl, aryl, heteroaryl, alkylheteroaryl and heteroarylalkyl.
and wherein HAS″ is a remainder of HAS.
82. A hydroxyalkyl starch derivative obtainable or obtained by a method according to any of embodiments 70 to 80.
83. Pharmaceutical composition comprising a conjugate according to any of embodiments 1 to 42 or according to embodiment 69.
84. Hydroxyalkyl starch conjugate according to any of embodiments 1 to 42 or according to embodiment 69, or pharmaceutical composition according to embodiment 83 for use as medicament.
85. Hydroxyalkyl starch conjugate according to any of embodiments 1 to 42 or according to embodiment 69, or pharmaceutical composition according to embodiment 83 for the treatment of cancer.
86. Hydroxyalkyl starch conjugate according to any of embodiments 1 to 42 or according to embodiment 69, or pharmaceutical composition according to embodiment 83 for the treatment of cancer selected from the group consisting of breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.
87. Use of a hydroxyalkyl starch conjugate according to any of embodiments 1 to 42 or according to embodiment 69, or of a pharmaceutical composition according to embodiment 83 for the manufacture of a medicament for the treatment of cancer.
88. Use according to embodiment 87, wherein the cancer is selected from the group consisting up breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.
89. A method of treating a patient suffering from cancer comprising administering a therapeutically effective amount of a hydroxyalkyl starch conjugate according to any of embodiments 1 to 42 or according to embodiment 69, or of a pharmaceutical composition according to embodiment 83.
90. The method of embodiment 89 wherein the patient suffers from cancer selected from the group consisting of breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.

### DESCRIPTION OF THE FIGURES

FIG. 1: Time course of the RTV(T/C) values after administering Docetaxel conjugates CDc1 and CDc2 (dosage 100 mg/kg body weight; mouse tumor model MT-3)

FIG. 1 shows the time course of the relative tumor volume in the mouse tumor model for human breast carcinoma MT-3 treated with conjugates CDc1 and CDc2 vs. mice in the control group (untreated mice (saline)) as well as vs. mice treated with Taxotere®.

The following symbols are used:

▪=saline, [symbol] =Docetaxel, ◯=CDc1, ∇=CDc2.

The X-axis shows the time [d], the Y-axis shows the relative tumor volume, RTV [%].

Each measurement was carried out with a group of 8 mice. The conjugates CDc1 and CDc2 were administered once at a dosage of 100 mg/kg body weight on day 7. Taxotere® was administered 3 times at a dosage of 10 mg/kg body weight at days 7, 9 and 11. Median values are given. Further details are given in Table 18.

FIG. 2 Time course of the body weight change after administering Docetaxel conjugates CDc1 and CDc2 (dosage 100 mg/kg body weight; mouse tumor model MT-3)

FIG. 2 shows the time course of the body weight change in the mouse tumor model for human breast carcinoma MT-3 treated with conjugates CDc1 and CDc2 vs. mice in the control group (untreated mice (saline)) as well as vs. mice treated with Taxotere®.

The following symbols are used:

▪=saline, [symbol] =Docetaxel, ◯=CDc1, ∇=CDc2.

The X-axis shows the time [d], the Y-axis shows the body weight change, BWC [%].

Each measurement was carried out with a group of 8 mice. The conjugates CDc1 and CDc2 were administered once at a dosage of 100 mg/kg body weight on day 7. Taxotere® was administered 3 times at a dosage of 10 mg/kg body weight at days 7, 9 and 11. Median values are given. Further details are given in Table 18.

FIG. 3: Time course of the RTV(T/C) values after administering Docetaxel conjugates CDc3-CDc8 (dosage 75 mg/kg body weight; mouse tumor model MT-3)

FIG. 3 shows the time course of the relative tumor volume in the mouse tumor model for human breast carcinoma MT-3 with conjugates CDc3-CDc8 vs. mice in the control group (untreated mice (saline)) as well as vs. mice treated with Taxotere®.

The following symbols are used:

▪=saline, [symbol] =Docetaxel, ◯=CDc3, ∇=CDc4, [symbol] =CDc5, Δ=CDc6, [symbol] =CDc7, ⋄=CDc8.

The X-axis shows the time [d], the Y-axis shows the relative tumor volume, RTV [%].

Each measurement was carried out with a group of 8 mice. The conjugates CDc3 to CDc8 were administered once at a dosage of 75 mg/kg body weight on day 11. Taxotere® was administered 5 times at a dosage of 5 mg/kg body weight at days 11 to 15. Median values are given. Further details are given in Table 19.

FIG. 4: Time course of the body weight change after administering Docetaxel conjugates CDc3-CDc8 (dosage 75 mg/kg body weight; mouse tumor model MT-3)

FIG. 4 shows the time course of the body weight change in the mouse tumor model for human breast carcinoma MT-3 treated with conjugates CDc3 to CDc8 vs. mice in the control group (untreated mice (saline)) as well as vs. mice treated with Taxotere®.

The following symbols are used:

▪=saline, [symbol] =Docetaxel, ◯=CDc3, ∇=CDc4, [symbol] =CDc5, Δ=CDc6, [symbol] =CDc7, ⋄=CDc8.

The X-axis shows the time after start [d], the Y-axis shows the body weight change, BWC [%].

Each measurement was carried out with a group of 8 mice. The conjugates CDc3 to CDc8 were administered once at a dosage of 75 mg/kg body weight on day 11. Taxotere® was administered 5 times at a dosage of 5 mg/kg body weight at days 11 to 15. Median values are given. Further details are given in Table 19.

FIG. 5: Time course of the RTV(T/C) values after administering Docetaxel conjugates CDc1, CDc4 and CDc5 (dosage 75 mg/kg body weight; mouse tumor model PC3)

FIG. 5 shows the time course of the relative tumor volume in the mouse tumor model for human prostate carcinoma PC3 treated with conjugates CDc1, CDc4 and CDc5 vs. mice in the control group (untreated mice (saline)) as well as vs. mice treated with Taxotere®.

The following symbols are used:

▪=saline, [symbol] =Docetaxel, Δ=CDc1, ⋄=CDc4, ∇=CDc5.

The X-axis shows the time after start [d], the Y-axis shows the relative tumor volume, RTV [%].

Each measurement was carried out with a group of 8 mice. The conjugates CDc1, CDc4 and CDc5 were administered once at a dosage of 75 mg/kg body weight on day 6.

Taxotere® was administered 5 times at a dosage of 5 mg/kg body weight at days 6 to 10. Median values are given. Further details are given in Table 20.

FIG. 6: Time course of the body weight change after administering Docetaxel conjugates CDc1, CDc4 and CDc5 (dosage 75 mg/kg body weight; mouse tumor model PC3)

FIG. 6 shows the time course of the body weight change in the mouse tumor model for human prostate carcinoma PC3 treated with conjugates CDc1, CDc4 and CDc5 vs. mice in the control group (untreated mice (saline)) as well as vs. mice treated with Taxotere®.

The following symbols are used:

▪=saline, [symbol] =Docetaxel, Δ=CDc1, ⋄=CDc4, ∇=CDc5.

The X-axis shows the time after start [d], the Y-axis shows the body weight change, BWC [%].

Each measurement was carried out with a group of 8 mice. The conjugates CDc1, CDc4 and CDc5 were administered once at a dosage of 75 mg/kg body weight on day 6. Taxotere® was administered 5 times at a dosage of 5 mg/kg body weight at days 6 to 10. Median values are given. Further details are given in Table 20.

FIG. 7: Time course of the RTV(T/C) values after administering Docetaxel conjugates CDc1, CDc4 and CDc5 (dosage 75 mg/kg body weight; mouse tumor model A549)

FIG. 7 shows the time course of the relative tumor volume in the mouse tumor model for human lung carcinoma A549 treated with conjugates CDc1, CDc4 and CDc5 vs. mice in the control group (untreated mice (saline)) as well as vs. mice treated with Taxotere®.

The following symbols are used:

▪=saline, [symbol] =Docetaxel, Δ=CDc1, ⋄=CDc4, ∇=CDc5.

The X-axis shows the time after start [d], the Y-axis shows the relative tumor volume, RTV [%].

Each measurement was carried out with a group of 8 mice. The conjugates CDc1, CDc4 and CDc5 were administered once at a dosage of 75 mg/kg body weight on day 8. Taxotere® was administered 5 times at a dosage of 5 mg/kg body weight at days 8 to 12. Median values are given. Further details are given in Table 21.

FIG. 8: Time course of the body weight change after administering Docetaxel conjugates CDc1, CDc4 and CDc5 (dosage 75 mg/kg body weight; mouse tumor model A549)

FIG. 8 shows the time course of the body weight change in the mouse tumor model for human lung carcinoma A549 treated with conjugates CDc1, CDc4 and CDc5 vs. mice in the control group (untreated mice (saline)) as well as vs. mice treated with Taxotere®.

The following symbols are used:

▪=saline, [symbol] =Docetaxel, Δ=CDc1, ⋄=CDc4, ∇=CDc5.

The X-axis shows the time after start [d], the Y-axis shows the body weight change, BWC [%].

Each measurement was carried out with a group of 8 mice. The conjugates CDc1, CDc4 and CDc5 were administered once at a dosage of 75 mg/kg body weight on day 8. Taxotere® was administered 5 times at a dosage of 5 mg/kg body weight at days 8 to 12. Median values are given. Further details are given in Table 21.

FIG. 9: Time course of the RTV(T/C) values after administering Docetaxel conjugates CDc8, CDc10, CDc15 and CDc17 (dosage 75 mg/kg body weight; mouse tumor model MT-3)

FIG. 9 shows the time course of the relative tumor volume in the mouse tumor model for human breast carcinoma MT-3 treated with conjugates CDc8, CDc10, CDc15 and CDc17 vs. mice in the control group (untreated mice (saline)) as well as vs. mice treated with Taxotere®.

The following symbols are used:

▪=saline, [symbol] =Docetaxel, ⋄=CDc8, Δ=CDc10, ◯=CDc15, ∇=CDc17.

The X-axis shows the time after start [d], the Y-axis shows the relative tumor volume, RTV [%].

Each measurement was carried out with a group of 6 to 8 mice. The conjugates CDc8, CDc10, CDc15 and CDc17 were administered once at a dosage of 75 mg/kg body weight on day 11. Taxotere® was administered 5 times at a dosage of 5 mg/kg body weight at days 11 to 15. Median values are given. Further details are given in Table 22.

FIG. 10: Time course of the RTV(T/C) values after administering Docetaxel conjugates CDc9, CDc11, CDc12, CDc13, CDc14, and CDc16 (dosage 75 mg/kg body weight; mouse tumor model MT-3)

FIG. 10 shows the time course of the relative tumor volume in the mouse tumor model for human breast carcinoma MT-3 treated with conjugates CDc9, CDc11, CDc12, CDc13, CDc14, and CDc16 vs. mice in the control group (untreated mice (saline)) as well as vs. mice treated with Taxotere®.

The following symbols are used:

### ▪=Saline, =Docetaxel, =CDc9, =CDc11, ⋄=CDc12, ∇=CDc13, ◯=CDc14, Δ=CDc16.

The X-axis shows the time after start [d], the Y-axis shows the relative tumor volume, RTV [%].

Each measurement was carried out with a group of 6 to 8 mice. The conjugates CDc9, CDc11, CDc12, CDc13, CDc14, and CDc16 were administered once at a dosage of 75 mg/kg body weight on day 11. Taxotere® was administered 5 times at a dosage of 5 mg/kg body weight at days 11 to 15. Median values are given. Further details are given in Table 22.

FIG. 11: Time course of the body weight change after administering Docetaxel conjugates CDc8, CDc10, CDc15 and CDc17 (dosage 75 mg/kg body weight; mouse tumor model MT-3)

FIG. 11 shows the time course of the body weight change in the mouse tumor model for human breast carcinoma MT-3 treated with conjugates CDc8, CDc10, CDc15 and CDc17 vs. mice in the control group (untreated mice (saline)) as well as vs. mice treated with Taxotere®.

The following symbols are used:

▪=saline, [symbol] =Docetaxel, ⋄=CDc8, Δ=CDc10, ◯=CDc15, ∇=CDc17.

The X-axis shows the time after start [d], the Y-axis shows the body weight change, BWC [%].

Each measurement was carried out with a group of 6 to 8 mice. The conjugates CDc8, CDc10, CDc15 and CDc17 were administered once at a dosage of 75 mg/kg body weight on day 11. Taxotere® was administered 5 times at a dosage of 5 mg/kg body weight at days 11 to 15. Median values are given. Further details are given in Table 22.

FIG. 12: Time course of the body weight change after administering Docetaxel conjugates CDc11, CDc12, CDc13, CDc14 and CDc16 (dosage 75 mg/kg body weight; mouse tumor model MT-3)

FIG. 12 shows the time course of the body weight change in the mouse tumor model for human breast carcinoma MT-3 treated with conjugates CDc11, CDc12, CDc13, CDc14 and CDc16 vs. mice in the control group (untreated mice (saline)) as well as vs. mice treated with Taxotere®.

The following symbols are used:

▪=saline, [symbol] =Docetaxel, [symbol] =CDc11, ⋄=CDc12, ∇=CDc13, ◯=CDc14, Δ=CDc16.

The X-axis shows the time after start [d], the Y-axis shows the body weight change, BWC [%].

Each measurement was carried out with a group of 6 to 8 mice. The conjugates CDc11, CDc12, CDc13, CDc14 and CDc16 were administered once at a dosage of 75 mg/kg body weight on day 11. Taxotere® was administered 5 times at a dosage of 5 mg/kg body weight at days 11 to 15. Median values are given. Further details are given in Table 22.

FIG. 13: Time course of the RTV(T/C) values after administering Docetaxel conjugates CDc20, CDc21, CDc24 and CDc35 (dosage 75 mg/kg body weight; mouse tumor model MT-3)

FIG. 13 shows the time course of the relative tumor volume in the mouse tumor model for human breast carcinoma MT-3 treated with conjugates CDc20, CDc21, CDc24 and CDc35 vs. mice in the control group (untreated mice (saline)) as well as vs. mice treated with Taxotere®.

The following symbols are used:

▪=saline, [symbol] =Docetaxel, ⋄=CDc20, ◯=CDc21, ∇=CDc24, Δ=CDc35.

The X-axis shows the time after start [d], the Y-axis shows the relative tumor volume, RTV [%].

Each measurement was carried out with a group of 6 to 8 mice. The conjugates CDc20, CDc21, CDc24 and CDc35 were administered once at a dosage of 75 mg/kg body weight on day 7. Taxotere® was administered 5 times at a dosage of 5 mg/kg body weight at days 7 to 11. Median values are given. Further details are given in Table 23.

FIG. 14: Time course of the RTV(T/C) values after administering Docetaxel conjugates CDc18, CDc19, CDc23, CDc25, CDc26 and CDc27 (dosage 75 mg/kg body weight; mouse tumor model MT-3)

FIG. 14 shows the time course of the relative tumor volume in the mouse tumor model for human breast carcinoma MT-3 treated with conjugates CDc18, CDc19, CDc23, CDc25, CDc26 and CDc27 vs. mice in the control group (untreated mice (saline)) as well as vs. mice treated with Taxotere®.

The following symbols are used:

▪=saline, [symbol] =Docetaxel, ∇=CDc18, ◯=CDc19, Δ=CDc23, [symbol] =CDc25, ⋄=CDc26, [symbol] =CDc27.

The X-axis shows the time after start [d], the Y-axis shows the relative tumor volume, RTV [%].

Each measurement was carried out with a group of 6 to 8 mice. The conjugates CDc18, CDc19, CDc23, CDc25, CDc26 and CDc27 were administered once at a dosage of 75 mg/kg body weight on day 7. Taxotere® was administered 5 times at a dosage of 5 mg/kg body weight at days 7 to 11. Median values are given. Further details are given in Table 23.

FIG. 15: Time course of the body weight change after administering Docetaxel conjugates CDc20, CDc21, CDc24 and CDc35 (dosage 75 mg/kg body weight; mouse tumor model MT-3)

FIG. 15 shows the time course of the body weight change in the mouse tumor model for human breast carcinoma MT-3 treated with conjugates CDc20, CDc21, CDc24 and CDc35 vs. mice in the control group (untreated mice (saline)) as well as vs. mice treated with Taxotere®.

The following symbols are used:

▪=saline, [symbol] =Docetaxel, ⋄=CDc20, ◯=CDc21, ∇=CDc24, Δ=CDc35.

The X-axis shows the time after start [d], the Y-axis shows the body weight change, BWC [%].

Each measurement was carried out with a group of 6 to 8 mice. The conjugates CDc20, CDc21, CDc24 and CDc35 were administered once at a dosage of 75 mg/kg body weight on day 7. Taxotere® was administered 5 times at a dosage of 5 mg/kg body weight at days 7 to 11. Median values are given. Further details are given in Table 23.

FIG. 16: Time course of the body weight change after administering Docetaxel conjugates CDc18, CDc19, CDc25, CDc26 and CDc27 (dosage 75 mg/kg body weight; mouse tumor model MT-3)

FIG. 16 shows the time course of the body weight change in the mouse tumor model for human breast carcinoma MT-3 treated with conjugates CDc18, CDc19, CDc25, CDc26 and CDc27 vs. mice in the control group (untreated mice (saline)) as well as vs. mice treated with Taxotere®.

The following symbols are used:

▪=saline, [symbol] =Docetaxel, ∇=CDc18, ◯=CDc19, [symbol] =CDc25, ⋄=CDc26, [symbol] =CDc27.

The X-axis shows the time after start [d], the Y-axis shows the body weight change, BWC [%].

Each measurement was carried out with a group of 6 to 8 mice. The conjugates CDc18, CDc19, CDc25, CDc26 and CDc27 were administered once at a dosage of 75 mg/kg body weight on day 7. Taxotere® was administered 5 times at a dosage of 5 mg/kg body weight at days 7 to 11. Median values are given. Further details are given in Table 23.

FIG. 17: Time course of the body weight change after administering Docetaxel conjugates CDc29, CDc30, CDc31, CDc32, CDc33 and CDc34 (dosage 75 mg/kg body weight; mouse tumor model MT-3)

FIG. 17 shows the time course of the body weight change in the mouse tumor model for human breast carcinoma MT-3 treated with conjugates CDc29, CDc30, CDc31, CDc32, CDc33 and CDc34 vs. mice in the control group (untreated mice (saline)) as well as vs. mice treated with Taxotere®.

The following symbols are used:

▪=saline, [symbol] =Docetaxel, ∇=CDc29, □=CDc30, ⋄=CDc31, Δ=CDc32, [symbol] =CDc33, ◯=CDc34.

The X-axis shows the time after start [d], the Y-axis shows the relative tumor volume, RTV [%].

Each measurement was carried out with a group of 7 to 8 mice. The conjugates CDc29, CDc30, CDc31, CDc32, CDc33 and CDc34 were administered once at a dosage of 75 mg/kg body weight on day 8. Taxotere® was administered 5 times at a dosage of 5 mg/kg body weight at days 8 to 12. Median values are given. Further details are given in Table 24.

FIG. 18: Time course of the body weight change after administering Docetaxel conjugates CDc29, CDc30, CDc31, CDc32, CDc33 and CDc34 (dosage 75 mg/kg body weight; mouse tumor model MT-3)

FIG. 18 shows the time course of the body weight change in the mouse tumor model for human breast carcinoma MT-3 treated with conjugates CDc29, CDc30, CDc31, CDc32, CDc33 and CDc34 vs. mice in the control group (untreated mice (saline)) as well as vs. mice treated with Taxotere®.

The following symbols are used:

▪=saline, [symbol] =Docetaxel, ∇=CDc29, □=CDc30, ◯=CDc31, Δ=CDc32, [symbol] =CDc33, ◯=CDc34.

The X-axis shows the time after start [d], the Y-axis shows the body weight change, BWC [%].

Each measurement was carried out with a group of 7 to 8 mice. The conjugates CDc29, CDc30, CDc31, CDc32, CDc33 and CDc34 were administered once at a dosage of 75 mg/kg body weight on day 8. Taxotere® was administered 5 times at a dosage of 5 mg/kg body weight at days 8 to 12. Median values are given. Further details are given in Table 24.

FIG. 19: Time course of the RTV(T/C) values after administering Paclitaxel conjugates CPc1, CPc2 and CPc3 (dosage 100 mg/kg body weight; mouse tumor model MT-3)

FIG. 19 shows the time course of the relative tumor volume in the mouse tumor model for human breast carcinoma MT-3 treated with conjugates CPc1, CPc2 and CPc3 vs. mice in the control group (untreated mice (saline)) as well as vs. mice treated with Paclitaxel (not conjugated, Neotaxan, Neocorp/Sandoz).

The following symbols are used:

▪=saline, [symbol] =Paclitaxel, ◯=CPc1, ∇=CPc2, Δ=CPc3.

The X-axis shows the time [d], the Y-axis shows the relative tumor volume, RTV [%].

Each measurement was carried out with a group of 8 mice. The conjugates CPc1, CPc2 and CPc3 were administered once at a dosage of 100 mg/kg body weight on day 7. Paclitaxel (not conjugated) was administered 3 times at a dosage of 12.5 or 10 mg/kg body weight at days 7 to 9 or 10 to 11. Median values are given. Further details are given in Table 25.

FIG. 20: Time course of the body weight change after administering Paclitaxel conjugates CPc1, CPc2 and CPc3 (dosage 100 mg/kg body weight; mouse tumor model MT-3)

FIG. 20 shows the time course of the body weight change in the mouse tumor model for human breast carcinoma MT-3 treated with conjugates CPc1, CPc2 and CPc3 vs. mice in the control group (untreated mice (saline)) as well as vs. mice treated with Paclitaxel (not conjugated, Neotaxan, Neocorp/Sandoz).

The following symbols are used:

▪=saline, [symbol] =Paclitaxel, ◯=CPc1, ∇=CPc2, Δ=CPc3.

The X-axis shows the time [d], the Y-axis shows the body weight change, BWC [%].

Each measurement was carried out with a group of 8 mice. The conjugates CPc1, CPc2 and CPc3 were administered once at a dosage of 100 mg/kg body weight on day 7. Paclitaxel (not conjugated) was administered 3 times at a dosage of 12.5 or 10 mg/kg body weight at days 7 to 9 or 10 to 11. Median values are given. Further details are given in Table 25.

FIG. 21: Time course of the RTV(T/C) values after administering Paclitaxel conjugates CPc4, CPc5, CPc6 and CPc7 (dosage 80 mg/kg body weight; mouse tumor model MT-3)

FIG. 21 shows the time course of the relative tumor volume in the mouse tumor model for human breast carcinoma MT-3 treated with conjugates CPc4, CPc5, CPc6 and CPc7 vs. mice in the control group (untreated mice (saline)) as well as vs. mice treated with Paclitaxel (not conjugated, Neotaxan, Neocorp/Sandoz).

The following symbols are used:

▪=saline, [symbol] =Paclitaxel, Δ=CPc4, ◯=CPc5, ⋄═CPc6, ∇=CPc7.

The X-axis shows the time after start [d], the Y-axis shows the relative tumor volume, RTV [%].

Each measurement was carried out with a group of 8 mice. The conjugates CPc4, CPc5, CPc6 and CPc7 were administered once at a dosage of 80 mg/kg body weight on day 10. Paclitaxel (not conjugated) was administered 5 times at a dosage of 10 mg/kg body weight at days 10 to 14. Median values are given. Further details are given in Table 26.

FIG. 22: Time course of the body weight change after administering Paclitaxel conjugates CPc4, CPc5, CPc6 and CPc7 (dosage 80 mg/kg body weight; mouse tumor model MT-3)

FIG. 22 shows the time course of the body weight change in the mouse tumor model for human breast carcinoma MT-3 treated with conjugates CPc4, CPc5, CPc6 and CPc7 vs. mice in the control group (untreated mice (saline)) as well as vs. mice treated with Paclitaxel (not conjugated, Neotaxan, Neocorp/Sandoz).

The following symbols are used:

▪=saline, [symbol] =Paclitaxel, Δ=CPc4, ◯=CPc5, ⋄═CPc6, ∇=CPc7.

The X-axis shows the time after tumor transplantation [d], the Y-axis shows the body weight change, BWC [%].

Each measurement was carried out with a group of 8 mice. The conjugates CPc4, CPc5, CPc6 and CPc7 were administered once at a dosage of 80 mg/kg body weight on day 10. Paclitaxel (not conjugated) was administered 5 times at a dosage of 10 mg/kg body weight at days 10 to 14. Median values are given. Further details are given in Table 26.

FIG. 23: Cleavage Kinetics of Docetaxel conjugates

FIG. 23 shows the cleavage kinetics of conjugates of 5 mg/mL of CDc2, CDc4, CDc10, CDc18, CDc24 and CDc19 in PBS buffer, pH 7.4, measured at 37° C. and determined by RP-HPLC.

The following symbols are used:

### ▪=CDc2, ♦=CDc4, +=CDc10, ▴=CDc18, =CDc24, X=CDc19.

The X-axis shows the time [h], the Y-axis shows the conjugate [%].

### EXPERIMENTAL

### 1.1 Materials and Methods

### 1.1.1 General Techniques

Centrifugation was performed using a Sorvall Evolution RC centrifuge (Thermo Scientific) equipped with a SLA-3000 rotor (6×400 ml vessels) at 9000 g and 4° C. for 5-10 min.
Ultrafiltration was performed using a Sartoflow Slice 200 Benchtop (Sartorius AG) equipped with two Hydrosart Membrane cassettes (10 kDa Cutoff, Sartorius). Pressure settings: p1=2 bar, p2=0.5 bar.
Filtration: Solutions were filtered prior to size exclusion chromatography and HPLC using syringe filters (0.45 μm, GHP-Acrodisc, 13 mm) or Steriflip (0.45 μm, Millipore).
Analytical HPLC spectra were measured on an Ultimate 3000 (Dionex) using a LPG-3000 pump, a DAD-3000a diode array detector and a C18 reverse phase column (Dr. Maisch, Reprosil Gold 300A, C18, 5 μm, 150×4.6 mm). Eluents were purified water (Millipore)+0.1% TFA (Uvasol, MERCK) and acetonitrile (HPLC grade, MERCK)+0.1% TFA. Standard gradient was: 2% ACN to 98% ACN in 30 min.
Size exclusion chromatography was performed using an Äkta Purifier (GE-Healthcare) system equipped with a P-900 pump, a P-960 sample pump using an UV-900 UV detector and a pH/IC-900 conductivity detector. A HiPrep 26/10 desalting column (53 ml, GE-Healthcare) was used together with a HiTrap desalting column as pre-column (5 ml, GE-Healthcare). Fractions were collected using the Frac-902 fraction collector.
Freeze-drying: Samples were frozen in liquid nitrogen and lyophylized using a Christ alpha 1-2 LD plus (Martin Christ, Germany) at p=0.2 mbar.
UV-vis absorbances were measured at a Cary 100 BIO (Varian) in either plastic cuvettes (PMMA, d=10 mm) or quarz cuvettes (d=10 mm, Hellma, Suprasil, 100-QS) using the Cary Win UV simple reads software.

### 1.1.2 Reagents

TABLE 2
Hydroxyalkyl starch used (obtained from Fresenius Kabi Linz (Austria))

| Name | Lot | Mw | Mn | PDI | MS |
|---|---|---|---|---|---|
| HES1 | 055231 | 51.7 | 44.5 | 1.16 | 1.0 |
| HES2 | 073421 | 89.1 | 78.1 | 1.14 | 0.4 |
| HES3 | 080511 | 77.1 | 62.2 | 1.24 | 0.7 |
| HES4 | 17090621 | 95.7 | 74.3 | 1.29 | 0.8 |
| HES5a | 063711 | 77.5 | 63.2 | 1.23 | 1.0 |
| HES5b | 070341 | 80.3 | 64.5 | 1.24 | 1.0 |
| HES6 | 073121 | 84.5 | 55.2 | 1.47 | 1.3 |
| HES7 | 17091931 | 273.8 | 214.5 | 1.28 | 0.5 |
| HES8 | 17091071 | 275.8 | 200.2 | 1.38 | 0.7 |
| HES9 | 1709443 | 247.6 | 181.3 | 1.37 | 1.0 |
| HES10 | 084721 | 243.9 | 183.6 | 1.33 | 1.3 |
| HES11 | 17091331 | 985.0 | 500.4 | 1.97 | 0.5 |
| HES12 | 17091241 | 700.8 | 375.9 | 1.87 | 0.7 |
| HES13 | 17091131 | 694.4 | 441.7 | 1.57 | 1.0 |
| HES14 | 17090821 | 769.5 | 498.6 | 1.54 | 1.3 |
| HES15 | 17091431 | 2110.0 | 878.1 | 2.40 | 0.5 |
| HES16 | 17091511 | 2379.5 | 708.4 | 3.36 | 0.7 |
| HES17 | 1711011 | 92.4 | 66.4 | 1.39 | 1.0 |

TABLE 3
Reagents used

| | | | | |
|---|---|---|---|---|
| Entry | Name | Quality | Supplier | Lot# |

General procedure 1

| | | | | |
|---|---|---|---|---|
| 1 | 4-nitrophenyl | 96% | Aldrich | 02107CH-029 |
| | chloroformate | | | |
| 2 | Dimethyl | dry, SeccoSolv | Merck | K39250731 |
| | sulfoxide | | | |
| 3 | Pyridine | puriss. | Merck | K37206362 |
| 4 | Cystamine | 98% | Aldrich | MKAA1973 |
| | dihydrochloride | | | |
| 5 | DL-Dithiothreitol (DTT) | >99% | Sigma | 128K1092 |
| 6 | Sodium borohyride | >96% | Fluka | S3871434806003 |

General procedure 2

| | | | | |
|---|---|---|---|---|
| 7 | Sodium hydride (NaH) | 60% w/w in paraffin | Merck | S4977752 |
| 8 | Allyl bromide (AllBr) | reagent grade 97% | Aldrich | S77053-109 |
| 9 | Potassium | technical grade | Aldrich | 82070 |
| | monopersulfate Triplesalt | | | |
| | (Oxone ®) | | | |
| 10 | Sodium bicarbonate | puriss. | Merck | 26533223 |
| 11 | Tetrahydrothiopyran-4- | 99% | Aldrich | 1370210 |
| | one | | | 42708159 |
| 12 | Sodium thiosulfate | p.a. | Acros | A0204915001 |
| | pentahydrate | | | |
| 13 | Ethanedithiol | 99% | Fluka | 01391947 |

General procedure 3

| | | | | |
|---|---|---|---|---|
| 14 | Methanesulfonyl chloride | >99% | Aldrich | S28114-079 |
| 15 | Potassium thioacetate | | Aldrich | BCBB6780 |
| 16 | Diisopropyl ethyl amine | >98% | Fluka | 448324/1 |
| 17 | 2,4,6-trimethyl pyridine, | | Fluka | 0001404791 |
| | collidine | | | |
| 18 | Sodium hydrogensulfide | | Aldrich | 03396TK040 |
| 19 | Aqueous ammonia | extra pure, | Acros | AO240617 |
| | | 25% in water | | |

General procedure 5

| | | | | |
|---|---|---|---|---|
| 20 | Iodoacetic acid | synthesis grade | Merck | S06291 |

Analytics

| | | | |
|---|---|---|---|
| 5,5′-Dithiobis(2- | >97.5% | Fluka | 1334177 |
| nitrobenzoic acid), | | | |
| Ellman's reagent | | | |

Solvents

| | | | |
|---|---|---|---|
| Isopropanol | puriss ACS | Fluka | |
| Methyl tert.-butyl ether | 99% | Acros | |
| Dimethyl formamide | pept. syn. grade | Acros | A0256931 |
| Trifluoroethanol (TFE) | reagent plus >99% | Aldrich | S57348-458 |
| Dimethyl formamide | extra dry 99.8% | Acros | A00954967 |
| Formamide | spectophotometric grade | Aldrich | 59096HK |
| | >99% | | |
| Acetic acid | >99.8% | Fluka | 91190 |

### 1.2 Synthesis and Characterization of Drug Derivatives

### 1.2.1 2′-(Bromoacetyl)-docetaxel (Doc1)

A 1 l three-neck flask equipped with a magnetic stirring bar and inside thermometer was loaded with 500 ml of DCM (dichloromethane) and 5.0 g (6.19 mmol) docetaxel. The mixture was cooled by means of an ice-salt bath to 0° C. and was allowed to stir for 30 minutes. Bromoacetic anhydride (1.95 g, 7.49 mmol) was added followed by diisopropyl ethyl amine (1.3 ml, 7.49 mmol). The reaction was allowed to stir for 15 h and allowed to warm up to room temperature. The progress of the reaction was monitored by TLC (thin layer chromatography). After completion of the reaction, the solution was washed twice with 0.1 N hydrochloric acid, once with 300 ml of water and once with 100 ml of saturated sodium bicarbonate solution. The organic phase was dried with sodium sulfate and the solvent removed under reduced pressure. The crude product was applied on silica and purified by column chromatography on silica (hexane/ethyl acetate 1:1). The yield was 4.20 g (4.52 mmol, 73%) of a colorless solid.

¹H-NMR: (CDCl₃, 200 MHz) δ=8.17-8.08 (m, 2H); 7.67-7.27 (m, 8H); 6.32-6.19 (m, 1H); 5.74-5.65 (m, 1H); 5.55-5.34 (m, 3H); 5.26-5.18 (m, 1H); 5.01-4.92 (m, 1H); 4.38-3.85 (m, 7H); 2.45-1.12 (m, 30H).

TLC: (hexane/ethyl acetate 1:1)=0.50.

MS: (ESI; MeOH) 952.3 [M(⁸¹Br)+Na⁺] 950.3 [M(⁷⁹Br)+Na⁺]; 550.2; 549.2; 426.1, 424.1.

### 1.2.2 2′-(5-Bromopentanoyl)-docetaxel (Doc2)

A 500 ml three-neck flask equipped with a magnetic stirring bar and inside thermometer was charged with 300 ml DCM and 1.5 g (1.85 mmol) docetaxel. 507 mg (2.8 mmol) of 5-bromovaleric acid were added and the mixture was allowed to stir for 15 minutes. The flask was cooled in an ice-water bath to 0° C. 102 mg (0.83 mmol) DMAP [4-(dimethylaminopyridine)] and 514 μl (1.59 mmol) EDC (1-ethyl-3-[3-dimethylaminopropyl]carbodiimide) were added and the reaction mixture allowed to warm up to room temperature. The course of the reaction was monitored by TLC. After completion of the reaction, the reaction mixture was washed twice with 100 ml of a 0.5% sodium bicarbonate solution, once with 200 ml of water and once with 200 ml of 0.1 N hydrochloric acid. The organic phase was further washed with 200 ml of water followed by 200 ml of brine, dried over sodium sulphate and evaporated to dryness. The crude product was applied on silica and purified by column chromatography on silica (hexane/ethyl acetate 1:1). The yield was 1.08 g (1.11 mmol, 60%) of a colorless solid.

TLC: (hexane/ethyl acetate 1:1) R_{f}=0.55.

MS (ESI, MeOH): m/z=994.4 [M(⁸¹Br)+Na]⁺, 992.4 [M(⁷⁹Br)+Na]⁺.

### 1.2.3 2′-(3-maleimidopropionyl)-docetaxel (Doc3)

A 250 ml three-neck flask equipped with a magnetic stirring bar and inside thermometer was charged with 175 ml DCM and 861 mg (1.06 mmol) docetaxel. 270 mg (1.59 mmol) of N-maleoyl-β-alanine were added and the mixture was allowed to stir for 15 minutes. The flask was cooled in an ice-water bath to 0° C. 58 mg (0.48 mmol) DMAP [4-(dimethylaminopyridine)] and 247 mg (1.59 mmol) EDC (1-ethyl-3-[3-dimethylaminopropyl]carbodiimide) were added and the reaction mixture allowed to warm up to room temperature. The course of the reaction was monitored by TLC. After completion of the reaction, the reaction mixture was washed twice with 100 ml of a 0.5% sodium bicarbonate solution and twice with 100 ml of 0.1 N hydrochloric acid. The organic phase was further washed with 200 ml of water followed by 200 ml of brine, dried over sodium sulfate and evaporated to dryness. The crude product was applied on silica and purified by column chromatography on silica (DCM/ethyl acetate 1:1). The yield was 0.38 g (0.40 mmol, 38%) of a colorless solid.

¹H-NMR: (CDCl₃, 200 MHz) δ=8.09-8.00 (m, 2H); 7.59-7.18 (m, 8H); 6.62-6.57 (m, 2H); 6.22-6.03 (m, 1H); 5.73-5.56 (m, 2H); 5.46-5.32 (m, 1H); 5.32-5.18 (m, 1H); 5.18-5.09 (m, 1H); 4.94-4.81 (m, 1H); 4.32-4.08 (m, 3H); 3.92-3.57 (m, 3H); 2.72-2.60 (m, 2H); 2.60-1.00 (m, 31H).

TLC: (DCM/ethyl acetate 1:1) R_{f}=0.40.

MS: (ESI; MeOH) 1013.3 [M+Na⁺+MeOH], 981.3 [M+Na⁺]; 371.6 [100].

### 1.2.4 2′-(5-maleimido-3-thio-pentanoyl)-docetaxel (Doc4)

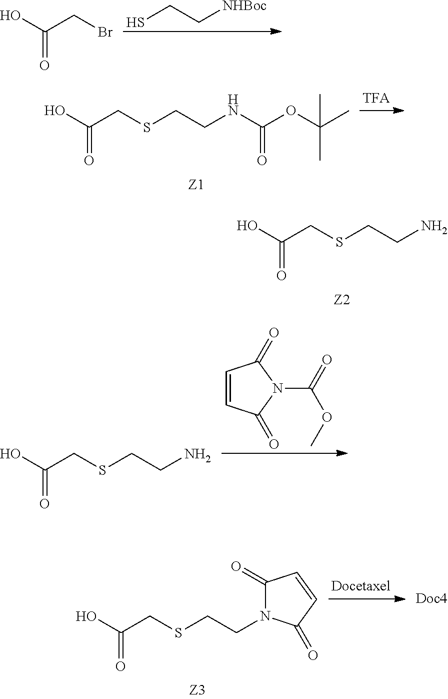

### (a) Synthesis of 5-tert.-butoxycarbonylamino-3-thiavaleric acid, Z1

A 250 ml 3-neck flask equipped with a magnetic stirring bar and inside thermometer was loaded with 84 ml of water and 3.53 g (42 mmol) of sodium bicarbonate. Bromoacetic acid (1.95 g, 14 mmol) was dissolved in this solution followed by addition of 4.76 ml (28 mmol) of N-Boc-cysteamine. The reaction mixture was stirred for 5 h. The basic solution (pH 10) was extracted three times with 100 ml of diethyl ether. The aqueous phase was acidified to pH 2 with 1 N hydrochloric acid and extracted three times with 100 ml of diethyl ether. The combined organic phases were washed with saturated sodium bicarbonate solution (2×50 ml) and brine (50 ml), dried over sodium sulfate and evaporated to dryness. The title compound (crude product) (3.196 g, 13.5 mmol, 96%) was used without further purification.

¹H-NMR: (CDCl₃, 200 MHz) δ=10.41 (bs, 1H); 6.31+5.04 (bs, 1H); 3.51-3.25 (m, 2H); 3.28 (s, 2H); 2.83-2.75 (m, 2H); 1.45 (s, 9H).

TLC: (hexane/ethyl acetate 2:1) R_{f}=0.35.

### (b) Synthesis of 5-amino-3-thiavaleric acid, TFA salt, Z2

A 250 ml two-neck flask equipped with a magnetic stirring bar and inside thermometer was charged with 3.2 g of 5-tert.-butoxycarbonylamino-3-thiavaleric acid (Z1, 13.5 mmol). The flask was cooled to 0° C. by means of an ice-water bath and 80 ml of pre-cooled TFA added. The reaction mixture was stirred for 1 h at 0° C. and the progress of the reaction monitored by TLC. The TFA was removed under reduced pressure until the weight remained constant. The yield was 2.24 g (quantitative yield+residual TFA).

¹H-NMR: (MeOD, 200 MHz) δ=3.40 (s, 2H); 3.28-3.16 (m, 2H); 3.02-2.91 (m, 2H).

TLC: (hexane/ethyl acetate 1:1) R_{f}=0.05.

### (c) Synthesis of 5-maleimido-3-thiavaleric acid, Z3

A 250 ml 3-neck flask equipped with a magnetic stirring bar and inside thermometer was charged with 80 ml of saturated sodium bicarbonate solution and 1.84 g (13.5 mmol) of 5-(amino-3-thiavaleric acid, TFA salt (Z2). The solution was cooled to 0° C. by means of an ice-salt bath. Then, 2,5-dioxo-2,5-dihydropyrrol-1-carboxylic acid methyl ester (2.09 g, 13.5 mmol) was added in one portion. The cooled reaction mixture was stirred for 30 minutes. The ice bath was removed and the reaction stirred for additional 3 h at ambient temperature. The reaction mixture was acidified to pH 2 with 1 N hydrochloric acid under constant cooling. The aqueous phase was extracted three times with diethyl ether (250 ml). The combined organic phases were washed twice with 150 ml of sodium bicarbonate solution and once with 150 ml of brine. The organic layer was dried over sodium sulfate and evaporated under reduced pressure without heating. The resulting solid was dissolved in 30 ml of methanol and crystallized at −18° C. The precipitate was filtered, washed with cold hexane and dried to give 1.97 g (9.15 mmol, 69%) of an off-white solid.

¹H-NMR: (MeOD, 200 MHz) δ=6.87 (s, 2H); 3.79 (t, J=6.6 Hz, 2H); 3.31 (s, 2H); 2.89 (t, J=6.6 Hz, 2H).

TLC (hexane/ethyl acetate 1:1): R_{f}=0.40.

### (d) Synthesis of (5-maleimido-3-thia-valeroyl)-2′-docetaxel (Doc4)

A 500 ml three-neck flask equipped with a magnetic stirring bar and inside thermometer was charged with 240 ml DCM, 2.5 g (3.09 mmol) of docetaxel and 996 mg (4.64 mmol) of 5-maleimido-3-thiavaleric acid (Z3). The solution was cooled to 0° C. by means of an ice-water bath and stirred for 30 min. 850 μl (4.64 mmol) of EDC and 170 mg (1.40 mmol) of DMAP were added and the reaction mixture stirred for 4 h at 0° C. The reaction was allowed to warm up to room temperature and the conversion monitored by TLC. The reaction mixture was washed twice with 250 ml of 0.1 N hydrochloric acid, once with water and once with saturated sodium bicarbonate solution. The organic phase was dried over sodium sulfate and the solvents were evaporated under reduced pressure. The crude product was purified by column chromatograohy on silica (DCM/ethyl acetate 1:1). The yield was 758 mg (0.76 mmol, 24%) of a colorless solid.

TLC (hexane/ethyl acetate 2:1): R_{f}=0.45.

MS (ESI; MeOH): m/z=1027.35 [M+Na]⁺; 1043.36 [M K]⁺

### 1.2.5 2′-(5-maleimido-3-oxo-pentanoyl)-docetaxel (Doc5)

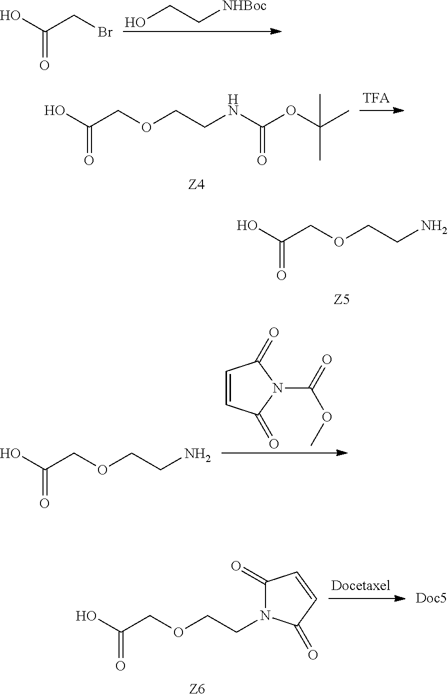

### (a) Synthesis of 5-tert.-butoxycarbonylamino-3-oxavaleric acid, Z4

A 100 ml 2-neck flask equipped with a magnetic stirring bar and inside thermometer was loaded with bromoacetic acid (1.00 g, 7.19 mmol), N-Boc-ethanolamine (2.32 g, 14.39 mmol) and 25 ml of THF. The reaction mixture was cooled down to 0° C. by means of an ice-water bath. 0.863 mg (21.59 mmol) of sodium hydride (60% w/w in paraffin) were added, the cooling bath was removed and the reaction mixture stirred for 2 h at room temperature. The reaction was quenched by addition of 150 ml of water and the basic solution (pH 10) extracted three times with 100 ml of diethyl ether. The aqueous phase was acidified to pH 2 with 1 N hydrochloric acid and extracted three times with 100 ml of diethyl ether. The combined organic phases were washed with saturated sodium bicarbonate solution (2×50 ml) and brine (50 ml), dried over sodium sulphate and evaporated to dryness. The title compound (1.53 g, 6.98 mmol, 97%) was used without further purification.

1H-NMR: (CDCl₃, 200 MHz) δ=10.18 (bs, 1H); 6.50+5.26 (bs, 1H); 4.13 (s, 2H), 3.68-3.57 (m, 2H); 3.42-3.27 (m, 2H); 1.45 (s, 9H).

TLC: (hexane/ethyl acetate 1:1) R_{f}=0.20.

### (b) Synthesis of 5-amino-3-oxavaleric acid, TFA salt, Z5

A 250 ml two-neck flask equipped with a magnetic stirring bar and inside thermometer was charged with 1.5 g of 5-(tert.-butoxycarbonylamino)-3-oxavaleric acid (Z4, 6.85 mmol). The flask was cooled to 0° C. by means of an ice-water bath and 80 ml of pre-cooled TFA were added. The reaction mixture was stirred for 1 h at 0° C. and the progress of the reaction monitored by TLC. The TFA was removed under reduced pressure until the weight remained constant. The yield was 0.94 g (quantitative yield+residual TFA).

¹H-NMR: (MeOD, 200 MHz) δ=4.21 (s, 2H); 3.84-3.76 (m, 2H); 3.24-3.11 (m, 2H).

TLC: (hexane/ethyl acetate 1:1) R_{f}=0.05.

### (c) Synthesis of 5-maleimido-3-oxavaleric acid, Z6

A 100 ml 3-neck flask equipped with a magnetic stirring bar and inside thermometer was charged with 35 ml of saturated sodium bicarbonate solution and 816 mg (6.85 mmol) of 5-amino-3-oxavaleric acid, TFA salt (Z2). The solution was cooled to 0° C. by means of an ice-salt bath. Then, 2,5-dioxo-2,5-dihydropyrrol-1-carboxylic acid methyl ester (990 mg, 6.85 mmol) was added in one portion. The cooled reaction mixture was stirred for 30 minutes. The ice bath was removed and the reaction stirred for additional 3 h at ambient temperature. The reaction mixture was acidified to pH 2 with 1 N hydrochloric acid under constant cooling. The aqueous phase was extracted three times with diethyl ether (250 ml). The combined organic phases were washed twice with 150 ml of sodium bicarbonate solution and once with 150 ml of brine. The organic layer was dried over sodium sulfate and evaporated under reduced pressure without heating to give 1.03 g (5.17 mmol, 75%) of an colorless oil, which was used in the next step without further purification.

¹H-NMR: (MeOD, 200 MHz) δ=6.64 (s, 2H); 4.16-4.06 (m, 2H); 3.76-3.69 (m, 2H); 3.64 (s, 2H).

TLC (hexane/ethyl acetate 1:1): R_{f}=0.35.

### (d) Synthesis of (5-maleimido-3-oxa-pentanoyl)-2′-docetaxel (Doc5)

A 250 ml three-neck flask equipped with a magnetic stirring bar and inside thermometer was charged with 80 ml DCM, 1.5 g (1.86 mmol) of docetaxel and 554 mg (2.79 mmol) of 5-maleimido-3-oxavaleric acid (Z6). The solution was cooled to 0° C. by means of an ice-salt bath and stirred for 20 min. 500 μl (2.79 mmol) of EDC and 102 mg (0.84 mmol) of DMAP were added and the reaction mixture was stirred for 4 h at 0° C. The reaction was allowed to warm up to room temperature and the conversion monitored by TLC. The reaction mixture was washed twice with 250 ml of 0.1 N hydrochloric acid, once with water and once with saturated sodium bicarbonate solution. The organic phase was dried over sodium sulfate and the solvents were evaporated under reduced pressure. The crude product was purified by column chromatography on silica (DCM/ethyl acetate 1:1). The yield was 407 mg (0.41 mmol, 22%) of a colorless solid.

TLC (hexane/ethyl acetate 2:1): R_{f}=0.45.

MS (ESI; MeOH): m/z=1011.37 [M+Na]⁺; 1027.38 [M+K]⁺.

### 1.2.6 2′-(6-maleimido-3-oxo-hexanoyl)-docetaxel (Doc6)

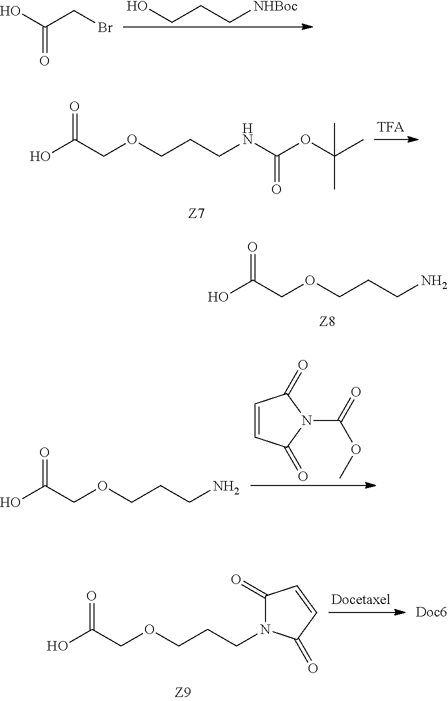

### (a) Synthesis of 6-tert.-butoxycarbonylamino-3-oxahexanoic acid, Z7

A 250 ml 2-neck flask equipped with a magnetic stirring bar and inside thermometer was loaded with bromoacetic acid (3.04 g, 21.9 mmol), 3-Boc-aminopropanol (7.4 ml, 43.8 mmol) and 130 ml of THF. The reaction mixture was cooled down to 0° C. by means of an ice-water bath. 2.63 mg (65.7 mmol) of sodium hydride (60% w/w in paraffin) were added, the cooling bath was removed and the reaction mixture stirred for 2 h at room temperature. The reaction was quenched by addition of 200 ml of water and the basic solution (pH 10) was extracted three times with 200 ml of ethyl acetate. The aqueous phase was acidified to pH 2 with 1 N hydrochloric acid and extracted three times with 100 ml of diethyl ether. The combined organic phases were washed with saturated sodium bicarbonate solution (2×125 ml) and brine (100 ml), dried over sodium sulfate and evaporated to dryness. The title compound (5.06 g, 21.6 mmol, 99%) was used without further purification.

¹H-NMR: (CDCl₃, 200 MHz) δ=10.81 (bs, 1H); 6.26+5.15 (bs, 1H); 4.10 (s, 2H); 3.61 (t, J=6.9 Hz, 2H); 3.35-3.17 (m, 2H); 1.87-1.71 (m, 2H); 1.44 (s, 9H).

TLC: (hexane/ethyl acetate 1:1) R_{f}=0.25.

### (b) Synthesis of 6-amino-3-oxahexanoic acid, TFA salt, Z8

A 250 ml two-neck flask equipped with a magnetic stirring bar and inside thermometer was charged with 4.99 g of 5-(tert.-butoxycarbonylamino)-3-oxahexanoic acid (Z7, 21.4 mmol). The flask was cooled to 0° C. by means of an ice-water bath and 80 ml of pre-cooled TFA added. The reaction mixture was stirred for 1 h at 0° C. and the progress of the reaction was monitored by TLC. The TFA was removed under reduced pressure until the weight remained constant. The yield was 3.01 g (quantitative yield+residual TFA).

¹H-NMR: (MeOD, 200 MHz) δ=4.16 (s, 2H); 3.77-3.67 (m, 2H); 3.23-3.11 (m, 2H); 2.06-1.89 (m, 2H).

TLC: (hexane/ethyl acetate 1:1) R_{f}=0.05.

### (c) Synthesis of 5-maleimido-3-oxahexanoic acid, Z9

A 250 ml 3-neck flask equipped with magnetic stirring and inside thermometer was charged with 100 ml of saturated sodium bicarbonate solution and 2.85 g (21.4 mmol) of 6-amino-3-oxahexanoic acid, TFA salt (Z8). The solution was cooled to 0° C. by means of an ice-salt bath. Then, 2,5-dioxo-2,5-dihydropyrrol-1-carboxylic acid methyl ester (3.32 g, 21.4 mmol) was added in one portion. The cooled reaction mixture was stirred for 30 minutes. The ice bath was removed and the reaction stirred for additional 3 h at ambient temperature. The reaction mixture was acidified to pH 2 with 1 N hydrochloric acid under constant cooling. The aqueous phase was extracted three times with ethyl acetate (250 ml). The combined organic phases were washed twice with 150 ml of sodium bicarbonate solution and once with 150 ml of brine. The organic layer was dried over sodium sulfate and evaporated under reduced pressure without heating to give 4.52 g (21.2 mmol, 98%) of an oil, which was used in the next step without further purification.

¹H-NMR: (MeOD, 200 MHz) δ=6.83 (s, 2H); 3.71-3.61 (m, 2H); 3.64 (s, 2H); 3.61-3.52 (m, 2H); 1.96-1.81 (m, 2H).

TLC (hexane/ethyl acetate 1:1): R_{f}=0.30.

### (d) Synthesis of (6-maleimido-3-oxa-hexanoyl)-2′-docetaxel (Doc6)

A 500 ml three-neck flask equipped a with magnetic stirring bar and inside thermometer was charged with 200 ml DCM, 3.0 g (3.71 mmol) of docetaxel and 1.186 mg (5.57 mmol) of 6-maleimido-3-oxahexanoic acid (Z9). The solution was cooled to 0° C. by means of an ice-salt bath and stirred for 20 min. 1.02 ml (5.57 mmol) of EDC and 203 mg (1.67 mmol) of DMAP were added and the reaction mixture stirred for 4 h at 0° C. To reach total conversion of the docetaxel, additional 0.395 g (1.85 mmol) of Z9 were added. The reaction was allowed to warm up to room temperature and the conversion monitored by TLC. The reaction mixture was washed twice with 250 ml of 0.1 N hydrochloric acid, once with water and once with saturated sodium bicarbonate solution. The organic phase was dried over sodium sulfate and the solvents evaporated under reduced pressure. The crude product was purified by column chromatography on silica (DCM/ethyl acetate 1:1). Yield was 612 mg (0.61 mmol, 17%) of a colorless solid.

TLC (hexane/ethyl acetate 2:1): R_{f}=0.45.

MS (ESI; MeOH): m/z=1025.39 [M+Na]⁺.

### 1.2.7 2′-(bromoacetyl)-paclitaxel (Pac1)

A 100 ml flask equipped with a magnetic stirring bar and an inert gas inlet was charged with 60 ml DCM and 300 mg (351 μmol) paclitaxel in the absence of light. 95 mg (525 μmol) of 5-bromovaleric acid were added and the mixture was allowed to stir for 15 minutes. The flask was cooled in an ice-water bath to 0° C. 19 mg (157 μmol) DMAP and 81.5 mg (525 μol) EDC were added and the reaction mixture was allowed to warm up to room temperature. The course of the reaction was monitored by TLC. After completion of the reaction, the reaction mixture was washed twice with 100 ml of a 0.5% sodium bicarbonate solution and twice with 100 ml of 0.1 N hydrochloric acid. The organic phase was further washed with 100 ml of water followed by 100 ml of brine, dried over sodium sulfate and evaporated to dryness. The crude product was applied on silica and purified by column chromatography on silica (DCM/ethyl acetate 2:1). Yield was 0.27 g (334 μmol, 75%) of a colorless solid.

### 1.2.8 2′-(5-bromopentanoyl)-paclitaxel (Pac2)

A 100 ml flask equipped with a magnetic stirring bar and inert gas inlet was charged with 60 ml DCM and 300 mg (351 μmol) paclitaxel in the absence of light. The flask was cooled in an ice-water bath to 0° C. 110 mg (425 bromoacetic anhydride and 75 (425 μmol) diisopropyl ethylamine were added and the reaction mixture was allowed to warm up to room temperature. The course of the reaction was monitored by TLC. After completion of the reaction, the reaction mixture was washed twice with 100 ml of a 0.5% sodium bicarbonate solution and twice with 100 ml of 0.1 N hydrochloric acid. The organic phase was further washed with 100 ml of water followed by 100 ml of brine, dried over sodium sulfate and evaporated to dryness. The crude product was applied on silica and purified by column chromatography on silica (DCM/ethyl acetate 2:1). The yield was 0.26 g (328 μmol, 73%) of a colorless solid.

¹H-NMR: (CDCl₃, 400 MHz) δ=8.16-8.13 (m, 2Hₐᵣₒ); 7.76-7.73 (m, 2Hₐᵣₒ); 7.64-7.59 (m, 1Hₐᵣₒ); 7.54-7.49 (m, 3Hₐᵣₒ); 7.46-7.34 (m, 7Hₐᵣₒ); 6.86 (d, J=9.2 Hz, 1H, NH); 6.29 (s, 1H, H10); 6.27 (dd, j=9.0 Hz, J=7.8 Hz, 1H, H13); 6.01 (dd, J=9.2 Hz, J=3.1 Hz, 1H, H3′); 5.68 (d, J=7.1 Hz, 1H, H2); 5.54 (d, J=3.1 Hz, 1H, H2′); 4.97 (dd, J=9.6 Hz, J=2.0 Hz, 1H, H5); 4.44 (ddd, J=10.7 Hz, J=6.5 Hz, J=4.2 Hz, 1H, H7); 4.32 (d, 0.1=8.5 Hz, 1H, 1-H16); 4.20 (d, J=8.5 Hz, 1H, 1-H16); 3.95 (d, J=12.6 Hz, 1H, H5′); 3.91 (d, J=12.6 Hz, 1H, H5′); 3.82 (d, J=7.1 Hz, 1H, H3); 2.57 (ddd, J=14.8 Hz, J=9.1 Hz, J=6.6 Hz, 1H, H6); 2.49 (d, J=4.11 Hz, 1H, OH); 2.45 (s, 3H, H9″); 2.39 (dd, J=15.4 Hz, J=9.3 1H, H14); 2.23 (s, 3H, H11″); 2.19 (dd, J=15.6 Hz, J=8.8 Hz, 1H, H14); 1.93 (dd, J=1.3 Hz, 3H, H20); 1.88 (ddd, J=14.4 Hz, J=10.7 Hz, J=2.3 Hz, 1H, H6); 1.68 (s, 3H, H19); 1.23 (s, 3H, H18); 1.14 (s, 3H, H17).

¹³C-NMR: (CDCl₃, 100 MHz) δ=203.76 (C_{q}, C9, keton); 171.25 (C_{q}, C10″, carbonyl); 169.77 (C_{q}, C8″, carbonyl); 167.35 (C_{q}, C1′, carbonyl); 167.06 (C_{q}, carbonyl); 167.01 (C_{q}, carbonyl); 166.25 (C_{q}, C4′, carbonyl); 142.55 (C_{q}, C12, olefin); 136.53 (C_{q}, Cₐᵣₒ); 133.70 (CH, Cₐᵣₒ), 133.51 (C_{q}, Cₐᵣₒ); 132.90 (C_{q}, C12, olefin); 132.09 (CH, Cₐᵣₒ); 130.23 (2 CH, Cₐᵣₒ); 129.18 (4 CH, Cₐᵣₒ); 129.15 (C_{q}, Cₐᵣₒ); 128.76 (2 CH, Cₐᵣₒ)_{;} 128.69 (CH, Cₐᵣₒ); 127.09 (2 CH, Cₐᵣₒ); 126.61 (2 CH, Cₐᵣₒ); 84.44 (CH, C5); 81.09 (C_{q}, C4); 79.19 (C_{q}, C1); 76.45 (CH₂, C16); 75.56 (CH, C10); 75.36 (CH, C2′); 75.08 (CH, C2); 72.15 (2 CH, C7, C13); 58.53 (C_{q}, C8); 52.74 (CH, C3″); 45.56 (CH, C3); 43.18 (C_{q}, C15); 35.53 (2 CH₂, C6, C14); 26.83 (CH₃, C18); 24.56 (CH₂, C5′); 22.72 (CH₃, C9″); 22.11 (CH₃, C17); 20.81 (CH₃, C11″); 14.82 (CH₃, C20); 9.59 (CH₃, C19).

MS: (ESI; MeOH): m/z=996 [M(⁷⁹Br)+Na⁺], 998 [M(⁸¹Br)+Na⁺].

MS: (ESI; MeOH): m/z=1038 [M(⁷⁹Br)+Na⁺], 1040 [M(⁸¹Br)+Na⁺].

### 1.2.9 2′-(3-maleimidopropionyl)-paclitaxel (Pac3)

A 500 ml three-neck flask equipped with a magnetic stirring bar and inside thermometer was charged with 300 ml DCM and 1.5 g (1.76 mmol) paclitaxel. 445 mg (2.64 mmol) of N-maleoyl-β-alanine were added and the mixture was allowed to stir for 15 minutes. The flask was cooled in an ice-water bath to 0° C. 97 mg (0.79 mmol) DMAP and 408 mg (2.64 mmol) EDC were added to the reaction mixture and the mixture was allowed to warm up to room temperature. The course of the reaction was monitored by TLC. After completion of the reaction, the reaction mixture was washed twice with 150 ml of a 0.5% sodium bicarbonate solution and twice with 150 ml of 0.1 N hydrochloric acid. The organic phase was further washed with 300 ml of water followed by 300 ml of brine, dried over sodium sulfate and evaporated to dryness. The crude product was applied on silica and purified by column chromatography on silica (DCM/ethyl acetate 1:1). Yield was 1.12 g (1.11 mmol, 63%) of a colorless solid.

¹H-NMR: (CDCl₃, 200 MHz) δ=8.17-8.04 (m, 2H); 7.88-7.74 (m, 2H); 7.60-7.17 (m, 10H); 6.48-6.38 (m, 2H); 6.26-5.93 (m, 4H); 5.67-5.55 (m, 1H); 5.46-5.37 (m, 1H); 4.97-4.82 (m, 1H); 4.47-4.07 (m, 3H); 3.98-3.57 (m, 2H); 2.85-2.60 (m, 2H); 2.60-0.95 (m, 26H).

TLC: (DCM/ethyl acetate 1:1) R_{f}=0.55.

### 1.2.10 Synthesis of 2′-bromoacetyl cabazitaxel (Ctx-1)

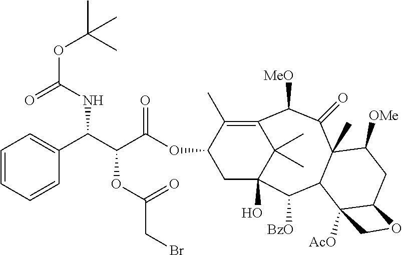

To a solution of cabazitaxel (500 mg; 0.598 mmol) in DCM (3.0 ml) were added DMAP (21.5 mg; 0.176 mmol), bromoacetic acid (99 mg; 0.712 mmol) and diisopropyl-carbodiimide (113.4 mg; 0.898 mmol) at 20-25° C. The reaction mixture was stirred at 20-25° C. and monitored by TLC analysis using ethyl aceate:hexane (1:1). After the completion of the reaction (˜30 minutes) the reaction mixture was quenched with water. The organic layer was washed with saturated aqueous NaHCO₃ solution and concentrated to dryness in vacuo. The residue thus obtained was purified by column chromatography over silica gel using 30% ethyl acetate in hexane to furnish 2′-bromoacetyl cabazitaxel (389 mg; 68%; 0.406 mmol) as colorless solid.

¹H NMR: (400 MHz; DMSO-d₆): δ=1.19 (s, 3H), 1.21 (s, 3H), 1.35 (s, 9H), 1.71 (s, 3H), 1.78 & 2.70 (2×m, 2H), 1.98 (s, 3H), 2.20 & 2.30 (2×m, 2H), 2.43 (s, 3H), 3.30 (s, 3H), 3.43 (s, 3H), 3.84 (d, 1H, J=7.2 Hz), 3.86 (m, 1H), 3.89 (br s, 2H), 4.17 & 4.31 (2×d, 2H, J=8.4 Hz), 4.82 (s, 1H), 4.99 (br d, 1H, J=9.6 Hz), 5.36 (br s, 2H), 5.49 (br m, 1H, CONH), 5.64 (d, 1H, J=7.2 Hz), 6.25 (br t, 1H), 7.32 (m, 3H), 7.40 (m, 2H), 7.49 (t, 2H, J=7.6 Hz), 7.60 (t, 1H, J=7.6 Hz), 8.10 (d, 2H, J=7.6 Hz).

MS (ESI): m/z=956 (M (⁷⁹Br)+H)⁺, 958 (M (⁸⁰Br)+H)⁺.

### 1.3 Special Procedures

### 1.3.1 Synthesis of Multi-Thio-HES (D1)

### a) Activation

In a dry three-neck round bottom flask equipped with a magnetic stirring bar, inert gas inlet and temperature probe, 15 g HES6 was dissolved in 60 ml of a 1:1 mixture of dry DMSO and pyridine under inert atmosphere. The solution was cooled to 10° C. by means of an ice-salt bath (inner temperature −8° C.). Solid 4-nitrophenyl chloroformat (9.6 g) was added in small portions while stirring (5 min). The resulting, highly viscous solution was allowed to stir for additional 30 min at −8° C. and then slowly poured into 900 ml of isopropanol. The resulting precipitate was collected by filtration over a pore 4 sinter funnel and washed with 4×100 ml of isopropanol followed by 2×150 ml MTBE. The precipitate was used in the next step without further purification.

b) Reaction with Cystamine

The activated HES from the last step was filled into a 250 ml glass bottle and dissolved in 150 ml of a 1:1 mixture of DMSO and pyridine. 28.6 g of cystamine dihydrochlorid were added and the resulting yellow suspension allowed to stirr over night in the closed bottle. After that reaction time, the solution was partitioned and a sample of 130 ml (⅔ of total volume, containing 10 g of HES) was centrifuged. The precipitate (excess linker) was discarded and the clear supernatant precipitated in 770 ml isopropanol. The mixture was centrifuged and the precipitated HES collected and re-dissolved in 240 ml of water. The product was further purified by ultrafiltration (concentrated to 100 ml, 20 volume exchanges with water, concentrated to 50 ml). The retentate was freeze-dried and the lyophilisate used directly in the next step.

c1) Reduction with DTT

In a 250 ml round bottom flask, the lyophilized intermediate from the last step (7.85 g) was dissolved in 70 ml of a borate buffer (pH 8.15). A solution of 605 mg of DTT in 8.5 ml of borate buffer was added and the resulting reaction mixture reacted at 40° C. under magnetic stirring. The mixture was precipitated in 600 ml of isopropanol and the HES collected by centrifugation. The precipitate was re-dissolved in 90 ml of 20 mM acetic acid+2 mM EDTA and subjected to ultrafiltration (15 volume exchanges with 20 mM acetic acid+2 mM EDTA followed by 5 volume exchanges with 20 mM acetic acid). The retentate was collected and freeze-dried to give 7.22 g (72%) of a colorless solid. As GPC analysis revealed a substantial amount of crosslinked HES, the product was reduced using sodium borohydride.

c2) Reduction with Sodium Borohydride

In a 250 ml round bottom flask, 6.47 g of the partially crosslinked thio-HES were dissolved in 65 ml of water. The flask was flushed with argon, then 647 mg of sodium borohydride were added (evolution of hydrogen gas) and the resulting solution was allowed to stir under argon for 3 h. The reaction was quenched by addition of 2 ml of acetic acid and the resulting mixture purified by ultrafiltration (dilution to 100 ml total volume, then 15 volume exchanges with 20 mM acetic acid+2 mM ETDA buffer followed by 5 exchanges with 20 mM acetic acid). The retentate was collected and freeze-dried to yield 6.16 g (62% referring to starting material) of derivative D1. Thiol loading: 121.5 nmol/mg. Mw=112 kDa, Mn=72 kDa.

### 1.3.2 Synthesis of Multi-Thio-HES (D3)

### a) Activation

The reaction was performed analog to D1 starting from 15 g of HES6. Cooling was achieved using a mixture of dry ice in ethanol maintaining the temperature between 25 and −15° C. The activated HES was immediately used in the next step.

b) Reaction with Cystamine

The reaction was performed analog to D1. The solution was not partitioned and resulted in 12.3 g of an off-white product.

c) Reduction with DTT

The reaction was performed analog to D1 (12.3 g HES, 949 mg DTT, 123 ml borate buffer pH 8.15). The yield was 11.2 g (75%) of a colorless solid. GPC analysis revealed a fraction of ˜5% of high molecular weight impurities (with Mw>10⁷ Dalton) which were depleted by fractionate precipitation.

### d) Fractionated Precipitation (1.4)

10.4 g of the product from the reduction step were dissolved in 100 ml of DMF (peptide syn. grade) in a 400 ml beaker. Under constant magnetic stirring, isopropanol was added until the solution became cloudy. After addition of 95 ml isopropanol, the mixture was centrifuged, the precipitate discarded and the supernatant treated with additional isopropanol. After addition of further 8 ml, the mixture was centrifuged again, resulting in a second, minor fraction of gel-like, high molecular weight HES. Further addition of isopropanol to the supernatant resulted in precipitation of the last fraction of HES, which was collected, dissolved in water and subjected to ultrafiltration (15 volume exchanges with water). The yield was 2.72 g (18% referring to starting material) and the thiol loading was 148.3 nmol/mg. Mw=71 kDa, Mn=47 kDa.

### 1.4 General Procedures

### 1.4.1 General Procedure for the Synthesis of Multi-Allyl HES (GP1.1)

Hydroxyethyl starch used in the preparation was thoughtfully dried prior to use either on an infra-red heated balance at 80° C. until the mass remained constant or by leaving in a drying oven over night at 80° C. A 10% solution of the dry HES in dry DMF or formamide (photochemical grade) was prepared in a round bottom flask equipped with a magnetic stirring bar and a rubber septum under an inert gas atmosphere. Sodium hydride (60% w/w in paraffin) was added in one portion and the resulting cloudy solution was allowed to stir for 1 h at room temperature followed by addition of allyl bromide. The reaction mixture was allowed to stir over night, resulting in a colorless-light brown, clear solution. The solution was then slowly poured into 7-10 times the volume of isopropanol and the precipitate was collected by centrifugation. The precipitated polymer was re-dissolved in water and subjected to ultrafiltration (15-20 volume exchanges with water). Freeze-drying of the retentate yielded a colorless solid.

### 1.4.2 General Procedure for the Synthesis of Multi-Epoxy HES (GP1.2)

In a glass beaker, multi-allyl-HES was dissolved in a 4*10⁻⁴ M EDTA solution (10-15 ml/g HES). Tetrahydrothiopyran-4-one was added and the solution allowed to stir on a magnetic stirring plate. Oxone® and sodium hydrogen carbonate were mixed in dry state and the mixture was added in small portions to the HES-solution resulting in the formation of a thick foam. The mixture was allowed to stir at ambient temperature for 2 h, diluted with water to a volume of 100 ml and then directly purified by ultrafiltration (15-20 volume exchanges with water). The resulting retentate was collected and directly used in the next step.

### 1.4.3 General Procedure for the Synthesis of Multi-MHP HES (GP1.3)

The solution of epoxidized HES obtained from GP1.2 was filled into a round bottom flask equipped with a magnetic stirring bar and a stopper. Sodium thiosulfate was added and, in certain experiments, acetic acid (50 μl/g HES) was added to keep the pH at 7 or below (without addition of acetic acid, the pH shifted to 10-11 during the course of the reaction). The resulting clear solution was allowed to stir for two days at ambient temperature. The polymer was purified by ultrafiltration (15-20 volume exchanges with water), the retentate was concentrated to 100 ml and directly subjected to the reduction reaction according to GP1.5.

### 1.4.4 General Procedure for the Synthesis of Multi-EtThio HES (GP1.4)

The solution of epoxidized HES obtained from GP1.2 was slowly poured into 7-10 times the volume of isopropanol. The precipitate was collected by centrifugation and re-dissolved in formamide (photochemical grade). An equal volume of DMF (peptide synthesis grade) was added and the mixture transferred into a reaction vessel equipped with a magnetic stirring bar and a rubber septum. A stream of inert gas was passed through the solution by means of a cannula for ˜10 min followed by addition of ethanedithiol. In case of formation of an emulsion, the mixture was homogenized by addition of DMF. The reaction was started by addition of a 0.1 M solution of Na₂CO₃ and the resulting solution was allowed to stir for two days under inert gas atmosphere. Finally, the mixture was slowly poured into 7-10 times the volume of cooled isopropanol (4° C.). The precipitate was collected by centrifugation, the polymer re-dissolved in water (white emulsion due to residual ethanedithiol) and purified by ultrafiltration (15-20 volume exchanges with water), resulting in a clear retentate. The retentate was concentrated to 100 ml and directly reduced according to GP1.5.

### 1.4.5 General Procedure for the Reduction of Multi-EtThio (GP1.5)

The HES-solution from the previous step was transferred into a round bottom flask equipped with a magnetic stirring bar and a rubber septum. A stream of inert gas was passed through the solution by means of a cannula for ˜10 min, followed by the addition of sodium borohydride (100 mg/g HES). The reaction was allowed to stir for 2 h or over night under an inert atmosphere. It was quenched by acidification with acetic acid (0.5 ml/g HES) under evolution of hydrogen. The neutralized/acidified solution was purified by ultrafiltration (15-20 volume exchanges with 20 mM acetic acid). The retentate was freeze dried to yield a colorless solid (yield: in the range of from 75 to 95%).

### 1.4.6 General Procedure for the Synthesis of Thioacetyl HES (GP2.1)

Hydroxyethyl starch as used in the preparation was thoughtfully dried prior to use either on an infra-red heated balance at 80° C. until the mass remained constant or by leaving in a drying oven over night at 80° C. The HES was dissolved in a round bottom flask equipped with a magnetic stirring bar and a rubber septum under inert gas using a 1:1 mixture of dry DMF and photochemical grade formamide to give a 10% HES-solution. After the addition of the base, the clear solution was cooled in an ice-water bath. In another reaction vessel, methanesulfonyl chloride was dissolved in five times the volume of dry DMF, the mixture was immediately transferred into a syringe and added drop-wise over a period of 5 min to the cooled HES solution under constant stirring. The reaction mixture was kept in the ice bath for ˜1 h, then the cooling bath was removed and the solution allowed to warm to room temperature. After additional 1-3 h of stirring, potassium thioacetate was added as a solid and the resulting amber solution was allowed to stir over night at the given temperature. In some cases (see table 8), 1-2 ml of mercaptoethanol were added as capping agent for residual mesylates and stirring was continued for an additional hour. The mixture was then poured in isopropanol (7-10 times the volume of the HES solution) and the precipitate collected by centrifugation. The crude product was diluted in 100 ml of water and purified by ultrafiltration (15-20 volume exchanges with water). Freeze-drying of the retentate yielded a colorless solid, which was directly used for saponification/reduction.

### 1.4.7 General Procedure for the Synthesis of SH-HES by Saponification of Thioacetyl HES Using Aqueous Ammonia (GP2.2a)

A 10% (w/v) solution of multi-thioacetyl HES derived from GP2.1 in water was prepared in a round bottom flask equipped with a magnetic stirring bar and a rubber septum under an inert gas atmosphere. The solution was degassed by passing a stream of inert gas through the mixture under stirring for ˜10 minutes. DTT was added resulting in a 50 mM solution. Then, an aliquot of equal volume aqueous ammonia (25%) was added and the resulting clear solution allowed to stir for 2 h at room temperature. The reaction was terminated by neutralisation with acetic acid (˜same volume as aqueous ammonia) under constant cooling with an ice-water bath. The neutralized mixture (pH 5-7) was diluted with water to a total volume of 100-200 ml and directly subjected to ultrafiltration (15-20 volume exchanges with a 20 mM solution of acetic acid in water). Freeze-drying of the retentate afforded multi-SH-HES as a colourless solid.

### 1.4.8 General Procedure for the Synthesis of SH-HES by Saponification of Thioacetyl HES Using Sodium Hydroxide (GP2.2b)

A 10% (w/v) solution of multi-thioacetyl HES derived from GP 2.1 in water was prepared in a round bottom flask equipped with a magnetic stirring bar and a rubber septum under an inert gas atmosphere. The solution was degassed by passing a stream of inert gas through the mixture while continous stirring for ˜10 minutes. A 1 M sodium hydroxide solution was added (10% of total volume), followed by addition of solid sodium borohydride (10% w/w of HES). The resulting solution was allowed to stir under inert gas for 4 h. The reaction was quenched by addition of acetic acid (˜0.5 ml/gram HES, pH=5-7) and diluted with water to a volume of 100-200 ml. The product was purified by ultrafiltration (15-20 volume exchanges with a 20 mM solution of acetic acid in water). Freeze-drying of the retentate afforded multi-SH-HES as a colorless solid.

### 1.4.9 General Procedure for the Synthesis of SH-HES Using Sodium Sulfide as Nucleophile (GP2.3)

Hydroxyethyl starch used in the preparation was thoughtfully dried prior to use either on an infra-red heated balance at 80° C. until the mass remained constant or by leaving in a drying oven over night at 80° C. The HES was dissolved in a round bottom flask equipped with a magnetic stirring bar and a rubber septum under an inert gas atmosphere using a 1:1 mixture of dry DMF and photochemical grade formamide to give a 10% solution of HES. After the addition of the base, the clear solution was cooled in an ice-water bath. In another reaction vessel, methanesulfonyl chloride was dissolved in five times the volume of dry DMF, the mixture immediately transferred into a syringe and added drop-wise over a period of ˜5 min to the cooled HES solution under constant stirring. The reaction mixture was kept in the ice bath for ˜1 h, then the cooling bath was removed and the solution allowed to warm to room temperature. After additional 1-3 h of stirring, solid sodium sulfide was added, the solution purged with inert gas and allowed to react over night at ambient temperature. The resulting clear, yellow-green solution was precipitated in 7-10 times the amount of isopropanol and the precipitate was collected by centrifugation. The precipitate was dissolved in 100-200 ml of water and further purified by ultrafiltration (5 volume exchanges with a 20 mM DTI solution containing 4 mM EDTA, followed by 15-20 volume exchanges with water). The retentate was concentrated to a volume of 50-100 ml and transferred into a round bottom flask. The solution was purged with inert gas for ˜10 min, sodium borohydride was added (100 mg/g HES) and the resulting solution was allowed to stir under an inert gas atmosphere at ambient temperature over night. The reduction reaction was quenched by acidification with acetic acid and directly subjected to ultrafiltration (20 volume exchanges with 20 mM acetic acid in water). The retentate was freeze-dried to give the title product as colorless solid.

### 1.4.10 General Procedure for the Synthesis of HES-Docetaxel Conjugates (GP3a)

In a round bottom flask equipped with a magnetic stirring bar and a rubber septum, the thiolated HES derivative was dissolved in DMF (peptide synthesis grade, 22.96 ml/g HES derivative). The solution was purged with inert gas for several minutes. The appropriate docetaxel derivative was added, followed by water (3.4 ml/g HES derivative) and a 0.1 M citrate buffer (pH 6.4, 2.2 ml/g HES derivative). The resulting solution was allowed to stir at room temperature for two hours under an inert gas atmosphere. Iodoacetic acid was added and the mixture was allowed to stir for an additional hour at room temperature under the absence of light. The conjugate was precipitated in 7 times the volume of isopropanol and centrifuged. The precipitate was isolated, re-dissolved in DMF (peptide synthesis grade, 30 ml/g HES) and precipitated again in isopropanol. Precipitation from DMF was repeated once and the precipitate dissolved in water (giving a 2-5% solution). The conjugate solution was filtered and purified via size exclusion chromatography. The fractions containing the polymer (1^{st} elution peak) were pooled and freeze-dried to yield the HES-docetaxel conjugate as a colorless solid.

### 1.4.11 General Procedure for the Synthesis of HES-Docetaxel Conjugates (GP3b)

In a round bottom flask equipped with a magnetic stirring bar and a rubber septum, the thiolated HES derivative was dissolved in DMF (peptide synthesis grade, 18 ml/g HES derivative). The solution was purged with inert gas for several minutes. The appropriate docetaxel derivative was added, followed by a 0.1 M phosphate buffer containing 5 mM EDTA (pH 7.5, 2 ml/g HES derivative). The resulting solution was allowed to stir at room temperature for two hours under an inert gas atmosphere. Iodoacetic acid was added and the mixture was allowed to stir for an additional hour at room temperature under the absence of light. The conjugate was precipitated in 7 times the volume of isopropanol and centrifuged. The precipitate was isolated, re-dissolved in DMF (peptide synthesis grade, 30 ml/g HES) and precipitated in isopropanol again. Precipitation from DMF was repeated once and the precipitate was dissolved in water (giving a 2-5% solution). The conjugate solution was filtered and purified via size exclusion chromatography. The fractions containing the polymer (1^{st} elution peak) were pooled and freeze-dried to yield the HES-docetaxel conjugate as a colorless solid.

### 1.4.12 General Procedure for the Synthesis of HES-Docetaxel Conjugates (GP3c)

In a round bottom flask equipped with a magnetic stirring bar and a rubber septum, the thiolated HES derivative was dissolved in DMF (peptide synthesis grade, 22.8 ml/g HES derivative). The solution was purged with inert gas for several minutes. The appropriate docetaxel derivative was added, followed by water (3.38 ml/g HES derivative) and a saturated sodium bicarbonate solution (pH 8, 2.2 ml/g HES derivative). The resulting solution was allowed to stir at room temperature for two hours under an inert gas atmosphere. Iodoacetic acid was added and the mixture was allowed to stir for an additional hour at room temperature under the absence of light. The conjugate was precipitated in 7 times the volume of isopropanol and centrifuged. The precipitate was isolated, re-dissolved in DMF (peptide synthesis grade, 30 ml/g HES) and precipitated again in isopropanol. Precipitation from DMF was repeated once and the precipitate was dissolved in water (giving a 2-5% solution). The conjugate solution was filtered and purified via size exclusion chromatography. The fractions containing the polymer (1^{s1} elution peak) were pooled and freeze-dried to yield the HES-docetaxel conjugate as a colorless solid.

### 1.4.13 General Procedure for the Determination of Thiol Content Using the Ellman Reagent (GP4)

A stock solution of 4 mg/ml of 5,5′-dithio-bis(2-nitrobenzoic acid), Ellman's reagent, in 0.1 M sodium phosphate buffer+1 mM EDTA (pH 8) buffer was freshly prepared. A 0.2 mg/ml solution of sample in buffer was prepared and 1 ml of this solution was filled into a 2 ml vial. An additional vial containing 1 ml of plain buffer was used as blank. The samples were treated with 100 μl of the reagent stock solution, placed into a mixer and mixed at 750 rpm at 21° C. for 15 minutes. The sample solutions were transferred into plastic cuvettes (d=10 mm) and measured for absorbance at 412 nm. The amount of thiols present in the vial was calculated according to the following formula (A=absorbance of sample, A⁰=absorbance of blank):

$$c [ µmol \text{/} {cm}^{3} ] = \frac{1.1 * ( {A}_{412} - {A}_{412}^{0} )}{14.150 \frac{{cm}^{2}}{µmol} * 1 cm}$$

considering the concentration of 0.2 mg/ml and 1 cm³=1 ml:

$$Loading [ nmol \text{/} mg ] = \frac{1000 * c}{0.2 \frac{mg}{ml}}$$

The final value was calculated as the average loading from the three samples.

### 1.4.14 General Procedure for the Determination of Drug Content Via UV Absorption (GP5)

A stock solution of the drug conjugate sample in the appropriate solvent (see table 4) was prepared (c_{conjugate}=0.1-0.5 mg/ml). An equally concentrated sample of the HES derivative used for the preparation of the conjugate was used as a blank. The absorbance at the absorbance maximum (see table 4) was measured and the drug content calculated using the following formula:

$${c}_{drug} [ µmol \text{/} {cm}^{3} ] = \frac{( {A}_{λ} - {A}_{λ}^{0} )}{{ɛ}_{λ} * 1 cm}$$

considering the concentration of the stock solution:

$$Loading [ µmol \text{/} g ] = \frac{1000 * {c}_{drug} [ µmol \text{/} ml ]}{{c}_{conjugate} [ mg \text{/} ml ]}$$

Taking into account the molecular weight of the drug:

Loading[mg/g]=Loading[μmol/g]*MW_{drug}[μg/μmol]/1000

The final value is calculated as an average value of 3 to 5 independent measurements.

The molar extinction coefficients were obtained from a calibration curve of the drugs in the specific solvents at the specific absorbance maximum.

### 1.4.15 General Procedure for the Determination of the Cleaving Tendency of Certain Tested Linker Compounds

The cleaving tendency of certain linker compounds were determined by incubating certain hydroxyethyl starch conjugates (see table 15a) in PBS buffer at pH 7.4 at 37° C. for 45 h. After 45 h the amount of cleaved hydroxyalkyl starch conjugate was determined using HPLC. The results are shown in table 15a.

### 1.4.16 General Procedure for the Determination of the Cleaving Tendency of Certain Tested Linker Compounds

The “mean molecular weight” as used in the context of the present invention relates to the weight as determined according to MALLS-GPC (Multiple Angle Laser Light Scattering). For the determination, 2 Tosoh BioSep GMPWXL columns connected in line (13 μm particle size, diameter 7.8 mm, length 30 cm, Art. no. 08025) were used as stationary phase. The mobile phase was prepared as follows: In a volumetric flask 3.74 g Na-Acetate*3H₂O, 0.344 g NaN₃ are dissolved in 800 ml Milli-Q water and 6.9 ml acetic acid anhydride are added and the flask filled up to 1 l. Approximately 10 mg of the hydroxyalkyl starch derivative were dissolved in 1 ml of the mobile phase and particle filtrated with a syringe filter (0.22 mm, mStarII, CoStar Cambridge, Mass.). The measurement was carried out at a flow rate of 0.5 ml/min. As detectors a multiple-angle laser light scattering detector and a refractometer maintained at a constant temperature, connected in series, were used. Astra software (Vers. 5.3.4.14, Wyatt Technology Cooperation) was used to determine the mean M_{w} and the mean Mₙ of the sample using a dn/dc of 0.147. The value was determined at λ=690 nm (solvent NaOAc/H₂O/0.02% NaN₃, T=20° C.) in accordance with literature (W. M. Kulicke, U. Kaiser, D. Schwengers, R. Lemmes, Starch, Vol. 43, Issue 10 (1991), 392-396).

TABLE 4
Extinction coefficients at 230 nm for paclitaxel, docetaxel and
cabazitaxel determined from calibration curves in TFE and TFE/H₂O

| | | | | | M_{w} |
|---|---|---|---|---|---|
| # | Drug | Solvent | λ [nm] | ε_{λ} [cm²/μmol] | [g/mol] |
| 1 | Paclitaxel | TFE | 230 | 28.535 | 853.91 |
| 2 | Docetaxel | TFE | 230 | 15.912 | 807.88 |
| 3 | Docetaxel | TFE/H₂O 1:1 | 230 | 16.307 | 807.88 |
| 4 | Cabazitaxel | TFE/H₂O 9:1 | 232 | 14.746 | 835.9 |

TABLE 5
Synthesis of multi-Allyl-HES intermediates
(I1-I16) according to GP1.1

| | | | | NaH | AIIBr | Yield | Mw | Mn |
|---|---|---|---|---|---|---|---|---|
| # | HES | m[g] | Solvent | m[mg] | V [μl] | [%] | [kD] | [kD] |
| I1 | HES14 | 5.0 | DMF | 270 | 470 | 92 | n.d. | n.d. |
| I2 | HES6 | 5.0 | DMF | 203 | 580 | n.d. | 87.4 | 59.4 |
| I3 | HES6 | 10.0 | DMF | 271 | 470 | 91 | n.d. | n.d. |
| I4 | HES6 | 10.0 | DMF | 271 | 470 | 87 | n.d. | n.d. |
| I5 | HES14 | 10.0 | DMF | 271 | 470 | 84 | 759 | 561 |
| I6 | HES2 | 10.0 | FA | 498 | 862 | 97 | 90 | 74 |
| I7 | HES7 | 10.0 | FA | 486 | 841 | 99 | 275 | 216 |
| I10 | HES8 | 10.0 | FA | 464 | 802 | 97 | 275 | 201 |
| I11 | HES9 | 10.0 | FA | 433 | 750 | 93 | 249 | 178 |
| I12 | HES3 | 10.0 | FA | 470 | 803 | 87 | 75 | 65 |
| I14 | HES5a | 10.0 | DMF | 292 | 500 | 94 | 86 | 72 |
| I8 | HES11 | 10.2 | FA | 500 | 850 | 93 | n.d. | n.d. |
| I9 | HES12 | 9.9 | FA | 450 | 750 | 88 | n.d. | n.d. |
| I13 | HES13 | 10.2 | DMF | 380 | 630 | 92 | n.d. | n.d. |
| I15 | HES6 | 20.2 | DMF | 602 | 950 | 93 | 84.1 | 58.1 |
| I16 | HES6 | 20.1 | DMF | 630 | 940 | 94 | n.d. | n.d. |

TABLE 6
Synthesis of multi-EtThio and multi-MHP-HES derivatives according to GP1

| | | | GP1.2 | | | GP1.3 | | GP1.4 | | | GP1.5 |
|---|---|---|---|---|---|---|---|---|---|---|---|
| | Allyl HES | | Oxone ® | NaHCO₃ | THTP^{a} | Na₂S₂O₃ | HOAc | Ethanedithiol | buffer | V_{DMF/FA} | NaBH₄ |
| # | | m[g] | m[g] | m[g] | m[mg] | m[g] | V [μl] | V[ml] | V[ml] | V[ml] | m[g] |
| D2 | I1 | 4.41 | 5.52 | 2.32 | 35 | 3.36 | — | — | — | — | 1.31 |
| D4 | I2 | 5.00 | 6.28 | 2.68 | 39 | 1.68^{b} | — | — | — | — | 1.25 |
| D5 | I3 | 4.15 | 2.07 | 0.88 | 27 | — | — | 9.42^{b} | 3.0^{b} | 20/0^{b} | 0.21^{b} |
| D6 | I4 | 4.00 | 2.00 | 0.85 | 25 | 10.8^{b} | 60^{b} | — | — | — | 0.40^{b} |
| D7 | I5 | 4.00 | 2.00 | 0.85 | 25 | 13.5^{b} | 30^{b} | — | — | — | 0.40^{b} |
| D8 | I5 | 2.08 | 1.00 | 0.45 | 7 | — | — | 11.45 | 4.0 | 30/0 | 0.50 |
| D9 | I6 | 5.00 | 4.60 | 1.95 | 30 | — | — | 41.90 | 15.0 | 150/0 | 0.50 |
| D10 | I7 | 9.64 | 8.63 | 3.67 | 55 | — | — | 40.0^{b} | 5.0^{b} | 55/60^{b} | 0.37^{b} |
| D11 | I8 | 9.34 | 8.33 | 3.65 | 55 | — | — | 76.4 | 10.0 | 135/175 | 1.02 |
| D12 | I9 | 8.67 | 7.12 | 3.05 | 45 | — | — | 32.5^{b} | 5.0^{b} | 45/50^{b} | 0.52^{b} |
| D13 | I10 | 9.71 | 8.72 | 3.68 | 56 | — | — | 40.0^{b} | 5.0^{b} | 60/50^{b} | 0.49^{b} |
| D14 | I11 | 9.11 | 8.17 | 3.46 | 53 | — | — | 33.2^{b} | 5.0^{b} | 50/50^{b} | 0.46^{b} |
| D15 | | | | | | 9.80^{b} | 103^{b} | — | — | — | 0.46^{b} |
| D16 | I12 | 9.00 | 7.70 | 3.26 | 96 | — | — | 35.0^{b} | 5.0^{b} | 40/60^{b} | 0.45^{b} |
| D17 | I13 | 8.76 | 5.89 | 2.46 | 37 | — | — | 27.0^{b} | 5.0^{b} | 100/0^{b} | 0.50^{b} |
| D18 | I14 | 9.00 | 7.32 | 1.91 | 57 | — | — | 20.5^{b} | 7.5^{b} | 75/0^{b} | 0.20^{b} |
| D19 | | | | | | 24.0^{b} | 70^{b} | — | — | — | 0.20^{b,c} |
| D20 | I15 | 5.50 | 5.49 | 2.33 | 35 | 14.8 | 80 | — | — | — | 0.60 |
| D21 | I16 | 10.00 | 5.04 | 2.11 | 35 | — | — | 23^{b} | 7^{b} | 50/0^{b} | 0.5^{b} |

^{a}tetrahydrothiopyran-4-one,
^{b}Amounts refer to ½ the starting amount of HES. The retentate of GP2.2 was used for 2 independent preparations,
^{c}GP2.5 was performed twice due to unexpected oxidative crosslinking after the first reduction.

TABLE 7
Characterization of multi-EtThio and multi-MHP-HES derivatives

| | Yield | Loading^{a} | Mw | Mn |
|---|---|---|---|---|
| # | [%] | [nmol/mg] | [kD] | [kD] |
| D2 | 76 | 318 | 1112 | 608 |
| D4 | | 229 | 102 | 66 |
| D5 | 50 | 241 | 110 | 65 |
| D6 | 91 | 224 | 99 | 58 |
| D7 | 83 | 171 | 1014 | 523 |
| D8 | 71 | 119 | 688 | 302 |
| D9 | 87 | 195 | 98 | 81 |
| D10 | 98 | 229 | 321 | 234 |
| D11 | 65 | 213 | 838 | 498 |
| D12 | 64 | 172 | 816 | 404 |
| D13 | 78 | 218 | 311 | 213 |
| D14 | 76 | 195 | 262 | 185 |
| D15 | 86 | 196 | 272 | 185 |
| D16 | 94 | 224 | 92 | 71 |
| D17 | 72 | 182 | 435 | 372 |
| D18 | 58 | 213 | 201 | 113 |
| D19 | 96 | 214 | 159 | 66 |
| D20 | 77 | 234 | 114 | 65 |
| D21 | 75 | 223 | 86 | 59 |

^{a}determined according to GP4

TABLE 8
Synthesis and characterization of Thiol-
HES-derivatives according to GP2.1

| | HES | | Base | | MsCl | | KSAc |
|---|---|---|---|---|---|---|---|
| | | m | | V | m | Mesylation^{a} | m |
| | | [g] | | [ml] | [g] | conditions | [g] |
| D28 | HES1 | 3.0 | DIPEA | 0.61 | 0.27 | 2 h 0° C.-RT | 1.98 |
| D29 | HES5a | 5.0 | collidine | 0.96 | 0.57 | 4 h 0° C.-RT | 4.13 |
| D23 | HES5a | 5.0 | collidine | 0.96 | 0.56 | 3.5 h 0° C.-RT | 4.13 |
| D24 | HES9 | 2.0 | DIPEA | 0.5 | 0.23 | 1.5 h 0° C.-RT | 1.65 |
| D26 | HES9 | 5.0 | collidine | 0.96 | 0.57 | 3 h 0° C.-RT | 4.13 |
| D30 | HES5b | 1.0 | DIPEA | 0.38 | 0.17 | 1 h 0° C.-RT | 1.26 |

| | Temp. | | | Yield | Loading^{d} | Mw | Mn |
|---|---|---|---|---|---|---|---|
| | [° C.] | Capping^{b} | Sap.^{c} | [%] | [nmol/mg] | [kD] | [kD] |
| D28 | RT | no | GP2.2a | 83 | 230 | 54 | 44 |
| D29 | 50 | 1 h, 50° C. | GP2.2b | 82 | 117 | 84 | 62 |
| D23 | RT | 4 h, RT | GP2.2a | 80 | 128 | 85 | 63 |
| D24 | RT | no | GP2.2a | 99 | 190 | 247 | 183 |
| D26 | 50 | 1 h, 50° C. | GP2.2a | 69 | 169 | 247 | 176 |
| D30 | RT | no | GP2.2a | 72 | 235 | 83 | 67 |
| D31 | 50 | no | GP2.2b^{e} | 91 | 172 | 94 | 67 |

^{a}reaction time and temperature after addition of mesyl chloride
^{b}addition of mercaptoethanol after reaction with KSAc and capping conditions
^{c}Saponification conditions, GP2.2
^{d}determined according to GP4
^{e}Thioacetyl-HES from retentate was saponified without prior isolation

TABLE 9
Synthesis and characterization of Thiol-HES-derivatives according to GP2.3

| | HES | | Base | | MsCl | Mesylation^{a} | NaSH | Yield | Loading^{b} | Mw | Mn |
|---|---|---|---|---|---|---|---|---|---|---|---|
| # | | m [g] | | V [ml] | V[ml] | conditions | m[g] | [%] | [nmol/mg] | [kD] | [kD] |
| D22 | HES4 | 5.0 | TEA | 0.628 | 0.351 | 4 h 0° C.-RT | 2.54 | 86 | 231 | 109 | 76 |
| D25 | HES6 | 5.0 | TEA | 0.48 | 0.27 | 4 h 0° C.-RT | 3.89 | 86 | 173 | 103 | 63 |
| D27 | HES5b | 2.0 | DIPEA | 1.00 | 0.45 | 4 h 0° C.-RT | 0.81 | 73 | 318 | 94 | 71 |

^{a}reaction time and temperature after addition of mesyl chloride
^{b}determined according to GP4

TABLE 10
Synthesis and characterization of HES-docetaxel conjugates CDc1-CDc35

| | | HES | | Docetaxel | | IAA | Yield^{a} | Purity^{b} | Loading^{c} | | Mw | Mn |
|---|---|---|---|---|---|---|---|---|---|---|---|---|
| # | GP | derivative | m[g] | derivative | m[mg] | m[mg] | [%] | [%] | [mg Doc/g] | [μmol/g] | [kD] | [kD] |
| CDc1 | GP3a | D1 | 1.00 | Doc1 | 115 | 276 | 87 | >99.9 | 75.1¹ | 92.9¹ | 170 | 88 |
| CDc2 | GP3c | D1 | 1.00 | Doc2 | 481 | 276 | 78 | >99.9 | 66.6¹ | 82.4¹ | 129 | 73 |
| CDc3 | GP3a | D3 | 1.00 | Doc1 | 138 | 331 | 88 | >99.9 | 91.4¹ | 113.1¹ | 217 | 62 |
| CDc4 | GP3b | D4 | 0.50 | Doc3 | 121 | 256 | 77 | >99.9 | 139.4¹ | 172.5¹ | 323 | 139 |
| CDc5 | GP3a | D5 | 0.48 | Doc1 | 107 | 257 | 91 | >99.9 | 142.1¹ | 175.9¹ | 187 | 123 |
| CDc6 | GP3b | D7 | 0.50 | Doc3 | 90 | 191 | 83 | >99.9 | 104.9¹ | 129.8¹ | 3634 | 1224 |
| CDc7 | GP3a | D8 | 0.50 | Doc1 | 55 | 132 | 78 | >99.9 | 66.3¹ | 82.1¹ | 1036 | 441 |
| CDc8 | GP3a | D9 | 0.50 | Doc1 | 91 | 218 | 92 | >99.9 | 110.7² | 137.0² | 175 | 136 |
| CDc9 | GP3a | D10 | 0.50 | Doc1 | 109.5 | 256 | 86 | >99.9 | 122.7² | 151.9² | 442 | 321 |
| CDc10 | GP3a | D21 | 0.50 | Doc1 | 104 | 249 | 76 | >99.9 | 125.7¹ | 155.6¹ | 144 | 99 |
| CDc11 | GP3a | D11 | 0.51 | Doc1 | 99 | 239 | 59 | >99.9 | 124.3² | 153.8² | 1300 | 714 |
| CDc12 | GP3a | D12 | 0.50 | Doc1 | 81 | 192 | 90 | >99.9 | 102.2² | 126.5² | 1188 | 552 |
| CDc13 | GP3a | D13 | 0.51 | Doc1 | 104 | 247 | 86 | >99.9 | 131.02 | 162.12 | 436 | 247 |
| CDc14 | GP3a | D14 | 0.50 | Doc1 | 91 | 216 | 87 | >99.9 | 125.1² | 154.8² | 355 | 249 |
| CDc15 | GP3a | D16 | 0.50 | Doc1 | 105 | 251 | 86 | >99.9 | 142.2² | 176.0² | 164 | 137 |
| CDc16 | GP3a | D17 | 0.50 | Doc1 | 84 | 205 | 86 | >99.9 | 110.1² | 136.3² | 1216 | 625 |
| CDc17 | GP3a | D18 | 0.50 | Doc1 | 100 | 240 | 87 | >99.9 | 131.5² | 162.7² | 203 | 152 |
| CDc18 | GP3b | D20 | 0.50 | Doc4 | 152 | 262 | 85 | >99.9 | 169.3¹ | 209.5¹ | 286 | 155 |
| CDc19 | GP3b | D20 | 0.27 | Doc5 | 105 | 141 | 78 | >99.9 | 141.6¹ | 175.2¹ | 199 | 119 |
| CDc20 | GP3a | D22 | 0.50 | Doc1 | 100 | 240 | 84 | 99.90 | 136.1¹ | 168.4¹ | 306 | 198 |
| CDc21 | GP3a | D23 | 0.50 | Doc1 | 62 | 244 | 83 | 99.85 | 78.2¹ | 96.8¹ | 115 | 74 |
| CDc22 | GP3b | D24 | 0.50 | Doc4 | 113 | 217 | 58 | >99.9 | 107.8¹ | 133.4¹ | 392 | 284 |
| CDc23 | GP3b | D15 | 0.50 | Doc6 | 136 | 220 | 79 | >99.9 | 129.4¹ | 160.1¹ | 426 | 287 |
| CDc24 | GP3a | D25 | 0.50 | Doc1 | 89 | 196 | 87 | >99.9 | 104.3¹ | 129.1¹ | 384 | 170 |
| CDc25 | GP3b | D15 | 0.50 | Doc4 | 114 | 219 | 82 | >99.9 | 140.2¹ | 173.5¹ | 407 | 279 |
| CDc26 | GP3b | D26 | 0.50 | Doc4 | 99 | 189 | 94 | >99.9 | 123.2¹ | 152.5¹ | 374 | 283 |
| CDc27 | GP3b | D26 | 0.50 | Doc6 | 116 | 189 | 87 | >99.9 | 121.7¹ | 150.6¹ | 391 | 280 |
| CDc28 | GP3a | D27 | 0.50^{d} | Doc1 | 149^{b} | 357 | 68 | >99.9 | 199.9¹ | 247.4¹ | 182 | 145 |
| CDc29 | GP3a | D18 | 0.50 | Doc1 | 101 | 240 | 90 | >99.9 | 137.8¹ | 170.5¹ | 165 | 122 |
| CDc30 | GP3b | D25 | 0.50 | Doc4 | 101 | 196 | 86 | >99.9 | 129.2¹ | 159.9¹ | 201 | 79 |
| CDc31 | GP3b | D25 | 0.51 | Doc6 | 118 | 196 | 93 | >99.9 | 116.7¹ | 144.4¹ | 258 | 145 |
| CDc32 | GP3a | D28 | 0.50 | Doc1 | 119 | 258 | 89 | >99.9 | 132.2¹ | 163.3¹ | 385 | 126 |
| CDc33 | GP3b | D19 | 0.50 | Doc4 | 126 | 241 | 80 | >99.9 | 152.6¹ | 188.9¹ | 307 | 133 |
| CDc34 | GP3a | D29 | 0.50 | Doc1 | 66 | 132 | 92 | >99.9 | 84.7¹ | 104.8¹ | 105 | 74 |
| CDc35 | GP3a | D30 | 0.50 | Doc1 | 109 | 262 | 74 | >99.9 | 129.9¹ | 160.8¹ | 153 | 114 |

^{a}calculated as [100*m_{conjugate}]/[m_{derivative}*(1 + Loading/1000)]
^{b}determined by HPLC
^{c}determined according to GP5
^{d}derivative D22 and Doc1 dissolved in 30 ml DMF/g HES
¹measured in TFE (see Table 3, entry 2)
²measured in TFE/water 1:1 (see Table 3 entry 3)

TABLE 11a
Synthesis and characterization of HES-paclitaxel conjugates CPc1-CPc7

| | | HES | | Paclitaxel | | IAA | Yield^{a} | Purity^{b} | Loading^{c} | | Mw | Mn |
|---|---|---|---|---|---|---|---|---|---|---|---|---|
| # | GP | derivative | m[g] | derivative | m[mg] | m[mg] | [%] | [%] | [mg Pac/g] | [μmol/g] | [kD] | [kD] |
| CPc1 | GP3a | D1 | 1.00 | Pac1 | 121 | 276 | 88 | 99.9 | 63.6 | 74.5 | 262 | 123 |
| CPc2 | GP3c | D2 | 1.00 | Pac2 | 1281 | 703 | 74 | >99.9 | 60.6 | 71.0 | 3265 | 738 |
| CPc3 | GP3c | D1 | 1.00 | Pac2 | 504 | 256 | 71 | 99.7 | 54.1 | 63.3 | 158 | 82 |
| CPc4 | GP3c | D2 | 1.00 | Pac2 | 641 | 703 | 70 | >99.9 | 64.6 | 75.6 | 4775 | 795 |
| CPc5 | GP3b | D5 | 0.50 | Pac1 | 121 | 268 | 87 | >99.9 | 153.4 | 179.6 | 201 | 128 |
| CPc6 | GP3b | D6 | 0.50 | Pac3 | 124 | 250 | 87 | >99.9 | 153.5 | 179.7 | 205 | 132 |
| CPc7 | GP3b | D7 | 0.50 | Pac3 | 124 | 191 | 85 | >99.9 | 131.0 | 153.4 | 1540 | 676 |

^{a}calculated as [100*m_{conjugate}]/[m_{derivative}*(1 + Loading/1000)]
^{b}determined by HPLC
^{c}determined according to GP5

TABLE 11b
Synthesis and characterization of HES-cabazitaxel conjugate CCx1

| | | HES | | Cabazitaxel | | BrAcee^{a} | Yield^{b} | Purity^{c} | Loading^{d} | | Mw | Mn |
|---|---|---|---|---|---|---|---|---|---|---|---|---|
| # | GP | derivative | m[g] | derivative | m[mg] | m[μl] | [%] | [%] | [mg Ctx/g] | [μmol/g] | [kD] | [kD] |
| CCx1 | GP3a | D31 | 0.2 | Ctx1 | 32.2 | 18.7 | 78 | 99% | 127.4 | 152 | 144 | 98 |

^{a}ethyl bromoacetate used as capping reagent instead of iodoacetic acid
^{b}calculated as [100*m_{conjugate}]/[m_{derivative}*(1 + Loading/1000)]
^{c}determined by HPLC
^{d}determined according to GP5 (232 nm, TFE/H₂O 9:1)

TABLE 12
Overview over synthesized Docetaxel derivatives

| Code | Name | Formula |
|---|---|---|
| Doc1 | 2′-(bromoacetyl)-docetaxel | 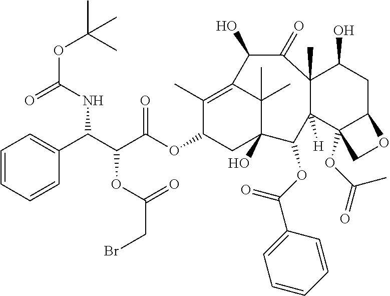 |
| Doc2 | 2′-(5-bromopentanoyl)- docetaxel | 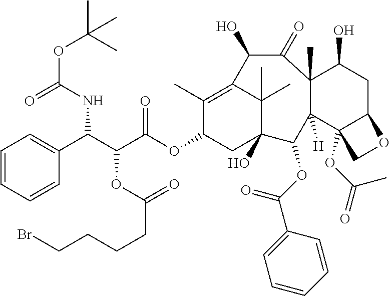 |
| Doc3 | 2′-(3-maleimidopropionyl)- docetaxel | 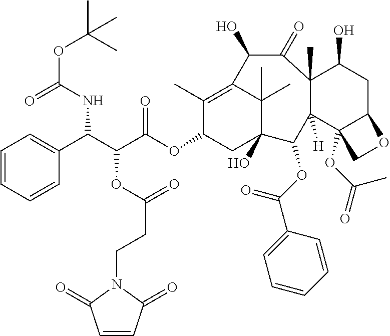 |
| Doc4 | 2′-(5-maleimido-3-thio- pentanoyl)-docetaxel | 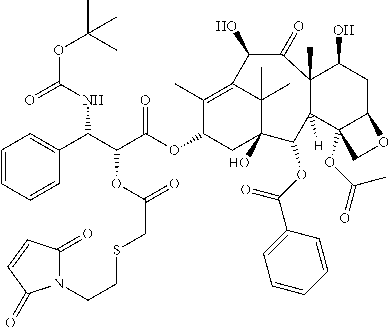 |
| Doc5 | 2′-(5-maleimido-3-oxo- pentanoyl)-docetaxel | 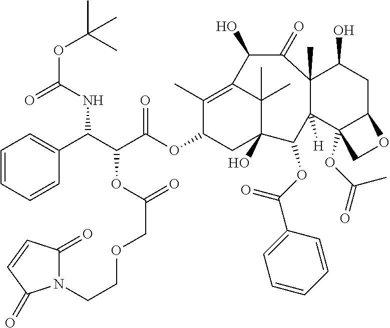 |
| Doc6 | 2′-(6-maleimido-3-oxo- hexanoyl)-docetaxel | 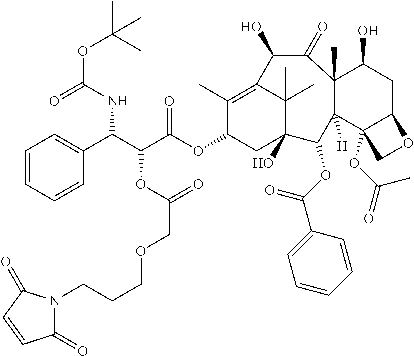 |

TABLE 13
Overview of synthesized Paclitaxel derivatives

| Code | Name | Formula |
|---|---|---|
| Pac1 | 2′-(bromoacetyl)-paclitaxel | 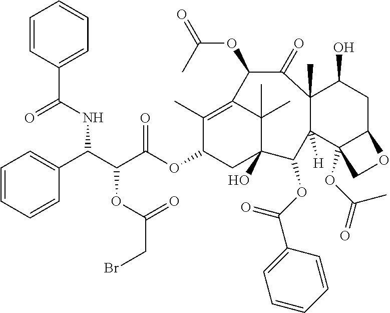 |
| Pac2 | 2′-(5-bromopentanoyl)- paclitaxel | 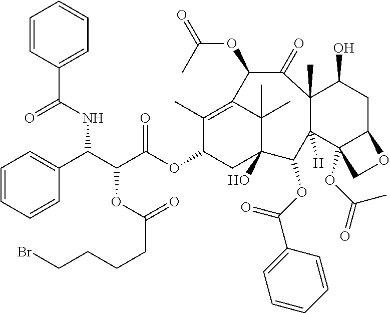 |
| Pac3 | 2′-(3-maleimidopropionyl)- paclitaxel | 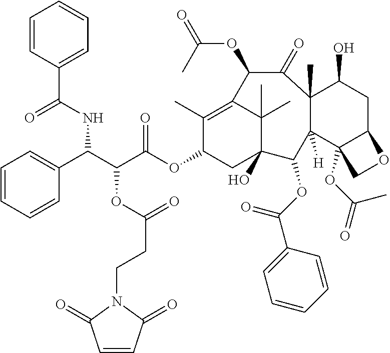 |

TABLE 14
Overview of synthesized hydroxyethyl starch derivatives

| | Structure | | | |
|---|---|---|---|---|
| Code | HES used | 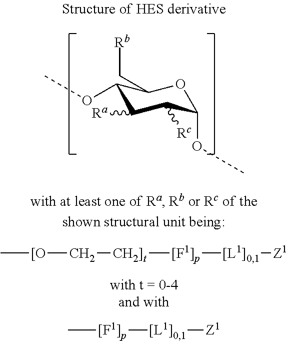 | Linking moiety L | Cytotoxic agent M |
| D1 | HES6 | —O—C(═O)—NH—CH₂—CH₂—SH | — | — |
| D2 | HES14 | —O—CH₂—CHOH—CH₂—SH | — | — |
| D3 | HES6 | —O—C(═O)—NH—CH₂—CH₂—SH | — | — |
| D4 | HES 6 | —O—CH₂—CHOH—CH₂—SH | — | — |
| D5 | HES 6 | —O—CH₂—CHOH—CH₂—S—CH₂—CH₂—SH | — | — |
| D6 | HES 6 | —O—CH₂—CHOH—CH₂—SH | — | — |
| D7 | HES14 | —O—CH₂—CHOH—CH₂—SH | — | — |
| D8 | HES14 | —O—CH₂—CHOH—CH₂—S—CH₂—CH₂—SH | — | — |
| D9 | HES2 | —O—CH₂—CHOH—CH₂—S—CH₂—CH₂—SH | — | — |
| D10 | HES7 | —O—CH₂—CHOH—CH₂—S—CH₂—CH₂—SH | — | — |
| D11 | HES11 | —O—CH₂—CHOH—CH₂—S—CH₂—CH₂—SH | — | — |
| D12 | HES12 | —O—CH₂—CHOH—CH₂—S—CH₂—CH₂—SH | — | — |
| D13 | HES 8 | —O—CH₂—CHOH—CH₂—S—CH₂—CH₂—SH | — | — |
| D14 | HES9 | —O—CH₂—CHOH—CH₂—S—CH₂—CH₂—SH | — | — |
| D15 | HES 9 | —O—CH₂—CHOH—CH₂—SH | — | — |
| D16 | HES 3 | —O—CH₂—CHOH—CH₂—S—CH₂—CH₂—SH | — | — |
| D17 | HES 13 | —O—CH₂—CHOH—CH₂—S—CH₂—CH₂—SH | — | — |
| D18 | HES5a | —O—CH₂—CHOH—CH₂—S—CH₂—CH₂—SH | — | — |
| D19 | HES5a | —O—CH₂—CHOH—CH₂—SH | — | — |
| D20 | HES6 | —O—CH₂—CHOH—CH₂—SH | — | — |
| D21 | HES6 | —O—CH₂—CHOH—CH₂—S—CH₂—CH₂—SH | — | — |
| D22 | HES 4 | —SH | — | — |
| D23 | HES 5a | —SH | — | — |
| D24 | HES 9 | —SH | — | — |
| D25 | HES6 | —SH | — | — |
| D26 | HES9 | —SH | — | — |
| D27 | HES5b | —SH | — | — |
| D28 | HES1 | —SH | — | — |
| D29 | HES5a | —SH | — | — |
| D30 | HES5b | —SH | — | — |
| D31 | HES17 | —SH | — | — |

TABLE 15
Overview of synthesized hydroxyethyl starch conjugates

| | Structure | | | |
|---|---|---|---|---|
| Code | HES used | 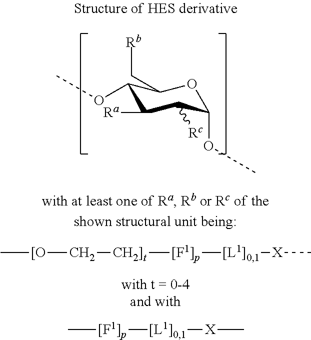 | Linking moiety L | Cytotoxic agent M |
| CDc1 | HES 1 | —O—C(═O)—NH—CH₂—CH₂—S— | —CH₂—C(═O)— | -2′-Docetaxel |
| CDc2 | HES 1 | —O—C(═O)—NH—CH₂—CH₂—S— | —CH₂—CH₂—CH₂—CH₂—C(═O)— | -2′-Docetaxel |
| CDc3 | HES 6 | —O—C(═O)—NH—CH₂—CH₂—S— | —CH₂—C(═O)— | -2′-Docetaxel |
| CDc4 | HES 6 | —O—CH₂—CHOH—CH₂—S— | 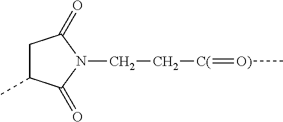 | -2′-Docetaxel |
| CDc5 | HES 6 | —O—CH₂—CHOH—CH₂—S—CH₂—CH₂—S— | —CH₂—C(═O)— | -2′-Docetaxel |
| CDc6 | HES 14 | —O—CH₂—CHOH—CH₂—S— | 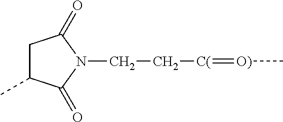 | -2′-Docetaxel |
| CDc7 | HES 14 | —O—CH₂—CHOH—CH₂—S—CH₂—CH₂—S— | —CH₂—C(═O)— | -2′-Docetaxel |
| CDc8 | HES 2 | —O—CH₂—CHOH—CH₂—S—CH₂—CH₂—S— | —CH₂—C(═O)— | -2′-Docetaxel |
| CDc9 | HES 7 | —O—CH₂—CHOH—CH₂—S—CH₂—CH₂—S— | —CH₂—C(═O)— | -2′-Docetaxel |
| CDc10 | HES 6 | —O—CH₂—CHOH—CH₂—S—CH₂—CH₂—S— | —CH₂—C(═O)— | -2′-Docetaxel |
| CDc11 | HES 11 | —O—CH₂—CHOH—CH₂—S—CH₂—CH₂—S— | —CH₂—C(═O)— | -2′-Docetaxel |
| CDc12 | HES 12 | —O—CH₂—CHOH—CH₂—S—CH₂—CH₂—S— | —CH₂—C(═O)— | -2′-Docetaxel |
| CDc13 | HES 8 | —O—CH₂—CHOH—CH₂—S—CH₂—CH₂—S— | —CH₂—C(═O)— | -2′-Docetaxel |
| CDc14 | HES 9 | —O—CH₂—CHOH—CH₂—S—CH₂—CH₂—S— | —CH₂—C(═O)— | -2′-Docetaxel |
| CDc15 | HES 3 | —O—CH₂—CHOH—CH₂—S—CH₂—CH₂—S— | —CH₂—C(═O)— | -2′-Docetaxel |
| CDc16 | HES 13 | —O—CH₂—CHOH—CH₂—S—CH₂—CH₂—S— | —CH₂—C(═O)— | -2′-Docetaxel |
| CDc17 | HES 5a | —O—CH₂—CHOH—CH₂—S—CH₂—CH₂—S— | —CH₂—C(═O)— | -2′-Docetaxel |
| CDc18 | HES 6 | —O—CH₂—CHOH—CH₂—S— | 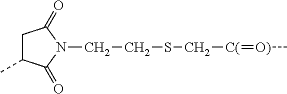 | -2′-Docetaxel |
| CDc19 | HES 6 | —O—CH₂—CHOH—CH₂—S— | 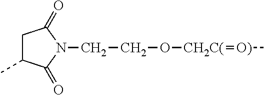 | -2′-Docetaxel |
| CDc20 | HES 4 | —S— | —CH₂—C(═O)— | -2′-Docetaxel |
| CDc21 | HES 5a | —S— | —CH₂—C(═O)— | -2′-Docetaxel |
| CDc22 | HES 9 | —S— | 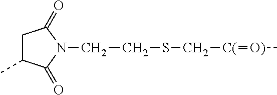 | -2′-Docetaxel |
| CDc23 | HES 9 | —O—CH₂—CHOH—CH₂—S— | 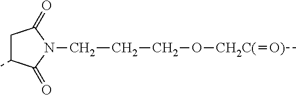 | -2′-Docetaxel |
| CDc24 | HES6 | —S— | —CH₂—C(═O)— | -2′-Docetaxel |
| CDc25 | HES 9 | —O—CH₂—CHOH—CH₂—S— | 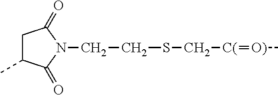 | -2′-Docetaxel |
| CDc26 | HES 9 | —S— | 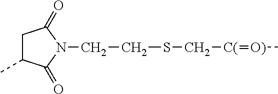 | -2′-Docetaxel |
| CDc27 | HES 9 | —S— | 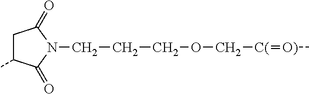 | -2′-Docetaxel |
| CDc28 | HES 5b | —S— | —CH₂—C(═O)— | -2′-Docetaxel |
| CDc29 | HES5a | —O—CH₂—CHOH—CH₂—S—CH₂—CH₂—S— | —CH₂—C(═O)— | -2′-Docetaxel |
| CDc30 | HES 6 | —S— | 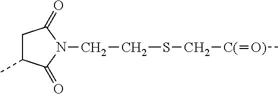 | -2′-Docetaxel |
| CDc31 | HEs 6 | —S— | 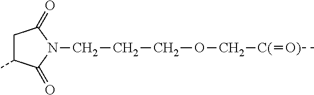 | -2′-Docetaxel |
| CDc32 | HES 1 | —S— | —CH₂—C(═O)— | -2′-Docetaxel |
| CDc33 | HES 5a | —O—CH₂—CHOH—CH₂—S— | 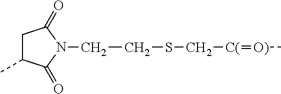 | -2′-Docetaxel |
| CDc34 | HES 5a | —S— | —CH₂—C(═O)— | -2′-Docetaxel |
| CDc35 | HES 5b | —S— | —CH₂—C(═O)— | -2′-Docetaxel |
| CPc1 | HES 6 | —O—C(═O)—NH—CH₂—CH₂—S— | —CH₂—C(═O)— | -2′-Paclitaxel |
| CPc2 | HES 14 | —O—CH₂—CHOH—CH₂—S— | —CH₂—C(═O)— | -2′-Paclitaxel |
| CPc3 | HES 6 | —O—C(═O)—NH—CH₂—CH₂—S— | —CH₂—CH₂—CH₂—CH₂—C(═O)— | -2′-Paclitaxel |
| CPc4 | HES 14 | —O—CH₂—CHOH—CH₂—S— | —CH₂—CH₂—CH₂—CH₂—C(═O)— | -2′-Paclitaxel |
| CPc5 | HES 6 | —O—CH₂—CHOH—CH₂—S—CH₂—CH₂—S— | —CH₂—C(═O)— | -2′-Paclitaxel |
| CPc6 | HES 6 | —O—CH₂—CHOH—CH₂—S— | 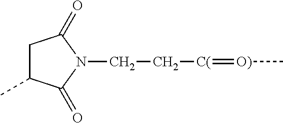 | -2′-Paclitaxel |
| CPc7 | HES 14 | —O—CH₂—CHOH—CH₂—S | 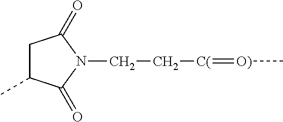 | -2′-Paclitaxel |
| CCx1 | HES17 | —S— | —CH₂—C(═O)— | -2′-Cabazitaxel |

TABLE 15a
Overview of the amount of cleaved hydroxyethyl starch conjugates in PBS
buffer pH 7.4 at 37° C. after 45 h, determined by HPLC

| | | | Conjugate |
|---|---|---|---|
| | | | cleaved |
| | Conjugate | Linker | [%] |
| 1 | CDc2 | —S—CH₂—CH₂—CH₂—CH₂—C(═O)— | 1.4 |
| 2 | CDc4 | 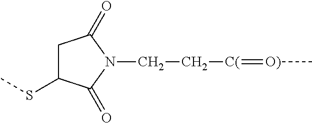 | 5.4 |
| 3 | CDc10 | —S—CH₂—C(═O)— | 29.3 |
| 4 | CDc24 | —S—CH₂—C(═O)— | 59.3 |
| 5 | CDc18 | 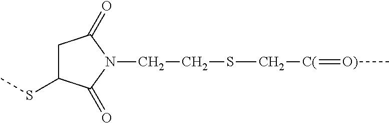 | 28.8 |
| 6 | CDc19 | 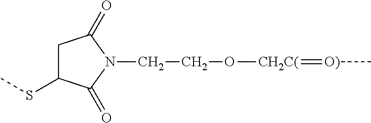 | 61.3 |

### In Vivo Studies

### 2.1 Test Animals

Adult male and female immunodeficient mice NMRI:nu/nu mice (TACONIC Europe, Lille Skensved, Denmark) were used throughout the study.

All mice were maintained under strictly controlled and standardized barrier conditions. They were housed—maximum four mice/cage—in individually ventilated cages (Macrolon Typ-II, system Techniplast, Italy). The mice were held under standardized environmental conditions: 22±1° C. room temperature, 50±10% relative humidity, 12 hour-light-dark-rhythm. They received autoclaved food and bedding (Ssniff, Soest, Germany) and acidified (pH 4.0) drinking water ad libitum.

The animals were randomly assigned to various experimental groups with 6 to 8 mice each. At treatment initiation the ears of the animals were marked and each cage was labeled with the cage number, study number and animal number per cage.

### 2.2 Tumor Models

The tumor lines A549, PC-3 and MT3 are lines which are commonly used for testing new anticancer drugs or novel therapeutic therapies. A549, PC-3 and MT3 xenografts are growing relatively fast and uniform.

Table 16 provides an overview of the tumor models used in studies described herein.

TABLE 16
Overview of the tumor models used in studies

| | | ATCC | |
|---|---|---|---|
| Name | tumor model | number | described in |
| A549 | human lung carcinoma | CCL-185 | Lieber, M. al., Int. J. |
| | | | Cancer 17: 62-70 (1976) |
| PC3 | human prostatic carcinoma | CRL-1435 | Kaighn ME. et al., Inv. |
| | | | Urol.17: 16-23 (1979) |
| MT3 | human breast cancer | | Naunhof H. et al. Breast |
| | | | Cancer Res Treat. 87-95 |
| | | | (1992) |

Tumor cells were thawed and grown under in vitro conditions. For experimental use, 10⁷ tumor cells/mouse were transplanted into the flank of the mice to be tested (male mice for PC-3, female mice for MT3 and A549). At palpable tumor size (30-100 mm³) treatment started (day 6). The application volume was 0.2 ml/20 g/mouse body weight. The test compounds, the vehicle controls and the reference compounds were all given intravenously (i.v.).

### 2.3 Therapeutic Evaluation

Tumor growth inhibition was used as therapeutic parameter. Additionally, body weight change was determined as signs for toxicity (particularly, potential hematological or gastrointestinal side effects).

### Tumor Measurement

Tumor diameters were measured twice weekly with a caliper. Tumor volumes were calculated according to V=(length×(width)²)/2. For calculation of the relative tumor volume (RTV) the tumor volumes at each measurement day were related to the day of first treatment. At each measurement day the median and mean tumor volumes per group and also the treated to control (T/C) values in percent were calculated.

### Body Weight

Individual body weights of mice were determined twice weekly and mean body weight per group was related to the initial value in percent (body weight change, BWC), see tables in appendix.

### End of Experiment

On the day of necropsy mice were sacrificed by cervical dislocation and inspected for gross organ changes.

### Statistics

Descriptive statistics was performed on the data of body weight and tumor volume. These data are reported in tables as median values, means and standard deviations, see tables in appendix. Statistical evaluation was performed with the U-test of Mann and Whitney with a significance level of p<0.05, using the Windows program STATISTICA 6.

### 2.4 Analysis of the Effects of Doxetaxel Conjugates on Tumor Growth and Body Weight

### 2.4.1 Tested Substances

The tested Docetaxel conjugates CDc1-CDc29 were obtained as described herein above and were kept in a freeze-dried form at −20° C. until use. Before administration, the conjugates were solved in saline solution by vortexing in combination with centrifugation until a clear solution of the necessary concentration of the drug was obtained. The obtained solutions were prepared and injected under sterile conditions.

The reference compound was Docetaxel (not conjugated, Taxotere®, Sanofi-Aventis Deutschland GmbH, Berlin, Germany). Docetaxel was stored in aliquots at 4° C. in the dark and diluted in saline before administration.

As a further control, saline solution was intravenously administered.

The above mentioned conjugates were tested in the MT3 tumor model (breast cancer). Three conjugates (CDc3, CDc5, CDc4) were additionally tested in the PC-3 (prostate cancer) and the A549 (lung cancer) model.

The following table provides an overview on the dosage scheme for the various tested substances. Usually, the Docetaxel conjugates were administered only once at a dosage of 75 mg/kg body weight. The conjugates CDc1 and CDc2 were administered once at a dosage of 100 mg/kg body weight. Usually, the reference compound Taxotere® was administered 5 times at a dosage of 5 mg/kg on 5 consecutive days each (or 3 times at dosage of 10 mg/kg body weight every second day). A more comprehensive overview on the dosage scheme can be found in tables 18-24.

TABLE 17
Treatment groups

| | | Dose | | Doses |
|---|---|---|---|---|
| Mice | | mg/kg body | | [application × |
| [n] | Substances | weight/appl | Route | mg/kg] |
| 6-8 | Saline | — | i.v. | — |
| 6-8 | Taxotere ® | 5 | i.v. | 5 × 5 |
| 6-8 | Docetaxel | 75-100 | i.v. | 1 × 75-100* |
| | conjugate | | | |

*amount of Docetaxel present in the conjugate

### 2.4.2 Test Results

Tables 18 to 24 summarize the results for the tested Docetaxel conjugates and the reference compound Taxotere®. The table shows, inter alia, i) the tested compounds, ii) the tumor volume in mice at the day the control group was sacrificed (in cm³), iii) the lowest value of the relative tumor volume vs. the relative tumor volume of the control group (RTV T/C) together with the day, when this optimum was reached, iv) the maximum body weight loss in mice together with the day, when this minimum was reached. The loss of body weight is known to be an indicator of gastro-intestinal and hepatotoxicity of the tested compound.

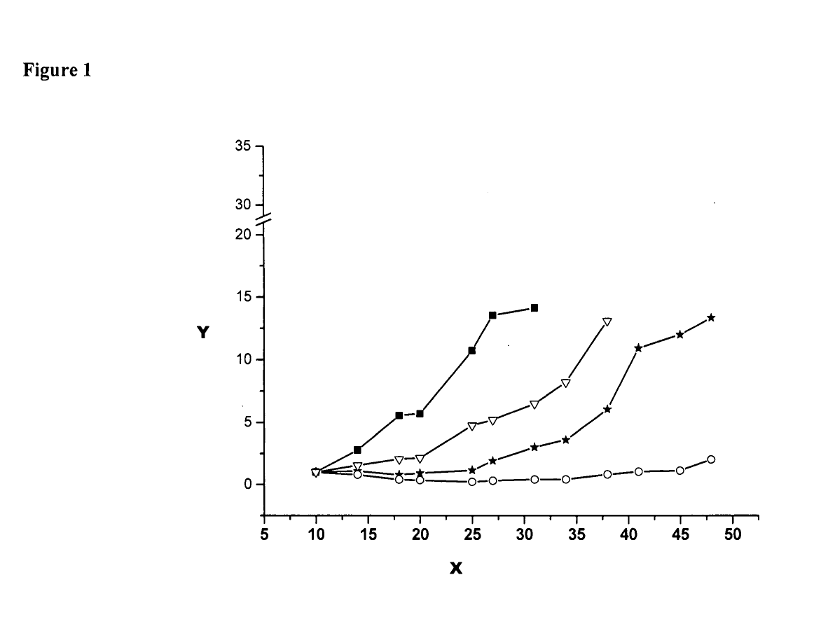

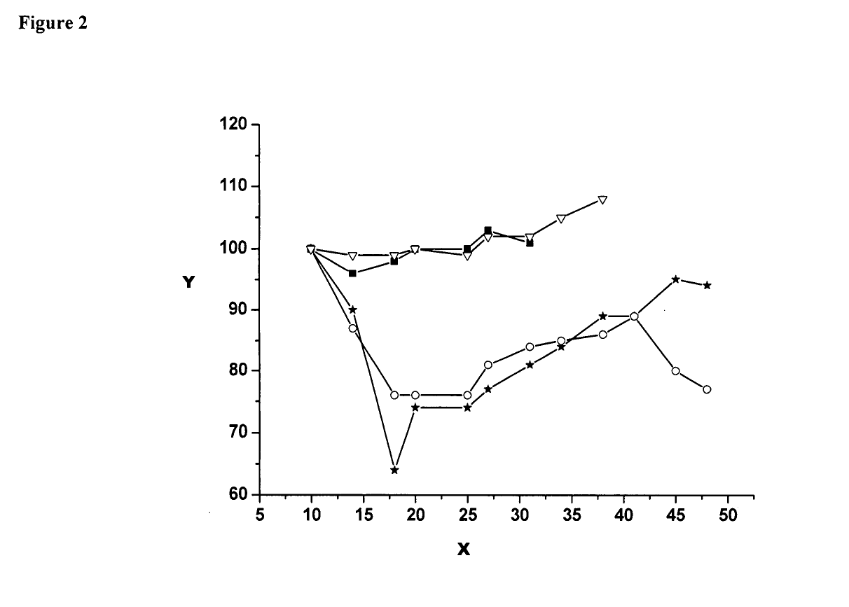

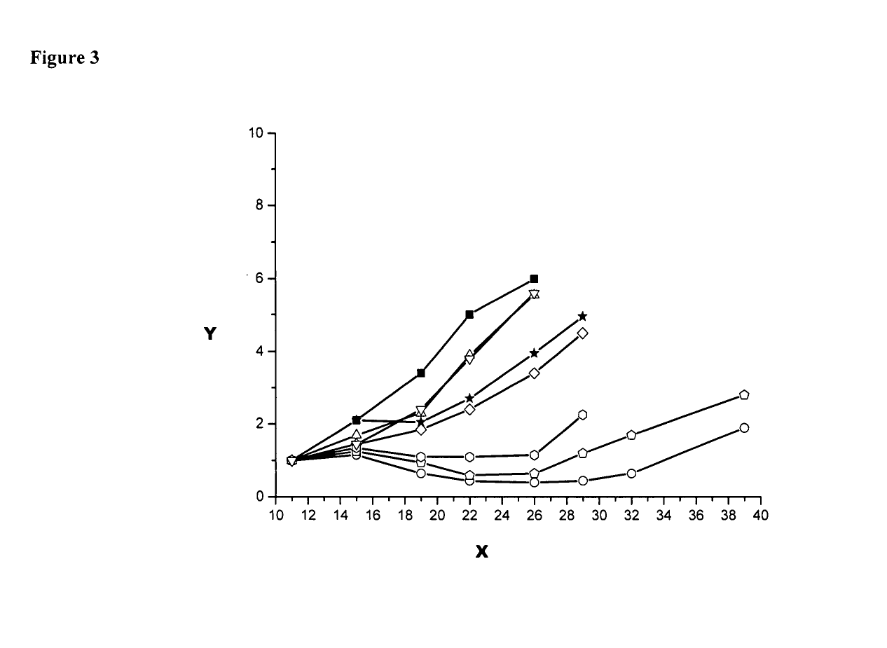

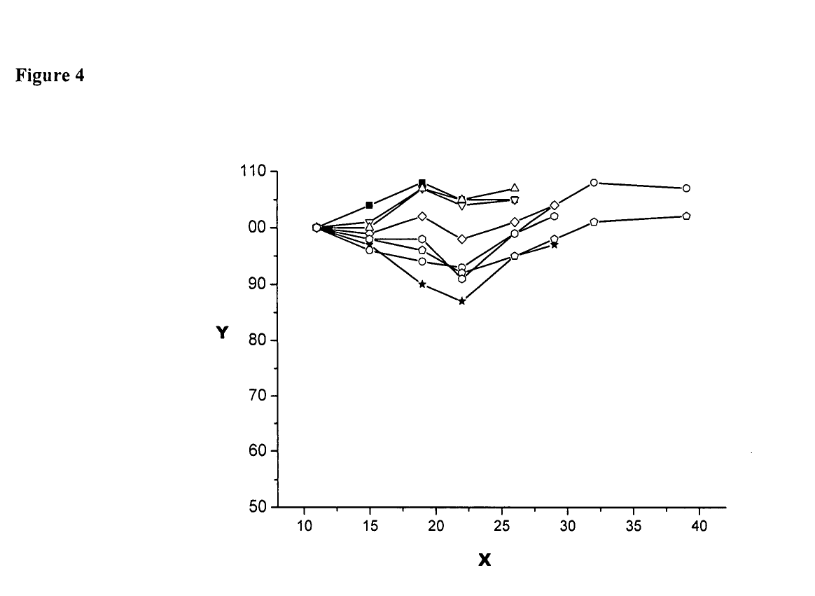

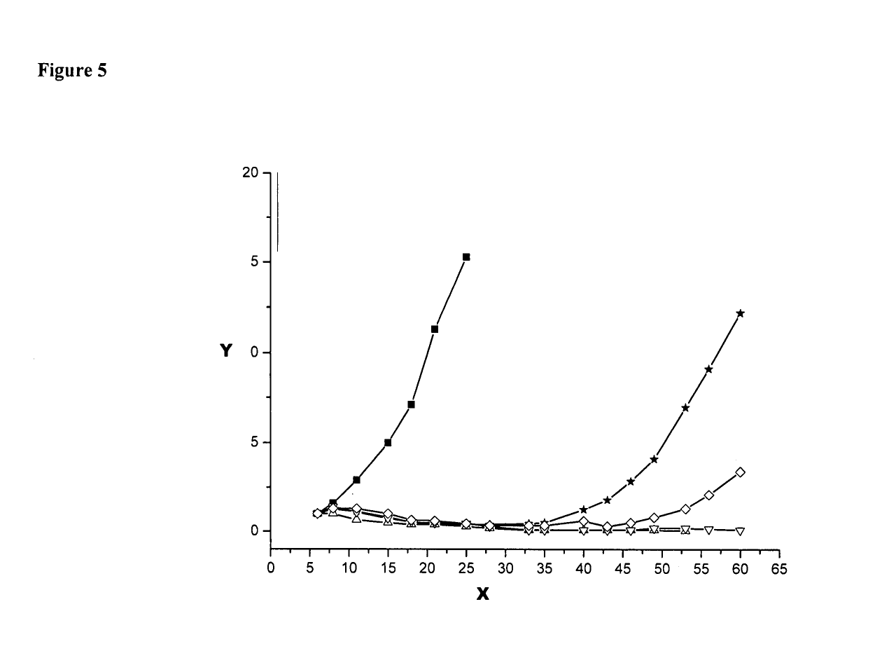

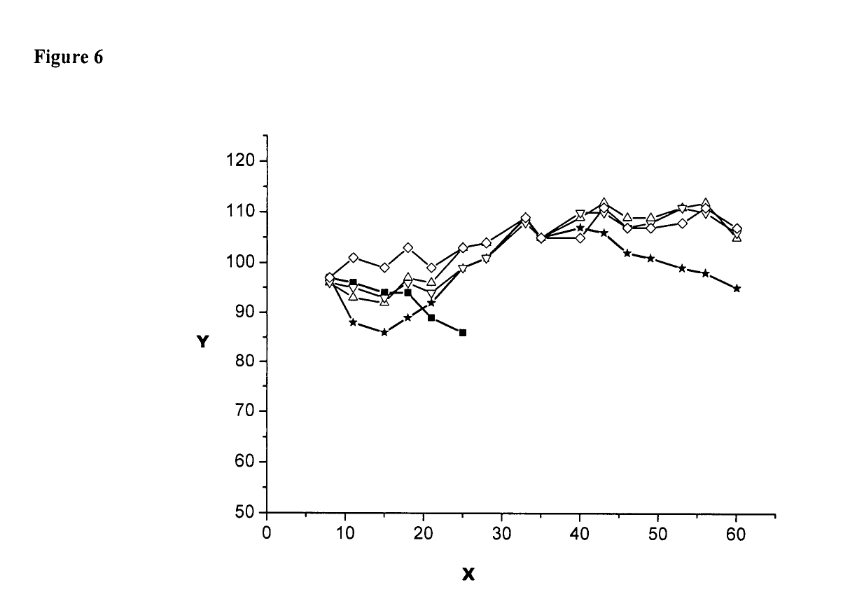

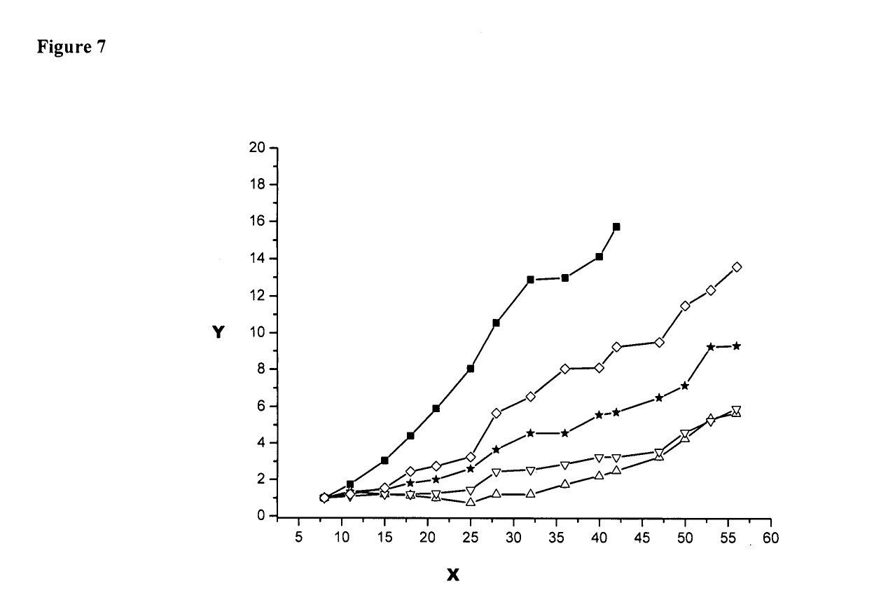

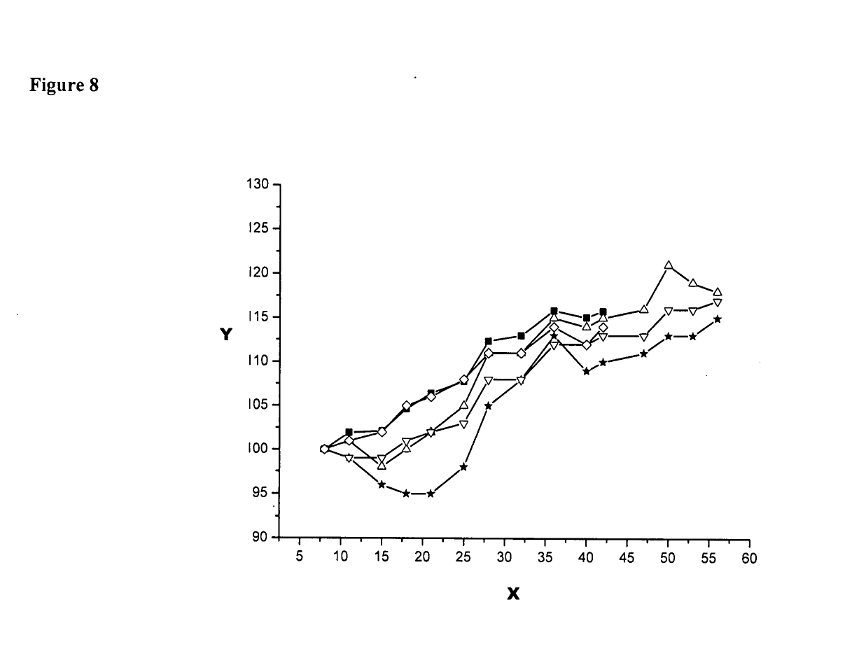

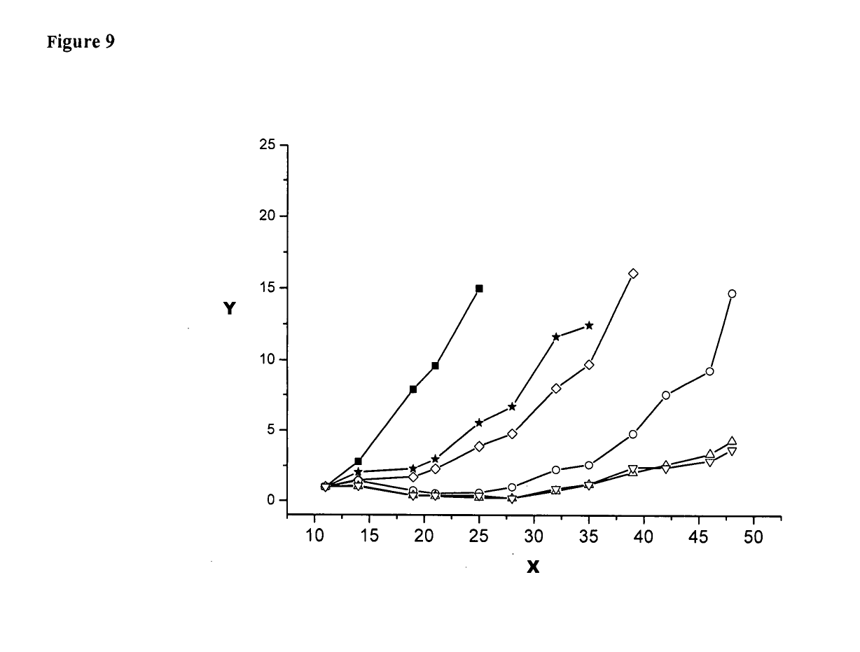

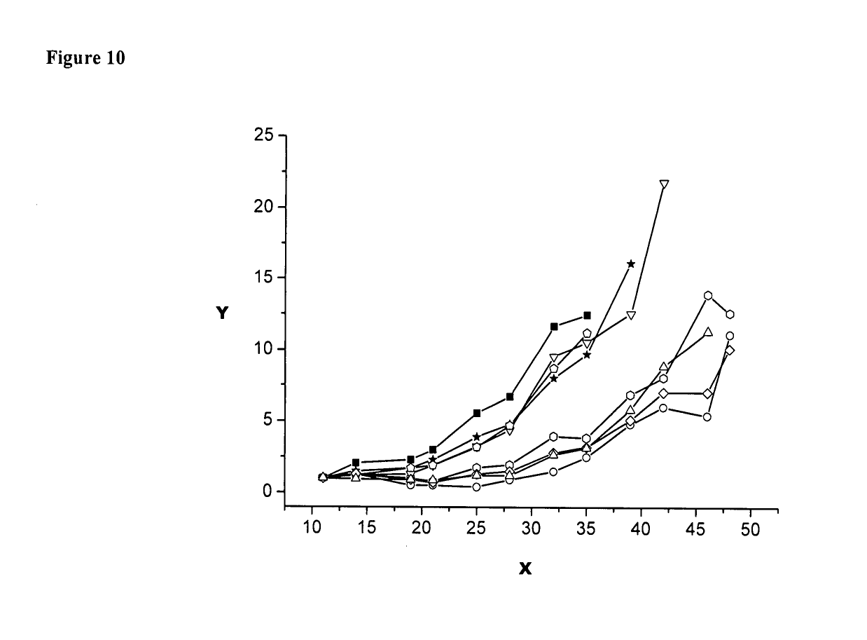

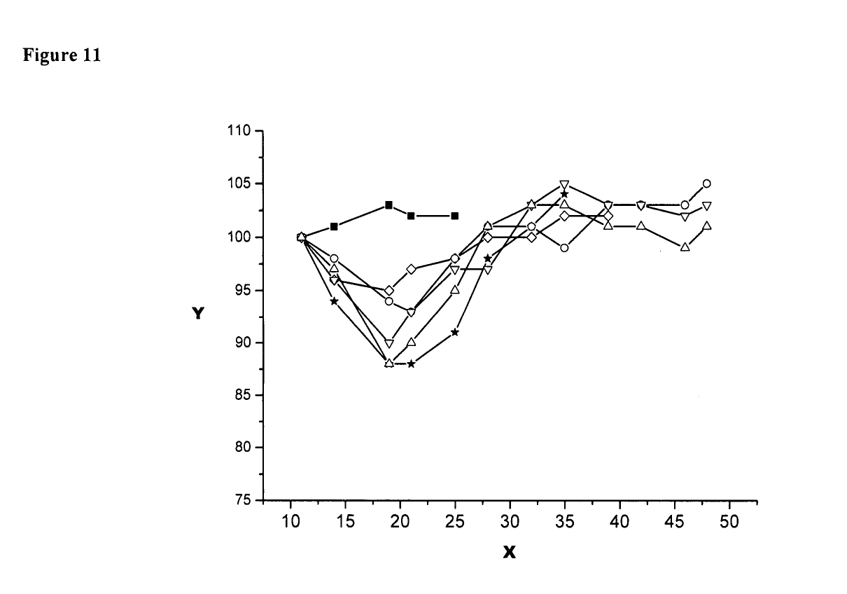

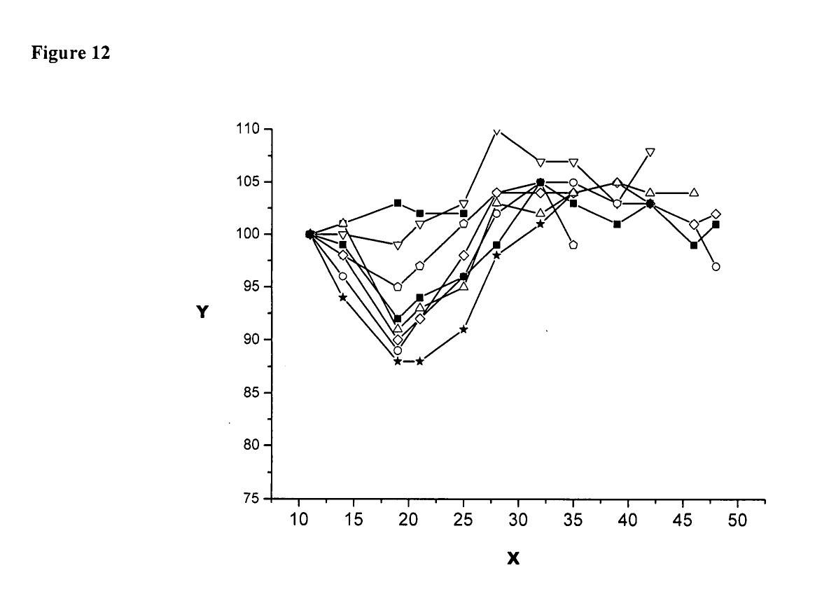

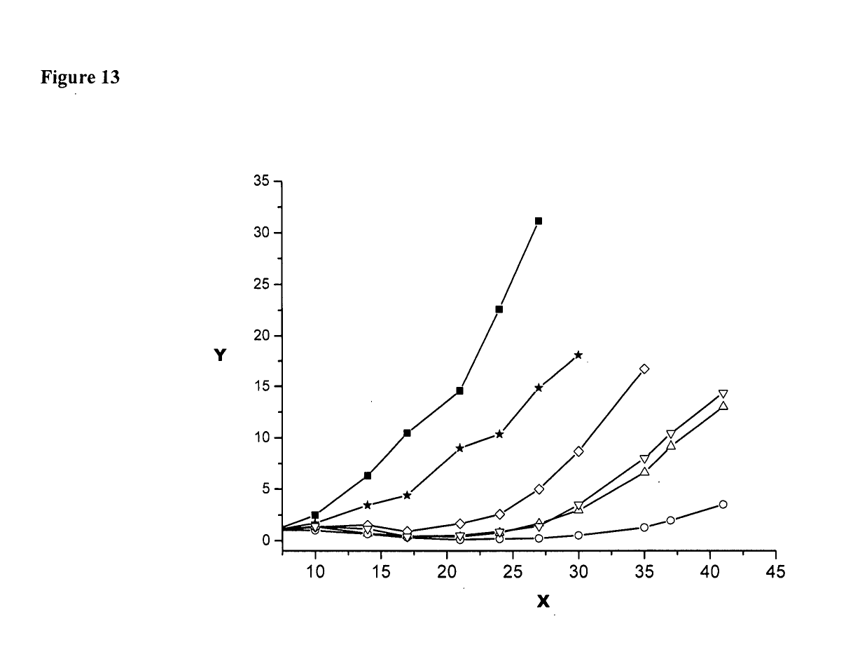

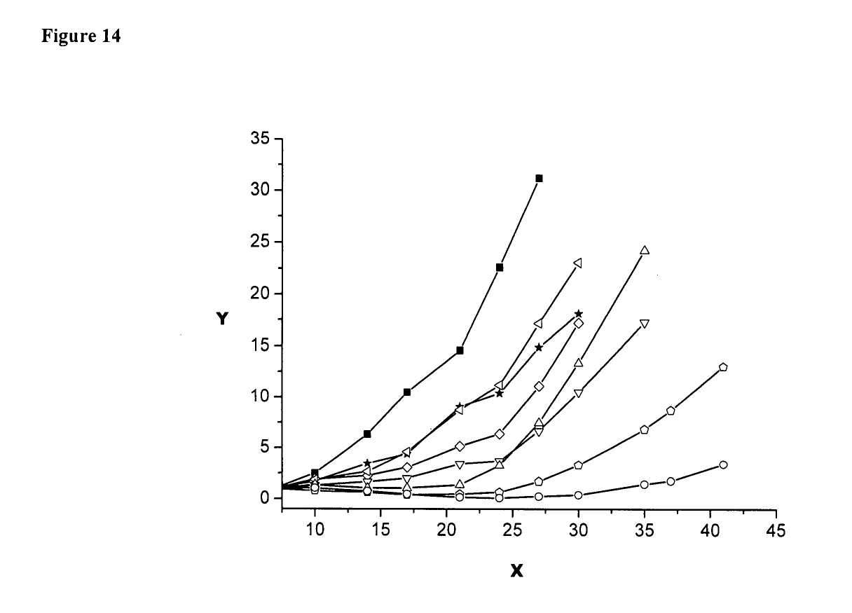

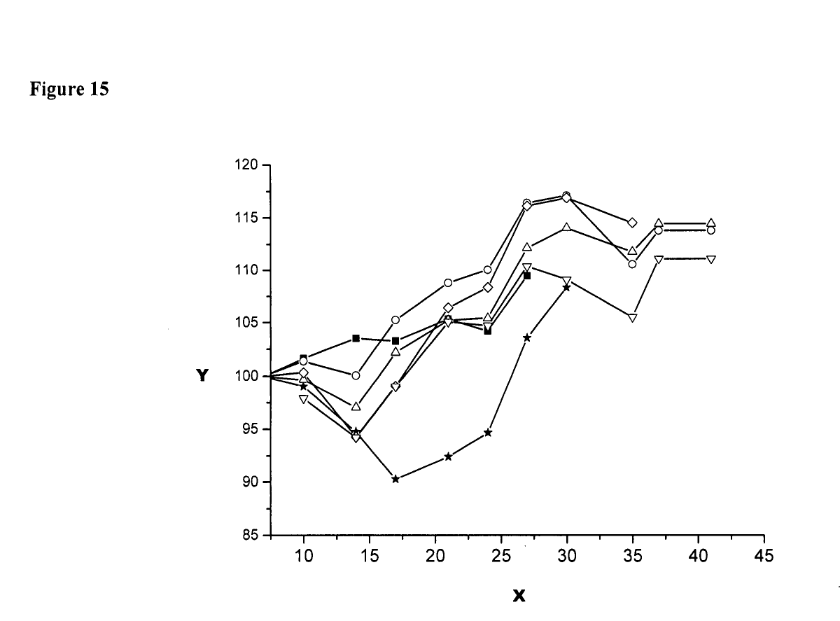

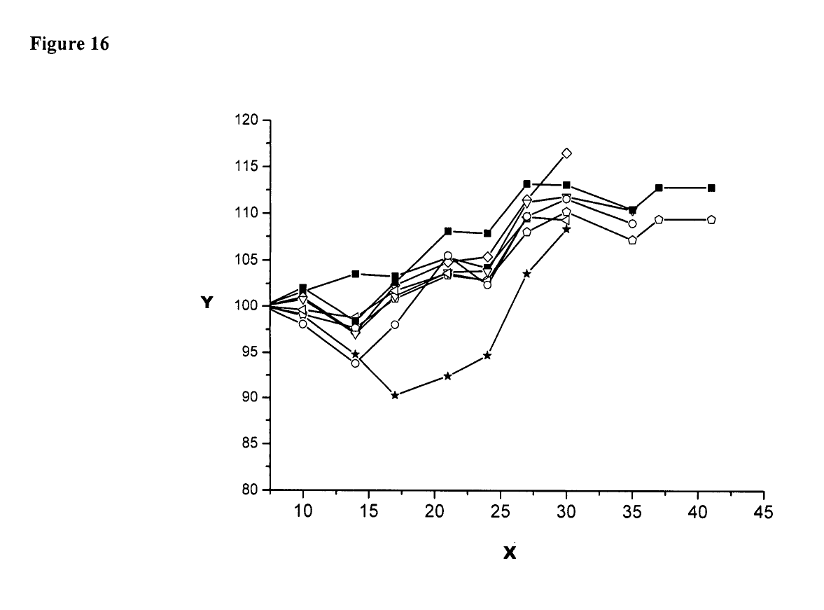

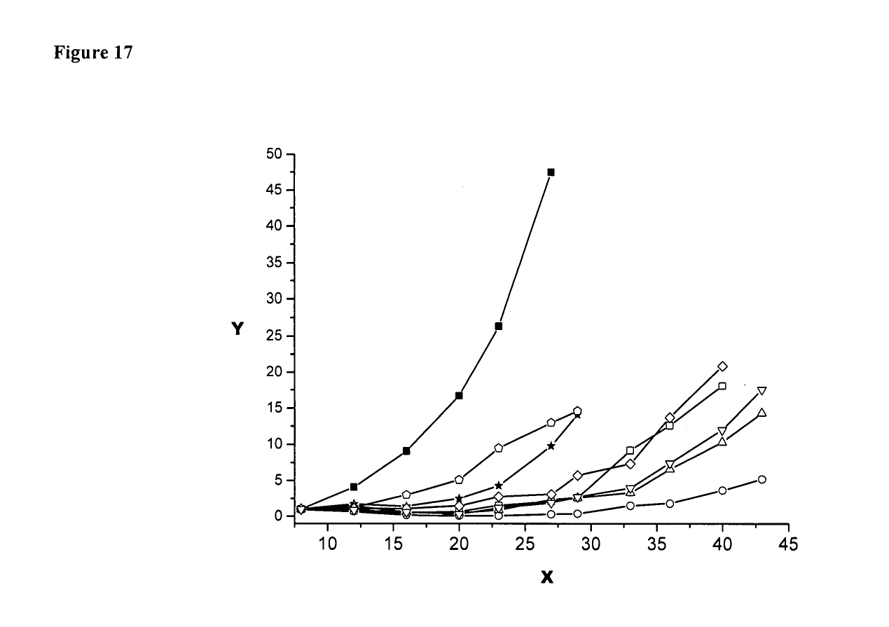

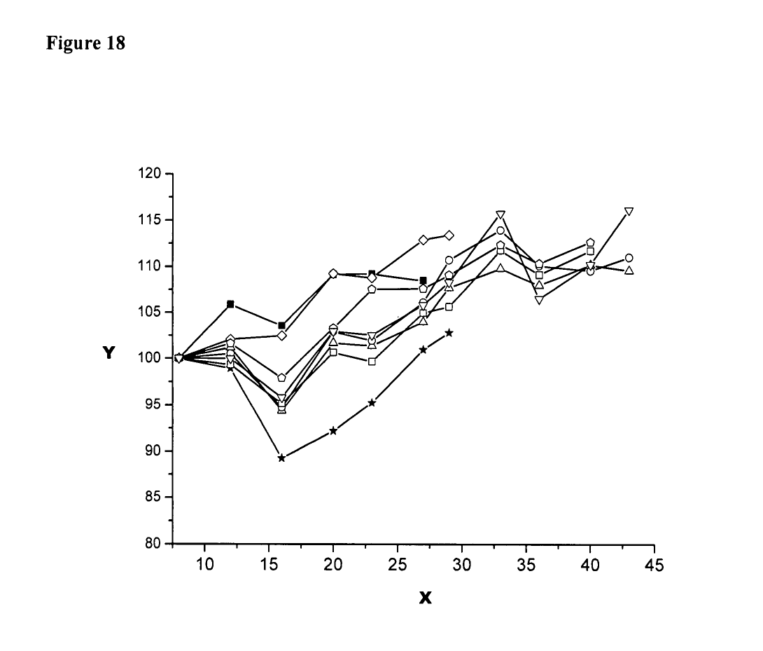

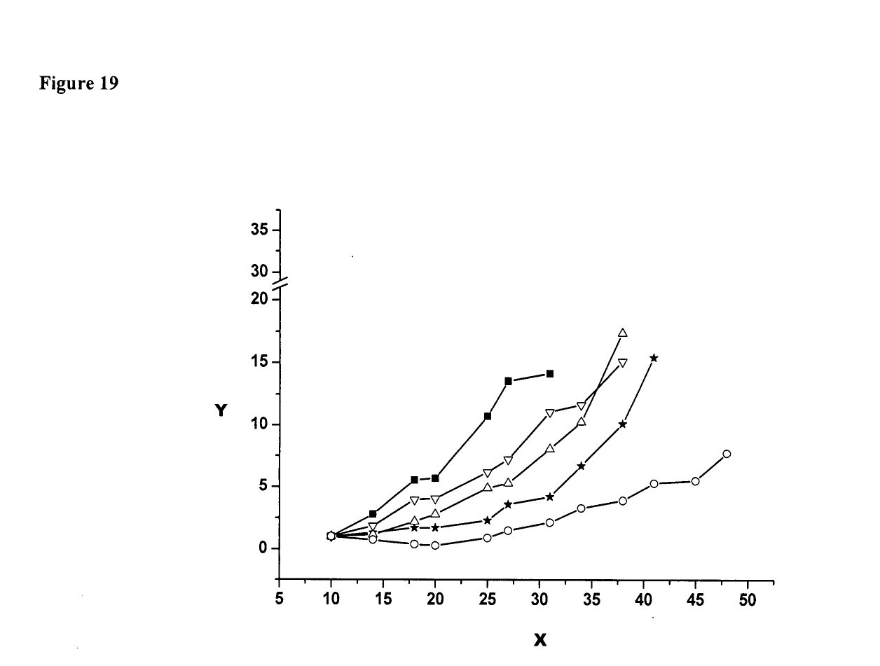

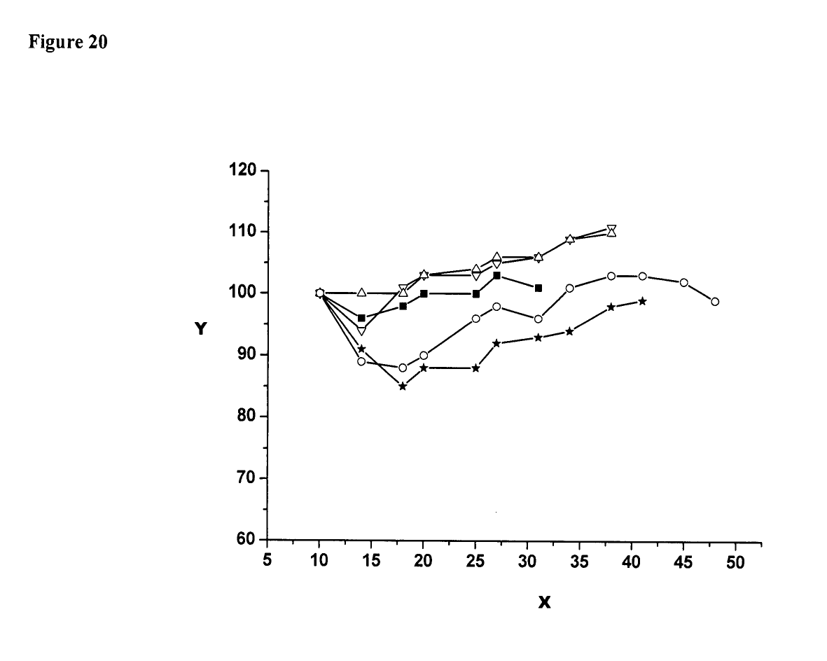

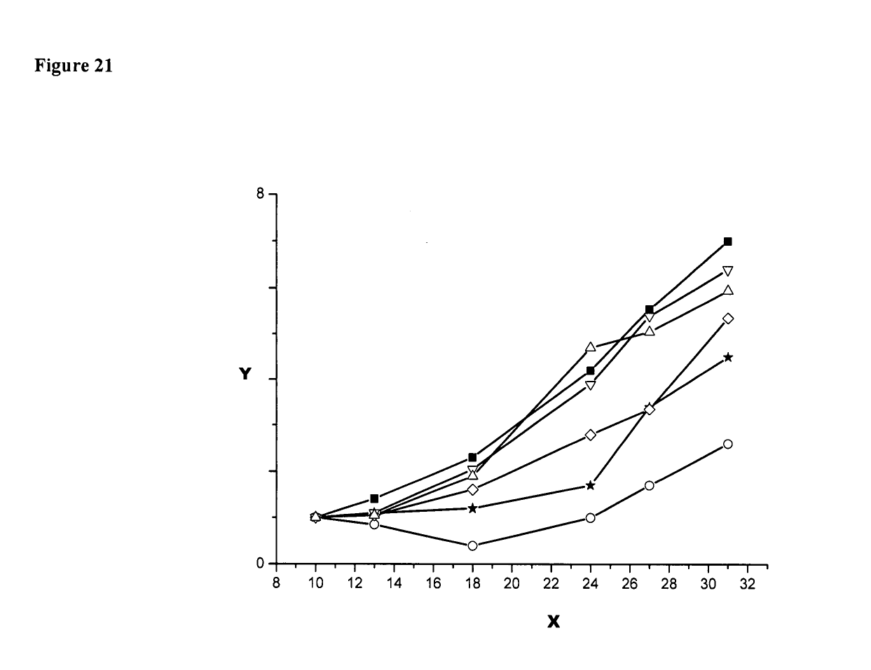

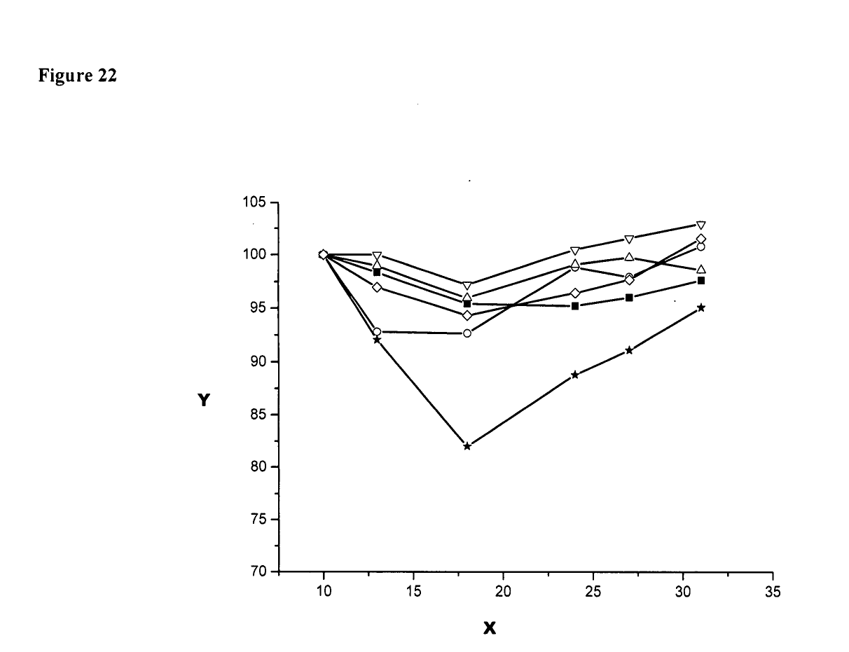

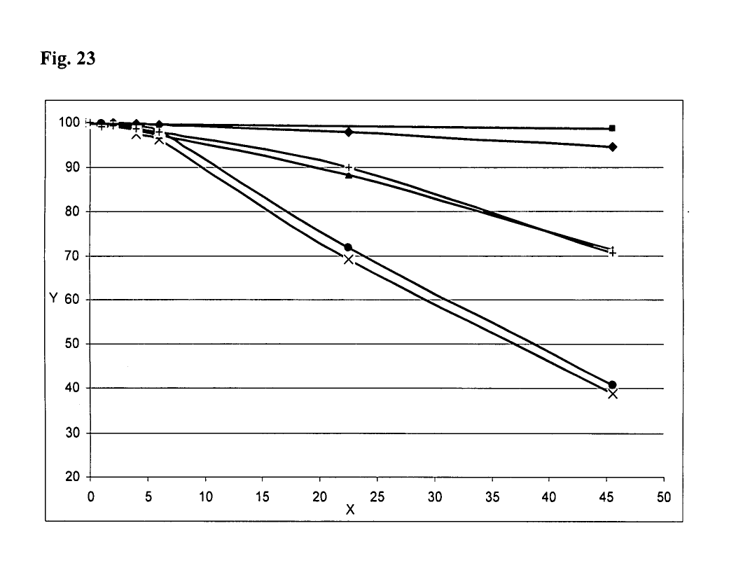

The time course of the body weight change as well as the relative tumor volume for the tested compounds and the reference compound Taxotere® (“referred to as “Docetaxel”) is shown in FIGS. 1 to 18.

As it can be seen from the tables 18 to 23 and the FIGS. 1 to 16, the administration of a Docetaxel conjugate i) allows for a more efficient reduction of tumor size and/or ii) is less toxic (as indicated by the body weight change) than the administration of non-conjugated Docetaxel. Moreover, PC-3 mice treated with the CDc1 and the CDc5 conjugate could be cured.

### 2.5 Analysis of the Effects of Paclitaxel Conjugates on Tumor Growth and Body Weight

### 2.5.1 Tested Substances

The tested Paclitaxel conjugates CPc1-CPc7 were obtained as described herein above and were kept in a freeze-dried form at −20° C. until use. Before administration, the same steps were carried out as described for the Docetaxel conjugates herein above.

The reference compound was Paclitaxel (not conjugated, for example, available as Neotaxan, Neocorp/Sandoz)

As a further control, saline solution was intravenously administered.

Further, the compounds were compared to ABRAXANE® (Paclitaxel, non-covalently bound to albumin nano particles, which, for example, is commercially available from Abraxis Bioscience).

The above mentioned conjugates were tested in the MT3 tumor model (breast cancer).

The Paclitaxel conjugates were administered only once at a dosage of 80 or 100 mg/kg body weight (with respect to the amount of Paclitaxel present in the conjugate). The conjugates CPc1-CPc3 were administered once at a dosage of 100 mg/kg body weight. The conjugates CPc5-CPc7 were administered once at a dosage of 80 mg/kg body weight. The reference compound Paclitaxel was administered 5 times at five consecutive days at a dosage of 10 or 12.5 mg/kg, A more comprehensive overview on the dosage scheme can be found in table 19.

### 2.5.2 Test Results

Tables 25 and 26 summarize the results for the tested Paclitaxel conjugates and the reference compounds. The tables show, inter alia, i) the tested compounds, ii) the tumor volume in mice at the day the control group was sacrificed (in cm³), iii) the lowest value of the relative tumor volume vs. the relative tumor volume of the control group (RTV T/C) together with the day, when this optimum was reached, iv) the maximum body weight loss in mice together with the day, when this minimum was reached. The loss of body weight is known to be an indicator of gastro-intestinal and hepatotoxicity of the tested compound.

The time course of the body weight change as well as the relative tumor volume for the tested compounds and the reference compounds are shown in FIGS. 19 to 22.

As it can be seen from tables 25 and 26 and the FIGS. 19 to 22, the administration of Paclitaxel conjugates allows for an efficient reduction of tumor size. Moreover, the conjugates are less toxic than the reference compounds (as indicated by the body weight change).

TABLE 18
Summary of the results for the tested Docetaxel conjugates (mouse tumor model MT-33)

| | | | | Toxic | BWC | Tumor | RTV T/C (%) |
|---|---|---|---|---|---|---|---|
| | Mice | Treatment | Dose | deaths | [%] | volume | Optimum |
| Group | n | (d) | (mg/kg/inj.) | (at day) | (at day) | cm³/d 31 | (at day) |
| Saline | 8 | 7-11 | | 0 | −4 | 1.48 +/− 1.10 | |
| | | | | | (14) | | |
| Docetaxel | 8 | 7, 9, 11 | 10 | 4 | −36 | 0.18 +/− 0.10 | 10.7 |
| | | | | (3x17, 20) | (18) | | (25) |
| CDc1 | 8 | 7 | 100 | 3 | −24 | 0.02 +/− 0.01** | 1.9 |
| | | | | (15, 17, 23) | (18-25) | | (25) |
| CDc2 | 8 | 7 | 100 | 0 | −1 | 0.61 +/− 0.23* | 36.9 |
| | | | | | (18-25) | | (18) |

*significantly different to saline (p < 0.05)
**significantly different to docetaxel (p < 0.05)

TABLE 19
Summary of the results for the tested Docetaxel conjugates (mouse tumor model MT-3)

| | | | | Group | Tumor | BWC | RTV T/C (%) |
|---|---|---|---|---|---|---|---|
| | Mice | Treatment | Dose | sacrif. | volume | [%] | Optimum |
| Group | n | (d) | (mg/kg/inj.) | (at day) | cm³/d 26 | (at day) | (at day) |
| Saline | 8 | 11 | | 26 | 1.534 +/− 0.267 | | |
| Docetaxel | 8 | 11-15 | 5 | 29 | 0.815 +/− 0.383* | −13 | 54.0 |
| | | | | | | (22) | (22) |
| CDc3 | 8 | 11 | 75 | 39 | 0.103 +/− 0.057* | −7 | 6.7 |
| | | | | | | (22) | (26) |
| CDc4 | 8 | 11 | 75 | 26 | 1.670 +/− 1.300 | +1 | 70.6 |
| | | | | | | (18) | (19) |
| CDc5 | 8 | 11 | 75 | 39 | 0.206 +/− 0.170* | −8 | 10.8 |
| | | | | | | (22) | (26) |
| CDc6 | 8 | 11 | 75 | 26 | 1.756 +/− 1.262 | | 67.6 |
| | | | | | | | (19) |
| CDc7 | 8 | 11 | 75 | 29 | 0.373 +/− 0.204* | −9 | 19.2 |
| | | | | | | (22) | (26) |
| CDc8 | 8 | 11 | 75 | 29 | 0.929 +/− 0.445* | −2 | 48.0 |
| | | | | | | (22) | (22) |

*significantly different to saline (p < 0.05)

TABLE 20
Summary of the results for the tested Docetaxel conjugates (mouse tumor model PC3)

| | | | | Toxic | Group | BWC | Tumor | RTV T/C (%) |
|---|---|---|---|---|---|---|---|---|
| | Mice | Treatment | Dose | death | sacrif. | [%] | volume | Optimum |
| Group | n | (d) | (mg/kg/inj.) | (at day) | (at day) | (at day) | cm³/d 25 | (at day) |
| Saline | 8 | 6 | | | 26 | −6 | 1.26 +/− 0.54 | |
| | | | | | | (15) | | |
| Docetaxel | 8 | 6-10 | 5 | | 60 | −14 | 0.04/−0.02* | 2.6 |
| | | | | | | (15) | | (25) |
| CDc1 | 8 | 6 | 75 | | 60 | −8 | 0.03 +/− 0.02* | cured |
| | | | | | | (15) | | |
| CDc5 | 8 | 6 | 75 | | 60 | −7 | 0.04 +/− 0.02* | cured |
| | | | | | | (15) | | |
| CDc4 | 8 | 6 | 75 | 1 | 60 | −3 | 0.05 +/− 0.03* | 2.9 |
| | | | | (40) | | (8) | | (25) |

*statistically different to saline

TABLE 21
Summary of the results for the tested Docetaxel conjugates (mouse tumor model A549)

| | | | | Group | Tumor | BWC | RTV T/C (%) |
|---|---|---|---|---|---|---|---|
| | Mice | Treatment | Dose | sacrif. | volume | [%] | Optimum |
| Group | n | (d) | (mg/kg/inj.) | (at day) | cm³/d 42 | (at day) | (at day) |
| Saline | 8 | 8 | | 42 | 0.879 +/− 0.313 | | |
| Docetaxel | 8 | 8-12 | 5 | 60 | 0.334 +/− 0.207* | −5 | 32.3 |
| | | | | | | (18-21) | (25) |
| CDc1 | 8 | 8 | 75 | 56 | 0.193 +/− 0.175* | −2 | 9.6 |
| | | | | | | (15) | (25.32) |
| CDc5 | 8 | 8 | 75 | 56 | 0.215 +/− 0.129* | −1 | 18.0 |
| | | | | | | (11-15) | (25) |
| CDc4 | 8 | 8 | 75 | 56 | 0.544 +/− 0.342 | 0 | 40.4 |
| | | | | | | | (25) |

TABLE 22
Summary of the results for the tested Docetaxel conjugates (mouse tumor model MT-3)

| | | | | Group | BWC | Toxic | Tumor | RTV T/C (%) |
|---|---|---|---|---|---|---|---|---|
| | Mice | Treatment | Dose | sacrif. | [%] | death | volume | Optimum |
| Group | n | (d) | (mg/kg/inj.) | (at day) | (at day) | (at day) | cm3/d 25 | (at day) |
| Saline | 8 | 11 | | 26 | | 1 | 1.306 +/− 0.450 | |
| | | | | | | 24 | | |
| Docetaxel | 8 | 11-15 | 5 | 35 | −12 | | 0.517 +/− 0.202* | 31.8 |
| | | | | | (19-21) | | | (19) |
| CDc8 | 7 | 11 | 75 | 39 | −5 | | 0.428 +/− 0.153* | 25.6 |
| | | | | | (19) | | | (19) |
| CDc9 | 6 | 11 | 75 | 48 | −8 | | 0.153 +/− 0.099** | 9.3 |
| | | | | | (21) | | | (21) |
| CDc10 | 7 | 11 | 75 | 48 | −12 | | 0.033 +/− 0.017** | 2.5 |
| | | | | | (21) | | | (25) |
| CDc11 | 7 | 11 | 75 | 35 | −5 | | 0.376 +/− 0.288* | 21.8 |
| | | | | | (21) | | | (19) |
| CDc12 | 7 | 11 | 75 | 48 | −10 | | 0.131 +/− 0.071** | 10.0 |
| | | | | | (21) | | | (25) |
| CDc13 | 8 | 11 | 75 | 42 | −1 | | 0.261 +/− 0.136** | 16.5 |
| | | | | | (21) | | | (19) |
| CDc14 | 7 | 11 | 75 | 48 | −11 | | 0.047 +/− 0.029** | 3.6 |
| | | | | | (21) | | | (25) |
| CDc15 | 8 | 11 | 75 | 48 | −7 | | 0.087 +/− 0.077** | 6.6 |
| | | | | | (21) | | | (25) |
| CDc16 | 8 | 11 | 75 | 46 | −9 | | 0.216 +/− 0.158** | 13.4 |
| | | | | | (21) | | | (21) |
| CDc17 | 8 | 11 | 75 | 48 | −10 | | 0.030 +/− 0.022** | 2.3 |
| | | | | | (21) | | | (25) |

*significantly different to saline (p < 0.05),
**Significantly different to docetaxel (p < 0.05)

TABLE 23
Summary of the results for the tested Docetaxel conjugates (mouse tumor model MT-3)

| | | | | Group | BWC | Toxic | Tumor | RTV T/C (%) |
|---|---|---|---|---|---|---|---|---|
| | Mice | Treatment | Dose | sacrif. | [%] | death | volume | Optimum |
| Group | n | (d) | (mg/kg/inj.) | (at day) | (at day) | (at day) | cm³/d 27 | (at day) |
| Saline | 8 | 7 | | 27 | | 0 | 1.673 +/− 0.693 | |
| Docetaxel | 8 | 7-11 | 5 | 31 | −10 | 0 | 0.873 +/− 0.390* | 43.0 |
| | | | | | (10) | | | (17) |
| CDc18 | 8 | 7 | 75 | 35 | −3 | 0 | 0.505 +/− 0.453* | 22.0 |
| | | | | | (17) | | | (17) |
| CDc19 | 8 | 7 | 75 | 41 | −2 | 0 | 0.028 +/− 0.033** | 1.0 |
| | | | | | (17) | | | (24) |
| CDc20 | 8 | 7 | 75 | 35 | −6 | 0 | 0.360 +/− 0.173** | 8.0 |
| | | | | | (17) | | | (17) |
| CDc21 | 6 | 7 | 75 | 41 | −0 | 0 | 0.015 +/− 0.015** | 0.6 |
| | | | | | (14) | | | (24) |
| CDc23 | 8 | 7 | 75 | 35 | −6 | 0 | 0.351 +/− 0.131** | 9.1 |
| | | | | | (17) | | | (17) |
| CDc24 | 8 | 7 | 75 | 41 | −6 | 0 | 0.100 +/− 0.092** | 3.1 |
| | | | | | (17) | | | (21) |
| CDc25 | 8 | 7 | 75 | 31 | −1 | 0 | 0.955 +/− 0.438 | 41.3 |
| | | | | | (17) | | | (14) |
| CDc26 | 8 | 7 | 75 | 31 | −3 | 0 | 0.601 +/− 0.246* | 28.4 |
| | | | | | (17) | | | (17) |
| CDc27 | 8 | 7 | 75 | 41 | −2 | 0 | 0.138 +/− 0.133** | 5.1 |
| | | | | | (17) | | | (21) |
| CDc35 | 8 | 7 | 75 | 41 | −3 | 0 | 0.099 +/− 0.080** | 2.2 |
| | | | | | (17) | | | (21) |

*significantly different to saline (p < 0.05),
**Significantly different to docetaxel (p < 0.05)

TABLE 24
Summary of the results for the tested Docetaxel conjugates (mouse tumor model MT-3)

| | | | | Group | BWC | Toxic | Tumor | RTV T/C (%) |
|---|---|---|---|---|---|---|---|---|
| | Mice | Treatment | Dose | sacrif. | [%] | death | volume | Optimum |
| Group | n | (d) | (mg/kg/inj.) | (at day) | (at day) | (at day) | cm³/d 27 | (at day) |
| Saline | 8 | 8 | | 27 | | | 2.081 +/− 0.776 | |
| Docetaxel | 7 | 8-12 | 5 | 30 | −11 | | 0.542 +/− 0.353* | 23.1 |
| | | | | | (16) | | | (20) |
| CDc29 | 8 | 8 | 75 | 43 | −4 | | 0.106 +/− 0.051** | 2.5 |
| | | | | | (16) | | | (20) |
| CDc30 | 7 | 8 | 75 | 40 | −5 | | 0.185 +/− 0.080** | 5.7 |
| | | | | | (16) | | | (20) |
| CDc31 | 8 | 8 | 75 | 30 | +2 | 1 | 0.660 +/− 0.450* | 31.7 |
| | | | | | (12-16) | (13) | | (27) |
| CDc32 | 8 | 8 | 75 | 43 | −6 | | 0.143 +/− 0.097** | 3.7 |
| | | | | | (16) | | | (20) |
| CDc33 | 8 | 8 | 75 | 40 | −2 | | 0.193 +/− 0.108** | 9.3 |
| | | | | | (16) | | | (27) |
| CDc34 | 7 | 8 | 75 | 43 | −5 | | 0.035 +/− 0.036** | 0.7 |
| | | | | | (16) | | | (23) |

*significantly different to saline (p < 0.05)
**significantly different to docetaxel (p < 0.05)

TABLE 25
Summary of the results for the tested Paclitaxel conjugates (mouse tumor model MT-3)

| | | | | Toxic | BWC | Tumor | RTV T/C (%) |
|---|---|---|---|---|---|---|---|
| | Mice | Treatment | Dose | deaths | [%] | volume | Optimum |
| Group | n | (d) | (mg/kg/inj.) | (at day) | (at day) | cm³/d 31 | (at day) |
| Saline | 8 | 7-11 | | 0 | −4 | 1.48 +/− 1.10 | |
| | | | | | (14) | | |
| Paclitaxel | 8 | 7-9; | 12.5 | 1 | −15 | 0.41 +/− 0.21* | 21.5 |
| | | 10-11 | 10 | (12) | (18) | | (25) |
| CPc1 | 8 | 7 | 100 | 2 | −12 | 0.23 +/− 0.15* | 5.3 |
| | | | | (17, 22) | (18) | | (25) |
| CPc2 | 8 | 7 | 100 | 0 | 0 | 1.01 +/− 0.76 | 53.1 |
| | | | | | (10-18) | | (20) |
| CPc3 | 8 | 7 | 100 | 0 | −6 | 0.78 +/− 0.55 | 39.1 |
| | | | | | (14) | | (27) |

*significantly different to saline (p < 0.05)

TABLE 26
Summary of the results for the tested Paclitaxel conjugates (mouse tumor model MT-3)

| | | | | Toxic | BWC | Tumor | RTV T/C (%) |
|---|---|---|---|---|---|---|---|
| | Mice | Treatment | Dose | deaths | [%] | volume | Optimum |
| Group | n | (d) | (mg/kg/inj.) | (at day) | (at day) | cm³/d 31 | (at day) |
| Saline | 8 | 10 | | 0 | −5 | 0.958 +/− 0.223 | |
| | | | | | (18-24) | | |
| Paclitaxel | 8 | 10-14 | 10 | 0 | −18 | 0.673 +/− 0.226* | 40.5 |
| | | | | | (18) | | (24) |
| CPc4 | 8 | 10 | 80 | 0 | −4 | 1.011 +/− 0.467 | 75.0 |
| | | | | | (18) | | (13) |
| CPc5 | 8 | 10 | 80 | 1 | −7 | 0.367 +/− 0.251* | 17.4 |
| | | | | (18) | (13-18) | | (18) |
| CPc6 | 8 | 10 | 80 | 0 | −3 | 0.780 +/− 0.400 | 60.4 |
| | | | | | (18) | | (27) |
| CPc7 | 8 | 10 | 80 | 1 | −6 | 1.197 +/− 0.736 | 78.6 |
| | | | | (31) | (18) | | (13) |

*significantly different to saline (p < 0.05)

1-50. (canceled)

51. A hydroxyalkyl starch (HAS) conjugate comprising a hydroxyalkyl starch derivative and a cytotoxic agent, said conjugate having a structure according to the following formula
HAS′(-L-M)ₙ
wherein
M is a residue of a cytotoxic agent, wherein the cytotoxic agent comprises a secondary hydroxyl group,
L is a linking moiety,
HAS′ is a residue of the hydroxyalkyl starch derivative,
n is greater than or equal to 1, preferably in the range of from 3 to 200,
and wherein the hydroxyalkyl starch derivative has a mean molecular weight MW above the renal threshold, preferably in the range of from 60 to 800 kDa, more preferably of from 80 to 800 kDa,
and a molar substitution MS in the range of from 0.6 to 1.5,
and wherein the linking moiety L is linked to the secondary hydroxyl group of the cytotoxic agent, and wherein the cytotoxic agent is a taxane.

52. The conjugate according to claim 51, wherein the hydroxyalkyl starch derivative has
a mean molecular weight MW in the range of from 90 to 350 kDa, preferably in the range of from 95 to 150 kDa, and/or
a molar substitution MS in the range of from 0.70 to 1.45, more preferably in the range of 0.80 to 1.40, more preferably in the range of from 0.85 to 1.35, more preferably in the range of from 0.90 to 1.10, most preferably in the range of from 0.95 to 1.05.

53. The conjugate according to claim 51, wherein the linking moiety L has a structure -L′-F³—, wherein F³ is a functional group linking L′ with the secondary hydroxyl group of the cytotoxic agent thereby forming a —F³—O— bond, preferably wherein F³ is a —C(═Y)— group, with Y being O, NH or S, with Y being in particular O or S, and wherein L′ is a linking moiety, preferably wherein the conjugate comprises an electron-withdrawing group in alpha or beta position to each F³ group, preferably wherein the electron-withdrawing group is a group selected from the group consisting of —NH—C(═O)—, —C(═O)—NH—, —NH—, —O—, —S—, —SO—, —SO₂— and -succinimide-.

54. The conjugate according to claim 53, wherein L′ has a structure according to the following formula
-[F²]_{q}-[L²]_{g}-[E]ₑ—[CR^{m}Rⁿ]_{f}-
wherein E is an electron-withdrawing group, preferably selected from the group consisting of —C(═O)—NH—, —NH—, —O—, —S—, —SO—, -succinimide- and —SO₂—,
L² is a linking moiety, preferably selected from the group consisting of alkyl, alkenyl, alkylaryl, arylalkyl, aryl, heteroaryl, alkylheteroaryl and heteroarylalkyl,
F² is a group consisting of —Y¹—, —C(═Y²)—, —C(═Y²)—NR^{F2}—, 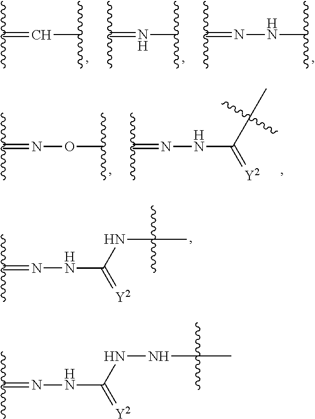
and —CH₂—CH₂—C(═Y²)—NR^{F2}—,
wherein Y′ is selected from the group consisting of —S—, —O—, —NH—, —NH—NH—, —CH₂—CH₂—SO₂—NR^{F2}—, —CH₂—CHOH—, and cyclic imides, and wherein Y² is selected from the group consisting of NH, S and O, and wherein R^{F2} is selected from the group consisting of hydrogen, alkyl, alkylaryl, arylalkyl, aryl, heteroaryl, alkylheteroaryl or heteroarylalkyl group,
f is 1, 2 or 3, preferably 1 or 2, most preferably 1, g is 0 or 1, q is 0 or 1, e is 0 or 1,
and wherein R^{m} and Rⁿ are, independently of each other, H or alkyl, preferably H or methyl, in particular H.

55. The conjugate according to claim 51, wherein the hydroxyalkyl starch derivative comprises at least one structural unit according to the following formula, preferably at least 3 to 200 structural units according to the following formula (I) 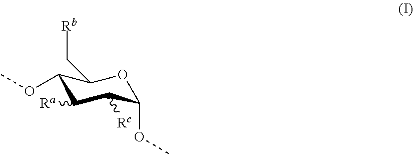
wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—X—, —[O—(CR^{w}R^{x})—(CR^{y}R^{z})]_{y}—[F¹]ₚ—L¹—X—, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{c} is —[O—(CR^{w}R^{x})—(CR^{y}R^{z})]_{y}—X— or —[O—(CR^{w}R^{x})—(CR^{y}R^{z})]_{y}-[F¹]ₚ-L¹-X—,
preferably wherein R^{a}, R^{b} and R^{c} are independently of each other selected from the group consisting of —O—HAS″, —[O—CH₂—CH₂]ₛ—OH, —[O—CH₂—CH₂]ₜ—X— and —[O—CH₂—CH₂]ₜ—[F¹]ₚ—L¹—X—, wherein t is in the range of from 0 to 4, and wherein s is in the range of from 0 to 4 and wherein at least one of R^{a}, R^{b} and R^{c} is —[O—CH₂—CH₂]ₜ—X— or —[O—CH₂—CH₂]ₜ-[F¹]ₚ-L¹-X—,
and wherein X is selected from the group consisting of —Y^{xx}—, —C(═Y^{x})—, —C(═Y^{x})—NR^{xx}—, 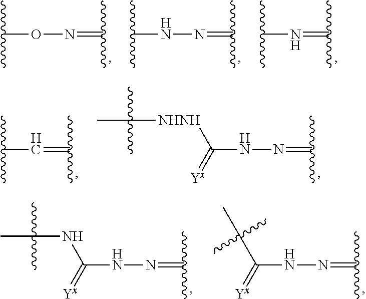
and —CH₂—CH₂—C(═Y^{x})—NR^{xx}—, wherein Y^{xx} is selected from the group consisting of —S—, —O—, —NH—, —NH—NH—, —CH₂—CH₂—SO₂—NR^{xx}—, and cyclic imides, such as succinimide, and wherein Y^{x} is selected from the group consisting of NH, S and O, and wherein R^{xx} is selected from the group consisting of hydrogen, alkyl, alkylaryl, arylalkyl, aryl, heteroaryl, alkylheteroaryl or heteroarylalkyl group, preferably wherein X is —S—,
F¹ is a functional group, preferably selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, -succinimide, —NH—NH—, —NH—O—, —CH═N—O—, —O—N═CH—, —CH═N—, —N═CH—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,
L¹ is a linking moiety, preferably selected from the group consisting of alkyl, alkenyl, alkylaryl, arylalkyl, aryl, heteroaryl, alkylheteroaryl and heteroarylalkyl, and wherein HAS″ is a remainder of HAS, preferably wherein
L′, is covalently linked to the —[O—CH₂—CH₂]ₜ—X— group or the —[O—CH₂—CH₂]ₜ—[F¹]ₚ—L¹—X— group,
more preferably wherein at least one of R^{a}, R^{b} and R^{c} is
(i)-[O—CH₂—CH₂]ₜ—X— and X is —S—, or
(ii)-[O—CH₂—CH₂]ₜ-[F¹]ₚ-L¹-X— with X being —S—, preferably with p being 1 and F¹ being —O—,
and wherein the structural unit -L-M is linked directly to the group X via the linking moiety L.

56. The conjugate according to claim 51, wherein the cytotoxic agent is docetaxel or paclitaxel, more preferably wherein the conjugate has a structure according to the following formula 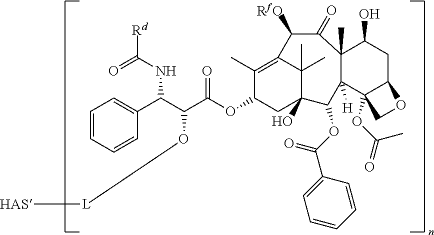
wherein R^{d} is preferably phenyl or O-t-butyl, and wherein R^{f} is preferably H or acetyl.

57. The conjugate according to claim 54, wherein L¹ has a structure according to the following formula
—[F²]_{q}—[L²]_{g}—[E]ₑ—[CR^{m}Rⁿ]_{f}—
wherein e is 1, and wherein E is —O— or —S—.

58. The conjugate according to claim 55, wherein HAS′ comprises at least one structural unit, preferably 3 to 200 structural units, according to the following formula (I) 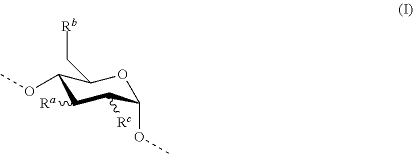
wherein R^{a}, R^{b} and R^{c} are
(i) independently of each other selected from the group consisting of —O—HAS″, —[O—CH₂—CH₂]ₛ—OH and —[O—CH₂—CH₂]ₜ—X—, with X being —S— wherein at least one of R^{a}, R^{b} and R^{c} is —[O—CH₂—CH₂]ₜ—X, or
(ii) independently of each other selected from the group consisting of —O—HAS″, —[O—CH₂—CH₂]ₛ-—OH and —[O—CH₂—CH₂]ₜ-[F¹]ₚ-L¹-X, with p being 1, and with X being —S—, wherein at least one of R^{a}, R^{b} and R^{c} is —[O—CH₂—CH₂]ₜ-[F¹]ₚ-L¹-X—,
and wherein t is in the range of from 0 to 4, and wherein s is in the range of from 0 to 4 and wherein L¹ is linked directly to the group X, and wherein F³ is —C(═O)—, and wherein F³ is being attached to the secondary hydroxyl group of the cytotoxic agent, thereby forming a —C(═O)—O— bond.

59. The conjugate according to claim 54 having a structure according to the following formula
HAS′(—[F²]_{q}-[L²]_{g}—[E]ₑ—[CR^{m}Rⁿ]_{f}—F³-M)ₙ
wherein q is 0, g is 0, e is 0, and wherein HAS′ comprises at least one structural unit, preferably 3 to 200 structural units, according to the following formula (I) 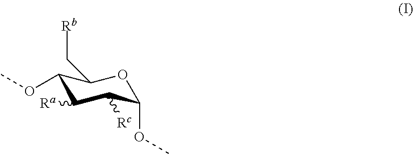
wherein at least one of R^{a}, R^{b} and R^{c} is —[O—CH₂—CH₂]-X— and X is —S— and the functional group X is directly linked to the —[CR^{m}Rⁿ]_{f}— group, and wherein the hydroxyalkyl starch derivative comprises at least n functional groups X,
preferably wherein f is 1,
more preferably wherein f is 1 and R^{m} and Rⁿ are H,
most preferably wherein the conjugate has a structure according to the following formula 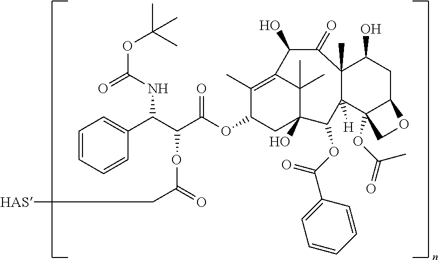
or the following formula

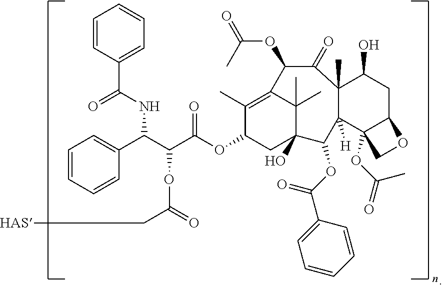

60. The conjugate according to claim 54, the conjugate having a structure according to the following formula
HAS′(—[F²]_{q}-[L²]_{g}-[E]ₑ—[CR^{m}Rⁿ]_{f}—F³-M)ₙ
wherein HAS′ comprises at least one structural unit, preferably 3 to 200 structural units, according to the following formula (I) 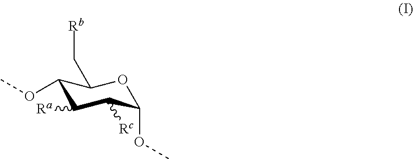
wherein at least one of R^{a}, R^{b} and R^{c} is —[O—CH₂—CH₂]ₜ—X— with X being —S—, wherein e is 1 and E is —S— or —O—, and wherein g and q are both 1,
preferably wherein F² is —S— or -succinimide-, in particular -succinimide-, most preferably wherein L² is CH₂—CH₂— and the conjugate has the structure
HAS′(-succinimide-CH₂—CH₂-E-[CR^{m}Rⁿ]_{f}—C(═O)-M)ₙ,
wherein R^{m} and Rⁿ are both H and f is 1.

61. The conjugate according to claim 54, the conjugate having a structure according to the following formula,
HAS′(—[F²]_{q}-[L²]_{g}-[E]ₑ—[CR^{m}Rⁿ]_{f}—F³-M)ₙ
wherein HAS′ comprises at least one structural unit, preferably 3 to 200 structural units, according to the following formula (Ib) 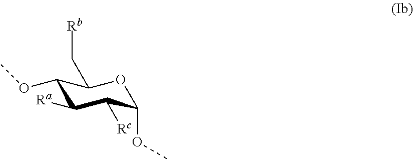
wherein at least one of R^{a}, R^{b} and R^{c} is —[O—CH₂—CH₂]ₜ-[F¹]ₚ-L¹-X— with X being —S—, preferably with p being 1 and F¹ being —O—,
wherein L¹ is preferably an alkyl chain, more preferably L¹ has a structure according to the following formula —{[CR^{d}R^{f}]ₕ-[F⁴]ᵤ—[CR^{dd}R^{ff}]_{z}}ₐₗₚₕₐ—, wherein F⁴ is a functional group, preferably a group selected from the group consisting of —S—, —O— and —NH—, in particular —S—,
wherein z is in the range of from 0 to 20, more preferably of from 0 to 10, more preferably 0 to 3, most preferably 0 to 2, and wherein h is in the range of from 1 to 5, preferably in the range of from 1 to 3, more preferably 3,
and wherein u is 0 or 1,
integer alpha is in the range of from 1 to 10,
and wherein R^{d}, R^{f}, R^{dd} and R^{ff} are, independently of each other, selected from the group consisting of H, alkyl, hydroxyl, and halogen, preferably selected from the group consisting of H, methyl and hydroxyl,
and wherein each repeating unit of —[CR^{d}R^{f}]ₕ—[F⁴]ᵤ—[CR^{dd}R^{ff}]_{z}— may be the same or may be different,
more preferably wherein L¹ has a structure selected from the group consisting of —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—S—CH₂—CH₂—, —CH₂—CH₂—S—CH₂—CH₂—, —CH₂—CH₂—O—CH₂—CH₂—, —CH₂—CH₂—O—CH₂—CH₂—O—CH₂—CH₂—, —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂—, —CH₂—CHOH—CH₂—NH—CH₂—CH₂—CH₂—, —CH₂—CHOH—CH₂—O—CH₂—CHOH—CH₂—, —CH₂—CHOH—CH₂—O—CH₂—CHOH—CH₂—S—CH₂—CH₂—, —CH₂—CH(CH₂OH)— and —CH₂—CH(CH₂OH)—S—CH₂—CH₂—, more preferably from the group consisting of —CH₂—CHOH—CH₂—, —CH₂—CHOH—CH₂—S—CH₂—CH₂—, —CH₂—CHOH—CH₂—S—CH₂—CH₂—CH₂—, —CH₂—CHOH—CH₂—NH—CH₂—CH₂— and —CH₂—CHOH—CH₂—NH—CH₂—CH₂—CH₂—,
more preferably from the group consisting of —CH₂—CHOH—CH₂—, —CH₂—CHOH—CH₂—S—CH₂—CH₂— and —CH₂—CHOH—CH₂—S—CH₂—CH₂—CH₂—,
more preferably wherein f is 1 and wherein R^{m} and Rⁿ are both H, and wherein q, g and e are 0 and wherein L¹ is preferably —CH₂—CHOH—CH₂—S—CH₂—CH₂—, wherein F³ is preferably —C(═O)— and the cytotoxic agent is preferably docetaxel or paclitaxel.

62. The conjugate according to claim 61, having the structure
HAS′(—CH₂—C(═O)-M)ₙ
wherein HAS′ comprises at least one structural unit, preferably 3 to 200 structural units, according to the following formula (Ib) 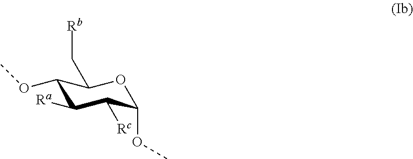
wherein R^{a}, R^{b} and R^{c} are independently of each other selected from the group consisting of —O—HAS″, —[O—CH₂—CH₂]ₛ—OH and —[O—CH₂—CH₂]ₜ—O—CH₂—CHOH—CH₂—S—CH₂—CH₂—S— and wherein at least one of R^{a}, R^{b} and R^{c} is —[O—CH₂—CH₂]ₜ—O—CH₂—CHOH—CH₂—S—CH₂—CH₂—S—, wherein t is in the range of from 0 to 4, and wherein s is in the range of from 0 to 4,
preferably having a structure according to the following formula 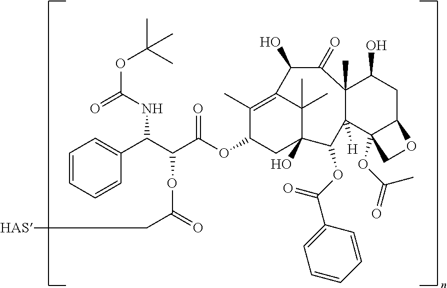
or the following formula

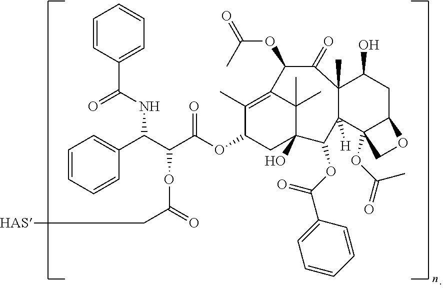

63. The conjugate according to claim 54, wherein q is 1 and F² is succinimide, preferably wherein E is —O— or —S—, more preferably wherein f is 1 and wherein R^{m} and Rⁿ are preferably both H, the conjugate more preferably having the formula
HAS′(-succinimide-[L²]_{g}-S—CH₂—C(═O)-M)ₙ
wherein g is preferably 1 and L² has preferably a structure selected from the group consisting of —CH₂—CH₂—, —CH₂—CH₂—CH₂— and —CH₂—CH₂—CH₂—CH₂—, most preferably wherein the conjugate has the structure
HAS′(-succinimide-CH₂—CH₂—S—CH₂—C(═O)-M)ₙ
wherein the succinimide is linked to the functional group —X— and —X— is —S—.

64. The conjugate according to claim 54, the conjugate having a structure according to the following formula,
HAS′(—[F²]_{q}-[L²]_{g}-[E]ₑ—[CR^{m}Rⁿ]_{f}—F³-M)ₙ
wherein HAS′ comprises at least one structural unit, preferably 3 to 200 structural units, according to the following formula (I) 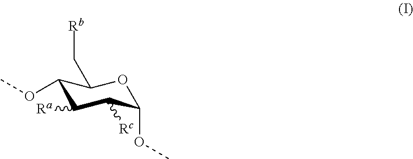
wherein at least one of R^{a}, R^{b} and R^{c} is —[O—CH₂—CH₂]ₜ-[F¹]ₚ-L¹-X— with —X— being —S—,
with p being 1 and
F¹ being selected from the group consisting of —Y⁷—, —Y⁷—C(═Y⁶)—, —C(═Y⁶)—, —Y⁷—C(═Y⁶)—Y⁸—, —C(═Y⁶)—Y⁸—, wherein Y⁷ is selected from the group consisting of —NR^{Y7}—, —O—, —S—, —NH—NH—, —NH—O—, —CH═N—O—, —O—N═CH—, —CH═N—, —N═CH and cyclic imides, such as —succinimide—, Y⁸ is selected from the group consisting of —NR^{Y8}—, —S—, —O—, —NH—NH— and Y⁶ is selected from the group consisting of NR^{Y6}, O and S, wherein R^{Y6} is H or alkyl, preferably H, and wherein R^{Y7} is H or alkyl, preferably H, and wherein R^{Y8} is H or alkyl, preferably H,
preferably with F¹ being —Y⁷—C(═Y⁶)—Y⁸—, more preferably —O—C(═O)—NH—, and wherein L¹ is preferably an alkyl group.

65. The conjugate according to claim 64, having a structure according to the following formula
HAS′(—[F²]_{q}-[L²]_{g}-[E]ₑ—[CR^{m}Rⁿ]_{f}—F³-M)ₙ
wherein f is 1 and wherein R^{m} and Rⁿ are both H, and wherein q, g and e are 0, preferably wherein F³ is —C(═O)— and wherein M is a residue of a cytotoxic agent, and the cytotoxic agent is docetaxel or paclitaxel, more preferably wherein the conjugate has the structure
HAS′(—CH₂—C(═O)-M)ₙ
and wherein HAS′ comprises at least one structural unit, preferably 3 to 200 structural units, according to the following formula (Ib) 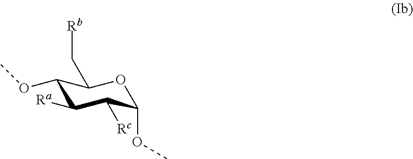
wherein R^{a}, R^{b} and R^{c} are independently of each other selected from the group consisting of —O—HAS″, —[O—CH₂—CH₂]ₛ—OH and —[O—CH₂—CH₂]ₜ—O—C(═O)—NH—CH₂—CH₂—S—, wherein t is in the range of from 0 to 4 and wherein s is in the range of from 0 to 4, and wherein at least one of R^{a}, R^{b} and R^{c} is —[O—CH₂—CH₂]ₜ—O—C(═O)—NH—CH₂—CH₂—S—.

66. The conjugate according to claim 64, wherein q is 1 and F² is succinimide, preferably wherein e is 1 and E is —O— or —S—,
more preferably wherein f is 1 and wherein R^{m} and Rⁿ are preferably both H, the conjugate more preferably having the formula
HAS′(-succinimide-[L²]_{g}-E-CH₂—C(═O)-M)ₙ,
wherein g is preferably 1 and L² has preferably a structure selected from the group consisting of —CH₂—CH₂—, —CH₂—CH₂—CH₂— and —CH₂—CH₂—CH₂—CH₂—.

67. A method for preparing a hydroxyalkyl starch (HAS) conjugate comprising a hydroxyalkyl starch derivative and a cytotoxic agent, said conjugate having a structure according to the following formula
HAS′(-L-M)ₙ
wherein
M is a residue of a cytotoxic agent, said cytotoxic agent comprising a secondary hydroxyl group, and wherein said cytotoxic agent is a taxane,
L is a linking moiety,
HAS′ is a residue of the hydroxyalkyl starch derivative,
and n is greater than or equal to 1, preferably wherein n is in the range of from 3 to 200,
said method comprising
(a) providing a hydroxyalkyl starch derivative having a mean molecular weight MW above the renal threshold, preferably in the range of from 60 to 800 kDa, more preferably of from 80 to 800 kDa, and a molar substitution MS in the range of from 0.6 to 1.5, said hydroxyalkyl starch derivative comprising a functional group Z¹; and providing a cytotoxic agent comprising a secondary hydroxyl group,
(b) coupling the HAS derivative to the cytotoxic agent via an at least bifunctional crosslinking compound L comprising a functional group K¹ and a functional group K², wherein K² is capable of being reacted with Z¹ comprised in the HAS derivative and wherein K¹ is capable of being reacted with the secondary hydroxyl group comprised in the cytotoxic agent,
wherein the cytotoxic agent is preferably reacted with the at least one crosslinking compound L via the functional group K¹ comprised in the crosslinking compound L, wherein said functional group K¹ preferably comprises the structural unit —C(═Y)—, with Y being O, NH or S, more preferably, wherein K¹ is a carboxylic acid group or a reactive carboxy group.

68. The method according to claim 67, wherein the crosslinking compound L has a structure according to the following formula
K²-L′-K¹
wherein K¹ is a functional group comprising the structural unit —C(═Y)— and L′ is a linking moiety,
preferably wherein K² is reacted with the functional group Z¹ comprised in the HAS derivative, wherein Z¹ is selected from the group consisting of aldehyde groups, keto groups, hemiacetal groups, acetal groups, alkynyl groups, azides, carboxy groups, alkenyl groups, thiol reactive groups, —SH, —NH₂, —O—NH₂, —NH—O-alkyl, —(C=G)—NH—NH₂, -G-(C=G)-NH—NH₂, —NH—(C=G)-NH—NH₂, and —SO₂—NH—NH₂ where G is O or S and, if G is present twice, it is independently O or S, more preferably wherein Z¹ is a thiol group (—SH), preferably wherein the cytotoxic agent is reacted via a secondary hydroxyl group with the functional group K¹, thereby forming a functional group —F³—O—, wherein F³ is a —C(═Y)— group, with Y being O, NH or S, in particular with Y being O or S.

69. The method according to claim 67, wherein the at least one crosslinking compound L has a structure according to the following formula
K²-[L²]_{g}-[E]ₑ—[CR^{m}Rⁿ]_{f}—K¹
wherein E is an electron-withdrawing group, preferably selected from the group consisting of —NH—C(═O)—, —C(═O)—NH—, —NH—, —O—, —S—, —SO—, —SO₂— and -succinimide-,
L² is a linking moiety, preferably selected from the group consisting of alkyl, alkylaryl, arylalkyl, aryl, heteroaryl, alkylheteroaryl and heteroarylalkyl,
g is 0 or 1,
e is 0 or 1,
and f is 1, 2 or 3, preferably 1 or 2, most preferably 1,
and wherein R^{m} and Rⁿ are, independently of each other, H or alkyl, more preferably H or methyl, in particular H.

70. The method according to claim 67, wherein the hydroxyalkyl starch derivative provided in step (a) comprises at least one structural unit, preferably 3 to 200 structural units, according to the following formula (I) 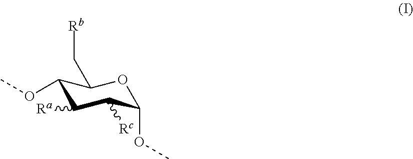
wherein at least one of R^{a}, R^{b} or R^{c} comprises the functional group Z¹,
preferably wherein R^{a}, R^{b} and R^{c} are, independently of each other, selected from the group consisting of —O—HAS″, —[O—(CR^{w}R^{x})—(CR^{y}R^{z})]ₓ—OH, —[O—(CR^{w}R^{x})—(CR^{y}R^{z})]_{y}—Z¹ and —[O—(CR^{w}R^{x})—(CR^{y}R^{z})]_{y}—[F¹]ₚ-L¹-Z¹, and wherein
R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
F¹ is a functional group,
p is 0 or 1,
HAS″ is a remainder of the hydroxyalkyl starch,
and L¹ is a linking moiety,
and wherein step (a) comprises the steps
(a1) providing a hydroxyalkyl starch (HAS) having a mean molecular weight MW above the renal threshold, preferably in the range of from 60 to 800 kDa, more preferably of from 80 to 800 kDa, and a molar substitution MS in the range of from 0.6 to 1.5, comprising the structural unit according to the following formula (II) 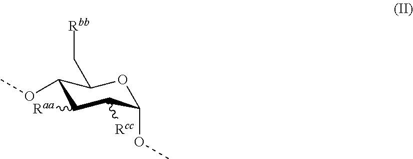
wherein R^{aa}, R^{bb} and R^{cc} are independently of each other selected from the group consisting —O—HAS″ and —[O—(CR^{w}R^{x})—(CR^{y}R^{z})]ₓ—OH,
wherein HAS″ is a remainder of the hydroxyalkyl starch,
R^{w}, R^{x}, R^{y} and R^{z} are independently of each other selected from the group consisting of hydrogen and alkyl,
x is an integer in the range of from 0 to 20, preferably in the range of from 0 to 4,
(a2) introducing at least one functional group Z¹ into the hydroxyalkyl starch by
(i) coupling the hydroxyalkyl starch via at least one hydroxyl group to at least one suitable linker comprising the functional group Z¹ or a precursor of the functional group Z¹, or
(ii) displacing a hydroxyl group present in the hydroxyalkyl starch in a substitution reaction with a precursor of the functional group Z¹ or with a bifunctional linker comprising the functional group Z¹ or a precursor thereof.

71. The method according to claim 70, wherein the HAS derivative formed in step (a2) comprises at least one structural unit, preferably 3 to 200 structural units, according to the following formula (I) 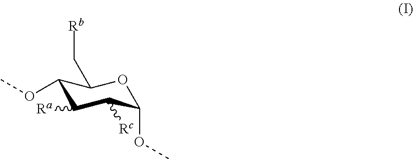
wherein R^{a}, R^{b} and R^{c} are independently of each other selected from the group consisting of —O—HAS″, —[O—CH₂—CH₂]ₛ—OH, —[O—CH₂—CH₂]ₜ—Z¹ and —[O—CH₂—CH₂]ₜ-[F¹]ₚ-L¹-Z¹,
with t being in the range of from 0 to 4,
with s being in the range of from 0 to 4,
p being 0 or 1,
and wherein at least one of R^{a}, R^{b} and R^{c} comprises the functional group Z¹,
and wherein HAS″ is a remainder of HAS, wherein in (a2)-(i) the hydroxyalkyl starch is preferably reacted with a suitable linker comprising the functional group Z¹ or a precursor of the functional group Z¹, and a functional group Z², the linker preferably having the structure Z²-L¹-Z¹ or Z²-L¹-Z¹*-PG, with Z² being a functional group capable of being reacted with the hydroxyalkyl starch or an activated hydroxyalkyl starch, thereby forming a hydroxyalkyl starch derivative comprising at least one structural unit, preferably 3 to 200 structural units, according to the following formula (I) 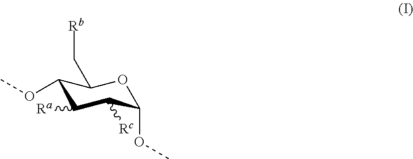
wherein at least one of R^{a}, R^{b} and R^{c} is —[O—CH₂—CH₂]ₜ-[F¹]ₚ-L¹-Z¹*-PG or —[O—CH₂—CH₂]ₜ-[F¹]ₚ-L¹-Z¹ and wherein PG is a suitable protecting group, more preferably Z¹ is —SH, Z¹* is —S—, and the group PG is a thiol protecting group, more preferably a protecting group forming together with Z¹* a thioether (e.g. trityl, benzyl, allyl), a disulfide (e.g. S-sulfonates, S-tert.-butyl, S-(2-aminoethyl)), or a thioester, and wherein in case the linker comprises a protecting group, the method further comprises a deprotection step.

72. The method according to claim 70, wherein step (a2)-(i) comprises
(aa) activating at least one hydroxyl group of the hydroxyalkyl starch with a reactive carbonyl compound having the structure R**—(C═O)—R*, wherein R* and R** may be the same or different, and wherein R* and R** are both leaving groups, wherein upon activation an activated hydroxyalkyl starch derivative comprising at least one structural unit, preferably 3 to 200 structural units, according to the following formula (Ib) 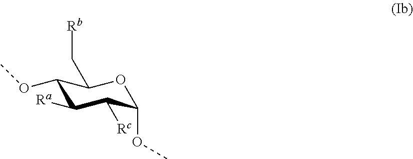
is formed, wherein R^{a}, R^{b} and R^{c} are independently of each other selected from the group consisting of —O—HAS″, —[O—CH₂—CH₂]ₛ—OH and —[O—CH₂—CH₂]ₜ—O—C(═O)—R*, wherein t is in the range of from 0 to 4, and wherein s is in the range of from 0 to 4, and wherein at least one of R^{a}, R^{b} and R^{c} comprises the group
—[O—CH₂—CH₂]ₜ—O—C(═O)—R*, and
(bb) reacting the activated hydroxyalkyl starch derivative according to step (aa) with the at least one suitable linker comprising the functional group Z¹ or a precursor of the functional group Z¹,
preferably wherein the reactive carbonyl compound R**-(C═O)—R* is selected from the group consisting of phosgene, diphosgene, triphosgene, chloroformates and carbonic acid esters, preferably wherein the reactive carbonyl compound is selected from the group consisting of p-nitrophenylchloroformate, pentafluorophenylchloroformate, N,N′-disuccinimidyl carbonate, sulfo-N,N′-disuccinimidyl carbonate, dibenzotriazol-1-yl carbonate and carbonyldiimidazol.

73. The method according to claim 70, wherein step (a2)-(i) comprises
(I) coupling the hydroxyalkyl starch via at least one hydroxyl group comprised in the hydroxyalkyl starch to a first linker comprising a functional group Z², Z² being capable of being reacted with a hydroxyl group of the hydroxyalkyl starch, thereby forming a covalent linkage, the first linker further comprising a functional group W, wherein the functional group W is an epoxide or a group which is transformed in a further step to give an epoxide, preferably wherein W is an alkenyl group and the method further comprises
(II) oxidizing the alkenyl group to give the epoxide, wherein potassium peroxymonosulfate is preferably employed as oxidizing agent, more preferably the method further comprises
(III) reacting the epoxide with a nucleophile comprising the functional group Z¹ or a precursor of the functional group Z¹, wherein the nucleophile is preferably a dithiol or a thiosulfate, thereby forming a hydroxyalkyl starch derivative comprising at least one structural unit, preferably 3 to 200 structural units, according to the following formula (Ib) 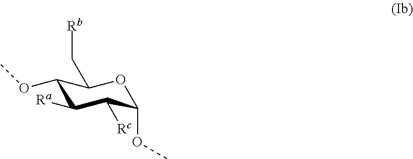
wherein R^{a}, R^{b} and R^{c} are independently of each other selected from the group consisting of —O—HAS″, —[O—CH₂—CH₂]ₛ—OH and —[O—CH₂—CH₂]ₜ-[F¹]ₚ-L¹-Z¹, wherein t is in the range of from 0 to 4, and s is in the range of from 0 to 4, and p is 1, and wherein at least one of R^{a}, R^{b} and R^{c} comprises the group —[O—CH₂—CH₂]ₜ-[F¹]ₚ-L¹-Z¹, and wherein L¹ is a linking moiety and wherein Z¹ is —SH,
preferably wherein the nucleophile is ethanedithiol or sodium thiosulfate.

74. The method according to claim 70, wherein in step (a2)-(ii), prior to the displacement of the hydroxyl group, a group R^{L} is added to at least one hydroxyl group thereby generating a group —O—R^{L}, wherein —O—R^{L} is the leaving group, in particular a —O-Mesyl (—OMs) or O-Tosyl (—OTs) group, preferably wherein Z¹ is a thiol group, and wherein in step (a2)-(ii) the hydroxyl group present in the hydroxyalkyl starch is displaced by a suitable precursor, the method further comprising converting the precursor after the substitution reaction to the functional group Z¹,
wherein the hydroxyalkyl starch derivative obtained according to step (a2)-(ii) preferably comprises at least one structural unit according to the following formula (I) 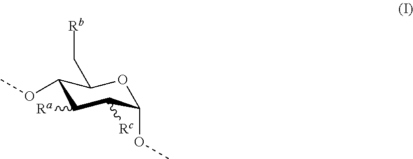
wherein R^{a}, R^{b} and R^{c} are independently of each other selected from the group consisting of —O—HAS″, —[O—CH₂—CH₂]ₛ—OH and —[O—CH₂—CH₂]ₜ—Z¹, wherein t is in the range of from 0 to 4, and s is in the range of from 0 to 4, and wherein at least one of R^{a}, R^{b} and R^{c} is —[O—CH₂—CH₂]ₜ—Z¹, with Z¹ being —SH, and wherein HAS″ is a remainder of HAS,
the method preferably further comprising reacting the hydroxyalkyl starch derivative in step (b) with a crosslinking compound L having a structure according to the formula K²-[L²]_{g}-[E]ₑ—[CR^{m}Rⁿ]_{f}—K¹ with g and e being 0, f is 1, 2 or 3, preferably 1 or 2, most preferably 1, wherein R^{m} and Rⁿ are, independently of each other H or alkyl, preferably H or methyl, in particular H, and wherein K² is a halogen.

75. A hydroxyalkyl starch conjugate obtained or obtainable by a method according to claim 67.

76. A pharmaceutical composition comprising a conjugate according to claim 51.

77. A hydroxyalkyl starch conjugate according to claim 51 for use as medicament, preferably for the treatment of cancer selected from the group consisting of breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.

78. Use of a hydroxyalkyl starch conjugate according to claim 51 for the manufacture of a medicament for the treatment of cancer, wherein the cancer is preferably selected from the group consisting of breast cancer, colorectal cancer, lung cancer, prostate cancer, ovarian cancer, liver cancer, renal cancer, gastric cancer, head and neck cancers, Kaposi's sarcoma and melanoma, in particular for the treatment of prostate cancer.
